SEQUENCE LISTING (32 sequences)

SEQ ID NO: 1 - protein, 226 aa, Hepatitis B virus
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 2 - protein, 149 aa, synthetic construct
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVV

SEQ ID NO: 3 - protein, 24 aa, synthetic construct
APVKQTLNFDLLKLAGDVESNPGP

SEQ ID NO: 4 - DNA, 72 bp, synthetic construct
  source 1..72: mol_type = other DNA
gcccctgtgaagcagaccctgaacttcgacctgctgaagctggccggcgacgtggagagcaatcccggccct

SEQ ID NO: 5 - protein, 435 aa, synthetic construct
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCAPVKQTLNFDLLKLAGDVESNPGPMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 6 - DNA, 1305 bp, synthetic construct
  source 1..1305: mol_type = other DNA
atggacatcgatccctacaaggaatttggcgccaccgtggagctgctgagcttcctgcccagcgacttcttccccagcgtgagggacctcctggacaccgccagcgccctgtacagggaggccctggaatctcccgagcactgcagcccacaccacaccgcactgaggcaggccatcctgtgctggggagagctgatgaccctcgccacctgggtgggcaacaacctggaggaccccgccagcagggacctggtggtgaactacgtcaacaccaacatgggcctgaagatcaggcagctgctgtggttccacatcagctgcctgaccttcggcagggagaccgtgctggagtacctggtgagcttcggcgtgtggatcaggacacctcccgcctacagaccccccaacgcccccatcctgagcaccctgcccgagaccacagtggtgaggaggagggacaggggcaggtcacccaggaggaggactccaagccccaggaggaggaggagccagagccccaggagaaggaggagccagagcagggagagccagtgcgcccctgtgaagcagaccctgaacttcgacctgctgaagctggccggcgacgtggagagcaatcccggccctatggagaacatcaccagcggcttcctgggccccctgctggtgctgcaggcaggcttcttcctgctgaccaggatcctgaccatcccccagagcctggacagctggtggaccagcctgaacttcctcggcgggagccccgtgtgcctgggccagaacagccagtctcccaccagcaatcacagccccaccagctgccccccaatctgtcctggctaccggtggatgtgcctgaggaggttcatcatcttcctgttcatcctgctcctgtgcctgatcttcctgctggtgctgctggactaccagggaatgctgccagtgtgtcccctgatccccggctcaaccaccactaacaccggcccctgcaaaacctgcaccacccccgctcagggcaacagcatgttcccaagctgctgctgcaccaagcccaccgacggcaactgcacctgcattcccatccccagcagctgggccttcgccaagtatctgtgggagtgggccagcgtgaggttcagctggctcagcctgctggtgcccttcgtccagtggtttgtgggcctgagccccaccgtgtggctgagcgccatctggatgatgtggtactggggccccagcctgtactccatcgtgagccccttcatccccctgctgcccattttcttctgcctgtgggtgtacatc

SEQ ID NO: 7 - protein, 229 aa, synthetic construct
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPKPVSKMRMATPLLMQALPMGALPQGPMQNATKYGNMTEDHVMHLLQNADPLKVYPPLKSFPENLRHLKNTMETIDWKVFESWMHHWLLFEMSRHSLEQKPTDAPPKESLELEDPSSGGVTKQDLGPVPM

SEQ ID NO: 8 - DNA, 696 bp, synthetic construct
  source 1..696: mol_type = other DNA
atgcacaggaggaggagcaggagctgcagggaggaccagaagcccgtgatggacgaccagcgcgacctgatcagcaacaacgagcagctgccaatgctgggcaggaggcccggagcacccgaaagcaagtgcagcaggggcgccctgtacaccggcttcagcatcctggtgaccctcctgctggccggccaggccaccaccgcctatttcctgtaccagcagcagggcaggctcgataagctgaccgtgacctcccagaacctgcagctggagaacctgaggatgaagctgcccaagccccccaagcccgtgagcaagatgaggatggccacccccctgctgatgcaggctctgcccatgggggccctgccccagggccccatgcagaacgccaccaaatacggcaacatgaccgaggaccacgtgatgcacctgctgcagaacgccgatcctctgaaggtgtacccacccctgaaaggcagcttccccgagaacctcaggcacctgaagaacaccatggagaccatcgactggaaggtgttcgagagctggatgcaccactggctgctgttcgagatgagccggcacagcctggagcagaagcccaccgacgcccctcccaaggagagcctcgagctcgaggacccaagcagcggcctgggcgtgaccaagcaggacctgggccccgtgcccatg

SEQ ID NO: 9 - protein, 628 aa, synthetic construct
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPKPVSKMRMATPLLMQALPMGALPQGPMQNATKYGNMTEDHVMHLLQNADPLKVYPPLKSFPENLRHLKNTMETIDWKVFESWMHHWLLFEMSRHSLEQKPTDAPPKESLELEDPSSGGVTKQDLGPVPMMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVAPVKQTLNFDLLKLAGDVESNPGPMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 10 - DNA, 1998 bp, synthetic construct
  source 1..1998: mol_type = other DNA
atgcacaggaggaggagcaggagctgcagggaggaccagaagcccgtgatggacgaccagcgcgacctgatcagcaacaacgagcagctgccaatgctgggcaggaggcccggagcacccgaaagcaagtgcagcaggggcgccctgtacaccggcttcagcatcctggtgaccctcctgctggccggccaggccaccaccgcctatttcctgtaccagcagcagggcaggctcgataagctgaccgtgacctcccagaacctgcagctggagaacctgaggatgaagctgcccaagccccccaagcccgtgagcaagatgaggatggccacccccctgctgatgcaggctctgcccatgggggccctgccccagggccccatgcagaacgccaccaaatacggcaacatgaccgaggaccacgtgatgcacctgctgcagaacgccgatcctctgaaggtgtacccacccctgaaaggcagcttccccgagaacctcaggcacctgaagaacaccatggagaccatcgactggaaggtgttcgagagctggatgcaccactggctgctgttcgagatgagccggcacagcctggagcagaagcccaccgacgcccctcccaaggagagcctcgagctcgaggacccaagcagcggcctgggcgtgaccaagcaggacctgggccccgtgcccatggacattgacccctacaaggagttcggcgccaccgtcgaactgctgagcttcctccccagcgacttcttcccctccgtgagggatctgctggacacagctagcgccctgtacagggaggccctggagagccccgagcactgcagcccccaccacacagccctgaggcaggccatcctctgttggggcgagctgatgaccctggccacctgggtgggcaataacctggaggaccccgccagcagggacctggtggtcaactacgtgaacaccaacatgggcctgaagatcaggcagctgctgtggttccacatcagctgcctgacctttggcagggagaccgtcctggagtacctggtgagcttcggcgtgtggatcaggactcccccagcctacaggccccctaacgcccccatcctgtctaccctgcccgagaccaccgtggtgaggaggagggacaggggcagaagccccaggagaaggacccctagccccaggaggaggaggagccagagccccaggaggaggaggagccagagccgggagagccagtgcgcccctgtgaagcagaccctgaacttcgacctgctgaagctggccggcgacgtggagagcaatcccggccctatggaaaacatcaccagcggcttcctgggccccctgctggtgctgcaggccggcttcttcctgctgaccaggatcctgaccattccccagtcactggacagctggtggaccagcctgaacttcctcggcgggagccccgtgtgcctgggccagaatagccagagccccaccagcaaccactctcccacttcctgcccccctatctgccccggctacaggtggatgtgcctgaggaggttcatcatcttcctgttcatcctgctgctgtgcctgatcttcctgctggtgctgctggactaccagggaatgctgcccgtgtgtcccctgatccccggaagcaccaccaccaacaccggcccctgcaagacctgcaccacccccgcccagggcaactctatgttccccagctgctgctgcaccaagcccaccgacggcaactgcacttgcattcccatccccagcagctgggccttcgccaaatatctgtgggagtgggccagcgtgaggtttagctggctgagcctgctggtgcccttcgtgcagtggtttgtgggcctgagccccaccgtgtggctgagcgccatctggatgatgtggtactggggcccctccctgtacagcatcgtgagccccttcatccccctcctgcccatcttcttctgcctgtgggtgtacatc

SEQ ID NO: 11 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 12 - protein, 231 aa, synthetic construct
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVP

SEQ ID NO: 13 - DNA, 693 bp, synthetic construct
  source 1..693: mol_type = other DNA
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtcccc

SEQ ID NO: 14 - DNA, 1998 bp, synthetic construct
  source 1..1998: mol_type = other DNA
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagagtcactggaactggaggacccgtcttctgggctgggtgtgaccaagcaggatctgggcccagtccccatggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgtgcccctgtgaagcagaccctgaacttcgacctgctgaagctggccggcgacgtggagagcaatcccggccctatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccaatacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatt

SEQ ID NO: 15 - protein, 666 aa, synthetic construct
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQCAPVKQTLNFDLLKLAGDVESNPGPMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 16 - DNA, 9999 bp, synthetic construct
  source 1..9999: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtgatgaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 17 - DNA, 2004 bp, synthetic construct
  source 1..2004: mol_type = other DNA
atgcatagaagaaggtccagaagctgcagagaagaccagaaacccgtgatggacgaccagagagacctcatctccaacaacgagcagctgcctatgctgggcagaaggcccggcgctcccgaatccaagtgttccagaggagccctctacaccggcttcagcatcctcgtcacactgctgctcgctggacaagccaccaccgcttactttctgtaccagcagcaaggaagactggataagctgaccgtcacctcccagaatctccaactggagaatctgaggatgaaactccccaaacctcctaagcccgtgtccaagatgaggatggctacacctctgctcatgcaagccctccccatgggagctctgccccaaggacccatgcagaatgccaccaagtacggcaacatgacagaggaccacgtgatgcatctgctccagaacgccgatcctctcaaggtctatccccctctgaagggctccttccccgagaatctgaggcatctcaaaaacaccatggagaccatcgattggaaggtgttcgagagctggatgcatcactggctgctgttcgagatgtctaggcactccctcgagcagaagcccacagatgcccctcccaaggagagcctcgagctggaagatccctccagcggactgggagtcacaaagcaagacctcggccccgtccccatggacatcgatccctacaaagagttcggcgctaccgtggagctgctgtcctttctgccttccgactttttcccctccgtcagagatctgctggacaccgcctccgctctgtatagggaggccctcgagtcccccgagcactgttcccctcatcacacagctctgagacaagccattctgtgctggggcgagctgatgacactggccacatgggtcggcaacaacctcgaagatcccgcctctagggatctggtggtcaactacgtgaacaccaacatgggactgaagattagacagctgctgtggttccacattagctgtctcacctttggcagagaaaccgtgctggagtatctggtgagcttcggagtgtggatcagaaccccccccgcctatagacctcccaatgcccccattctgtccacactgcccgagacaaccgtcgtcagaaggagggacagaggaagatcccctagaaggagaacccccagccctagaagaaggaggtcccagtcccccagaagaaggagaagccagagcagagaatcccagtgcgctcccgtcaaacagaccctcaacttcgatctgctcaagctggccggcgatgtggaatccaaccccggccctatggagaatatcaccagcggctttctcggccctctgctggtcctccaagctggcttctttctgctgacaaggattctgacaatcccccaatctctggacagctggtggacatccctcaactttctgggcggaagccccgtgtgcctcggccaaaactcccagagccccacatccaatcactcccccaccagctgcccccctatttgccccggctacagatggatgtgtctgagaaggttcatcatcttcctcttcattctcctcctctgcctcatctttctgctggtgctcctcgactaccaaggcatgctgcccgtgtgccctctgatccccggcagcaccaccacaaataccggcccttgtaagacatgcaccacacccgcccaaggcaacagcatgtttcctagctgctgctgcacaaagcctacagacggcaactgcacatgcattcctatccccagcagctgggccttcgctaagtatctgtgggaatgggcttccgtgaggttcagctggctctctctgctggtgcccttcgtgcaatggtttgtgggcctcagccctaccgtgtggctgtccgctatttggatgatgtggtattggggaccctctctgtacagcatcgtgtcccccttcatccctctgctgcccatttttttctgtctgtgggtgtatatttgatga

SEQ ID NO: 18 - DNA, 11997 bp, synthetic construct
  source 1..11997: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgcatagaagaaggtccagaagctgcagagaagaccagaaacccgtgatggacgaccagagagacctcatctccaacaacgagcagctgcctatgctgggcagaaggcccggcgctcccgaatccaagtgttccagaggagccctctacaccggcttcagcatcctcgtcacactgctgctcgctggacaagccaccaccgcttactttctgtaccagcagcaaggaagactggataagctgaccgtcacctcccagaatctccaactggagaatctgaggatgaaactccccaaacctcctaagcccgtgtccaagatgaggatggctacacctctgctcatgcaagccctccccatgggagctctgccccaaggacccatgcagaatgccaccaagtacggcaacatgacagaggaccacgtgatgcatctgctccagaacgccgatcctctcaaggtctatccccctctgaagggctccttccccgagaatctgaggcatctcaaaaacaccatggagaccatcgattggaaggtgttcgagagctggatgcatcactggctgctgttcgagatgtctaggcactccctcgagcagaagcccacagatgcccctcccaaggagagcctcgagctggaagatccctccagcggactgggagtcacaaagcaagacctcggccccgtccccatggacatcgatccctacaaagagttcggcgctaccgtggagctgctgtcctttctgccttccgactttttcccctccgtcagagatctgctggacaccgcctccgctctgtatagggaggccctcgagtcccccgagcactgttcccctcatcacacagctctgagacaagccattctgtgctggggcgagctgatgacactggccacatgggtcggcaacaacctcgaagatcccgcctctagggatctggtggtcaactacgtgaacaccaacatgggactgaagattagacagctgctgtggttccacattagctgtctcacctttggcagagaaaccgtgctggagtatctggtgagcttcggagtgtggatcagaaccccccccgcctatagacctcccaatgcccccattctgtccacactgcccgagacaaccgtcgtcagaaggagggacagaggaagatcccctagaaggagaacccccagccctagaagaaggaggtcccagtcccccagaagaaggagaagccagagcagagaatcccagtgcgctcccgtcaaacagaccctcaacttcgatctgctcaagctggccggcgatgtggaatccaaccccggccctatggagaatatcaccagcggctttctcggccctctgctggtcctccaagctggcttctttctgctgacaaggattctgacaatcccccaatctctggacagctggtggacatccctcaactttctgggcggaagccccgtgtgcctcggccaaaactcccagagccccacatccaatcactcccccaccagctgcccccctatttgccccggctacagatggatgtgtctgagaaggttcatcatcttcctcttcattctcctcctctgcctcatctttctgctggtgctcctcgactaccaaggcatgctgcccgtgtgccctctgatccccggcagcaccaccacaaataccggcccttgtaagacatgcaccacacccgcccaaggcaacagcatgtttcctagctgctgctgcacaaagcctacagacggcaactgcacatgcattcctatccccagcagctgggccttcgctaagtatctgtgggaatgggcttccgtgaggttcagctggctctctctgctggtgcccttcgtgcaatggtttgtgggcctcagccctaccgtgtggctgtccgctatttggatgatgtggtattggggaccctctctgtacagcatcgtgtcccccttcatccctctgctgcccatttttttctgtctgtgggtgtatatttgatgaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 19 - DNA, 1311 bp, synthetic construct
  source 1..1311: mol_type = other DNA
atggacatcgatccctacaaagagttcggcgctaccgtggagctgctgtcctttctgccttccgactttttcccctccgtcagagatctgctggacaccgcctccgctctgtatagggaggccctcgagtcccccgagcactgttcccctcatcacacagctctgagacaagccattctgtgctggggcgagctgatgacactggccacatgggtcggcaacaacctcgaagatcccgcctctagggatctggtggtcaactacgtgaacaccaacatgggactgaagattagacagctgctgtggttccacattagctgtctcacctttggcagagaaaccgtgctggagtatctggtgagcttcggagtgtggatcagaaccccccccgcctatagacctcccaatgcccccattctgtccacactgcccgagacaaccgtcgtcagaaggagggacagaggaagatcccctagaaggagaacccccagccctagaagaaggaggtcccagtcccccagaagaaggagaagccagagcagagaatcccagtgcgctcccgtcaaacagaccctcaacttcgatctgctcaagctggccggcgatgtggaatccaaccccggccctatggagaatatcaccagcggctttctcggccctctgctggtcctccaagctggcttctttctgctgacaaggattctgacaatcccccaatctctggacagctggtggacatccctcaactttctgggcggaagccccgtgtgcctcggccaaaactcccagagccccacatccaatcactcccccaccagctgcccccctatttgccccggctacagatggatgtgtctgagaaggttcatcatcttcctcttcattctcctcctctgcctcatctttctgctggtgctcctcgactaccaaggcatgctgcccgtgtgccctctgatccccggcagcaccaccacaaataccggcccttgtaagacatgcaccacacccgcccaaggcaacagcatgtttcctagctgctgctgcacaaagcctacagacggcaactgcacatgcattcctatccccagcagctgggccttcgctaagtatctgtgggaatgggcttccgtgaggttcagctggctctctctgctggtgcccttcgtgcaatggtttgtgggcctcagccctaccgtgtggctgtccgctatttggatgatgtggtattggggaccctctctgtacagcatcgtgtcccccttcatccctctgctgcccatttttttctgtctgtgggtgtatatttgatga

SEQ ID NO: 20 - DNA, 11304 bp, synthetic construct
  source 1..11304: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggacatcgatccctacaaagagttcggcgctaccgtggagctgctgtcctttctgccttccgactttttcccctccgtcagagatctgctggacaccgcctccgctctgtatagggaggccctcgagtcccccgagcactgttcccctcatcacacagctctgagacaagccattctgtgctggggcgagctgatgacactggccacatgggtcggcaacaacctcgaagatcccgcctctagggatctggtggtcaactacgtgaacaccaacatgggactgaagattagacagctgctgtggttccacattagctgtctcacctttggcagagaaaccgtgctggagtatctggtgagcttcggagtgtggatcagaaccccccccgcctatagacctcccaatgcccccattctgtccacactgcccgagacaaccgtcgtcagaaggagggacagaggaagatcccctagaaggagaacccccagccctagaagaaggaggtcccagtcccccagaagaaggagaagccagagcagagaatcccagtgcgctcccgtcaaacagaccctcaacttcgatctgctcaagctggccggcgatgtggaatccaaccccggccctatggagaatatcaccagcggctttctcggccctctgctggtcctccaagctggcttctttctgctgacaaggattctgacaatcccccaatctctggacagctggtggacatccctcaactttctgggcggaagccccgtgtgcctcggccaaaactcccagagccccacatccaatcactcccccaccagctgcccccctatttgccccggctacagatggatgtgtctgagaaggttcatcatcttcctcttcattctcctcctctgcctcatctttctgctggtgctcctcgactaccaaggcatgctgcccgtgtgccctctgatccccggcagcaccaccacaaataccggcccttgtaagacatgcaccacacccgcccaaggcaacagcatgtttcctagctgctgctgcacaaagcctacagacggcaactgcacatgcattcctatccccagcagctgggccttcgctaagtatctgtgggaatgggcttccgtgaggttcagctggctctctctgctggtgcccttcgtgcaatggtttgtgggcctcagccctaccgtgtggctgtccgctatttggatgatgtggtattggggaccctctctgtacagcatcgtgtcccccttcatccctctgctgcccatttttttctgtctgtgggtgtatatttgatgaggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 21 - protein, 416 aa, synthetic construct
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 22 - DNA, 1248 bp, synthetic construct
  source 1..1248: mol_type = other DNA
atgcacagaagaagaagcagatcatgcagggaggatcagaaaccagtgatggacgaccagagggacctgatcagcaacaatgagcaactgccaatgctgggccgaagacccggcgcccccgagtcaaagtgcagccgaggcgccttatacaccggcttcagcatcctggttacgctgctcctggcaggccaggccaccaccgcatatttcctgtaccaacagcaaggcagactggacaagctaaccgtgacctcacagaacctacagctggaaaatctgaggatgaagctcccaaagccccctaagcccgtgagcaagatgagaatggccacacccctgctgatgcaggccctccccatgggagccctgccacagggacccatgcagaatgccaccaagtacggcaacatgaccgaggaccacgtgatgcacctgctgcagaacgccgaccccctgaaggtgtacccccccctgaagggcagcttccccgagaacctgagacacctgaagaacaccatggagaccatcgactggaaggtgttcgagagctggatgcaccactggctgctgttcgagatgagcagacacagcctggagcagaagcccaccgacgccccccccaaggagtccctggagctggaggaccccagcagcggcctgggcgtgaccaagcaggacctgggccccgtgcccatggacatcgacccctacaaggagttcggcgccacagtggagctgctgagcttcctgcccagcgacttcttccccagcgtgagagacctgctggacaccgcctccgccctgtacagagaggccctggagagccccgagcactgcagcccccaccacaccgccctgagacaggccatcctgtgctggggggagctgatgaccctggccacctgggtgggcaacaacctggaggaccccgccagcagagacctggtggtgaactacgtgaacaccaacatgggcctgaagatcaggcagctgctgtggttccacatcagctgcttgaccttcggcagagagaccgtgctggagtacctggtgagcttcggcgtgtggatccgcacccctcccgcatacagaccccccaacgcccccatcctgagcaccctgcccgagaccaccgtggtgagacgcagagacagaggccggagcccccgcagaagaacccccagccccaggagaagacggagccagagccccagaagacggcgcagccagagcagagagagccagtgc

SEQ ID NO: 23 - DNA, 3688 bp, synthetic construct
  source 1..3688: mol_type = other DNA
acgcgtgcctaaattaatacgactcactataaggagaagctgtctatcgggctccagcggtcatgcacagaagaagaagcagatcatgcagggaggatcagaaaccagtgatggacgaccagagggacctgatcagcaacaatgagcaactgccaatgctgggccgaagacccggcgcccccgagtcaaagtgcagccgaggcgccttatacaccggcttcagcatcctggttacgctgctcctggcaggccaggccaccaccgcatatttcctgtaccaacagcaaggcagactggacaagctaaccgtgacctcacagaacctacagctggaaaatctgaggatgaagctcccaaagccccctaagcccgtgagcaagatgagaatggccacacccctgctgatgcaggccctccccatgggagccctgccacagggacccatgcagaatgccaccaagtacggcaacatgaccgaggaccacgtgatgcacctgctgcagaacgccgaccccctgaaggtgtacccccccctgaagggcagcttccccgagaacctgagacacctgaagaacaccatggagaccatcgactggaaggtgttcgagagctggatgcaccactggctgctgttcgagatgagcagacacagcctggagcagaagcccaccgacgccccccccaaggagtccctggagctggaggaccccagcagcggcctgggcgtgaccaagcaggacctgggccccgtgcccatggacatcgacccctacaaggagttcggcgccacagtggagctgctgagcttcctgcccagcgacttcttccccagcgtgagagacctgctggacaccgcctccgccctgtacagagaggccctggagagccccgagcactgcagcccccaccacaccgccctgagacaggccatcctgtgctggggggagctgatgaccctggccacctgggtgggcaacaacctggaggaccccgccagcagagacctggtggtgaactacgtgaacaccaacatgggcctgaagatcaggcagctgctgtggttccacatcagctgcttgaccttcggcagagagaccgtgctggagtacctggtgagcttcggcgtgtggatccgcacccctcccgcatacagaccccccaacgcccccatcctgagcaccctgcccgagaccaccgtggtgagacgcagagacagaggccggagcccccgcagaagaacccccagccccaggagaagacggagccagagccccagaagacggcgcagccagagcagagagagccagtgctgataagccgccgctccagctttgcacgtttcgatcccaaaggccctttttagggccgaccattcattgcaaaaaaaaaaaaaaaaaaaaaaaaaaaaaagcatatgactaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcac

SEQ ID NO: 24 - DNA, 678 bp, synthetic construct
  source 1..678: mol_type = other DNA
atggagaatatcaccagcggcttcctcggccccctcttagtgctgcaggccggcttcttcctcctgacacggatcctgaccatcccgcagtccctggactcatggtggacctccctgaacttcctgggcggctcccccgtgtgcctgggccagaactcccagagccccaccagcaaccactcccccaccagctgcccccccatctgccccggctacaggtggatgtgcctgcggcggttcatcatcttcctgttcatcctgctgctgtgcctgatcttcctgctggtgctgctggactaccagggcatgctgcccgtgtgccccctgatccccggctccaccaccaccaacaccggcccctgcaagacctgcaccacccccgcccaggggaacagcatgttcccctcctgctgctgcaccaagcccaccgacggcaactgcacctgcatccccatcccctccagctgggccttcgccaagtacctgtgggagtgggcctccgtgcggttcagctggctgagcctgctggtgcccttcgtgcagtggttcgtgggcctgtcccccaccgtgtggctgtccgccatctggatgatgtggtactggggccccagcctgtacagcatcgtgagccccttcatccccctgctgcccatcttcttctgcctgtgggtgtacatc

SEQ ID NO: 25 - DNA, 3118 bp, synthetic construct
  source 1..3118: mol_type = other DNA
acgcgtgcctaaattaatacgactcactataaggagaagctgtctatcgggctccagcggtcatggagaatatcaccagcggcttcctcggccccctcttagtgctgcaggccggcttcttcctcctgacacggatcctgaccatcccgcagtccctggactcatggtggacctccctgaacttcctgggcggctcccccgtgtgcctgggccagaactcccagagccccaccagcaaccactcccccaccagctgcccccccatctgccccggctacaggtggatgtgcctgcggcggttcatcatcttcctgttcatcctgctgctgtgcctgatcttcctgctggtgctgctggactaccagggcatgctgcccgtgtgccccctgatccccggctccaccaccaccaacaccggcccctgcaagacctgcaccacccccgcccaggggaacagcatgttcccctcctgctgctgcaccaagcccaccgacggcaactgcacctgcatccccatcccctccagctgggccttcgccaagtacctgtgggagtgggcctccgtgcggttcagctggctgagcctgctggtgcccttcgtgcagtggttcgtgggcctgtcccccaccgtgtggctgtccgccatctggatgatgtggtactggggccccagcctgtacagcatcgtgagccccttcatccccctgctgcccatcttcttctgcctgtgggtgtacatctgataagccgccgctccagctttgcacgtttcgatcccaaaggccctttttagggccgaccattcattgcaaaaaaaaaaaaaaaaaaaaaaaaaaaaaagcatatgactaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcac

SEQ ID NO: 26 - protein, 457 aa, synthetic construct
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 27 - DNA, 1371 bp, synthetic construct
  source 1..1371: mol_type = other DNA
atgcacagaagacgctccaggagctgccgagaggatcagaaacctgtgatggacgaccagcgcgacctgatcagcaacaatgagcagttgcccatgctgggacgccgccccggcgcaccggagagcaagtgctcccgcggcgccttatacaccggattcagcatcctcgttaccctgttactggccgggcaggccaccacggcctacttcctctaccaacaacagggcaggttagacaagctgaccgtgacctcacagaatctccagttagagaacctgagaatgaagctgccaaagccacctaagcctgtgagcaagatgcggatggccacccccctgctgatgcaggccctgcctatgggcgccctgccacagggccccatgcagaatgccaccaagtacggcaatatgaccgaggaccacgtgatgcacctgctgcagaatgccgaccctctgaaggtgtaccctcctctgaagggctccttcccagagaatctgcggcacctgaagaacaccatggagaccatcgactggaaggtgttcgagagctggatgcaccactggctgctgttcgagatgtccaggcactctctggagcagaagcccaccgacgcccctccaaaggagagcctggagctggaggaccccagcagcggcctgggcgtgaccaagcaggacctgggcccagtgccaatggagaacatcaccagcggcttcctgggccccctgctggtgctgcaggccggcttcttcctgctgaccaggatcctgaccatccctcagagcctggacagctggtggaccagcctgaatttcctgggcggcagcccagtgtgcctgggccagaacagccagtccccaaccagcaaccacagccctaccagctgccctcccatctgcccaggctacaggtggatgtgcctgaggcggttcatcatcttcctgttcatcctgctgctgtgcctgatcttcctgctggtgctgctggactaccagggcatgctgccagtgtgccccctgatccccggcagcaccaccaccaacaccggcccctgcaagacctgcaccacccccgcccagggcaacagcatgttcccctcgtgctgctgcaccaagcccaccgacggcaattgcacctgcatccccatccccagcagctgggccttcgccaagtacctgtgggagtgggccagcgtgcggttcagctggctcagcctgctggtgcccttcgtgcagtggttcgtgggcctgtcccctaccgtgtggctgtccgcaatctggatgatgtggtactggggcccatccctctacagcatcgtgagcccattcatcccactgctgcctatcttcttctgcttgtgggtgtacatc

SEQ ID NO: 28 - DNA, 3811 bp, synthetic construct
  source 1..3811: mol_type = other DNA
acgcgtgcctaaattaatacgactcactataaggagaagctgtctatcgggctccagcggtcatgcacagaagacgctccaggagctgccgagaggatcagaaacctgtgatggacgaccagcgcgacctgatcagcaacaatgagcagttgcccatgctgggacgccgccccggcgcaccggagagcaagtgctcccgcggcgccttatacaccggattcagcatcctcgttaccctgttactggccgggcaggccaccacggcctacttcctctaccaacaacagggcaggttagacaagctgaccgtgacctcacagaatctccagttagagaacctgagaatgaagctgccaaagccacctaagcctgtgagcaagatgcggatggccacccccctgctgatgcaggccctgcctatgggcgccctgccacagggccccatgcagaatgccaccaagtacggcaatatgaccgaggaccacgtgatgcacctgctgcagaatgccgaccctctgaaggtgtaccctcctctgaagggctccttcccagagaatctgcggcacctgaagaacaccatggagaccatcgactggaaggtgttcgagagctggatgcaccactggctgctgttcgagatgtccaggcactctctggagcagaagcccaccgacgcccctccaaaggagagcctggagctggaggaccccagcagcggcctgggcgtgaccaagcaggacctgggcccagtgccaatggagaacatcaccagcggcttcctgggccccctgctggtgctgcaggccggcttcttcctgctgaccaggatcctgaccatccctcagagcctggacagctggtggaccagcctgaatttcctgggcggcagcccagtgtgcctgggccagaacagccagtccccaaccagcaaccacagccctaccagctgccctcccatctgcccaggctacaggtggatgtgcctgaggcggttcatcatcttcctgttcatcctgctgctgtgcctgatcttcctgctggtgctgctggactaccagggcatgctgccagtgtgccccctgatccccggcagcaccaccaccaacaccggcccctgcaagacctgcaccacccccgcccagggcaacagcatgttcccctcgtgctgctgcaccaagcccaccgacggcaattgcacctgcatccccatccccagcagctgggccttcgccaagtacctgtgggagtgggccagcgtgcggttcagctggctcagcctgctggtgcccttcgtgcagtggttcgtgggcctgtcccctaccgtgtggctgtccgcaatctggatgatgtggtactggggcccatccctctacagcatcgtgagcccattcatcccactgctgcctatcttcttctgcttgtgggtgtacatctgataagccgccgctccagctttgcacgtttcgatcccaaaggccctttttagggccgaccattcattgcaaaaaaaaaaaaaaaaaaaaaaaaaaaaaagcatatgactaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcac

SEQ ID NO: 29 - DNA, 750 bp, synthetic construct
  source 1..750: mol_type = other DNA
tgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgacc

SEQ ID NO: 30 - DNA, 1248 bp, synthetic construct; identical to SEQ ID NO: 22
  source 1..1248: mol_type = other DNA

SEQ ID NO: 31 - DNA, 678 bp, synthetic construct; identical to SEQ ID NO: 24
  source 1..678: mol_type = other DNA

SEQ ID NO: 32 - DNA, 1371 bp, synthetic construct; identical to SEQ ID NO: 27
  source 1..1371: mol_type = other DNA

(19) United States
(12) Patent Application Publication
(10) Pub. No.: US 2026/0207736 A1
(43) Pub. Date: Jul. 23, 2026

(54) Hepatitis B Compositions

(71) Applicant: GlaxoSmithKline Biologicals SA, Rixensart (BE)

(72) Inventors: Babak BAYAT, Rixensart (BE); Kambiz MOUSAVI, Rockville, MD (US); Ventzislav VASSILEV, Rixensart (BE)

(21) Appl. No.: 19/139,098

(22) PCT Filed: Dec. 18, 2023
(86) PCT No.: PCT/EP2023/086486

§ 371 Date: Jun. 13, 2025

Related U.S. Application Data
(60) Provisional application No. 63/433,643, filed on Dec. 19, 2022.

(51) Int. Cl. A61K 39/29; A61K 39/00; A61P 37/04

(52) U.S. Cl. CPC A61K 39/292; A61P 37/04; A61K 2039/53; A61K 2039/545; A61K 2039/55555; A61K 2039/55572; A61K 2039/55577

(57) ABSTRACT

A composition for treating chronic hepatitis B infection comprising mRNA encoding a hepatitis B virus antigen, wherein the mRNA is encapsulated in a lipid nanoparticle (LNP).

### TECHNICAL FIELD

The present invention relates to compositions for use in the treatment of chronic hepatitis B, wherein the compositions comprise mRNA encoding one or more hepatitis B antigens, and to related aspects.

### BACKGROUND

Hepatitis B virus (HBV) infection is a major public health problem. Globally, the WHO estimates that 296 million people were living with chronic hepatitis B infection in 2019, with 1.5 million new infections each year (WHO, 2021). The clinical course and outcome of HBV infection is largely driven by the age at which the infection is acquired, and a complex interaction between the virus and an individual's immune response. Thus, exposure to HBV may lead to acute hepatitis that resolves spontaneously, or it may progress to various forms of chronic infection, including the inactive hepatitis B surface antigen (HBsAg) carrier state, chronic hepatitis, cirrhosis and hepatocellular carcinoma (HCC). It is thought that between 15-40% of persons with chronic infection (defined as serum HBsAg being detected for more than 6 months) will develop liver sequelae, of which liver cirrhosis (LC), hepatic decompensation and hepatocellular carcinoma (HCC) are the major complications.

Although implementation of universal prophylactic hepatitis B immunization in infants has been highly effective in reducing the incidence and prevalence of hepatitis B in many endemic countries, it has not yet led to a strong decrease in the prevalence of chronic hepatitis B (CHB) in adolescents and adults, and it is not expected to impact on HBV-related deaths until several decades after introduction. In 2019, the World Health Organization estimated that there were 820,000 deaths from HBV-related causes, mostly from cirrhosis and hepatocellular carcinoma (primary liver cancer) (WHO, 2021).

Clinical management of chronic hepatitis B aims to improve survival and quality of life by preventing disease progression, and consequently HCC development. The current treatment strategy is mainly based on the long-term suppression of HBV DNA replication to achieve the stabilisation of HBV-induced liver disease and to prevent progression. Serum HBV DNA level is a cornerstone endpoint of all current treatment modalities. Although achieving loss of (detectable) hepatitis B e-antigen (HBeAg) is a valuable biomarker, HBsAg loss, with or without anti-HBs seroconversion, is generally considered an optimal endpoint representing “functional cure”, as it indicates profound suppression of HBV replication and viral protein expression (Revill, 2019; Block, 2017; Cornberg, 2017). Currently, there are two main treatment options for CHB patients: treatment with pegylated interferon alpha (PeglFNa), or with nucleo(s)tide analogues (NA) (EASL, 2017). PegIFNa aiming at induction of a long-term immune control with a finite duration treatment may achieve sustained off-treatment control, but durable virological response and hepatitis B surface antigen (HBsAg) loss is limited to a small proportion of patients. In addition, owing to its poor tolerability and long-term safety concerns, a significant number of patients are ineligible for this type of treatment.

NAs act by suppressing DNA replication through inhibition of HBV polymerase reverse transcriptase activity. The NAs approved in Europe for HBV treatment include entecavir (ETV), tenofovir disoproxil fumarate (TDF) and tenofovir alafenamide (TAF) that are associated with high barrier against HBV resistance as well as lamivudine (LAM), adefovir dipivoxil (ADV) and telbivudine (TBV) that are associated with low barrier to HBV resistance. The main advantage of treatment with a potent NA with high barrier to resistance is its predictable high long-term antiviral efficacy leading to HBV DNA suppression in the vast majority of compliant patients as well as its favourable safety profile. The disadvantage of NA treatment is that it is a long-term therapeutic regimen, because a NA does not usually achieve HBV eradication and NA discontinuation may lead to HBV relapse.

Because of a low rate of HBsAg seroclearance and a high risk of off-NA viral relapse, most patients are maintained under long-term or even indefinite NA therapy, which could be associated with reduction in patient compliance to therapy, increase in financial costs and increased risk for drug toxicity and drug resistance mutations upon long-term exposure. A new strategy is therefore necessary to achieve a “functional cure” with a finite regimen.

Messenger RNA (mRNA) is a single-stranded RNA molecule that corresponds to the genetic sequence of a gene and is read by ribosomes in the process of producing a protein. mRNA based vaccines provide an alternative vaccination approach to traditional strategies involving live attenuated/inactivated pathogens or subunit vaccines (Zhang, 2019). mRNA vaccines may utilise non-replicating mRNA (mRNA) or self-replicating RNA (also referred to as self-amplifying mRNA or SAM). Non-replicating mRNA-based vaccines typically encode an antigen of interest and contain 5′ and 3′ untranslated regions (UTRs), a 5′ cap and a poly(A) tail, whereas self-amplifying RNAs also encode viral replication machinery that enables intracellular RNA amplification (Pardi, 2018).

The present invention aims to help address the need for an HBV treatment which can clear HBsAg in order to allow patients to safely discontinue NA therapy without viral or clinical relapse.

### SUMMARY OF THE DISCLOSURE

The present disclosure provides a composition for treating chronic hepatitis B infection comprising a mRNA encoding at least a hepatitis B virus core antigen (HBc), wherein the mRNA is encapsulated in a lipid nanoparticle (LNP). In embodiments, the HBc is fused to human invariant chain (hli).

In an aspect, the present disclosure provides composition for treating chronic hepatitis B infection comprising a mRNA encoding at least one hepatitis B surface antigen (HBsAg), wherein the mRNA is encapsulated in a lipid nanoparticle (LNP). In embodiments, the HBsAg is a hepatitis B small surface protein (HBs). In embodiments, the HBsAg is fused to human invariant chain (hli).

In a further aspect, the mRNA is non-replicating. In other aspect, the mRNA is self-replicating mRNA (SAM).

In other aspects, the composition comprising a mRNA is administered sequentially or concomitantly with one or more recombinant hepatitis B polypeptide(s). In an embodiment, the recombinant hepatitis B polypeptides include a recombinant hepatitis core protein (HBc), a recombinant hepatitis B small surface protein (HBs). In further embodiments, the one or more recombinant hepatitis B polypeptide(s) is administered with an adjuvant. The adjuvant can be AS01.

Also described herein is a method of treating chronic hepatitis B infection. This method may comprise a prime-boost regimen. The mRNA encoding a hepatitis B virus antigen may be administered as a priming dose, and one or more recombinant hepatitis B polypeptide(s) may be administered as a booster dose. In an embodiment, the one or more recombinant hepatitis B polypeptide(s) may be administered with an adjuvant as a booster dose. The adjuvant may be AS01.

Also described herein is a method of treating chronic hepatitis B infection in a human comprising the steps of: (a) administering to the human an adenoviral vector comprising a polynucleotide encoding a hepatitis B polypeptide; (b) administering to the human a mRNA encoding a hepatitis B virus antigen; and (c) administering to the human at least one recombinant hepatitis B polypeptide(s). Such a method may be a heterologous prime-boost regimen, comprising (a) administering the adenoviral vector as a priming dose; (b) administering the mRNA as a booster dose; and (c) administering the at least one recombinant hepatitis B polypetide(s) as one or more boosting doses. In one embodiment, the adenoviral vector is replication-defective chimpanzee adenoviral (ChAd) vector.

The present invention also provides a composition comprising an mRNA administered sequentially or concomitantly with one or more polypeptide(s). In further embodiments, the one or more polypeptide(s) are administered with an adjuvant. The adjuvant can be AS01.

In one aspect, there is a method comprising administering to a human a mRNA in combination with at least one polypeptide. The components (i.e. mRNA and polypeptide) may be administered sequentially in a heterologous prime-boost regimen. If a heterologous prime-boost regimen is used, the mRNA may be administered as a priming dose, and the at least one polypeptide is administered as a booster dose. In another aspect, the at least one polypeptide is administered as a priming dose, and the mRNA is administered as a booster dose. The at least one polypeptide can be administered with or without adjuvant. In a particular embodiment, the polypeptide is administered with an adjuvant. In one embodiment, the mRNA is administered sequentially with the adjuvanted polypeptide. In another embodiment, the mRNA is administered concomitantly (for example, at the same time in different locations) with the adjuvanted polypeptide. The adjuvant is preferably AS01.

### BRIEF DESCRIPTION OF THE SEQUENCES

SEQ ID NO:1: Amino acid sequence of HBs
SEQ ID NO:2: Amino acid sequence of HBc truncate
SEQ ID NO:3: Amino acid sequence of spacer incorporating 2A cleavage region of foot and mouth virus
SEQ ID NO:4: Nucleotide sequence encoding spacer incorporating 2A cleavage region of foot and mouth virus
SEQ ID NO:5: Amino acid sequence of HBc-2A-HBs
SEQ ID NO:6: Nucleotide sequence encoding HBc-2A-HBs
SEQ ID NO:7: Amino acid sequence of hli
SEQ ID NO:8: Nucleotide sequence encoding hli
SEQ ID NO:9: Amino acid sequence of hli-HBc-2A-HBs
SEQ ID NO:10: Nucleotide sequence encoding hli-HBc-2A-HBs
SEQ ID NO:11: Amino acid sequence of HBc
SEQ ID NO:12: Amino acid sequence of hli alternate variant
SEQ ID NO:13: Nucleotide sequence encoding hli alternate variant
SEQ ID NO:14: Alternative nucleic acid sequence of hli-HBc-2A-HBs
SEQ ID NO:15: Alternative amino acid sequence of hli-HBc-2A-HBs
SEQ ID NO:16: Nucleic acid sequence of an empty SAM vector
SEQ ID NO: 17: Human codon optimized (Genewiz) nucleic acid sequence encoding the hli_HBc_2A_HBs SAM transgene
SEQ ID NO: 18: hli_HBc_2A_HBs SAM plasmid sequence
SEQ ID NO: 19: Human codon optimized (Genewiz) nucleic acid sequence encoding the HBc_2A_HBs SAM transgene
SEQ ID NO: 20: HBc_2A_HBs in SAM plasmid sequence
SEQ ID NO: 21: Amino acid sequence of hli-HBc
SEQ ID NO: 22: Nucleotide sequence encoding hli-HBc
SEQ ID NO: 23: hli-HBc mRNA plasmid sequence (UTR4)
SEQ ID NO: 24: Nucleotide sequence encoding HBs
SEQ ID NO: 25: HBs mRNA plasmid sequence (UTR4)
SEQ ID NO: 26: Amino acid sequence of hli-HBs
SEQ ID NO: 27: Nucleotide sequence encoding hli-HBs
SEQ ID NO: 28: hli-HBs mRNA plasmid sequence (UTR4)
SEQ ID NO: 29: IRES nucleotide sequence
SEQ ID NO: 30: Human codon optimized (CodeRNA2) nucleic acid sequence encoding the hli_HBc mRNA transgene
SEQ ID NO: 31: Human codon optimized (CodeRNA2) nucleic acid sequence encoding the HBs mRNA transgene
SEQ ID NO: 32: Human codon optimized (CodeRNA2) nucleic acid sequence encoding the hli_HBs mRNA transgene

### DESCRIPTION OF THE FIGURES

FIG. 1A: Shows the HBV core antigen (HBc) specific CD4+ T-cell response in the spleen after priming with ChAd155-hli-HBV and boosting with SAM-HBV (±hli). At 12/13 days post second immunization (12/13dpll), spleens were collected to evaluate HB core (HBc)-specific CD4+ T cells by intracellular cell staining. Each dot represents individual animals and the Geometric Mean (GM) represented by bars.

FIG. 1B: Shows the HBV surface antigen (HBs) specific CD4+ T-cell response in the spleen after priming with ChAd155-hli-HBV and boosting with SAM-HBV (±hli). At 12/13 days post second immunization (12/13dpll), spleens were collected to evaluate HB surface (HEs)-specific CD4+ T cells by intracellular cell staining. Each dot represents individual animals and the Geometric Mean (GM) represented by bars.

FIG. 2A: Shows the HBV core antigen (HBc) specific CD8+ T-cell response in the spleen after priming with ChAd155-hli-HBV and boosting with SAM-HBV (±hli). At 12/13 days post second immunization (12/13dpll), spleens were collected to evaluate HB core (HEc)-specific CD8+ T cells by intracellular cell staining. Each dot represents individual animals and the Geometric Mean (GM) represented by bars.

FIG. 2E: Shows the HBV surface antigen (HBs) specific CD8+ T-cell response in the spleen after priming with ChAd155-hli-HBV and boosting with SAM-HBV (±hli). At 12/13 days post second immunization (12/13dpll), spleens were collected to evaluate HB surface (HEs)-specific CD8+ T cells by intracellular cell staining. Each dot represents individual animals and the Geometric Mean (GM) represented by bars.

FIG. 3A: Shows the HBV core antigen (HBc) specific antibody response after priming with ChAd155-hli-HBV and boosting with SAM-HBV (±hli). At 12/13 days post second immunization, serum sample were collected to evaluate the anti-HBc IgG antibody titers by ELISA. For each group, each dot represents anti-HBc IgG antibody titer of individual animals and the Geometric Mean (GM) with 95% confidence intervals (CI) represented by columns.

FIG. 3E: Shows the HBV surface antigen (HBs) specific antibody response after priming with ChAd155-hli-HBV and boosting with SAM-HBV (±hli). At 12/13 days post second immunization, serum sample were collected to evaluate the anti-HBs IgG antibody titers by ELISA. For each group, each dot represents anti-HBs IgG antibody titer of individual animals and the Geometric Mean (GM) with 95% confidence intervals (CI) represented by columns.

FIG. 4A: Shows the HBV core antigen (HBc) specific CD8+ T-cell response in the spleen at 14dpll (i.e. 14 days post second dose) for all groups detailed in Table 2 (where C=ChAd155-hli-HBV, M=MVA-HBV, S=SAM-hli-HBV, P=HBc-HBs/AS01). At 14 days post second immunization (14dpll), spleens were collected to evaluate HB core (HEc)-specific CD8+ T cells by intracellular cell staining. Each dot represents individual animals and the Geometric Mean (GM) represented by columns.

FIG. 4E: Shows the HBV core antigen (HBc) specific CD8+ T-cell response in the spleen at 22dplV (i.e. 22 days post fourth dose) for all groups detailed in Table 2 (where C=ChAd155-hli-HBV, M=MVA-HBV, S=SAM-hli-HBV, P=HBc-HBs/AS01). At 22 days post fourth immunization (22dplV), spleens were collected to evaluate HB core (HEc)-specific CD8+ T cells by intracellular cell staining. Each dot represents individual animals and the Geometric Mean (GM) represented by columns.

FIG. 5A: Shows the HBV surface antigen (HBs) specific CD8+ T-cell response in the spleen at 14dpll (i.e. 14 days post second dose) for all groups detailed in Table 2 (where C=ChAd155-hli-HBV, M=MVA-HBV, S=SAM-hli-HBV, P=HBc-HBs/AS01). At 14 days post second immunization (14dplV), spleens were collected to evaluate HB surface (HEs)-specific CD8+ T cells by intracellular cell staining. Each dot represents individual animals and the Geometric Mean (GM) represented by columns.

FIG. 5E: Shows the HBV surface antigen (HBs) specific CD8+ T-cell response in the spleen at 22dplV (i.e. 22 days after fourth dose) for all groups detailed in Table 2 (where C=ChAd155-hli-HBV, M=MVA-HBV, S=SAM-hli-HBV, P=HBc-HBs/AS01). At 22 days post fourth immunization (22dplV), spleens were collected to evaluate HB surface (HEs)-specific CD8+ T cells by intracellular cell staining. Each dot represents individual animals and the Geometric Mean (GM) represented by columns.

FIG. 6A: Shows the HBV core antigen (HBc) specific CD4+ T-cell response in the spleen at 14dpll (i.e. 14 days post second dose) for all groups detailed in Table 2 (where C=ChAd155-hli-HBV, M=MVA-HBV, S=SAM-hli-HBV, P=HBc-HBs/AS01). At 14 days post second immunization (14dpll), spleens were collected to evaluate HB core (HEc)-specific CD4+ T cells by intracellular cell staining. Each dot represents individual animals and the Geometric Mean (GM) represented by columns.

FIG. 6E: Shows the HBV core antigen (HBc) specific CD4+ T-cell response in the spleen at 22dplV (i.e. 22 days after fourth dose) for all groups detailed in Table 2 (where C=ChAd155-hli-HBV, M=MVA-HBV, S=SAM-hli-HBV, P=HBc-HBs/AS01). At 22 days post fourth immunization (22dplV), spleens were collected to evaluate HB core (HEBc)-specific CD4+ T cells by intracellular cell staining. Each dot represents individual animals and the Geometric Mean (GM) represented by columns.

FIG. 7A: Shows the HBV surface antigen (HBs) specific CD4+ T-cell response in the spleen at 14dpll (i.e. 14 days post second dose) for all groups detailed in Table 2 (where C=ChAd155-hli-HBV, M=MVA-HBV, S=SAM-hli-HBV, P=HBc-HBs/AS01). At 14 days post second immunization (14dpll), spleens were collected to evaluate HB surface (HEs)-specific CD4+ T cells by intracellular cell staining. Each dot represents individual animals and the Geometric Mean (GM) represented by columns.

FIG. 7E: Shows the HBV surface antigen (HBs) specific CD4+ T-cell response in the spleen at 22dplV (i.e. 22 days after fourth dose) for all groups detailed in Table 2 (where C=ChAd155-hli-HBV, M=MVA-HBV, S=SAM-hli-HBV, P=HBc-HBs/AS01). At 22 days post fourth immunization (22dplV), spleens were collected to evaluate HB surface (HEs)-specific CD4+ T cells by intracellular cell staining. Each dot represents individual animals and the Geometric Mean (GM) represented by columns.

FIG. 8A: Shows the anti-HBc binding antibody titers measured at 13dpll/14dpll (i.e. 13/14 days after second dose) for all groups detailed in Table 2 (where C=ChAd155-hli-HBV, M=MVA-HBV, S=SAM-hli-HBV, P=HBc-HBs/AS01). At 13/14 days post second immunization, serum sample were collected to evaluate the anti-HBc IgG antibody titers by ELISA. For each group, each dot represents anti-HBc IgG antibody titer of individual animals and the Geometric Mean (GM) with 95% confidence intervals (CI) represented by columns.

FIG. 8E: Shows the anti-HBc binding antibody titers measured at 22dplV (i.e. 22 days after fourth dose) for all groups detailed in Table 2 (where C=ChAd155-hli-HBV, M=MVA-HBV, S=SAM-hli-HBV, P=HBc-HBs/AS01). At 22 days post fourth immunization, serum sample were collected to evaluate the anti-HBc IgG antibody titers by ELISA. For each group, each dot represents anti-HBc IgG antibody titer of individual animals and the Geometric Mean (GM) with 95% confidence intervals (CI) represented by columns.

FIG. 9A: Shows the anti-HBs binding antibody titers measured at 13dpll/14dpll (i.e. 13/14 days after second dose) for all groups detailed in Table 2 (where C=ChAd155-hli-HBV, M=MVA-HBV, S=SAM-hli-HBV, P=HBc-HBs/AS01). At 13/14 days post second immunization, serum sample were collected to evaluate the anti-HBs lgG antibody titers by ELISA. For each group, each dot represents anti-HBs IgG antibody titer of individual animals and the Geometric Mean (GM) with 95% confidence intervals (CI) represented by columns.

FIG. 9B: Shows the anti-HBs binding antibody titers measured at 22dplV (i.e. 22 days after fourth dose) for all groups detailed in Table 2 (where C=ChAd155-hli-HBV, M=MVA-HBV, S=SAM-hli-HBV, P=HBc-HBs/AS01). At 22 days post fourth immunization, serum sample were collected to evaluate the anti-HBs IgG antibody titers by ELISA. For each group, each dot represents anti-HBs IgG antibody titer of individual animals and the Geometric Mean (GM) with 95% confidence intervals (CI) represented by columns.

FIG. 10: Shows the kinetic of circulating HBs antigen titers detected in different groups. Geometric mean (GMs) of circulating HBs antigen titer are represented by squares with the 95% confidence intervals.

FIG. 11: Shows the circulating HBs antigen titers post second & fourth immunization compared to pre-immunization titers in different groups. Geometric mean ratios are represented by squares with the 90% confidence intervals.

FIG. 12: Shows the kinetic of AST and ALT levels detected in different groups. Geometric mean ratios (GMRs) of AST and ALT titers as compared to Pre-immunization titers are represented by triangles with the 95% confidence intervals.

FIG. 13A: Shows the cytokine co-expression profile of HBc-specific CD8+ T-cells. The frequency of HBc-specific CD8+ T cells expressing at least one, two or three cytokines (IL-2, IFN-γ and TNF-α) has been assessed by by intracellular staining at 14 days post-second immunization. The median values per group were plotted.

FIG. 13B: Shows the cytokine co-expression profile of HBs-specific T-cells. The frequency of HBs-specific CD8+ T cells expressing at least one, two or three cytokines (IL-2, IFN-γ and TNF-α) has been assessed by intracellular staining at 14 days post-second immunization. The median values per group were plotted.

FIG. 14: Shows the SAM-HBV constructs used in the Examples. The SAM constructs contain the genetic elements of VEEV TC-83 necessary for RNA amplification (non-structural protein sequences, nsP1-4). The sequences encodign for structural proteins have been replaced by the transgene encoding HBV polypeptides, which is under the control of the subgenomic promoter. The empty SAM plasmid is shown in SEQ ID NO: 16. The insert starts after nucleotide 7561 of SEQ ID NO: 16. Two different HBV constructs are shown. In both constructs, the HBc and HBs proteins are separated by 2A sequence. In one construct, human invariant chain (hli) is fused to HBc.

FIG. 15: Shows the hli_HBc_2A_HBs SAM plasmid map of the SAM plasmid sequence of SEQ ID NO: 18.

FIG. 16: Shows the HBc_2A_HBs SAM plasmid map of the SAM plasmid sequence of SEQ ID NO: 20.

FIG. 17: Shows the HBV-specific CD8+ and CD4+ T cell responses after co-administration of 3 different mRNAs.

FIG. 18: Shows the HBc-specific and HBs-specific CD8+ T cell responses observed in Example 3.

FIG. 19: Shows a comparison of the Geometric Mean Ratio (GMR) of the CD8+ Tell responses of FIG. 18 for the combination of (hli-HBc+ hli-HBs) mRNA constructs vs hli-HBc and hli-HBs alone.

FIG. 20: Shows the HBc-specific and HBs-specific CD4+ T cell responses observed in Example 3.

FIG. 21: Shows the HBc-specific IgG responses observed in Example 3.

### DETAILED DESCRIPTION OF THE INVENTION

### HBV Antigens

At least ten genotypes (A through J) of HBV have been identified (Liu, 2021) Within a given HBV genotype, multiple subgenotypes have also been identified. For example, genotypes A, B, C, D, and F have been further split into subgenotypes. The antigens for use in the disclosed compositions and methods are selected to provide immunological coverage across all HBV genotypes. The HBV genome contains four overlapping open-reading frames (ORF) that encode (i) the viral polymerase (Pol), (ii) the viral surface proteins (L-HBs, M-HBs and HBs), (iii) the PreCore/Core protein (HBe and HBc), and (iiii) the X protein (HBx).

The hepatitis B viral surface proteins (HBsAg) consist of three related yet different proteins—the large (L) surface protein, the medium (M) surface protein, and the small (S) surface protein. The HBV surface proteins (L, M, and S) are derived from alternate translation of the same ORF. The large surface protein is composed of three domains: preS1 (having 108/118/119 amino acids, depending on genotype; the genotype A preS1 domain is 119 amino acids), preS2 (having 55 amino acids), and the small surface protein (HBs, having 226 amino acids). The medium surface protein is composed of two domains: preS2 and the small surface protein (HBs). The small surface protein (HBs) does not contain preS1 or preS2 and is 226 amino acids long.

The hepatitis B core protein antigen (HBc) is highly conserved across genotypes and geno-subtypes and the hepatitis B small surface protein antigen (HBs) sequence is selected to include key cross-genotype-preserved B-cell epitopes which allow for induction of broad neutralizing responses. Suitably, the sequences of the HBc and of the HBs for use in the disclosed methods and compositions are based upon those from genotype/subtype A2.

Suitably, the HBV surface protein antigen for use in the disclosed methods and compositions is derived from the small (S) surface antigen protein. In particular, the HBV surface antigen for use herein can be derived from HBs.

In particular, a suitable HBV surface protein antigen comprises the small (S) protein (HBs) of 15 HBV adw2 strain, genotype A. For example, a suitable HBs antigen has at least 90%, 95%, 98% or 99% identity to the amino acid sequence set forth in SEQ ID NO: 1. In a preferred embodiment, a suitable HBs antigen has the 226 amino acids of amino acid sequence SEQ ID NO:1. In an aspect, the HBs antigen can be fused to hli. In an embodiment, the hli-HBs antigen has at least 90%, 95%, 98% or 99% identity to the amino acid sequence set forth in SEQ ID NO: 26. In a preferred embodiment, the hli-HBs has the amino acid sequence of SEQ ID NO: 26.

The hepatitis B core protein (HBc) is the major component of the nucleocapsid shell packaging the viral genome. This protein (183-185 aa long) is expressed in the cytoplasm of infected cells. HBc comprises a 149 residue assembly domain and a 34-36 residue RNA-binding domain at the C terminus. The HBc antigen for use in the disclosed methods and compositions may be full length or may comprise a C-terminally truncated protein (lacking the RNA-binding C-terminus), for example including amino acids 1-145 of a wild-type core antigen protein, e.g. amino acids 1-145, 1-146, 1-147, 1-148 or amino acids 1-149 of a wild-type hepatitis B core antigen protein. The truncated protein retains the ability to assemble into nucleocapsid particles. A suitable HBc antigen for use in the disclosed methods and compositions has an amino acid sequence from HBV adw2 strain, genotype A. When used as recombinant polypeptide, the recombinant HBc protein is suitably truncated from the wild-type at the C-terminus. In particular, the recombinant HBc protein has at least 90%, 95%, 98% or 99% identity to the amino acid sequence set forth in SEQ ID NO: 2. In a preferred embodiment, the recombinant HBc protein has the amino acid sequence of SEQ ID NO:2. When expressed in mRNA or from a viral vector, the HBc antigen is suitably a full-length HBc antigen. In particular, the HBc antigen has at least 90%, 95%, 98% or 99% identity to the amino acid sequence set forth in SEQ ID NO: 11. In an embodiment, the HBc antigen has the amino acid sequence of SEQ ID NO: 11. In an aspect, the HBc antigen can be fused to hli. In an embodiment, the hli-HBc has at least 90%, 95%, 98% or 99% identity to the amino acid sequence set forth in SEQ ID NO: 21. In a preferred embodiment, the hli-HBc has the amino acid sequence of SEQ ID NO: 21.

### Invariant Chain

Antigens are substances which induce an immune response in the body, especially the production of antibodies. Antigens may be of foreign, i.e. pathogenic, origin or stem from the organism itself, the latter are referred to as self- or auto antigens. Antigens can be presented on the surface of antigen presenting cells by MHC molecules. There are two classes of MHC molecules, MHC class I (MHC-I) and MHC class II (MHC-II). The MHC-II molecules are membrane-bound receptors which are synthesized in the endoplasmic reticulum and leave the endoplasmic reticulum in a MHC class II compartment. In order to prevent endogenous peptides, i.e. self-antigens, from binding to the MHC-II molecule and being presented to generate an immune response, the nascent MHC-II molecule interacts with another protein, the invariant chain, which blocks the peptide-binding cleft of the MHC-II molecule.

The human invariant chain (hli, also known as CD74 when expressed at the plasma membrane), is an evolutionarily conserved type II membrane protein which has several roles within the cell and throughout the immune system (Borghese, 2011). When the MHC class II compartment fuses to a late endosome containing phagocytosed and degraded foreign proteins, the invariant chain is cleaved to leave only the CLIP region bound to the MHC-II molecule. In a second step, CLIP is removed by an HLA-DM molecule leaving the MHC-II molecule free to bind fragments of the foreign proteins. Said fragments are presented on the surface of the antigen-presenting cell once the MHC class II compartment fuses with the plasma membrane, thus presenting the foreign antigens to other cells, primarily T-helper cells.

It is known that the immune response against an antigen is increased when an adenovirus expression system encoding a fusion of invariant chain and said antigen is used for vaccination (see WO2007/062656, which also published as US2011/0293704 and is incorporated by reference for the purpose of disclosing invariant chain sequences), i.e. the invariant chain enhances the immunogenicity of the antigen. Moreover, said adenoviral construct has proven useful for priming an immune response in the context of prime-boosting vaccination regimens (see WO2014/141176, which also published as US2016/0000904; and WO2010/057501, which also published as US2010/0278904 and is incorporated by reference for the purpose of disclosing invariant chain sequences and adenoviral vectors encoding invariant chain sequences).

In the present invention, the mRNA encoding a hepatitis B virus antigen includes a nucleotide sequence coding for invariant chain (Ii), preferably human invariant chain (hli). Two amino acid sequences for hli are set forth in SEQ ID NO:7 and SEQ ID NO:12. In a preferred embodiment, the invariant chain has SEQ ID NO:12. Suitably, a nucleotide sequence coding 10 for hli is N-terminally fused to the nucleotide sequence coding for the HBc antigen, and/or HBs antigen.

In the present invention, there is a composition for treating chronic hepatitis B infection comprising a mRNA encoding at least a hepatitis B virus core antigen (HBc), wherein the mRNA is encapsulated in a lipid nanoparticle (LNP), wherein the N-terminal of the nucleotide sequence encoding HBc is fused to human invariant chain (hli).

In the present invention, there is also a composition for treating chronic hepatitis B infection comprising a first mRNA encoding a hepatitis B virus core antigen (HBc), and a second mRNA encoding a hepatitis B virus surface antigen (HBs), wherein the first and second mRNA are encapsulated in lipid nanoparticles (LNP), and wherein the N-terminals of the nucleotide sequences encoding HBc and HBs are fused to human invariant chain (hli).

In one embodiment, the mRNA encodes the amino acid sequence of SEQ ID NO: 9 and SEQ 25 ID NO: 15 (preferably SEQ ID NO: 15). SEQ ID NO: 15 is the fusion of the hli reported in SEQ ID NO: 12, the HBc reported in SEQ ID NO: 11, the 2A reported in SEQ ID NO: 3 and the HBs reported in SEQ ID NO: 1.

In certain embodiments, the adenoviral vector (Ad), for example, a chimpanzee adenoviral 30 vector (ChAd), for use in the methods and compositions disclosed herein may include a nucleotide sequence coding for hli. Two amino acid sequences for hli as included in the disclosed adenoviral vector are set out in SEQ ID NO:7 and SEQ ID NO:12, and nucleotide sequences encoding these amino acid sequences are set out in SEQ ID NO:8 and SEQ ID NO:13 respectively. In a preferred embodiment, the invariant chain has SEQ ID NO:12. Suitably, the nucleotide sequence coding for hli is N-terminally fused to the nucleotide sequence coding for the HBc antigen.

Messenger RNA (mRNA)
Non-Replicating mRNA

The present disclosure provides compositions comprising recombinant messenger RNA (mRNA) having an open reading frame encoding at least one hepatitis B virus antigen. As used herein, the term “recombinant messenger RNA” (mRNA) refers to any recombinantly-produced polynucleotide which encodes at least one polypeptide of interest and which is capable of being translated to produce the encoded polypeptide of interest in vitro, in vivo, in situ, or ex vivo. mRNA typically contains a segment that encodes a polypeptide of interest (i.e. a segment that encodes a heterologous polypeptide, such as a hepatitis B virus antigen), a 5′ untranslated region (5′ UTR), an optional 3′ untranslated region (3′ UTR), an 3′ poly(adenosine monophosphate) (3′ poly(A)) tail, and a 5′ cap. The 5′ UTR is upstream (i.e. 5′) of the polypeptide of interest; whereas, the 3′ UTR is downstream (i.e. 3′) of the polypeptide of interest. The 5′ UTR begins at the transcription start site and ends one nucleotide before the translation initiation sequence (i.e. a sequence of 5′-adenosine, uridine, guanosine-3′ (5′-AUG-3′)) of the coding region of the polypeptide of interest. The 3′ UTR follows the translation termination codon of the coding region of the polypeptide of interest.

In an embodiment, the mRNA of the present disclosure may be structurally modified or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide. In one embodiment, the mRNA of the present disclosure may have uniform chemical modification of all or any of the same nucleoside type, or a measured percent of a chemical modification of all or any of the same nucleoside type but with random incorporation, such as where all uridines are replaced by a uridine analog, e.g., pseudouridine. In another embodiment, the mRNA may have a uniform modification of two, three, or four of the same nucleoside type throughout the entire polynucleotide (such as all uridines and all cysteine, etc. are modified in the same way.) When the polynucleotides of the presently disclosed mRNA are chemically or structurally modified, the polynucleotides may be referred to as “modified polynucleotides.” In an embodiment, the mRNA has the configuration: 5′cap/5′UTR/hli/HBc/3′UTR/polyA.

In an embodiment, the mRNA has the configuration: 5′cap/5′UTR/HBc/3′UTR/polyA.

In an embodiment, the mRNA has the configuration: 5′cap/5′UTR/hli/HBs/3′UTR/polyA.

In an embodiment, the mRNA has the configuration: 5′cap/5′UTR/HBs/3′UTR/polyA.

In some embodiments, the mRNA comprises a 5′ cap. In some embodiments, the mRNA further comprises a 7-methylguanosine, a 5′ first ribonucleoside, an optional 5′ second ribonucleoside, and an optional tri-phosphate bridge. In some embodiments, the 7-methylguanosine is linked directly or indirectly 5′-to-5′ to the 5′ first ribonucleoside. In some embodiments, the 7-methylguanosine is linked 5′-to-5′ to the 5′ first ribonucleoside by the triphosphate bridge. In some embodiments, the 5′ first ribonucleoside comprises a 2′-methylated ribose (2′-O-Me) (i.e. a cap-1 or cap-2). In some embodiments, the 5′ second ribonucleoside is bound to the 3′ end of the 5′ first ribonucleoside. In some embodiments, the 5′ second ribonucleoside comprises a 2′-methylated ribose (2′-O-Me) (i.e. a cap-2). In some embodiments, the 5′ first ribonucleoside comprises a 2′-methylated ribose (2′-O-Me) and the 5′ second ribonucleoside comprises a 2′-methylated ribose (2′-O-Me) (i.e. a cap-2). A 5′ cap comprises a guanosine connected to the RNA via a 5′ to 5′ triphosphate linkage by mRNA guanylyltransferase, and wherein the guanine of said guanosine is methylated at its 7 position. In this context and in some embodiments, a 5′ to 5′ triphosphate linkage occurs when the 5′ end of the ribose of said guanosine is linked to the 5′ end of the ribose of the mRNA via a triphophosphate group by mRNA guanylyltransferase. In some embodiments, thereafter, the guanine of said guanosine is methylated at its 7 position by (guanine-N7-)-methyltransferase. In some embodiments, the addition of the 7-methylguanosine 5′-to-5′ to the 5′ first ribonucleoside occurs at once, without addition of the 7-guanosine and further methylation thereof to obtain 7-methylguanosine (i.e. CLEANCAP@). In some embodiments, the addition of the 7-methylguanosine 5′-to-5′ to the 5′ first ribonucleoside and the addition of the 5′ first ribonucleoside comprising a 2′-methlyated ribose or 5′ second ribonucleoside comprising a 2′-methylated ribose occurs at once (i.e. CLEANCAP@). In some embodiments, the cap structure is preformed (i.e. as cap-1, cap-2, or cap-0, with or without the addition of the 7 methyl-group on the 5′ guanosine/7-methylguanosine) and added to the recombinant RNA molecule (i.e. by ligation). In some embodiments, the preformed cap structure is added with a 5′-AG-3′ initiating sequence as described in the CLEANCAP@AG product insert (Trilink catalog number N-7113), which is incorporated by reference).

Without further methylation, a 7-methylguanosine bound 5′-to-5′ to the 5′ first ribonucleoside is known as cap-0 and is expressed as 5′(m7Gp)(ppN)[pN]_{N}, wherein the former “N” indicates the first (5′) nucleobase of the mRNA, the “pN” indicates a further nucleotide in the RNA, and the addition of “[ . . . ]_{N}” in “[pN]_{N}” indicates the repeating polymeric structure of the RNA and thereby collectively each sequentially adjacent nucleotide in the RNA.

An additional oxygen-linked methylation by a 2′-O-methyltransferase to the 2′ carbon of the ribose of the nucleoside of the mRNA immediately adjacent to said 7-methylguanosine (i.e. the 5′ first ribonucleoside) results in a cap-1 structure, which is expressed as 5′(m7Gp)(ppm2N)[pN]_{N}, wherein the addition of the “m2” indicates the oxygen-linked methylation of the 2′ carbon of the ribose of the nucleoside immediately adjacent (via the triphosphate linkage) to said 7-methylguanosine. And further still, an additional methylation to the 5′ second ribonucleoside (i.e the next (3′) nucleoside immediately adjacent to the 5′ first nucleoside methylated in cap-1) results in a cap-2 structure, which is expressed as 5′(m7Gp)(ppm2N)(m2pN)[pN]n, wherein the addition of the latter “m2” indicates the methylation of the nucleotide immediately adjacent to the nucleotide methylated in cap-1. This cap-2 methylation is also to the 2′ carbon of the ribose of that immediately adjacent nucleotide (i.e. 2′-O-Me). In some embodiments, the 5′ cap is a cap-0, a cap-1, or a cap-2. In some embodiments, the 5′ cap is a cap-0. In some embodiments, the 5′ cap is a cap-1. In some embodiments, the 5′ cap is a cap-2.

In some embodiments, the 5′ first ribonucleoside or the 5′ second ribonucleoside is exogenously added to the mRNA. In some embodiments, the 5′ first ribonucleoside or the 5′ second ribonucleoside is native to the mRNA (i.e. if the native sequence is 5′-UUAAT-3′, then the addition of the m7Gp would result in 5′-m7Gp(ppUUAAT-3′ when there is a triphosphate bride; if the native sequence is the same, then the cap-1 structure would result in 5-′(m7Gp)(ppm2U)UAAT-3′ and the cap-2 structure being 5-′(m7Gp)(ppm2U)(m2U)AAT-3′).

Kits providing all of the materials for a 5′ cap, whether it is cap-1 or cap-2, and supplemental kits adding cap-1 and cap-2 capacity to a cap-0 kit can be used. The methods for 5′ capping can be carried out according to the manufacturer's instructions.

In some embodiments, the mRNA comprises a 3′ poly(adenosine monophosphate) (poly(A)) tail. In some embodiments, the 3′ poly(A) tail is 3′ from the 3′ UTR. In some embodiments, the 3′ poly(A)) tail is at the 3′ end of the mRNA.

The mRNA disclosed herein may be modified. The term “modified mRNA” or “RNA modification” as used herein may refer to chemical modifications comprising backbone modifications as well as sugar modifications or base modifications. In this context, a modified RNA molecule as defined herein may contain nucleotide analogues/modifications, e.g. backbone modifications, sugar modifications or base modifications. A backbone modification in connection with the present invention is a modification, in which phosphates of the backbone of the nucleotides contained in an RNA molecule as defined herein are chemically modified. A sugar modification in connection with the present invention is a chemical modification of the sugar of the nucleotides of the RNA molecule as defined herein. Furthermore, a base modification in connection with the present invention is a chemical modification of the base moiety of the nucleotides of the RNA molecule. In this context, nucleotide analogues or modifications are preferably selected from nucleotide analogues, which are applicable for transcription and/or translation.

In an aspect, the modified nucleosides and nucleotides, which may be incorporated into a modified RNA molecule as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

In another aspect, the phosphate backbone may further be modified in the modified nucleosides and nucleotides, which may be incorporated into a modified RNA molecule as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Non-limiting examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

The modified nucleosides and nucleotides, which may be used in the present invention, can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

The modified mRNA may comprise one or more modified nucleosides and nucleotides. The preparation of nucleosides and nucleotides, and modified nucleotides and nucleosides, are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

Modified nucleobases which can be incorporated into modified nucleosides and nucleotides and be present in the mRNA molecules include: pseudouridine; N1-methylpseudouridine; N1-ethylpseudouridine; 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl)adenine; N6-cis-hydroxy-isopentenyl-adenosine; .alpha.-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-lodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; .alpha.-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine: 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine: 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; .alpha.-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester, 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridine; 5-carboxymethylaminomethyluridine; 5-Carbamoylmethyluridine TP; 5-methoxycaeoonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl 30 ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; .alpha.-thio-uridine; 1 (aminoalkylamino-carbonylethylenyl)-2(thio)-pseudouridine; 1 (aminoalkylaminocarbonylethylenyl)-2,4-(dithio)pseudouridine; 1 (aminoalkylaminocarbonylethylenyl)-4 (thio)pseudouridine; 1 (aminoalkylaminocarbonylethylenyl)-pseudouridine; 1 (aminocazbonylethylenyl)-2(thio)-pseudouridine; 1 (aminocarbonylethylenyl)-2,4-(dithio)pseudouridine; 1 (aminocarbonylethylenyl)-4 (thio)pseudouridine; 1 (aminocarbonylethylenyl)-pseudouridine; 1 substituted 2(thio)-pseudouridine; 1 substituted 2,4-(dithio)pseudouridine; 1 substituted 4 (thio)pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouridine; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouridine; 4-(thio)pseudouridine; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouridine; 5-(alkyl)-2,4 (dithio)pseudouridine; 5-(alkyl)-4 (thio)pseudouridine; 5-(alkyl)pseudouridine; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouridine; 5-(methyl)-2,4 (dithio)pseudouridine; 5-(methyl)-4 (thio)pseudouridine; 5-(methyl)pseudouridine; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouridine; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (.+-.)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine 5 TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-lodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1-(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminoethoxy)-ethoxy]-propionyl}pseudouridine TP; 1-Acetylpseudouridine TP; I-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-lodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; 30 Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; or N6-(19-Amino-pentaoxanonadecyl)adenosine TP.

In some embodiments, the adenosine-substitutable modified nucleotides comprise: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl)adenine; N6-cis-hydroxy-isopentenyl-adenosine; .alpha.-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-lodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; Wybutosine; Hydroxywybutosine; 10 Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; O6-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; or N6-(19-Amino-pentaoxanonadecyl)adenosine TP.

In some embodiments, the uridine-substitutable modified nucleotides or the thymidine-substitutable modifified nucleotides comprise: pseudouridine; N1-methylpseudouridine; N1-ethylpseudouridine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester, 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridine; 5-carboxymethylaminomethyluridine; 5-Carbamoylmethyluridine TP; 5-methoxycaeoonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; .alpha.-thio-uridine; 1 (aminoalkylamino-carbonylethylenyl)-2(thio)-pseudouridine; 1 (aminoalkylaminocarbonylethylenyl)-2,4-(dithio)pseudouridine; 1 (aminoalkylaminocarbonylethylenyl)-4 (thio)pseudouridine; 1 (aminoalkylaminocarbonylethylenyl)-pseudouridine; 1 (aminocazbonylethylenyl)-2(thio)-pseudouridine; 1 (aminocarbonylethylenyl)-2,4-(dithio)pseudouridine; 1 (aminocarbonylethylenyl)-4 (thio)pseudouridine; 1 (aminocarbonylethylenyl)-pseudouridine; 1 substituted 2(thio)-pseudouridine; 1 substituted 2,4-(dithio)pseudouridine; 1 substituted 4 (thio)pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouridine; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouridine; 4-(thio)pseudouridine; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouridine; 5-(alkyl)-2,4 (dithio)pseudouridine; 5-(alkyl)-4 (thio)pseudouridine; 5-(alkyl)pseudouridine; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouridine; 5-(methyl)-2,4 (dithio)pseudouridine; 5-(methyl)-4 (thio)pseudouridine; 5-(methyl)pseudouridine; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouridine; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (.+-.)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-lodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1-(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminoethoxy)-ethoxy]-propionyl}pseudouridine TP; 1-Acetylpseudouridine TP; I-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-lodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; O6-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one 10 ribonucleoside; 2-Amino-riboside-TP; Formycin A TP; Formycin B TP; Pyrrolosine TP; 2′-OH-ara-cytidine TP; 2′-OH-ara-uridine TP TP; or 5-(2-carbomethoxyvinyl)uridine TP.

In some embodiments, the cytosine-substitutable modified nucleotides comprise 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; .alpha.-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine: 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine: 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 2′fluoro-cytidine; or 2′-OH-ara-cytidine TP.

In some embodiments, the modified nucleotides comprise: 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; .alpha.-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; or 2′-OH-ara-guanosine TP.

Many of these modified nucleobases and their corresponding ribonucleosides are available from commercial suppliers.

Self-Amplifying mRNA (SAM)

The mRNA disclosed herein may be replicating, also known as self-amplifying. A self-amplifying mRNA molecule may be an alphavirus-derived mRNA replicon. mRNA amplification can also be achieved by the provision of a non-replicating mRNA encoding an antigen in conjunction with a separate mRNA encoding replication machinery.

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see the following reference: WO2005/113782.

In certain embodiments, the self-replicating RNA molecule described herein encodes (i) a RNA-dependent RNA polymerase which can transcribe RNA from the self-replicating RNA molecule and (ii) an antigen. The polymerase can be an alphavirus replicase e.g. comprising one or more of alphavirus proteins nsPI, nsP2, nsP3 and nsP4 (wherein nsP stands for non-structural protein).

Whereas natural alphavirus genomes encode structural virion proteins in addition to the non-structural replicase polyprotein, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the sub-genomic transcript encodes the immunogen rather than the structural alphavirus virion proteins.

A self-replicating RNA molecule useful with the invention may have two open reading frames. The first (5) open reading frame encodes a replicase; the second (3′) open reading frame encodes one or more HBV antigens.

In certain embodiments, the self-replicating RNA molecule disclosed herein has a 5 cap (e.g. a 7-methylguanosine). This cap can enhance in vivo translation of the RNA. In some embodiments the 5′ sequence of the self-replicating RNA molecule must be selected to ensure compatibility with the encoded replicase.

A self-replicating RNA molecule may have a 3′ poly-A tail. It may also include a poly-A polymerase recognition sequence (e.g. AAUAAA) near its 3′ end.

Self-replicating RNA molecules can have various lengths, but they are typically 5000-25000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA. In another embodiment, a self-replicating RNA may comprise two separate RNA molecules, each comprising a nucleotide sequence derived from an alphavirus: one RNA molecule comprises a RNA construct for expressing alphavirus replicase, and one RNA molecule comprises a RNA replicon that can be replicated by the replicase in trans. The RNA construct for expressing alphavirus replicase comprises a 5′-cap. See WO2017/162265. The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. An RNA used with the invention ideally includes only phosphodiester linkages between nucleosides, but in some embodiments it can contain phosphoramidate, and/or methylphosphonate linkages.

The self-replicating RNA molecule may encode a single heterologous polypeptide antigen (i.e. the antigen) or, optionally, two or more heterologous polypeptide antigens linked together in a way that each of the sequences retains its identity (e.g., linked in series) when expressed as an amino acid sequence. The heterologous polypeptides generated from the self-replicating RNA may then be produced as a fusion polypeptide or engineered in such a manner to result in separate polypeptide or peptide sequences.

The self-replicating RNA molecules described herein may be engineered to express multiple nucleotide sequences, thereby allowing co-expression of proteins, such as one, two or more HBV antigens (e.g. surface and core antigens. The RNA molecules may express these proteins together with cytokines or other immunomodulators, which can enhance the generation of an immune response.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes an antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode an antigen can also be tested for ability to induce humoral immune responses, as evidenced, for example, by induction of B cell production of antibodies specific for the antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

In one embodiment, the self-replicating RNA of the present disclosure may include a sequence encoding a self-cleaving peptide. The self-cleaving peptide may be, but is not limited to, the 2A cleaving region of the foot and mouth disease virus (FMDV) (herein known as “2A”). The 2A peptide has the amino acid sequence of SEQ ID NO: 3. In an aspect, the 2A peptide cleaves between the last glycine and last proline. The 2A peptide makes the ribosome skip the synthesis of a peptide bond at the C-terminus of the 2A peptide, leading to separation (“cleavage”) between the end of the 2A sequence and the next peptide downstream. In an embodiment, the 2A peptide may be used to separate the coding region of two or more polypeptides of interest. As a non-limiting example, the nucleotide sequence encoding the 2A peptide may be between a first coding region A (e.g., encoding hli-HBc) and a second coding region B (e.g., encoding HBs).

In another embodiment, self-replicating RNA of the present disclosure may include a sequence encoding an internal ribosome entry site (IRES). The IRES element acts like an additional ribosome recruitment site, allowing translation to occur at an internal region of the mRNA, thereby resulting in the downstream ORF being translated separately from the upstream ORF.

In an embodiment, IRES may be used to separate the coding region of two or more polypeptides of interest. As a non-limiting example, the nucleotide sequence encoding IRES may be between a first coding region A (e.g., encoding hli-HBc) and a second coding region B (e.g., encoding HBs).

In an embodiment, the self-replicating RNA has the configuration: 5′cap/5′UTR-non-structural proteins (NSP) 1-4/subgenomic promoter/hli/HBc/2A/HBs/3′UTR/polyA.

In an embodiment, the self-replicating RNA has the configuration: 5′cap/5′UTR-non-structural proteins (NSP) 1-4/subgenomic promoter/hli/HBc/IRES/HBs/3′UTR/polyA.

In an embodiment, the self-replicating RNA has the configuration: 5′cap/5′UTR-non-structural proteins (NSP) 1-4/subgenomic promoter/hli/HBc/3′UTR/polyA.

In an embodiment, the self-replicating RNA has the configuration: 5′cap/5′UTR-non-structural proteins (NSP) 1-4/subgenomic promoter/HBs/3′UTR/polyA.

In an embodiment, the self-replicating RNA has the configuration: 5′cap/5′UTR-non-structural proteins (NSP) 1-4/subgenomic promoter/hli/HBs/3′UTR/polyA.

In one embodiment the mRNA is non-replicating mRNA. In a second embodiment the mRNA is replicating mRNA.

Lipid Nanoparticles (LNP) Lipid nanoparticles (LNPs) are non-virion liposome particles in which mRNA can be encapsulated. RNA, by itself and unprotected, may be degraded by the subject's RNAses. LNPs provide a means to protect the mRNA by encapsulating within them an amount of the mRNA in the overall composition. LNP delivery systems and methods for their preparation are known in the art. The LNPs can include some external mRNA (e.g. on the surface of the LNP), but desirably at least half of the mRNA (and suitably at least 85%, especially at least 95%, such as all of it) is encapsulated.

### First Lipids

In some embodiments, the LNP comprises lipids comprising: a first lipid (i.e. a cation-ionizable lipid), an optional sterol (e.g. cholesterol), an optional polymer-conjugated lipid, and an optional second lipid (i.e. an optional anionic lipid or an optional neutral lipid, including zwitterionic lipids). In some embodiments, the optional neutral lipid comprises a neutral lipid zwitterionic lipid. In some embodiments, the polymer-conjugated lipid comprises a polyethylene glycol-conjugated lipid. In some embodiments, the LNP comprises a lipid from WO2012/006376, WO2012/030901, WO2012/031046, WO2012/031043, WO2012/006378, WO2011/076807, WO2013/033563, WO2013/006825, WO2014/136086, WO2015/095340, WO2015/095346, WO2016/037053, WO2017/075531, WO2018/081480, WO2015/074085, WO2018/1703322, U.S. Patent Application Publication Nos.: 20220081392, 20220072155, 20220040285, 20210395188, 20210251898, 20210128488, 20210122703, 20210122702, 20210107861, 20200283372, 20200172472, 20200163878, 20200121809, 20200046838, 20190359556, 20190314524, 20190274968, 20190270697, 20190022247, 20180185516, 20170283367, 20170157268, 20170119904, 20160376224, 20160317676, or 20150376115, U.S. Application Nos. 61/905,724 or 15/614,499, or U.S. Pat. Nos. 8,802,863, 9,458,090, 9,593,077, 9,567,296, 9,604,908, 9,643,916, 9,669,097, 9,670,487, 9,737,619, 9,738,593, 9,725,720, 9,796,977,10,106,490, 10,166,298, 10,221,127, 10,723,692, 11,040,112, 11,168,051, or 11,285,222 (including ionizable and PEG-lipids, as referred to therein).

In some embodiments, the cation-ionizable lipid comprises an amine that can be a tertiary amine, which can become charged depending upon the pH of the solution that the cation-ionizable lipid is in when compared to the pKa of the cation-ionizable lipid. In some embodiments, at least half of the cation-ionizable lipids are neutrally charged and the amine is a tertiary amine when the pH of the solvent that the cation-ionizable lipids are in is above the pKa; and at least half of the cation-ionizable lipids are positively charged when the pH of the solvent that the cation-ionizable lipids are in is below the pKa. In this regard, in some embodiments, but without being limited to a particular theory, it is considered that the positive charge of the ionizable lipid is distributed on the amine, and thereby the amine is positively charged when the pH of the solvent that the cation-ionizable lipids are in is below the pKa. Since the amine can vary between neutrally and positively charged depending upon the pH of the solution relative to the pKa of the cation-ionizable lipid and since, without being bound a particular theory, the amine is an ionizable amine.

The cation-ionizable lipid will be further described when the amine is tertiary and when the cation-ionizable lipid is neutrally charged, but such descriptions shall not limit the cation-ionizable lipid to lacking the ability to transition to being positively charged. That is, the lipid being in a tertiary amine state and having a neutral charged is hereby described, without having to describe the cation-ionizable lipid when the tertiary amine becomes charged. In some embodiments, the cation-ionizable lipid further comprises, in addition to the above-noted ionizable amine, a headgroup (RH) and a fatty acid tail (R^{FA1} or R^{FA2}). In some embodiments, the cation-ionizable lipid further comprises (in addition to the above-noted ionizable amine) a headgroup and at least two fatty acid tails (R^{FA1} and R^{FA2}), such as in Formula I.

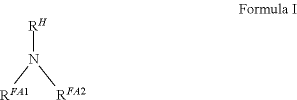

In some embodiments, the amine provides a branchpoint between the headgroup and a fatty acid tail. In some embodiments, the fatty acid tail (R^{FA}) or the at least two fatty acid tails (i.e. R^{FA1}, R^{FA2} . . . ) are immediately off of the ionizable amine. In some embodiments, the fatty acid tail comprises, or the at least two fatty acid tails comprise, a biodegradeable group (i.e. R^{BD1} or R^{BD2}), and the at least two fatty acid tails are the same or independent of one another. In some embodiments, the at least two fatty acid tails each comprise a biodegradeable group, such as in Formula II, and the biodegradeable groups are the same or independent of one another. In some embodiments, the biodegradeable group comprises, in order from the ionizable amine, —O(C═O)—, —(C═O)O—, —C(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a} C(═O)O—, wherein X is 0, 1, or 2, and wherein R^{a} is a hydrogen or a C₁-C₁₂ alkyl. In some embodiments, the fatty acid comprises, or the at least two fatty acids comprise, a C₁-C₁₂ alkyl, a C₁-C₁₂ alkylene, or a C₁-C₁₂ alkenylene (i.e. R^{FC1} and R^{FC2}) between the amine branchpoint and the biodegradeable group), such as in Formula II. In some embodiments, the fatty acid comprises, or the two or more fatty acids comprise, distal to the ionizable amine and the biodegradeable group, a C₆-C₂₄alkyl, a C₆-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈ alkyl, a C₁₂-C₁₈ alkylene, a C₁₃-C₁₇ alkyl, or a C₁₃-C₁₇ alkylene (i.e. R^{FC3} and R^{FC4}), such as in Formula II.

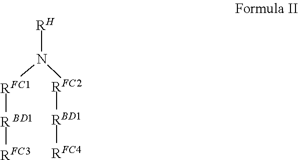

Wherein: R^{FC1} and R^{FC2} are each independently a C₁-C₁₂ alkyl, a C₁-C₁₂ alkylene, or a C₁-C₁₂ alkenylene;
R^{FC3} and R^{FC4} are each independently: a C₆-C₂₄ alkyl, a C₆-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈ alkyl, a C₁₂-C₁₈ alkylene, a C₁₃-C₁₇ alkyl, a C₁₃-C₁₇ alkylene;
R^{BD1} and R^{BD2} are each independently: —O(O═O)—, —(O═O)O—, —O(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a}C(═O)O—; wherein X is 0, 1, or 2, and wherein R^{a} is a hydrogen or a C₁-C₁₂ alkyl.

In some embodiments, the C₆-C₂₄ alkyl or the C₆-C₂₄ alkylene is connected to the biodegradeable group at C₆-C₁₂, C₇-C₁₁, C₈-C₁₀, or C₉ thereof. In some embodiments, the C₆-C₂₄ alkyl or the C₆-C₂₄ alkylene of each of the at least two fatty acid tails independently comprises:

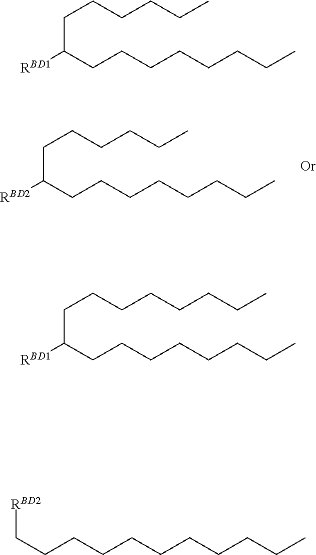

In some embodiments, the headgroup comprises, consists of, is, or has a first group (i.e. R^{H1}) and a second group (i.e. R^{H2}), wherein the first group is a C₁-C₂₄ alkyl, C₁-C₂₄ alkylene, a C₁-C₂₄ alkenylene, a C₃-C₈ cycloalkylene, or a C₃-C₈ cycloalkenylene, and the second group is H, —OH, CN, —C(═O)OR⁴, —OC(═O)OR⁴, —NR⁵C(═O)OR⁴, or OR⁵; wherein R⁴ is C₁-C₁₂ alkyl and R⁵ is H or C₁-C₆ alkyl. In some embodiments, the headgroup comprises a linear or branched form of: —(CH₂)₆OH, —(CH₂)₅OH, —(CH₂)₄OH, —(CH₂)₃OH, —(CH₂)₂OH, or —CH₂OH. In some embodiments, the cation-ionizable lipid comprises, consists of, or is [(4-Hydroxybutyl)azanediyl]di(hexane-6,1-diyl) bis(2-hexyldecanoate) or 9-Heptadecanyl 8-{(2-hydroxyethyl)[6-oxo-6-(undecyloxy)hexyl]amino}octanoate.

In some embodiments, the cation-ionizable lipid is:

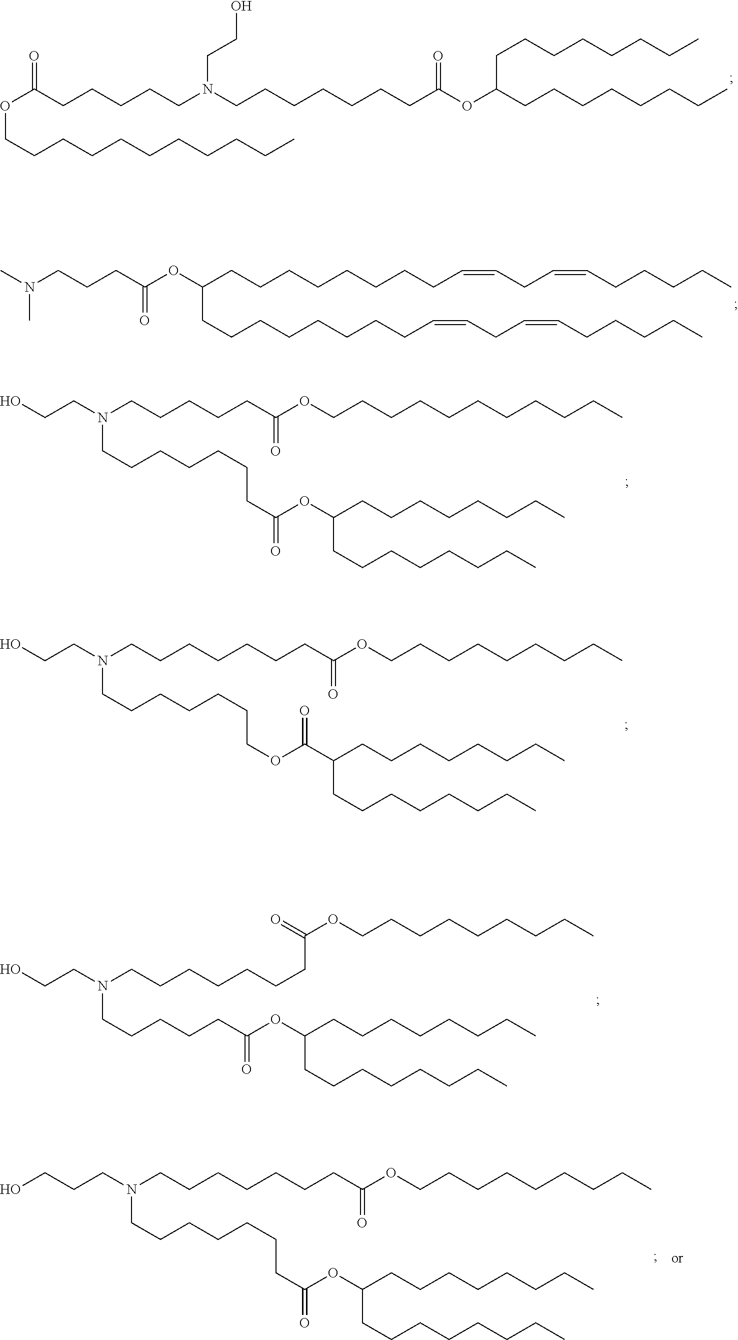

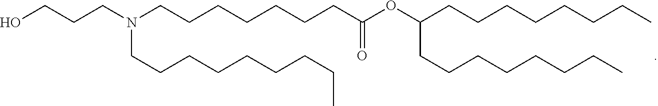 [SMILES: CCCCCCCCCN(CCCO)CCCCCCCC(=O)OC(CCCCCCCC)CCCCCCCC]

In an embodiment, the cation-ionizable lipid comprises, consists of, consists essentially of, or is RV28 having the following structure:

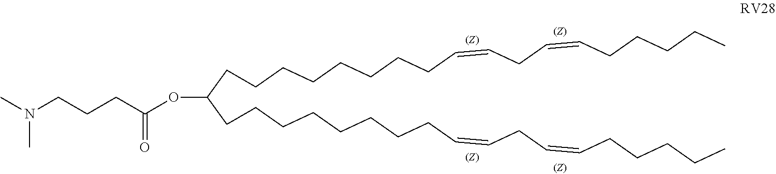 [SMILES: CCCCC/C=C\C/C=C\CCCCCCCCC(CCCCCCCC/C=C\C/C=C\CCCCC)OC(=O)CCCN(C)C]

In an embodiment, the cation-ionizable lipid comprises, consists of, consists essentially of, or is RV31 having the following structure:

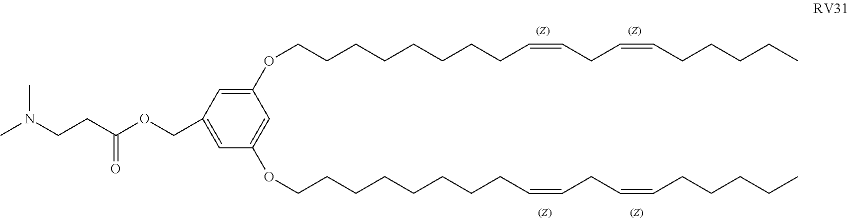 [SMILES: CCCCC/C=C\C/C=C\CCCCCCCCOc1cc(COC(=O)CCN(C)C)cc(OCCCCCCCC/C=C\C/C=C\CCCCC)c1]

In an embodiment, the cation-ionizable lipid comprises, consists of, consists essentially of, or is RV33 having the following structure:

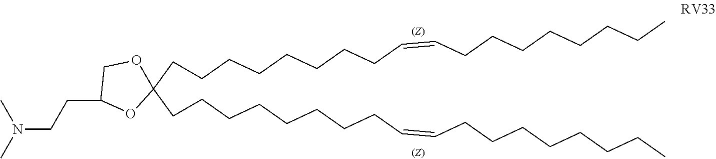 [SMILES: CCCCCCCC/C=C\CCCCCCCCC1(CCCCCCCC/C=C\CCCCCCCC)OCC(CCN(C)C)O1]

In an embodiment, the cation-ionizable lipid comprises, consists of, consists essentially of, or is RV37 having the following structure:

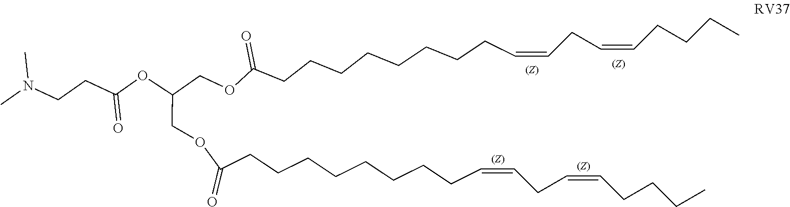 [SMILES: CCCC/C=C\C/C=C\CCCCCCCCC(=O)OCC(COC(=O)CCCCCCCC/C=C\C/C=C\CCCC)OC(=O)CCN(C)C]

In an embodiment, the cation-ionizable lipid comprises, consists of, consists essentially of, or is RV39, i.e., 2,5-bis((9Z,12Z)-octadeca-9,12-dien-1-yloxy)benzyl 4-(dimethylamino)butanoate):

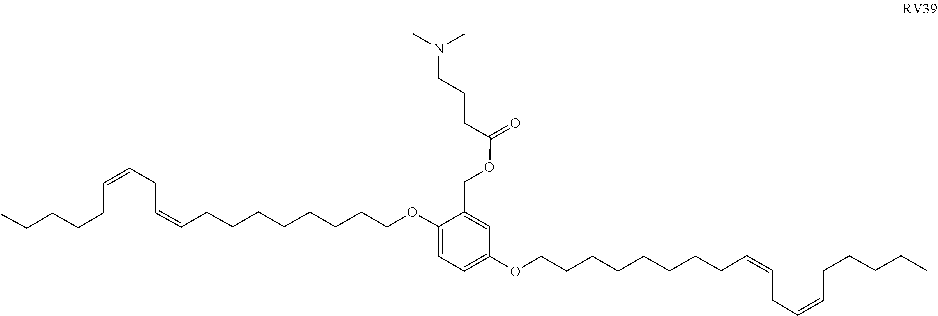 [SMILES: CCCCC/C=C\C/C=C\CCCCCCCCOc1ccc(OCCCCCCCC/C=C\C/C=C\CCCCC)c(COC(=O)CCCN(C)C)c1]

In an embodiment, the cation-ionizable lipid comprises, consists of, consists essentially of, or is RV42 having the following structure:

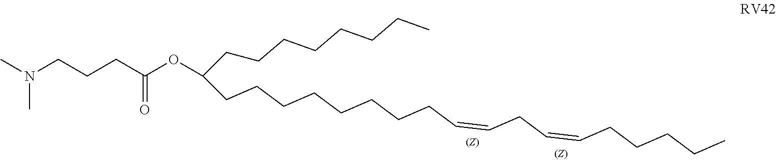 [SMILES: CCCCC/C=C\C/C=C\CCCCCCCCC(CCCCCCCC)OC(=O)CCCN(C)C]

In an embodiment, the cation-ionizable lipid comprises, consists of, consists essentially of, or is RV44 having the following structure:

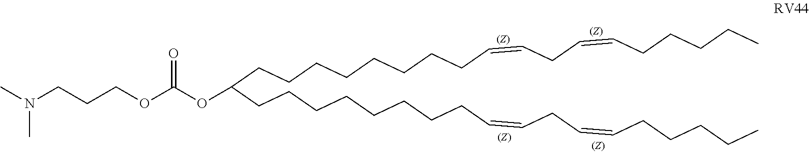 [SMILES: CCCCC/C=C\C/C=C\CCCCCCCCC(CCCCCCCC/C=C\C/C=C\CCCCC)OC(=O)OCCCN(C)C]

In an embodiment, the cation-ionizable lipid comprises, consists of, consists essentially of, or is RV73 having the following structure:

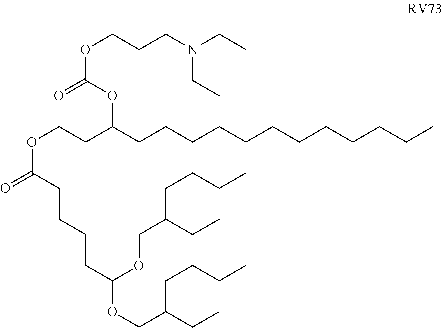 [SMILES: CCCCCCCCCCCCC(CCOC(=O)CCCCC(OCC(CC)CCCC)OCC(CC)CCCC)OC(=O)OCCCN(CC)CC]

In an embodiment, the cation-ionizable lipid comprises, consists of, consists essentially of, or is RV75 having the following structure:

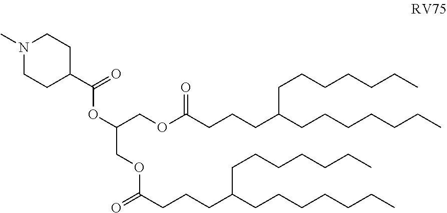 [SMILES: CCCCCCCC(CCCCCCC)CCCC(=O)OCC(COC(=O)CCCC(CCCCCCC)CCCCCCC)OC(=O)C1CCN(C)CC1]

In an embodiment, the cation-ionizable lipid comprises, consists of, consists essentially of, or is RV81 having the following structure:

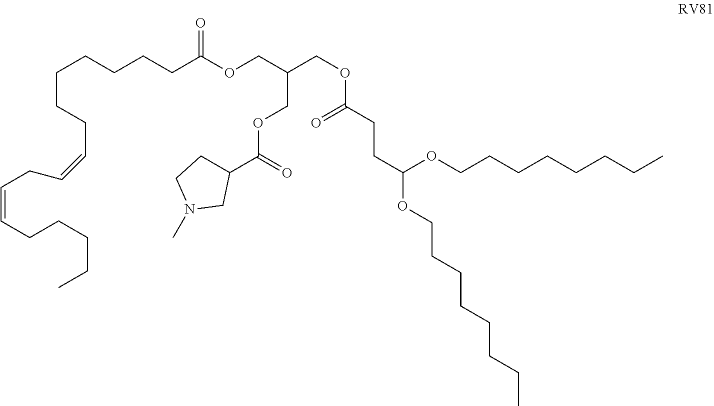 [SMILES: CCCCC/C=C\C/C=C\CCCCCCCC(=O)OCC(COC(=O)CCC(OCCCCCCCC)OCCCCCCCC)COC(=O)C1CCN(C)C1]

In an embodiment, the cation-ionizable lipid comprises, consists of, consists essentially of, or is RV84 having the following structure:

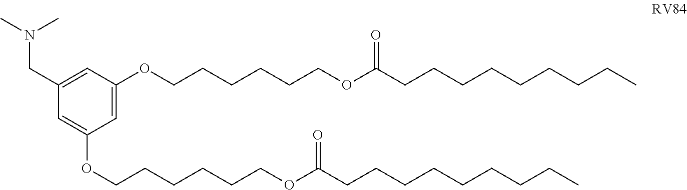 [SMILES: CCCCCCCCCC(=O)OCCCCCCOc1cc(CN(C)C)cc(OCCCCCCOC(=O)CCCCCCCCC)c1]

In an embodiment, the cation-ionizable lipid comprises, consists of, consists essentially of, or is RV85 having the following structure:

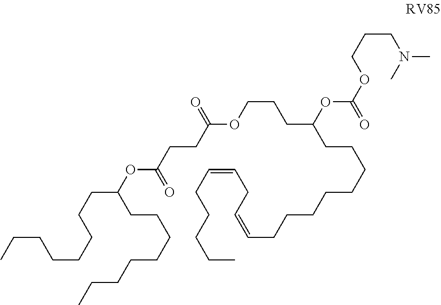 [SMILES: CCCCC/C=C\C/C=C\CCCCCCCCC(CCCOC(=O)CCC(=O)OC(CCCCCCCC)CCCCCCCC)OC(=O)OCCCN(C)C]

In an embodiment, the cation-ionizable lipid comprises, consists of, consists essentially of, or is RV86 having the following structure:

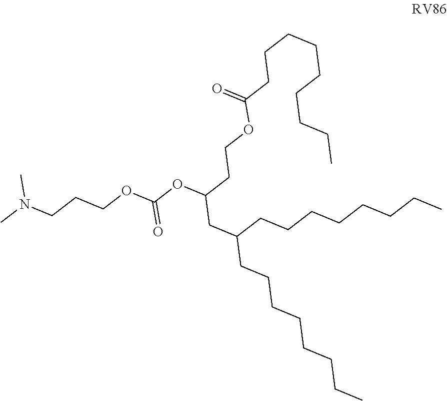 [SMILES: CCCCCCCCCC(=O)OCCC(CC(CCCCCCCC)CCCCCCCC)OC(=O)OCCCN(C)C]

In an embodiment, the cation-ionizable lipid comprises, consists of, consists essentially of, or is RV88 having the following structure:

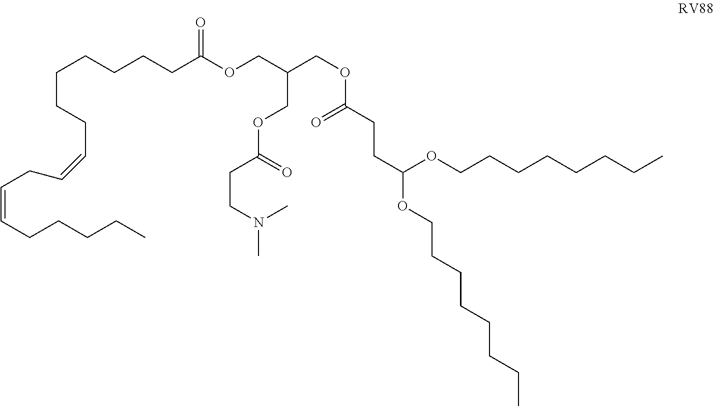 [SMILES: CCCCC/C=C\C/C=C\CCCCCCCC(=O)OCC(COC(=O)CCC(OCCCCCCCC)OCCCCCCCC)COC(=O)CCN(C)C]

In an embodiment, the cation-ionizable lipid comprises, consists of, consists essentially of, or is RV91 having the following structure:

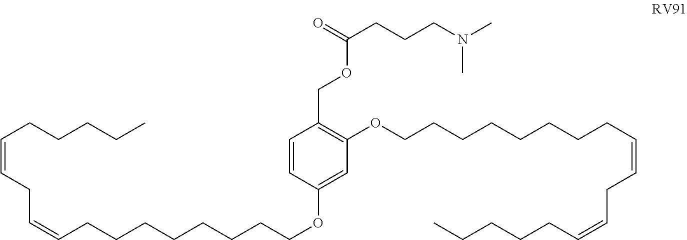 [SMILES: CCCCC/C=C\C/C=C\CCCCCCCCOc1ccc(COC(=O)CCCN(C)C)c(OCCCCCCCC/C=C\C/C=C\CCCCC)c1]

In an embodiment, the cation-ionizable lipid comprises, consists of, consists essentially of, or is RV92 having the following structure:

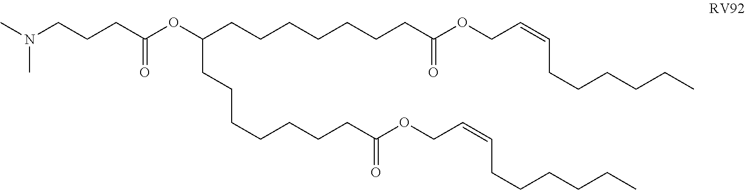 [SMILES: CCCCCC/C=C\COC(=O)CCCCCCCC(CCCCCCCC(=O)OC/C=C\CCCCCC)OC(=O)CCCN(C)C]

In an embodiment, the cation-ionizable lipid comprises, consists of, consists essentially of, or is RV93 having the following structure:

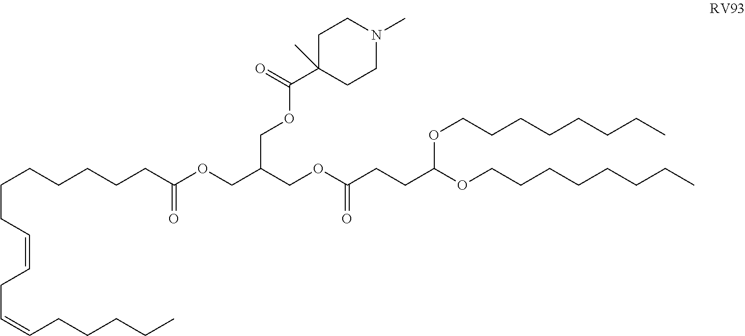 [SMILES: CCCCC/C=C\C/C=C\CCCCCCCC(=O)OCC(COC(=O)CCC(OCCCCCCCC)OCCCCCCCC)COC(=O)C1(C)CCN(C)CC1]

In an embodiment, the cation-ionizable lipid comprises, consists of, consists essentially of, or is 2-(5-((4-((1,4-dimethylpiperidine-4-carbonyl)oxy)hexadecyl)oxy)-5-oxopentyl)propane-1,3-diyl dioctanoate (RV94), having the following structure:

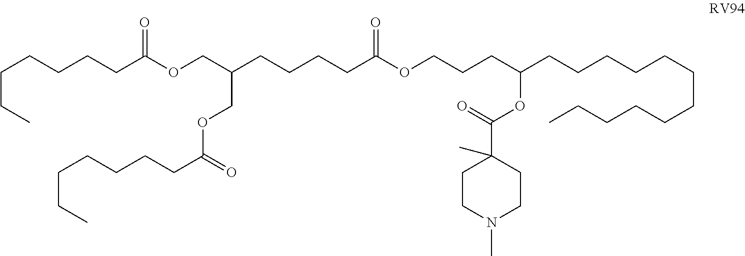 [SMILES: CCCCCCCCCCCCC(CCCOC(=O)CCCCC(COC(=O)CCCCCCC)COC(=O)CCCCCCC)OC(=O)C1(C)CCN(C)CC1]

In an embodiment, the cation-ionizable lipid comprises, consists of, consists essentially of, or is RV95 having the following structure:

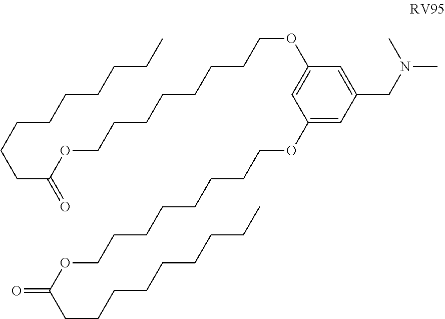 [SMILES: CCCCCCCCCC(=O)OCCCCCCCCOc1cc(CN(C)C)cc(OCCCCCCCCOC(=O)CCCCCCCCC)c1]

In an embodiment, the cation-ionizable lipid comprises, consists of, consists essentially of, or is RV96 having the following structure:

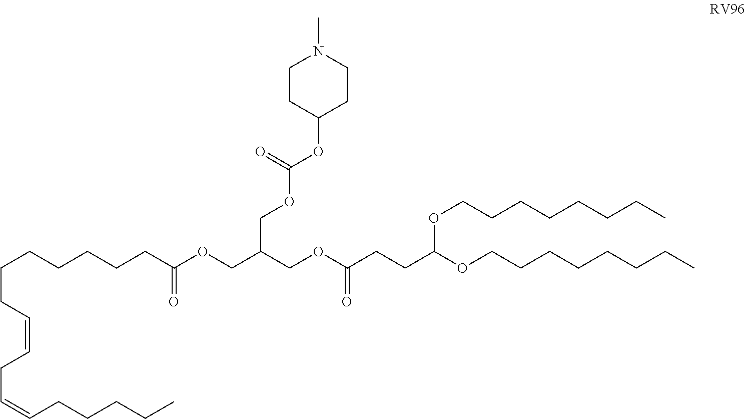 [SMILES: CCCCC/C=C\C/C=C\CCCCCCCC(=O)OCC(COC(=O)CCC(OCCCCCCCC)OCCCCCCCC)COC(=O)OC1CCN(C)CC1]

In an embodiment, the cation-ionizable lipid comprises, consists of, consists essentially of, or is RV97 having the following structure:

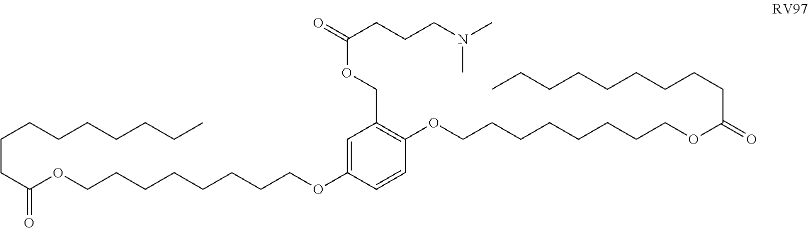 [SMILES: CCCCCCCCCC(=O)OCCCCCCCCOc1ccc(OCCCCCCCCOC(=O)CCCCCCCCC)c(COC(=O)CCCN(C)C)c1]

In an embodiment, the cation-ionizable lipid comprises, consists of, consists essentially of, or is RV99 having the following structure:

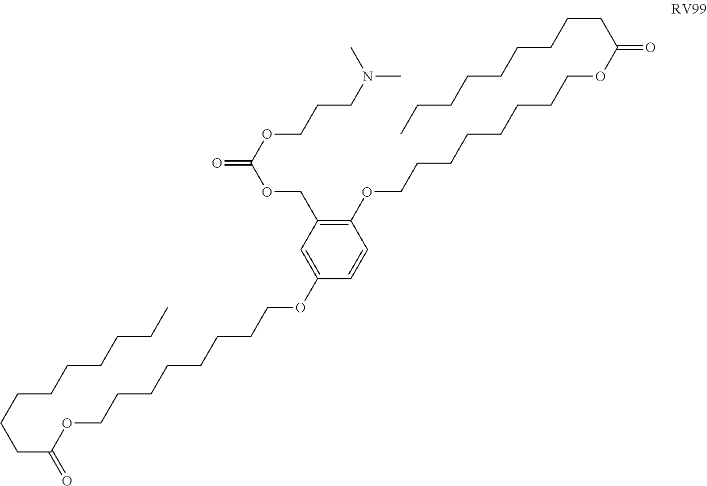 [SMILES: CCCCCCCCCC(=O)OCCCCCCCCOc1ccc(OCCCCCCCCOC(=O)CCCCCCCCC)c(COC(=O)OCCCN(C)C)c1]

In an embodiment, the cation-ionizable lipid comprises, consists of, consists essentially of, or is RV101 having the following structure:

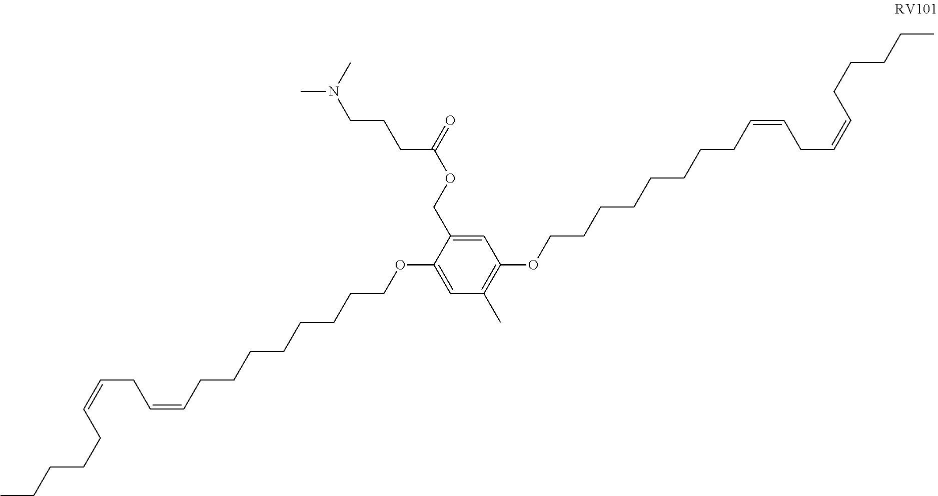 [SMILES: CCCCC/C=C\C/C=C\CCCCCCCCOc1cc(COC(=O)CCCN(C)C)c(OCCCCCCCC/C=C\C/C=C\CCCCC)cc1C]

In some embodiments, the cation-ionizable lipid comprise, consist of, consist essentially of: or are a lipid having the structure of Formula III:

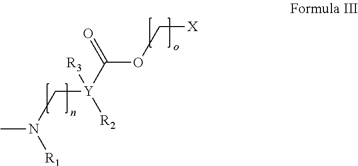

wherein n=an integer from 1 to 3 and
(i) R₁ is CH₃, R₂ and R₃ are both H, and Y is C; or
(ii) R₁ and R₂ are collectively CH₂—CH₂ and together with the nitrogen form a five-, six-, or seven-membered heterocycloalkyl, R₃ is CH₃, and Y is C; or
(iii) R₁ is CH₃, R₂ and R₃ are both absent, and Y is O;
wherein o is 0 or 1;
wherein X is:
(i)

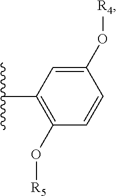

wherein R₄ and R₅ are independently a C₁₀₋₂₀ hydrocarbon chain having one or two cis alkene groups at either or both of the omega 6 and 9 positions; or
(ii) —CH(—R₆)—R₇, wherein
(1) R₆ is —(CH₂)ₚ—O—C(O)—R₈ or —Cₚ—R₃;
(2) R₇ is —(CH₂)ₚ—O—C(O)—R₈′ or —Cₚ—R₈′,
(3) p and p′ are independently 0, 1, 2, 3 or 4; and
(4) R₈ and R_{8′} are independently a
(A) —C₈₋₂₀ hydrocarbon chain having one or two cis alkene groups at either or both of the omega 6 and 9 positions;
(B) —C₁₋₃—C(—O—C₆₋₁₂)—O—C₆₋₁₂ saturated or unsaturated hydrocarbon chain;
(C) —C₆₋₁₆ saturated hydrocarbon chain;
(D) —C(—C₆₋₁₆)—C₆₋₁₆ saturated or unsaturated hydrocarbon chain;
(E) —C[—C—O—C(O)—C₄₋₁₂]—C—O—C(O)—C₄₋₁₂ saturated or unsaturated hydrocarbon chain; and
(F) —C₆₋₁₆ saturated or unsaturated hydrocarbon chain.

In an embodiment, R₁ is CH₃, R₂ and R₃ are both H, and Y is C. In some embodiments, R₁ and R₂ are collectively CH₂—CH₂ and together with the nitrogen form a five-, six-, or seven-membered heterocycloalkyl, R₃ is CH₃, and Y is C. In some embodiments, R₁ is CH₃, R₂ and R3 are both absent, and Y is O.

In an embodiment, X is

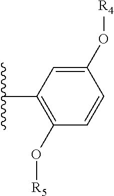

wherein R₄ and R₅ are independently a C₁₀₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or 10 unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —Cs-16 saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₈)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₃ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇—Cₚ—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₆₋₁₆ saturated hydrocarbon chain; and R₈′ is a —C(—C₆0.16)—C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —Cs-16 saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is Cₚ—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₈)—R₇, R₆ is —(CH₂)ₚ—O—C(O)—R₈, R₇ is —Cₚ—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —Cₚ—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₃ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆16 saturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C(—C₆0.16)—C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₈ is a —C₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₈)—R₇, R₈ is —Cₚ—R₃, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —C_{p′}—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₈)—R₇, R₆ is —Cₚ—R₃, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₈)—R₇, R₈ is —Cₚ—R₃, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₈)—R₇, R₈ is —Cₚ—R₃, R₇—C_{p′}—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₈)—R₇, R₈ is —Cₚ—R₃, R₇—C_{p′}—R₈′, p and p′ are independently 0, 1, 2, 3 or 4; and R₃ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —Cₚ—R₃, R₇—C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₃, R₇ is —C_{p′}—R₃″, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —Cₚ—R₃, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In some embodiments, the cation-ionizable lipid comprises a cationic lipid from WO2012/006376, WO2012/030901, WO2012/031046, WO2012/031043, WO2012/006378, WO2011/076807, WO2013/033563, WO2013/006825, WO2014/136086, WO2015/095340, WO2015/095346, WO2016/037053, WO2017/075531, WO2018/081480, WO2015/074085, WO2018/1703322, U.S. Patent Application Publication Nos.: 20220081392, 20220072155, 20220040285, 20210395188, 20210251898, 20210128488, 20210122703, 20210122702, 20210107861, 20200283372, 20200172472, 20200163878, 20200121809, 20200046838, 20190359556, 20190314524, 20190274968, 20190270697, 20190022247, 20180185516, 20170283367, 20170157268, 20170119904, 20160376224, 20160317676, or 20150376115, U.S. Application Nos. 61/905,724 or 15/614,499, or U.S. Pat. Nos. 8,802,863, 9,458,090, 9,593,077, 9,567,296, 9,604,908, 9,643,916, 9,669,097, 9,670,487, 9,737,619, 9,738,593, 9,725,720, 9,796,977,10,106,490, 10,166,298, 10,221,127, 10,723,692, 11,040,112, 11,168,051, 11,246,933, or 11,285,222 (called ionizable lipids therein).

In some embodiments, the cation-ionizable lipid comprises a first group and two biodegradable hydrophobic tails. In some embodiments, the first group comprises a central moiety and a head group, wherein the first group is capable of being positively charged. In some embodiments, the central moiety is directly bonded to each of the two biodegradable groups. In some embodiments, the central moiety is directly bonded to the head group. In some embodiments, the central moiety is selected from a central carbon atom, a central nitrogen atom, a central heteroaryl group, and a central heterocyclic group. In some embodiments, one of the two biodegradable hydrophobic tails, or each of the two biodegradable hydrophobic tails, has the formula of: -(a C₁-C₁₂ alkyl, a C₁-C₁₂ alkylene, or a C₁-C₁₂ alkenylene)-(the biodegradable group)-(a C₆-C₂₄ alkyl, a C₆-C₂₄ alkylene, a C₇-C₂₃ alkyl, a C₇-C₂₃ alkylene, a C₈-C₂₂ alkyl, a C₈-C₂₂ alkylene, a C₉-C₂₁ alkyl, a C₉-C₂₁ alkylene, a C₁₀-C₂₀ alkyl, a C₁₀-C₂₀ alkylene, a C₁₁-C₁₉ alkyl, a C₁₁-C₁₉ alkylene, a C₁₂-C₁₈ alkyl, a C₁₂-C₁₈ alkylene, a C₁₃-C₁₇ alkyl, a C₁₃-C₁₇ alkylene). In some embodiments, the each biodegradable group in the two biodegradable hydrophobic tails is independently selected from: —O(C═O)—, —(C═O)O—, —C(═O)—, —O—, —S(O)x-, —S—S—, —C(═O)S—, SC(═O)—, —NR^{a}C(═O)—, —C(═O)NR^{a}—, NR^{a}C(═O)NR^{a}—, —OC(═O)NR^{a}—, or —NR^{a}C(═O)O—; wherein X is 0, 1, or 2, and wherein R^{a} is a hydrogen or a C₁-C₁₂ alkyl. In some embodiments, in one of the two biodegradable tails, or each of the two biodegradable tails: 1) has a terminal hydrophobic chain, which is a branched alkyl group, and a terminus, 2) the branching of the branched alkyl group has an alpha-position relative to the biodegradable group, 3) 6 to 12 carbon atoms of the biodegradable hydrophobic tail separate the terminus from the biodegradable group.

In some embodiments, the cation-ionizable lipid comprises bis(2-methacryloyl)oxyethyl disulfide (DSDMA, CAS No. 36837-97-5), N,N-dioleyl-N,N-dimethylammonium chloride (DODAC), N,N-distearyl-N,N-dimethylammonium bromide (DDAB), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N, N-dimethylaminopropane (DLenDMA), 1,2-di-y-linolenyloxy-N,N-dimethylaminopropane (y-DLenDMA), 98N12-5,1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)b-utanoate (MC3), ALNY-100 ((3aR,5s,6aS)—N,N-dimethyl-2,2-di((9Z,12Z)-octadeca-9,12-dienyl)tetrahydr-o-3aH-cyclopenta[d][1,3]dioxol-5-amine)), 1,1′-(2-(4-(2-((2-(bis(2-hydroxydodecyl)amino)ethyl)(2-hydroxydodecyl)ami-no)ethyl)piperazin-1-yl)ethylazanediyl)didodecan-2-ol (C₁₂-200), 2,2-dilinoleyl-4-(2-dimethylaminoethyl)[1,3]-dioxolane (DLin-K—C₂-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N1,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethy-Ipropan-1-amine (MC3 Ether), 4-((6Z,9Z,28Z,31Z)-heptatriaconta-6,9,28,31-tetraen-19-yloxy)-N,N-dimethy-Ibutan-1-amine (MC4 Ether), LIPOFECTIN@(commercially available cationic liposomes comprising DOTMA and 1,2-dioleoyl-sn-3phosphoethanolamine (DOPE), from GIBCO/BRL, Grand Island, N.Y.); LIPOFECTAMINE@(commercially available cationic liposomes comprising N-(1-(2,3dioleyloxy)propyl)-N-(2-(sperminecarboxamido)ethyl)-N,N-dimethyl-ammonium trifluoroacetate (DOSPA), (DOPE), from GIBCO/BRL); or TRANSFECTAM@(commercially available cationic lipids comprising dioctadecylamidoglycyl carboxyspermine (DOGS) in ethanol from Promega Corp., Madison, Wis.) or any combination of any of the foregoing. Further suitable cationic include those described in international patent publications WO2010/053572 (and particularly, CI 2-200 described at paragraph [00225]) and WO2012/170930, both of which are incorporated herein by reference, HGT4003, HGT5000, HGTS001, HGT5001, HGT5002 (see US Patent Application Publication No. 20150140070A1).

Representative cation-ionizable lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl[1,3]-dioxolane (DLin-K-DMA), 2,2-dilinoleyl-4-(2-dimethylaminoethyl)[1,3]-dioxolane (DLin-KC2-DMA); dilinoleyl-methyl-4-dimethylaminobutyrate (DLin-MC3-DMA); MC3 (US Patent Publication No. 20100324120).

Various amphiphilic lipids can form bilayers in an aqueous environment to encapsulate a RNA-containing aqueous core as a LNP. These lipids can have an anionic, cationic, or zwitterionic hydrophilic head group. Some phospholipids are anionic whereas other are zwitterionic and others are cationic. Suitable classes of phospholipid include, but are not limited to, phosphatidylethanolamines, phosphatidylcholines, phosphatidylserines, and phosphatidyl-glycerols, and some useful phospholipids are listed in Table 1. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), and 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are 1,2-dipalmitoyl-sn-glycero-3-phosphocholine (DPPC), 1,2-dioleoyl-sn-glycero-3-phosphocholine (DOPC), and dodecylphosphocholine. The lipids can be saturated or unsaturated. The use of at least one unsaturated lipid for preparing liposomes is preferred. If an unsaturated lipid has two tails, both tails can be unsaturated, or it can have one saturated tail and one unsaturated tail.

Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

### Polyethylene Glycol-Conjugated Lipids

In some embodiments, the LNP comprises a polyethylene glycol-conjugated (PEG-conjugated) lipid. In some embodiments, the PEG-conjugated lipid comprises a polyethylene glycol (PEG) having various lengths and molecular weights.

In some embodiments, the PEGs in the PEG-conjugated lipids have a median molecular weight of: 0.5 kDa, 0.6 kDa, 0.7 kDa, 0.8 kDa, 0.9 kDa, 1.0 kDa, 1.1 kDa, 1.2 kDa, 1.3 kDa, 1.4 kDa, 1.5 kDa, 1.6 kDa, 1.7 kDa, 1.8 kDa, 1.9 kDa, 2.0 kDa, 2.1 kDa, 2.2 kDa, 2.3 kDa, 2.4 kDa, 2.5 kDa, 2.6 kDa, 2.7 kDa, 2.8 kDa, 2.9 kDa, 3.0 kDa, 3.1 kDa, 3.2 kDa, 3.3 kDa, 3.4 kDa, 3.5 kDa, 3.6 kDa, 3.7 kDa, 3.8 kDa, 3.9 kDa, 4.0 kDa, 4.1 kDa, 4.2 kDa, 4.3 kDa, 4.4 kDa, 4.5 kDa, 4.6 kDa, 4.7 kDa, 4.8 kDa, 4.9 kDa, 5.0 kDa, 5.1 kDa, 5.2 kDa, 5.3 kDa, 5.4 kDa, 5.5 kDa, 5.6 kDa, 5.7 kDa, 5.8 kDa, 5.9 kDa, or 6.0 kDa.

In some embodiments, the PEG-conjugated lipid comprises 2-[(polyethylene glycol)-2000]—N,N-ditetradecylacetamide or 1,2-dimyristoyl-rac-glycero-3-methoxypolyethylene glycol-2000. In 2-[(polyethylene glycol)-2000]—N,N-ditetradecylacetamide or 1,2-dimyristoyl-rac-glycero-3-methoxypolyethylene glycol-2000, the “2000” represents the median molecular weight in Daltons of the PEG. In some embodiments, the PEG-conjugated lipid comprises 1,2-dimyristoyl-sn-glycero-2-phosphoethanolamine-N-[methoxy(polyethylene glycol)]. In some embodiments, the PEG-conjugated lipid comprises 1,2-dimyristoyl-rac-glycerol-3-methoxypolyethylene glycol.

### Second Lipids

In some embodiments, the LNP further comprises a second lipid, which comprises an anionic lipid, a neutral lipid, or a zwitterionic lipid. In some embodiments, the neutral lipid comprises a neutral zwitterionic lipid. In some embodiments, the anionic lipid, a neutral lipid, or the zwitterionic lipid comprises a phospho-group (i.e. is a phospholipid), a choline, or a sphingolipid.

In some embodiments, the second lipid comprises 1,2-diheptadecanoyl-sn-glycero-3-phosphoethanolamine (17:0 PE), 1,2-dihexanoyl-sn-glycero-3-phosphoethanolamine (06:0 PE), 1,2-dioctanoyl-sn-glycero-3-phosphoethanolamine (08:0 PE), 1,2-didecanoyl-sn-glycero-3-phosphoethanolamine (10:0 PE), 1,2-dilauroyl-sn-glycero-3-phosphoethanolamine (12:0 PE), 1,2-dipentadecanoyl-sn-glycero-3-phosphoethanolamine (15:0 PE), 1,2-dipalmitoyl-sn-glycero-3-phosphoethanolamine (16:0 PE), 1,2-distearoyl-sn-glycero-3-phosphoethanolamine (18:0 PE), 1,2-dimyristoyl-sn-glycero-3-phosphoethanolamine (14:0 PE), 1,2-dipalmitoleoyl-sn-glycero-3-phosphoethanolamine (16:1 PE), 1,2-dioleoyl-sn-glycero-3-phosphoethanolamine (DOPE), 1,2-dielaidoyl-sn-glycero-3-phosphoethanolamine (18:1 (A9-Trans) PE), 1,2-dilinoleoyl-sn-glycero-3-phosphoethanolamine (18:2 PE), 1,2-dilinolenoyl-sn-glycero-3-phosphoethanolamine (18:3 PE), 1,2-didocosahexaenoyl-sn-glycero-3-phosphoethanolamine (22:6 PE), 1,2-diarachidonoyl-sn-glycero-3-phosphoethanolamine (20:4 PE), 1-pentadecanoyl-2-oleoyl-sn-glycero-3-phosphoethanolamine (15:0-18:1 PE), 1-palmitoyl-2-linoleoyl-sn-glycero-3-phosphoethanolamine (16:0-18:2 PE), 1-palmitoyl-2-oleoyl-sn-glycero-3-phosphoethanolamine (16:0-18:1 PE), 1-stearoyl-2-oleoyl-sn-glycero-3-phosphoethanolamine (18:0-18:1 PE), 1-palmitoyl-2-arachidonoyl-sn-glycero-3-phosphoethanolamine (16:0-20:4 PE), 1-palmitoyl-2-docosahexaenoyl-sn-glycero-3-phosphoethanolamine (16:0-22:6 PE), 1-stearoyl-2-linoleoyl-sn-glycero-3-phosphoethanolamine (18:0-18:2 PE), 1-stearoyl-2-arachidonoyl-sn-glycero-3-phosphoethanolamine (18:0-20:4 PE), 1-stearoyl-2-docosahexaenoyl-sn-glycero-3-phosphoethanolamine (18:0-22:6 PE), 1-oleoyl-2-hydroxy-sn-glycero-3-phosphoethanolamine (18:1 Lyso PE), 1-hydroxy-2-oleoyl-sn-glycero-3-phosphoethanolamine (2-18:1 Lyso PE), 1-palmitoyl-2-hydroxy-sn-glycero-3-phosphoethanolamine (16:0 Lyso PE), 1-tridecanoyl-sn-glycero-3-phosphoethanolamine (13:0 Lyso PE), 1-(10Z-heptadecenoyl)-sn-glycero-3-phosphoethanolamine (17:1 Lyso PE), 1-stearoyl-2-hydroxy-sn-glycero-3-phosphoethanolamine (18:0 Lyso PE), 1-myristoyl-2-hydroxy-sn-glycero-3-phosphoethanolamine (14:0 Lyso PE), L-α-phosphatidylethanolamine, 1,2-dibutyryl-sn-glycero-3-phosphocholine (04:0 PC), 1,2-dihexanoyl-sn-glycero-3-phosphocholine (DHPC), 1,2-diheptanoyl-sn-glycero-3-phosphocholine (7:0 PC), 1,2-dioctanoyl-sn-glycero-3-phosphocholine (8:0 PC), 1,2-dinonanoyl-sn-glycero-3-phosphocholine (9:0 PC), 1,2-didecanoyl-sn-glycero-3-phosphocholine (10:0 PC), 1,2-diundecanoyl-sn-glycero-3-phosphocholine (11:0 PC), 1,3-dipalmitoyl-rac-glycero-2-phosphocholine (16:0 2—PC), 1,2-dilauroyl-sn-glycero-3-phosphocholine (DLPC), 1,2-ditridecanoyl-sn-glycero-3-phosphocholine (13:0 PC), 1,2-dimyristoyl-sn-glycero-3-phosphocholine (DMPC), 1,2-dipentadecanoyl-sn-glycero-3-phosphocholine (15:0 PC), 1,2-dipalmitoyl-sn-glycero-3-phosphocholine (DPPC), 1,2-diheptadecanoyl-sn-glycero-3-phosphocholine (17:0 PC), 1,2-distearoyl-sn-glycero-3-phosphocholine (DSPC), 1,2-distearoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1,2-divaccenoyl-sn-glycero-3-phosphocholine (18:1 (11-cis) PC), 1,2-di[(8Z)octadecenoyl]-sn-glycero-3-phosphocholine (18:1 (8-cis) PC), 1,2-dimyristoleoyl-sn-glycero-3-phosphocholine (14:1 (A9-cis) PC), 1,2-dipetroselenoyl-sn-glycero-3-phosphocholine (18:1 (A6-cis) PC), 1,2-dioleoyl-sn-glycero-3-phosphocholine (DOPC), 1,2-dielaidoyl-sn-glycero-3-phosphocholine (18:1 (A9-trans) PC), 1,2-diheptadecanoyl-sn-glycero-3-phospho-(1′-rac-glycerol), 1,2-dihexanoyl-sn-glycero-3-phospho-(1′-rac-glycerol), 1,2-dioctanoyl-sn-glycero-3-phospho-(1′-rac-glycerol), 1,2-didecanoyl-sn-glycero-3-phospho-(1′-rac-glycerol), 1,2-dilauroyl-sn-glycero-3-phospho-(1′-rac-glycerol), 1,2-dimyristoyl-sn-glycero-3-phospho-(1′-rac-glycerol), 1,2-dipentadecanoyl-sn-glycero-3-phospho-(1′-rac-glycerol), 1,2-dipalmitoyl-sn-glycero-3-phospho-(1′-rac-glycerol), 1,2-distearoyl-sn-glycero-3-phospho-(1′-rac-glycerol), 1,2-dioleoyl-sn-glycero-3-phospho-(1′-rac-glycerol), 1,2-dielaidoyl-sn-glycero-3-phospho-(1′-rac-glycerol), 1,2-dilinoleoyl-sn-glycero-3-phospho-(1′-rac-glycerol), 1,2-dilinolenoyl-sn-glycero-3-phospho-(1′-rac-glycerol), 1,2-diarachidonoyl-sn-glycero-3-[phospho-rac-(1-glycerol)], 1,2-didocosahexaenoyl-sn-glycero-3-[phospho-rac-(1-glycerol)], 1,2-dilinoleoyl-sn-glycero-3-phosphocholine (DLPC), 1,2-dilinolenoyl-sn-glycero-3-phosphocholine (18:3 (cis) PC), 1,2-dieicosenoyl-sn-glycero-3-phosphocholine (20:1 (cis) PC), 1,2-diarachidonoyl-sn-glycero-3-phosphocholine (20:4 (cis) PC), 1,2-dierucoyl-sn-glycero-3-phosphocholine (22:1 (cis) PC), 1,2-dinervonoyl-sn-glycero-3-phosphocholine (24:1 (cis) PC), 1,2-didocosahexaenoyl-sn-glycero-3-phosphocholine (22:6 (cis) PC), 1-pentadecanoyl-2-oleoyl-sn-glycero-3-phosphocholine (15:0-18:1 PC), 1-myristoyl-2-palmitoyl-sn-glycero-3-phosphocholine (14:0-16:0 PC), 1-myristoyl-2-stearoyl-sn-glycero-3-phosphocholine (14:0-18:0 PC), 1-palmitoyl-2-myristoyl-sn-glycero-3-phosphocholine (16:0-14:0 PC), 1-palmitoyl-2-stearoyl-sn-glycero-3-phosphocholine (16:0-18:0 PC), 1-palmitoyl-2-oleoyl-glycero-3-phosphocholine (POPC), 1-palmitoyl-2-linoleoyl-sn-glycero-3-phosphocholine (16:0-18:2 PC), 1-palmitoyl-2-arachidonoyl-sn-glycero-3-phosphocholine (16:0-20:4 PC), 1-palmitoyl-2-docosahexaenoyl-sn-glycero-3-phosphocholine (16:0-22:6 PC), 1-stearoyl-2-myristoyl-sn-glycero-3-phosphocholine (18:0-14:0 PC), 1-stearoyl-2-palmitoyl-sn-glycero-3-phosphocholine (18:0-16:0 PC), 1-stearoyl-2-oleoyl-sn-glycero-3-phosphocholine (18:0-18:1 PC), 1-stearoyl-2-linoleoyl-sn-glycero-3-phosphocholine (18:0-18:2 PC), 1-stearoyl-2-arachidonoyl-sn-glycero-3-phosphocholine (18:0-20:4 PC), 1-stearoyl-2-docosahexaenoyl-sn-glycero-3-phosphocholine (18:0-22:6 PC), 1-oleoyl-2-myristoyl-sn-glycero-3-phosphocholine (18:1-14:0 PC), 1-oleoyl-2-palmitoyl-sn-glycero-3-phosphocholine (18:1-16:0 PC), 1-oleoyl-2-stearoyl-sn-glycero-3-phosphocholine (18:1-18:0 PC), 1-(8Z-octadecenoyl)-2-palmitoyl-sn-glycero-3-phosphocholine (18:1(n10)-16:0 PC), 1-palmitoyl-2-acetyl-sn-glycero-3-phosphocholine (16:0-02:0 PC), 1-palmitoyl-2-[12′-(palmitoyloxy)octadecanoyl]-sn-glycero-3-phosphoholine (16:0-(12-PAHSA) PC), 1-oleoyl-2-hydroxy-sn-glycero-3-phosphocholine, 1-hexanoyl-2-hydroxy-sn-glycero-3-phosphocholine, 1-heptanoyl-2-hydroxy-sn-glycero-3-phosphocholine, 1-octanoyl-2-hydroxy-sn-glycero-3-phosphocholine, 1-nonanoyl-2-hydroxy-sn-glycero-3-phosphocholine, 1-decanoyl-2-hydroxy-sn-glycero-3-phosphocholine, 1-undecanoyl-2-hydroxy-sn-glycero-3-phosphocholine, 1-lauroyl-2-hydroxy-sn-glycero-3-phosphocholine, 1-tridecanoyl-2-hydroxy-sn-glycero-3-phosphocholine, 1-myristoyl-2-hydroxy-sn-glycero-3-phosphocholine, 1-pentadecanoyl-2-hydroxy-sn-glycero-3-phosphocholine, 1-hydroxy-2-palmitoyl-sn-glycero-3-phosphocholine, 1-palmitoyl-2-hydroxy-sn-glycero-3-phosphocholine, 1-heptadecanoyl-2-hydroxy-sn-glycero-3-phosphocholine, 1-(10Z-heptadecenoyl)-2-hydroxy-sn-glycero-3-phosphocholine, 1-hydroxy-2-oleoyl-sn-glycero-3-phosphocholine, 2-stearoyl-sn-glycero-3-phosphocholine, 1-stearoyl-2-hydroxy-sn-glycero-3-phosphocholine, 1-nonadecanoyl-2-hydroxy-sn-glycero-3-phosphocholine, 1-arachidoyl-2-hydroxy-sn-glycero-3-phosphocholine, 1-behenoyl-2-hydroxy-sn-glycero-3-phosphocholine, 1-lignoceroyl-2-hydroxy-sn-glycero-3-phosphocholine, 1-hexacosanoyl-2-hydroxy-sn-glycero-3-phosphocholine, 1,2-dimyristoyl-sn-glycero-phosphocholine (DMPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-didocosahexaenoyl-sn-glycero-3-phosphoethanolamine, 1,2-dioleoyl-sn-glycero-3-phospho-rac-(1-glycerol) sodium salt (DOPG), N-lignoceroyl-D-erythro-sphingosylphosphoethanolamine, or sphingomyelin.

### Sterols

In some embodiments, the lipid nanoparticles further comprise a sterol. In some embodiments, the sterol comprises cholesterol, cholesterol sulfate, desmosterol, stigmasterol, lanosterol, 7-dehydrocholesterol, dihydrolanosterol, symosterol, lathosteriol, 14-demethyl-lanosterol, 8(9)-dehydrocholesterol, 8(14)-dehydrocholesterol, 14-demethyl-14-dehydrolanosterol (FF-MAS), diosgenin, dehydroepiandrosterone sulfate (DHEA sulfate), dehydroepiandrosterone, sitosterol, lanosterol-95, 4,4-dimethyl(d6)-cholest-8(9), 14-dien-3β-ol (dihydro-FF-MAS-d6), 4,4-dimethyl(d6)-cholest-8(9)-en-3β-ol (dihydro T-MAS-d6), zymostenol, sitostanol, campestanol, camperstanol, 7-dehydrodesmosterol, pregnenolone, 4,4-dimethyl-cholest-8(9)-en-3β-ol (dihyrdro T-MAS), Δ5-avensterol, brassicasterol, dihydro FF-MAS, 24-methylene cholesterol, oxysterols, deuterated sterols, fluorinated sterols, sulfonated sterols, phosphorylated sterols, A-ring substituted sterols, cholest-5-ene-3ß,4ß-diol, 5α-cholestan-3ß-ol, 4-cholesten-3-one, cholesta-8(9),24-dien-3-one, cholesta-8(9),24-dien-3-one, 2,2,3,4,4-pentadeuterio-5a-cholestan-3ß-ol, cholesteryl phosphocholine, cholesteryl-d7 pentadecanoate, cholesteryl-d7 palmitate, B-ring substituted sterols, cholestanol, 5ß,6ß-epoxy-d7, 3ß-hydroxy-5-cholestene-7-one, 6α-hydroxy-5α-cholestane, cholestanol, 5α,6α-epoxy, cholest-5-en-3ß,7α-diol, cholest-5-en-3ß,7ß-diol, cholestanol, 5α,6α-epoxy-d7, Δ5,7-cholesterol, cholesta-5,8(9)-dien-3ß-ol, cholesta-5,8(14)-dien-3ß-ol, 7α-hydroxy-4-cholesten-3-one, zymostenol-d7, zymostenol, 7-dehydrodesmosterol, 3b,5a-dihydroxy-cholestan-6-one, D-ring substituted sterols, 3ß-hydroxy-5α-cholest-8(14)-en-15-one, 3ß-hydroxy-5α-cholestane-15-one, 5α-cholest-8(14)-ene-3ß,15α-diol, 5α-cholest-8(14)-ene-3ß,15ß-diol, lanosterol-95, 5α-7,24-cholestadiene, 14-dehydro zymostenol, ergosta-5,7,9(11),22-tetraen-3ß-ol, cholest-5-ene-3ß,25-diol, cholest-(25R)-5-ene-3ß,27-diol, 24(R/S),25-epoxycholesterol, 24(S),25-epoxycholesterol, 24(R/S),25-epoxycholesterol-d6, cholest-5-ene-3ß,22(S)-diol, cholest-5-ene-3ß,22(R)-diol, cholest-5-ene-3ß,24(S)-diol, cholest-5-ene-3ß,24(R)-diol, 27-hydroxy-4-cholesten-3-one, campestanol, N,N-dimethyl-3ß-hydroxycholenamide, 25,27-dihydroxycholesterol, N,N-dimethyl-3ß-hydroxycholenamide, 25,27-dihydroxycholesterol, 5-cholestene-3β,20α-diol, 24S,25-epoxy-5α-cholest-8(9)-en-3β-ol, 24(S/R),25-epoxylanost-8(9)-en-3β-ol, 7-keto-27-hydroxycholesterol, 7α,27-dihydroxy-4-cholesten-3-one, 7α,27-dihydroxycholesterol, 7β,27-dihydroxycholesterol, 5α,6ß-dihydroxycholestanol, 7α,25-dihydroxycholesterol, 7β,25-dihydroxycholesterol, 7α,24(S)-dihydroxycholesterol, 7α,24(S)-dihydroxy-4-cholesten-3-one, 7-keto-25-hydroxycholesterol, 7α,24S,27-trihydroxycholesterol, dihydrotestosterone, testosterone, estrone, estrogen, estradiol, corticosterone, cortisol, or 24S,27-dihydroxycholesterol.

Making the LNPs Encapsulating the mRNA

As noted above, in some embodiments, the mRNA molecules are encapsulated within the LNPs. In some embodiments, the mRNA and lipids of the LNPs can be admixed and/or purified to thereby provide said comprising or encapsulating within. In some embodiments, the mRNA and lipids of the LNP can be admixed and/or purified to thereby provide the above-noted proportions of mRNA encapsulated within the LNPs.

In some aspects, a method of obtaining a composition is provided, the composition comprising the mRNA and LNPs, wherein the mRNA are encapsulated within the LNPs in the above-noted proportions, wherein the LNPs comprise the above-noted lipids; the method comprising admixing a first solution, which comprises the recombinant RNA molecules, and a second solution, which comprises the above-noted lipids. In some embodiments, the admixing is performed by at least a T-mixer, microfluidics, or an impinging jet mixer. In some embodiments, the first solution further comprises citrate buffer (e.g. sodium citrate) or acetate buffer (e.g. sodium acetate).

In some embodiments, the second solution further comprises an organic solvent. In some embodiments, the organic solvent comprises chloroform, dichloromethane, diethylether, cyclohexane, cyclopentane, benzene, toluene, methanol, benzyl alcohol, and aliphatic alcohols (e.g. C₁ to C₈ alcohols). In some embodiments, the aliphatic alcohols comprise ethanol, propanol, isopropanol, butanol, tert-buranol, isobutanol, pentanol, benzyl alcohol, and hexanol. In some embodiments, the organic solvent comprises an alcohol solution. In some embodiments, the organic alcohol solution comprises from 70 volume % to 100 volume % ethanol.

In some embodiments, the method comprises admixing a first solution, which comprises the recombinant RNA molecules and the above-noted lipids of the LNP, and a second solution, which is an aqueous solution. In some embodiments, the RNA and lipids of the LNP are admixed in an organic solvent. In some embodiments, the organic solvent comprises chloroform, dichloromethane, diethylether, cyclohexane, cyclopentane, benzene, toluene, methanol, benzyl alcohol, and aliphatic alcohols (e.g. C₁ to C₈ alcohols). In some embodiments, the aliphatic alcohols comprise ethanol, propanol, isopropanol, butanol, tert-buranol, isobutanol, pentanol, benzyl alcohol, and hexanol. In some embodiments, the organic solvent comprises an alcohol solution. In some embodiments, the organic alcohol solution comprises from 70 volume % to 100 volume % ethanol. In some embodiments, the organic alcohol solution comprises from 70 volume % to 100 volume % ethanol and 30 volume % to 0 volume % benzyl alcohol. In some embodiments, the aqueous solution comprises a citrate buffer (e.g. sodium citrate) or an acetate buffer (e.g. sodium acetate). In some embodiments, the first and second solution are admixed at a ratio from 1:1 to 5:1, from 2:1 to 4:1, from 2.5:1 to 3.5:1, or at 3:1.

In some embodiments, the admixing of the first and second solutions (i.e. of either of the two above-noted methods) is at a pH from 4.5 to the pKa of the first lipid (e.g. the cation-ionizable lipid), thereby obtaining a first admixture. In some embodiments, the admixing of the first and second solutions is at a pH from 4.5, 4.6, 4.7, 4.8, 4.9, 5.0, 5.1, 5.2, 5.3, 5.4, 5.5, 5.6, 5.7, 5.8, 5.9, or 6.0 to the pKa of the first lipid (e.g. the cation-ionizable lipid), thereby obtaining a first admixture. In some embodiments, the method further comprises a first increasing, which is increasing the pH of the first admixture to be equal to or above the pKa of the first lipid to thereby obtain a pH-adjusted first admixture. In some embodiments, the first increasing obtains a pH-adjusted first admixture with a pH from the pKa of the first lipid (e.g. cation-ionizable lipid) to: 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, or 7.0.

In some embodiments, the first increasing or purifying comprises cross-flow filtration or tangential-flow filtration. In some embodiments, the first increasing or purifying further comprises transferring the composition comprising the LNPs and the recombinant RNA molecules into a third solution, which differs from the first solution. In some embodiments, the third solution comprises phosphate-buffered saline. In some embodiments, the transferring comprises dialysis. In some embodiments, the tangential-flow filtration comprises the use of a hollow fiber filter. In some embodiments, the hollow fiber comprises a polyethersulfone hollow fiber filter or a polysulfone hollow fiber filter.
n some embodiments, the first increasing or purifying comprises, prior to the above-noted filtrations, passing the LNP/RNA mixture through an ion exchange solid-state support. In some embodiments, the ion exchange solid-state support comprises an anion exchange column or a cation exchange column.

In some embodiments, prior to the admixing of the mRNA and the lipids of the LNPs, the lipids of the LNP admixed with an organic solvent to obtain a concentrated stock (e.g. a stock lipid/organic solvent mixture). In some embodiments, the admixing is (e.g. the stock lipid/organic solvent mixture is stirred, rocked, vortexed, sonicated, or agitated at from 25° C. to 37° C.) for at least 5 min, 6 min, 7 min, 8 min, 9 min, 10 min, 11 min, 12 min, 13 min, 14 min, 15 min, 16 min, 17 min, 18 min, 19 min, 20 min, 25 min, 30 min, 35 min, or 40 min to form a homogeneous stock lipid/organic solvent mixture. In some embodiments, the admixing is (e.g. the stock lipid/organic solvent mixture is stirred, rocked, vortexed, sonicated, or agitated at from 25° C. to 37° C.) for no more than 10 min, 11 min, 12 min, 13 min, 14 min, 15 min, 16 min, 17 min, 18 min, 19 min, 20 min, 25 min, 30 min, 35 min, 40 min, 50 min, 1 hr, 1.1 hrs, 1.2 hrs, 1.3 hrs, 1.4 hrs, or 1.5 hrs to form a homogeneous stock lipid/organic solvent mixture. Taking the above-noted embodiments into account, it is contemplated and supported that any of the above-noted amounts of time “of at least” and amounts of time “of no more than” may be combined to provide an enclosed range (i.e. the stock lipid/organic solvent mixture is stirred, rocked, vortexed, sonicated, or agitated at from 25° C. to 37° C. for from 5 min to 19 min).

### Regimens

The present invention encompasses a method of treating chronic hepatitis B infection (CHB) by administering to a human a first mRNA encoding a first hepatitis B virus antigen, in combination with a second mRNA encoding a second hepatitis B virus antigen. In one example, the first and second mRNAs are co-administered. In a first example of co-administration, the first and second mRNAs are in separate LNP formulations which are mixed into a single composition prior to administration. This could take place at the bedside immediately prior to administration. In a second example of co-administration, the first and second mRNAs are co-formulated into a single LNP. In a third example of co-administration, the first and second mRNAs are co-filled into a single vial.

The present invention, therefore, encompasses immunogenic combinations comprising a first mRNA encoding a first hepatitis B virus antigen, and a second mRNA encoding a second hepatitis B virus antigen, wherein the first and second mRNAs are in separate LNP formulations. The invention also encompasses the resultant composition formed by mixing the separate LNP formulations.

The present invention additionally encompasses immunogenic compositions comprising a first mRNA encoding a first hepatitis B virus antigen, and a second mRNA encoding a second hepatitis B virus antigen. In such compositions, the first and second mRNAs may be encapsulated by separate LNPs, or the first and second mRNAs may be formulated in the same LNP.

The present invention encompasses a method of treating chronic hepatitis B infection (CHB) by administering to a human a mRNA encoding at least one hepatitis B virus antigen, in combination with at least one recombinant hepatitis B polypeptide. The components (e.g., mRNA and recombinant hepatitis B polypeptide) may be administered sequentially in a heterologous prime-boost regimen. If a heterologous prime-boost regimen is used, the mRNA is preferably administered as a priming dose, and the at least one recombinant hepatitis B polypeptide is administered as a booster dose. In this regimen, the method comprises first administering the mRNA, then administering the recombinant hepatitis B polypeptide. In another aspect, the at least one recombinant hepatitis B polypeptide is administered as a priming dose, and the mRNA is administered as a booster dose. In this regimen, the method comprises administering the recombinant hepatitis B polypeptide, then administering the mRNA. In a further aspect, the at least one recombinant hepatitis B polypeptide is administered as a priming dose, and the mRNA is administered along with adjuvanted recombinant proteins as a booster dose. In this regimen, the method comprises administering the recombinant hepatitis B polypeptide, then administering the mRNA with recombinant proteins.

In some embodiments, the at least one hepatitis B virus polypeptide is at least one of a recombinant hepatitis B surface antigen (HBs), a recombinant hepatitis B virus core antigen (HBc), or a combination thereof. The at least one recombinant hepatitis B polypeptide can be administered with or without adjuvant. In a preferred embodiment, the mRNA is administered sequentially with adjuvanted recombinant hepatitis B polypeptides, wherein the recombinant hepatitis B polypeptides include both the hepatitis B small surface (HBs) and hepatitis B virus core (HBc) antigens. In this embodiment, the adjuvant is preferably AS01.

In such regimens, there may be multiple prime and/or booster doses. In one embodiment, there is a single prime of the mRNA, and multiple subsequent doses of the at least one recombinant hepatitis B polypeptide. For example, two doses of the recombinant hepatitis B polypeptide(s). In another embodiment, there are multiple priming doses of the mRNA, and multiple subsequent doses of the recombinant hepatitis B polypeptide(s). For example, two doses of mRNA, followed by two doses of the recombinant hepatitis B polypeptide(s). In a further embodiment, there is a single prime of the at least one recombinant hepatitis B polypeptide, and multiple subsequent doses of the mRNA. In yet a further embodiment, there are multiple priming doses of the recombinant hepatitis B polypeptide, and multiple subsequent doses of the mRNA.

In other embodiments the mRNA is administered concomitantly with the at least one recombinant hepatitis B polypeptide. Further doses of these components may be administered subsequently at a later time. In some embodiments, the mRNA is administered concomitantly with at least one recombinant hepatitis B polypeptide. The at least one recombinant hepatitis B polypeptide is at least one of a recombinant hepatitis B surface antigen (HBs), a recombinant hepatitis B virus core antigen (HBc), or a combination thereof. The recombinant HBc can be full length or truncated, preferably truncated. The at least one recombinant hepatitis B polypeptide can be administered with or without adjuvant. In one embodiment, the mRNA is administered concomitantly with adjuvanted recombinant hepatitis B polypeptides, wherein the recombinant hepatitis B polypeptides include both the hepatitis B small surface (HBs) and hepatitis B virus core (HBc) antigens. In this case, the adjuvant is preferably AS01.

In these above embodiments, the recombinant hepatitis B surface antigen (HBs) may have the amino acid sequence of SEQ ID NO: 1. In such above embodiments, the recombinant hepatitis B virus core antigen (HBc) may have the amino acid sequence of SEQ ID NO: 2 or 11. Preferably, the HBc has the amino acid sequence of SEQ ID NO: 2.

In all of these regimens, the at least one recombinant hepatitis B polypeptide may be administered with a suitable adjuvant. Suitable adjuvants are those which can enhance the immune response in subjects with chronic conditions and subverted immune competence. CHB patients are characterised by their inability to mount an efficient innate and adaptive immune response to the virus, which rends efficient vaccine development challenging. In these patients, one key function of an adjuvanted vaccine formulation should aim to direct the cell-mediated immune response towards a T Helper 1 (Th1) profile recognised to be critical for the removal of intracellular pathogens.

Examples of suitable adjuvants include but are not limited to inorganic adjuvants (e.g. inorganic metal salts such as aluminium phosphate or aluminium hydroxide), organic non-peptide adjuvants (e.g. saponins, such as QS21, or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A (MPL), such as 3-de-O-acylated monophosphoryl lipid A (3D-MPL), or muramyl peptides), synthetic adjuvants (e.g. non-ionic block copolymers, muramyl peptide analogues, or synthetic lipid A), synthetic polynucleotides adjuvants (e.g. polyarginine or polylysine) and immunostimulatory oligonucleotides containing unmethylated CpG dinucleotides (“CpG”). In particular, the adjuvant(s) may be organic non-peptide adjuvants (e.g. saponins, such as QS21, or squalene) and/or bacterial adjuvants (e.g. monophosphoryl lipid A (MPL), such as 3-de-O-acylated monophosphoryl lipid A (3D-MPL).

One suitable adjuvant is monophosphoryl lipid A (MPL), in particular 3-de-O-acylated monophosphoryl lipid A (3D-MPL). Chemically it is often supplied as a mixture of 3-de-O-acylated monophosphoryl lipid A with either 4, 5, or 6 acylated chains. It can be purified and prepared by the methods taught in GB 2122204B, which reference also discloses the preparation of diphosphoryl lipid A, and 3-O-deacylated variants thereof. Other purified and synthetic lipopolysaccharides have been described [U.S. Pat. No. 6,005,099 and EP0729473B1; Hilgers, 1986; Hilgers, 1987; and EP0549074B1].

Saponins are also suitable adjuvants [Lacaille-Dubois, 1996]. For example, the saponin Quil A (derived from the bark of the South American tree Quillaja saponaria Molina), and fractions thereof, are described in U.S. Pat. No. 5,057,540 and Kensil, 1996; and EP 0 362 279 E1. Purified fractions of Quil A are also known as immunostimulants, such as QS21 and QS17; methods of their production are disclosed in U.S. Pat. No. 5,057,540 and EP 0 362 279 E1. Use of QS21 is further described in Kensil, 1991. Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

Adjuvants such as those described above may be formulated together with carriers, such as liposomes, oil in water emulsions, and/or metallic salts (including aluminum salts such as aluminum hydroxide). For example, 3D-MPL may be formulated with aluminum hydroxide (EP 0 689 454) or oil in water emulsions (WO 95/17210); QS21 may be formulated with cholesterol containing liposomes (WO 96/33739), oil in water emulsions (WO 95/17210) or alum (WO 98/15287).

Combinations of adjuvants may be utilized in the disclosed compositions, in particular a combination of a monophosphoryl lipid A and a saponin derivative (see, e.g., WO 94/00153; WO 95/17210; WO 96/33739; WO 98/56414; WO 99/12565; WO 99/11241), more particularly the combination of QS21 and 3D-MPL as disclosed in WO 94/00153, or a composition where the QS21 is quenched in cholesterol-containing liposomes (DQ) as disclosed in WO 96/33739. A potent adjuvant formulation involving QS21, 3D-MPL & tocopherol in an oil in water emulsion is described in WO 95/17210 and is another formulation which may find use in the disclosed compositions. Thus, suitable adjuvant systems include, for example, a combination of monophosphoryl lipid A, preferably 3D-MPL, together with an aluminium salt (e.g. as described in WO00/23105). A further exemplary adjuvant comprises QS21 and/or MPL and/or CpG. QS21 may be quenched in cholesterol-containing liposomes as disclosed in WO 96/33739.

Accordingly, a suitable adjuvant for use with the at least one recombinant hepatitis B polypeptide is AS01 (sometimes referred to as “AS-01”), a liposome based adjuvant containing MPL and QS-21. The liposomes, which are the vehicles for the MPL and QS-21 immuno-enhancers, are composed of dioleoyl phosphatidylcholine (DOPC) and cholesterol in a phosphate buffered saline solution. AS01_{B-4} is a particularly preferred variant of the AS01 adjuvant, composed of immuno-enhancers QS-21 (a triterpene glycoside purified from the bark of Quillaja saponaria) and MPL (3-D Monophosphoryl lipid A), with DOPC/cholesterol liposomes, as vehicles for these immuno-enhancers, and sorbitol in a PBS solution. In particular, a single human dose of AS01_{B-4} (0.5 mL) contains 50 μg of QS-21 and 50 μg of MPL. AS01_{E-4} corresponds to a two-fold dilution of AS01_{B-4}. i.e. it contains 25 μg of QS-21 and 25 μg of MPL per human dose.

In all of these regimens, in a preferred embodiment, the mRNA encodes a hepatitis B core (HBc) polypeptide, with or without hli fusion. The HBc encoded by mRNA can be full length or truncated, preferably full length. In a preferred embodiment, the HBc encoded by mRNA is full length and fused to hli. In another preferred embodiment, the mRNA encodes a full length hepatitis B core (HBc) antigen, with or without hli fusion, and a hepatitis B surface protein (HBsAg), with or without hli fusion. Preferably, the HBsAg is hepatitis B small surface protein (HBs), with or without hli fusion. In another preferred embodiment, the hepatitis B small surface protein (HBs) is fused to hli.

The HBc encoded by mRNA, and/or the HBs encoded by mRNA, are preferably fused to hli.

The present invention also encompasses treating chronic hepatitis B infection (CHB) by administering to a human an adenoviral vector comprising a polynucleotide encoding a hepatitis B polypeptide in combination with mRNA encoding at least one hepatitis B virus antigen. The components may be administered in a heterologous prime-boost regimen. If a prime-boost regimen is used, the adenoviral vector is preferably administered as a priming dose, and the mRNA is administered as a first booster dose. In such regimens, there may be multiple prime and/or booster doses. In one embodiment, there is a single prime of the adenoviral vector (such as a replication-defective chimpanzee adenoviral (ChAd) vector), and multiple subsequent booster doses comprising mRNA and/or recombinant HBV polypeptide.

In an alternative embodiment of a heterologous prime-boost regimen, the mRNA is used as the first priming dose, and the adenoviral vector (such as a replication-defective chimpanzee adenoviral (ChAd) vector) is used as the first boosting dose.

The present invention also encompasses treating chronic hepatitis B infection (CHB) by administering to a human (i) an adenoviral vector comprising a polynucleotide encoding a hepatitis B polypeptide, and (ii) a composition comprising a recombinant hepatitis B surface antigen (HBs), a recombinant hepatitis B core antigen (HBc) and an adjuvant, in combination with mRNA encoding at least one hepatitis B virus antigen.

The invention may also comprise administering multiple subsequent doses of a mRNA. In such embodiments, the mRNA used in the priming and boosting doses is preferably identical.

### Subjects

The present invention is intended for human subjects. The subject to be treated using the method of the invention may be of any age.

The methods of the invention are suitably intended for treatment of HBV, i.e. for administration to a subject who is infected with hepatitis B virus. The subject can be infected with hepatitis B virus alone, or with hepatitis B and hepatitis D virus.

### Formulation and Administration

The mRNA may be administered via various suitable routes, including parenteral, such as intramuscular or subcutaneous administration. Suitably the mRNA is administered intramuscularly.

The mRNA may be provided in liquid or dry (e.g. lyophilised) form. The preferred form will depend on factors such as the precise nature of the mRNA, e.g. if the mRNA is amenable to drying, or other components which may be present.

Preferably, the mRNA is provided in liquid form.

A composition comprising mRNA intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject.

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Osmolality may be measured according to techniques known in the art, such as by the use of a commercially available osmometer, for example the Advanced® Model 2020 available from Advanced Instruments Inc. (USA).

Liquids used for reconstitution will be substantially aqueous, such as water for injection, phosphate buffered saline and the like. As mentioned above, the requirement for buffer and/or tonicity modifying agents will depend on the on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄.

The mRNA may be provided in various physical containers such as vials or pre-filled syringes.

In some embodiments the mRNA is provided in the form of a single dose. In other embodiments the mRNA is provided in multidose form such containing 2, 5 or 10 doses.

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul.

Stabilisers may be present. Stabilisers may be of particular relevance where multidose containers are provided as doses of the final formulation(s) may be administered to subjects over a period of time.

Formulations are preferably sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the mRNA may therefore be part of a multi-dose administration regime. For example, the mRNA may be provided as a priming dose in a multidose regime, for example a two-, three-, four-, five-, six-, seven-, eight-, nine-, ten-, eleven-, twelve-dose or more regime, in particular a six-dose regime administered over six months. The mRNA may be provided as a boosting dose in a multidose regime, especially a two-, three-, four-, five-, six-, seven-, eight-, nine-, ten-, eleven-, twelve-dose or more regime, such as a six-dose regime administered over six months. In one particular example, the mRNA is administered as a four-dose regimen.

Priming and boosting doses may be homologous or heterologous. Consequently, the mRNA may be provided as a priming dose and boosting dose(s) in a homologous multidose regime, especially a two-, three-, four-, five-, six-, seven-, eight-, nine-, ten-, eleven-, twelve-dose or more regime, in particular a six-dose regime administered over six months. In one example, the mRNA is administered as a four-dose regimen. Alternatively, the mRNA may be provided as a priming dose or boosting dose in a heterologous multidose regime, especially a two-, three-, four-, five-, six-, seven-, eight-, nine-, ten-, eleven-, twelve-dose or more regime, in particular a six-dose regime administered over six months, and the boosting dose(s) may be different (e.g. mRNA; or an alternative antigen presentation such as protein or virally vectored antigen—with or without adjuvant, such as AS01 or squalene emulsion adjuvant). In one example, the mRNA is administered as a four-dose regimen.

The time between doses may be two weeks to six months, such as three weeks to three months. Preferably, two doses—one prime dose and one boost dose—are administered concomitantly every month for six months. Periodic longer-term booster doses may be also be provided, such as every 2 to 10 years.

The present invention encompasses immunogenic combinations or compositions comprising a first mRNA encoding a first hepatitis B virus antigen, and a second mRNA encoding a second hepatitis B virus antigen. In one example, the immunogenic combination comprises a first mRNA encoding a first hepatitis B virus antigen, and a second mRNA encoding a second hepatitis B virus antigen, wherein the first and second mRNAs are in separate LNP formulations.

In another example, the immunogenic composition comprises a first mRNA encoding a first hepatitis B virus antigen, and a second mRNA encoding a second hepatitis B virus antigen. The first and second mRNAs may be encapsulated by separate LNPs, or the first and second mRNAs may be co-formulated in the same LNP.

In some examples, the combination or composition comprises the first and second mRNAs in equal quantities by weight. However, in other examples, the combination or composition may contain unequal quantities of the first and second mRNAs by weight. In some embodiments, the first hepatitis B virus antigen is HBc, and the second hepatitis B virus antigen is HBs. In such embodiments, the combinations or compositions contain more first mRNA than second mRNA by weight.

In examples of formulations containing both HBc and HBs mRNA, the composition contains equal quantities (by weight) of the HBc and HBs mRNA. However, in one example, the composition contains more mRNA encoding HBc (“HBc-mRNA”) than mRNA encoding HBs (HBs-mRNA) by weight. In an example, the composition contains between 1.25 and 2 times the amount of HBc-mRNA when compared to the HBs-mRNA, for example, between 1.5 and 2 times the amount of mRNA. In one particular example, the composition contains 1.5 times as much HBc-mRNA as HBs-mRNA by weight.

In another example there is immunogenic combination comprising:
A first composition comprising an mRNA encoding a hepatitis B virus core antigen (HBc) encapsulated in a lipid nanoparticle (LNP), and an mRNA encoding a hepatitis B small surface protein (HBs) encapsulated in a lipid nanoparticle (LNP); and
A second composition comprising recombinant hepatitis B core protein (HBc) and recombinant hepatitis B small surface protein (HBs) and AS01.

The combination may be for use in a method of treating chronic hepatitis B (CHB) by sequential or concomitant administration of the first and second compositions. The first composition may comprise the mRNAs encoding HBc and HBs co-formulated into a single LNP. Alternatively, the mRNAs encoding HBc and HBs may be formulated into separate LNPs, and these LNPs co-filled into a single vial.

### Definitions

Unless defined otherwise, all technical and scientific terms used herein have the same meanings as commonly understood by one of ordinary skill in the art.

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given figure, such as x±5% of the given figure.

As used herein, the singular forms “a,” “an” and “the” include plural references unless the content clearly dictates otherwise.

Unless specifically stated, a process comprising a step of mixing two or more components does not require any specific order of mixing. Thus components can be mixed in any order. Where there are three components then two components can be combined with each other, and then the combination may be combined with the third component, etc.

The terms “protein”, “polypeptide”, “antigen”, and “peptide” are used interchangeably herein and refer to any peptide-linked chain of amino acids, regardless of length, co-translational or post-translational modification. A fusion protein (or “chimeric protein”) is a recombinant protein comprising two or more peptide-linked proteins. Fusion proteins are created through the joining of two or more genes that originally coded for the separate proteins. Translation of this fusion gene results in a single fusion protein.

The terms “polynucleotide” and “nucleic acid” are used interchangeably herein and refer to a polymeric macromolecule made from nucleotide monomers. Suitably the polynucleotides of the invention are recombinant. Recombinant means that the polynucleotide is the product of at least one of cloning, restriction or ligation steps, or other procedures that result in a polynucleotide that is distinct from a polynucleotide found in nature.

A heterologous nucleic acid sequence refers to any nucleic acid sequence that is not isolated from, derived from, or based upon a naturally occurring nucleic acid sequence found in the host organism. “Naturally occurring” means a sequence found in nature and not synthetically prepared or modified. A sequence is “derived” from a source when it is isolated from a source but modified (e.g., by deletion, substitution (mutation), insertion, or other modification), suitably so as not to disrupt the normal function of the source gene.

Suitably, the polynucleotides used in the present invention are isolated. An “isolated” polynucleotide is one that is removed from its original environment. For example, a naturally-occurring polynucleotide is isolated if it is separated from some or all of the coexisting materials in the natural system. A polynucleotide is considered to be isolated if, for example, it is cloned into a vector that is not a part of its natural environment or if it is comprised within cDNA.

“Concomitant” administration as used herein refers to administration during the same ongoing immune response. Preferably both components are administered at the same time (such as concomitant administration of a composition comprising a vector and a composition comprising a protein), however, one component could be administered within a few minutes (for example, at the same medical appointment or doctor's visit), or within a few hours. Such administration is also referred to as co-administration. In some embodiments, concomitant administration may refer to the administration of an adenoviral vector, and a protein component. In other embodiments, co-administration refers to the administration of an adenoviral vector and another viral vector, for example a poxvirus such as MVA. In other embodiments, co-administration refers to the administration of an adenoviral vector and a protein component, in which the protein component is adjuvanted.

“Sequential” administration refers to administration of a first composition, followed by administration of a second composition a significant time later, for example not during the ongoing immune response engendered by the first administration. Thus, sequential administration encompasses a first and a subsequent administration in a prime-boost setting. The period of time between two sequential administrations is, for example, 1 week, 2 weeks, 4 weeks, 6 weeks 8 weeks or 12 weeks. More particularly, it is 4 weeks or 8 weeks.

The term “adjuvant” refers to an agent that augments, stimulates, activates, potentiates, or modulates the immune response to an antigen of the composition at either the cellular or humoral level, e.g. immunologic adjuvants stimulate the response of the immune system to the antigen, but have no immunological effect by themselves. The immunogenic compositions disclosed herein may include an adjuvant as a separate ingredient in the formulation, whether or not a vector comprised in (or another component of) the composition also encodes a “genetic adjuvant” such as hli.

### EXAMPLES

Preclinical data in mice comparing the immunogenicity of SAM-HBV with or without human invariant chain has been generated (Example 1 below). In addition, preclinical data has also been generated which compares the immunogenicity of vaccination regimens using one or more of MVA-HBV and ChAd155-hli-HBV to vaccination regimens using at least one SAM-hli-HBV construct (Example 2 below). Preclinical data looking at the immunogenicity of co-administered LNP-mRNAs in HLA-A2/DRB1 naive mice has also been generated (Example 3 below).

These experiments show that LNP-formulated SAM-HBV containing human invariant chain (hli) fused to a hepatitis B core antigen (HBc), induced higher frequency of HBc-specific CD8+ T cell responses (i.e. a higher percentage of HBc-specific CD8+ cells were found to show a response) as compared to the construct without human invariant chain (Example 1 below). In addition, we have been able to show that the use of SAM-hli-HBV can induce higher frequency of HBc and HBs-specific CD8+ T cell responses in HBV chronic infection mouse model (Example 2 below). Furthermore, the experiments show a preferred ratio of co-administered LNP-formulated hli-HBc and hli-HBs mRNAs (Example 3 below).

In all of these experiments, HLA.A2/DRB1 mice (transgenic for the human HLA-A2 and HLA-DRB1 molecules) were used to evaluate the ability of the HBV mRNA vaccine to induce HBc-specific CD8+ T-cell responses. HBV specific CD4+ T-cells and antibodies were evaluated in the same HLA.A2/DRB1 mice.

### Production of the ChAd155-Hli-HBV Drug Substance:

The manufacturing of the ChAd155-hli-HBV Drug Substance involves culture of Procell-92.S cells to a defined cell density. The cells are then infected with ChAd155-hli-HBV Master Viral Seed (MVS) at a defined multiplicity of infection. The ChAd155-hli-HBV virus harvest is purified by a multi-step process based on anion exchange chromatography.

### Vaccine Formulation and Filling for ChAd155-Hli-HBV:

The purified ChAd155-hli-HBV bulk Drug Substance is subsequently processed as follows:
Dilution of the purified ChAd155-hli-HBV Drug Substance in the formulation buffer.
Sterile filtration.
Filling of the final containers.

The ChAd155-hli-HBV vaccine is a liquid formulation contained in vials.

### Production of the MVA-HBV Drug Substance:

The MVA-HBV Drug Substance is manufactured in primary cell cultures of chicken embryo fibroblast (CEF) cells to a defined cell density, and then infected with MVA-HBV Master Viral Seed (MVS) at a defined multiplicity of infection. The MVA-HBV virus harvest is purified by a multi-step process based on fractional gradient centrifugation.

### Vaccine Formulation and Filling for MVA-HBV:

The purified MVA-HBV bulk Drug Substance is subsequently processed as follows:
Dilution of the purified MVA-HBV DS in the formulation buffer.
Filling of the final containers.

The MVA-HBV vaccine is a liquid formulation contained in vials with 0.5 mL extractable volume.

### Production of HBc Drug Substance:

The HBc DS manufacturing process consists of inoculating a pre-culture flask using the recombinant E. coli working seed, followed by a fermentation process and a multi-step purification process including harvesting, extraction, clarification and multiple chromatography and filtration steps.

### Production of the HBs Drug Substance:

The HBs DS manufacturing process consists of inoculating a pre-culture flask using the recombinant S. cerevisiae working seed, followed by a fermentation process and a multi-step purification process including harvesting, extraction, clarification and multiple chromatography and filtration steps.

### Vaccine Formulation and Filling for HBc and HBs:

The purified HBs and HBc DS is diluted in the formulation buffer including sucrose as cryoprotectant and poloxamer as surfactant, filled and lyophilized in 4 mL clear glass vial.

### Doses of AS01 Adjuvant System:

The AS01 B-4 Adjuvant System is composed of immuno-enhancers QS-21 (a triterpene glycoside purified from the bark of Quillaja saponaria) and MPL (3-D Monophosphoryl lipid A), with liposomes as vehicles for these immuno-enhancers and sorbitol. In particular, a single clinical dose container of AS01B-4 (0.5 mL) contains 50 μg of QS-21 and 50 μg of MPL. 1/10th of a human dose i.e. 50 μl is the volume injected in mice (corresponding to 5 μg QS-21 and MPL).

Production of the mRNA Constructs:

The plasmids were linearized with the BspQI restriction enzyme to produce the DNA templates for in vitro transcription. mRNAs were produced by in vitro transcription with capping analogue, TRILINK CLEANCAP A/G and 100% N1-Methyl-Pseudouridine, followed with DNase I, phosphatase treatments and silica column purification. Newly synthesized mRNAs were validated by capillary gel electrophoresis and denaturing agarose gels.

### Production of the SAM-HBV and SAM-Hli-HBV Constructs:

The HBV and hli-HBV sequences were codon optimized for human protein expression, synthesized and cloned into SAM plasmid by GENEWIZ. RNAs were synthesized by in vitro transcription. Briefly, DNA plasmids encoding the SAM replicons were linearized by restriction digestion with BspQI at the 3′ end of the polyA tail and purified by phenol-chloroform extraction. Linearized DNAs were used as templates for in vitro transcription reaction using T7 RNA polymerase. After in vitro transcription, capping of RNAs was performed using a vaccinia capping kit and RNAs were purified by LiCl precipitation and resuspended in nuclease-free water.

Preparation of LNP with SAM followed established methods of preparing LNP through microfluidic mixing, where lipids (cationic lipid, zwiterionic lipid, cholesterol, and PEG-lipid conjugate) were dissolved in an ethanolic solution and SAM was in an aqueous buffered solution. The ethanolic and aqueous solutions were rapidly mixed together using a microfludic mixing chamber. The SAM-entrapped lipid nanoparticles form spontaneously through nucleation of supersaturated lipids in the mixture. Condensation and precipitation of the lipids entrapped SAM and formed lipid nanoparticles. Following a brief maturation of the LNP, the buffer of the SAM-LNP were then exchanged into a storage buffer. The SAM-LNP solutions were characterized for size, lipid content, RNA entrapment and in vitro potency.

The SAM vector VEE TC-83 was used as the background construct for cloning in the Examples. The background empty construct has the nucleic acid sequence of SEQ ID NO:16.

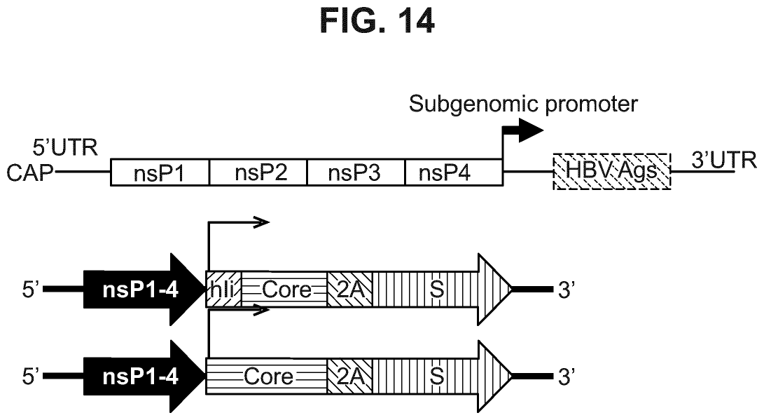

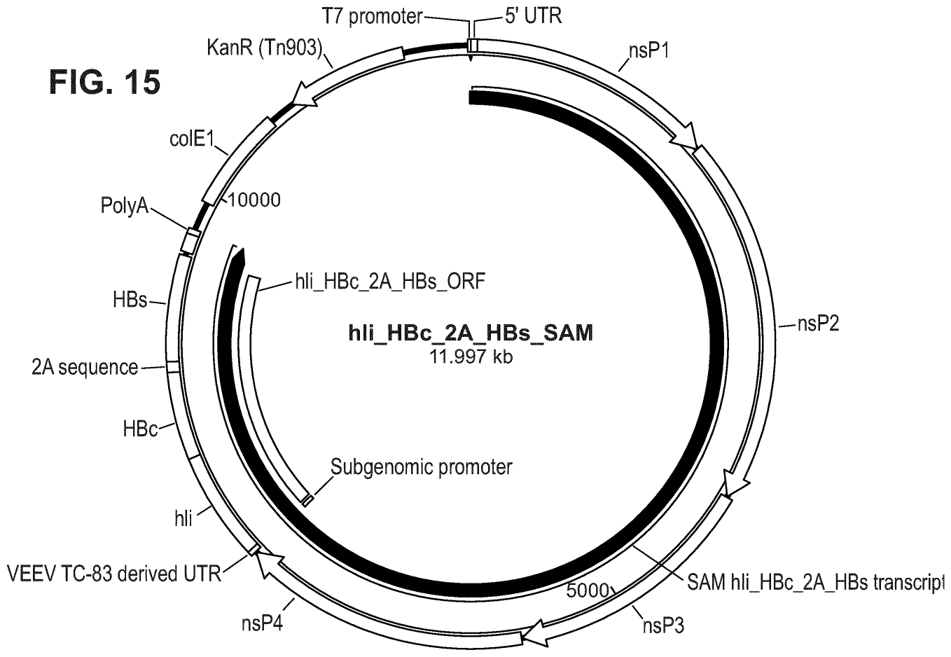

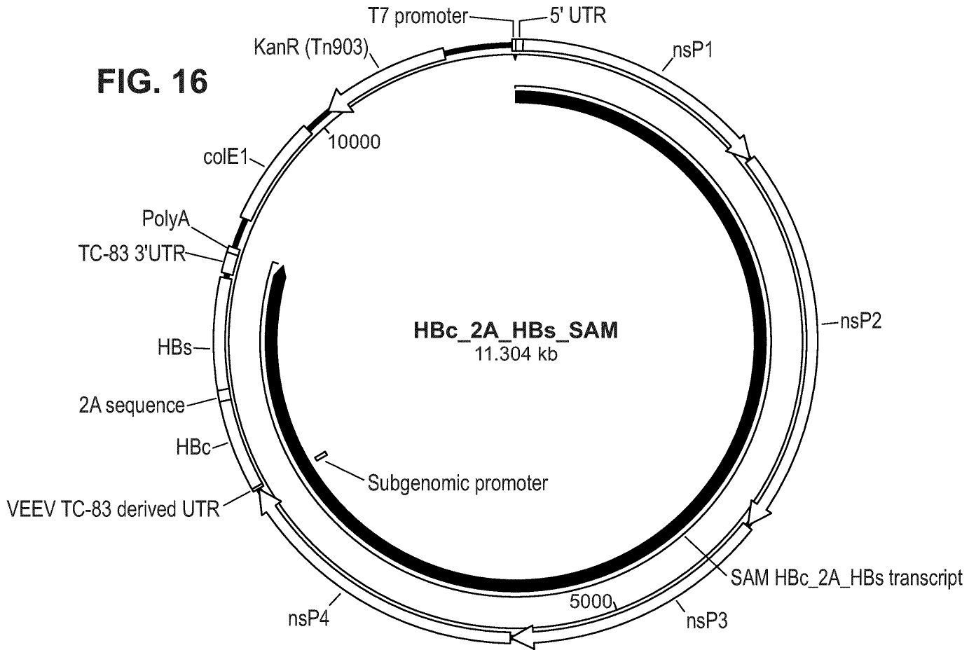

The design of the HBV-SAM construct of FIG. 14 includes cloning the sequence encoding the HBV antigens, under the subgenomic promoter in the SAM vector. Modifications to the SAM HBV constructs were made including codon optimisation of the coding sequence for the antigens.

The SAM constructs were evaluated for robust antigen production and antigenicity and further tested for their immunogenicity and efficacy using in vivo models.

SAM constructs having the sequence of SEQ ID NO:17 and 19 were designed and obtained for further characterisation and testing in the Examples below.

### Characterisation of the SAM-HBV and SAM-Hli-HBV Constructs:

### RNA Pattern Homogeneity Evaluation

In order to study RNA pattern homogeneity, RNA samples were analyzed in 1% agarose gel. RNA samples were prepared as following: 100-250 ng of RNA was mixed with 3 uL of loading buffer (50 mM EDTA pH 8, 30% w/v sucrose, 0.05% bromophenol blue) and water to a final volume of 10 uL. Samples were denatured for 20 minutes at 50° C. Agarose gel was run in NorthernMax-Gly Gel Running Buffer (Invitrogen™) for 45 min at 130 V. No major RNA degradation was observed and a similar pattern between both constructs was obtained.

### Protein Expression Evaluation by Western Blot

The ability of cells to express the given antigens from the different HBV SAM constructs was evaluated according to the following methods.

On Day 0, Baby hamster kidney (BHK) cells were plated at 1×107 in T225 flasks in growth media (DMEM high glucose (Gibco™), 1% L-glutamine, 1% Pen-Strep (Corning®), 5% FBS (Gibco™)). For trypsinization, media was removed and cells were washed with 5 mL of PBS. The PBS wash was removed, and 5 mL of pre-warmed trypsin was added and spread thoroughly across the plate. Trypsin was removed and plates were kept at 37° C. for 1-2 mins. Cells were then resuspended in 10 mL of growth media. Cells were counted and plated at required concentration into a new flask. The cells were then incubated at 37° C., 5% C₀₂ for about 20 hours.

On Day 1, plates were prepared by adding 2 mL of outgrowth media (DMEM high glucose, 1% 30 L-glutamine, 1% Pen-Strep, 1% FBS) to each well of a 6-well plate (one well per electroporation). Plates were kept warm in a 37° C. incubator. The electroporator was prepared to deliver 120V, 25 ms pulse, 0.0 pulse interval, 1 pulse for a 2 mm cuvette. Cuvettes were labeled and kept on ice. Cells in growth phase were harvested into BHK growth media and counted using a cell counter. Cells were trypsinized following the same trypsinization 35 protocol as above. Cells were then centrifuged at 462×g for 3 min. Media was aspirated, and cells were washed once with 20 mL cold Opti-MEM media (Gibco™). Cells were again centrifuged at 462×g for 5 mins. Media was aspirated and the cells were resuspended in Opti-MEM media to 0.25 mL per 1×106 cells per electroporation. Standards and negative control were also prepared.

For each sample, 2 ug of RNA was mixed with 250 μL cells, and the mixture was pipetted gently 4-5 times. The cells and RNA mixture were transferred to 2 mm cuvettes and subjected to one pulse of electroporation using the parameters described above. Cells were allowed to rest at room temperature for 10 min. Cells from one cuvette were added to one well of a pre-warmed 6-well plate, and the plate was tipped front and back and then side to side at a 45⁰ angle to distribute cells evenly. On Day 2 (17 h post-electroporation), cell culture supernatants were collected and analyzed by Western Blot at different concentrations. Cell monolayers were detached and resuspended in 1 mL of 20 mM HEPES, 150 mM NaCl, 5% Glycerol pH 7.6 buffer, complemented with cOmplete™ Protease Inhibitor Cocktail (Roche, Cat. 11697498001), prior to performing cell lysis by sonication. Following cell lysis, intracellular fractions were analysed by Western-Blot. Primary antibodies used were mouse anti-HBc monoclonal antibody and rabbit anti-HBs polyclonal sera (generated in-house).

In Vitro Potency of the SAM after LNP Formulation

An in vitro potency assay was also performed after LNP formulation.

### Cellular Immune Response—Intracellular Cytokine Staining (ICS):

Fresh pools of peripheral blood leukocytes (PBLs), splenocytes or liver infiltrating lymphocytes collected at different time points, were stimulated ex vivo for 6 hours with pools of 15-mers, overlapping of 11aa, covering the HBc or HBs sequence. The HBc and HBs-specific cellular responses were evaluated by ICS measuring the amount of CD4+ or CD8+ T-cells expressing IFN-γ and/or IL-2 and/or tumor necrosis factor (TNF)-α. The technical acceptance criteria to take into account ICS results include the minimal number of acquired CD8+T or CD4+ T cells being >3000 events.

### Humoral Immune Response—Enzyme-Linked Immunosorbent Assay (ELISA):

HBc- and HBs-specific antibody responses were measured by ELISA on sera from immunized mice at different time points. Briefly, 96-well Elisa plates were coated with purified Hepatitis B core antigen (HBc) or with purified Hepatitis B surface antigen (HBs). Sera from vaccinated mice were serially diluted and incubated. Serial dilutions of the standard and control material are used to calculate the anti-HBc or anti-HBs antibody standard titers of tested sera and to ensure validity of the test. Plates were washed with PBS 0.1% tween20 buffer after each incubation step. An horseradish peroxidase Goat anti-mouse IgG (H+L) antibody was then added and the antibody complex is revealed by incubation with Tetramethylbenzidine liquid Substrat (TMB). The Optical densities (OD) were recorded at 450-620 nm. The anti-HBc or HBs antibody titer of each individual mouse serum is determined from the standard curve of the ELISA using a regression model. Geometric Mean Titers (GMT) are then calculated for each group of mice. For each time point and each antigen (HBc, HBs), an analysis of variance (ANOVA) model was fitted on log 10 titres including group, study and interaction as fixed effects and using a heterogeneous variance model (identical variances were not assumed between groups). This model was used to estimate geometric means (and their 95% CIs) as well as the geometric mean ratios and their 95% CIs. As no pre-defined criteria were set, the analysis is descriptive and 95% CIs of ratios between groups were computed without adjustment for multiplicity.

### Alt/Ast Measure:

The levels of ALT and AST in mouse sera were quantified using the following commercial kits:
Alanine Aminotransferase Activity Assay Kit Sigma Aldrich Cat #MAK052
Aspartate Aminotransferase Activity Assay Kit Sigma Aldrich Cat #MAK055

### Serum HBs Antigen Quantification

The circulating HBs antigen in mouse sera was quantified using the Monolisa Anti-HBs PLUS commercial kit from BIO-RAD (cat #72566) and an international standard (Abbott Diagnostics).

### Example 1: SAM-HBV with or without Human Invariant Chain

In this experiment, male and female HLA.A2/DR1 naïve mice received an intramuscular injection on days 0 and 28. The compositions administered to the different groups on days 0 and 28 are detailed in Table 1 below.

TABLE 1

| Groups | N | Day 0 | Day 28 | Sacrifice |
|---|---|---|---|---|
| 1 | 14 | ChAd155-hli-HBV | SAM-hli-HBV - 2.5 μg | 14dPI and |
| | | 10⁸ vp | | 12/13dpII |
| 2 | 14 | ChAd155-hli-HBV | SAM-hli-HBV - 1 μg | 14dPI and |
| | | 10⁸ vp | | 12/13dpII |
| 3 | 14 | ChAd155-hli-HBV | SAM-hli-HBV - 0.1 μg | 14dPI and |
| | | 10⁸ vp | | 12/13dpII |
| 4 | 14 | ChAd155-hli-HBV | SAM-HBV - 2.5 μg | 14dPI and |
| | | 10⁸ vp | | 12/13dpII |
| 5 | 14 | ChAd155-hli-HBV | SAM-HBV - 1 μg | 14dPI and |
| | | 10⁸ vp | | 12/13dpII |
| 6 | 14 | ChAd155-hli-HBV | SAM-HBV - 0.1 μg | 14dPI and |
| | | 10⁸ vp | | 12/13dPII |
| 7 | 6 | NaCl | NaCl | 14dPI and |
| | | | | 12/13dpII |

(Note:
SAM-hli-HBV is the construct of SEQ ID NO: 17, and SAM-HBV is the construct of SEQ ID NO: 19)

Except for in control group 7, all groups used 14 mice (N=14). 14 days post the first injection (14dpl), 2 mice from each group were sacrificed so that a spleen sample and sera samples could be taken and T cell responses measured at this time point, and sera samples were taken from all mice. 12 and 13 days post the second injection (12/13dpll), the remaining animals were all sacrificed, and spleen, liver and sera samples taken.

For groups 1 to 6, all mice were primed with ChAd155-hli-HBV and boosted with SAM-HBV (±hli). The same dose of ChAd155-hli-HBV was administered: 10⁸ vp/mouse. However, three different doses were used for SAM-HBV (±hli): 2.5 μg, 1 μg and 0.1 μg. The specific dose that was used in each group is specified in Table 1 above.

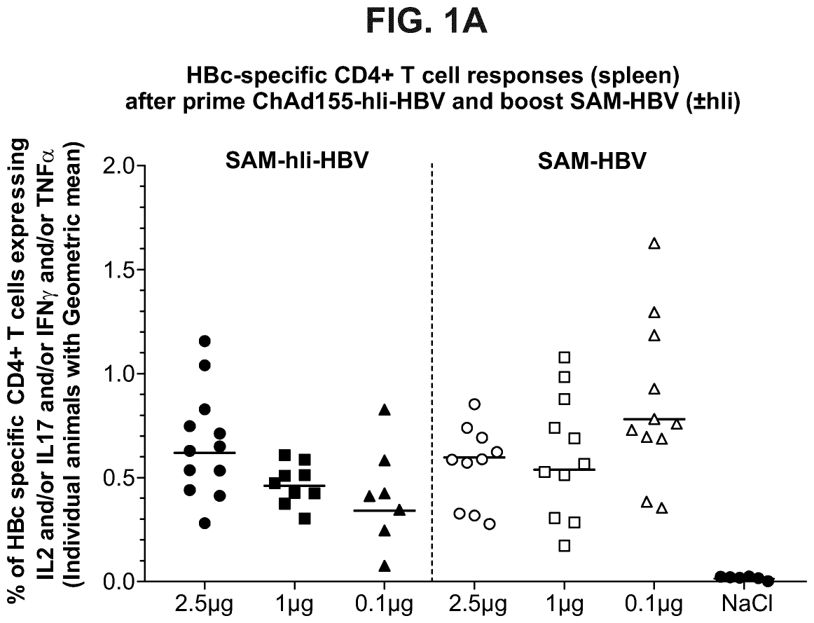

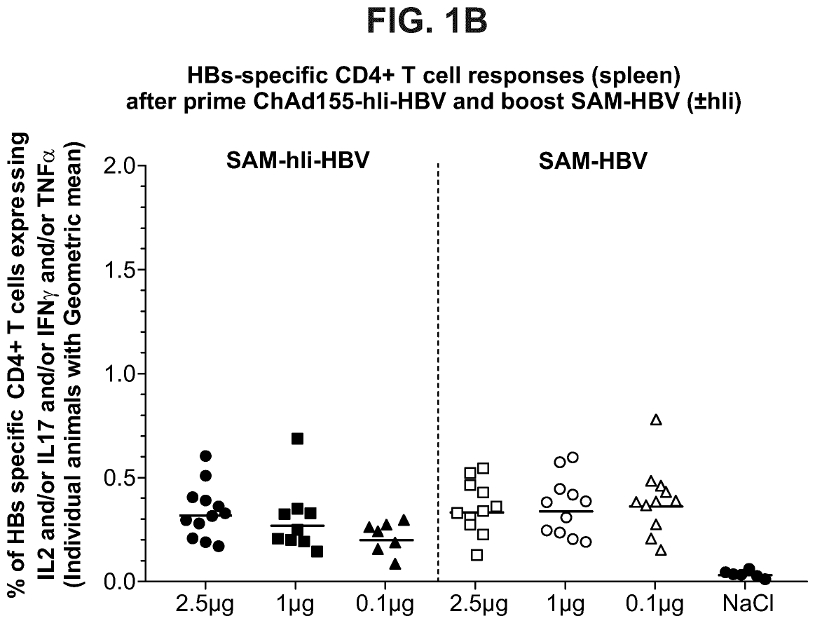

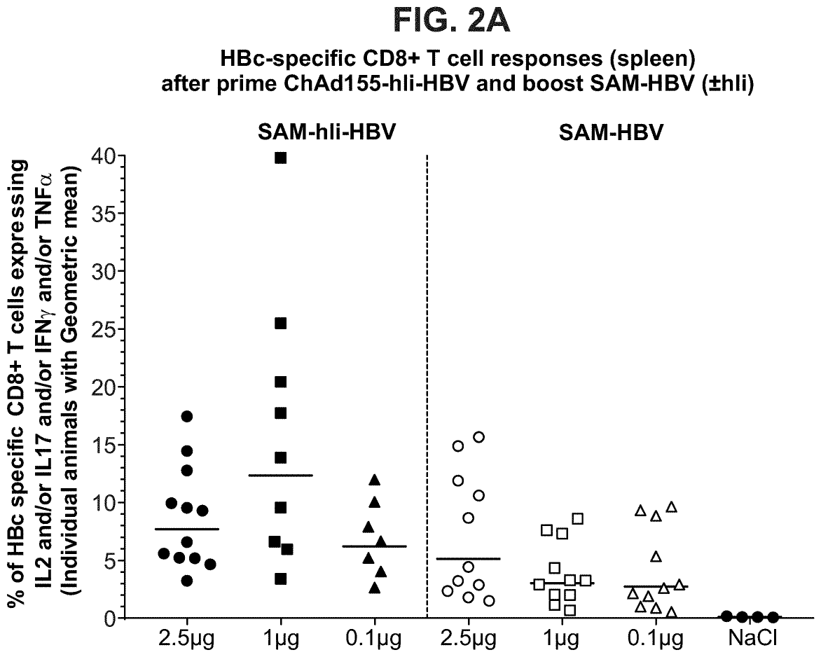

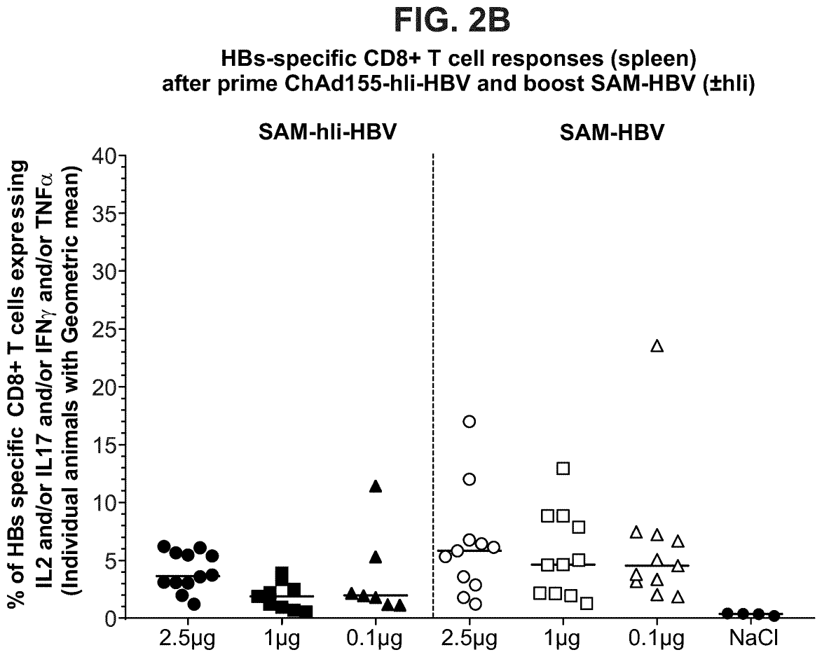

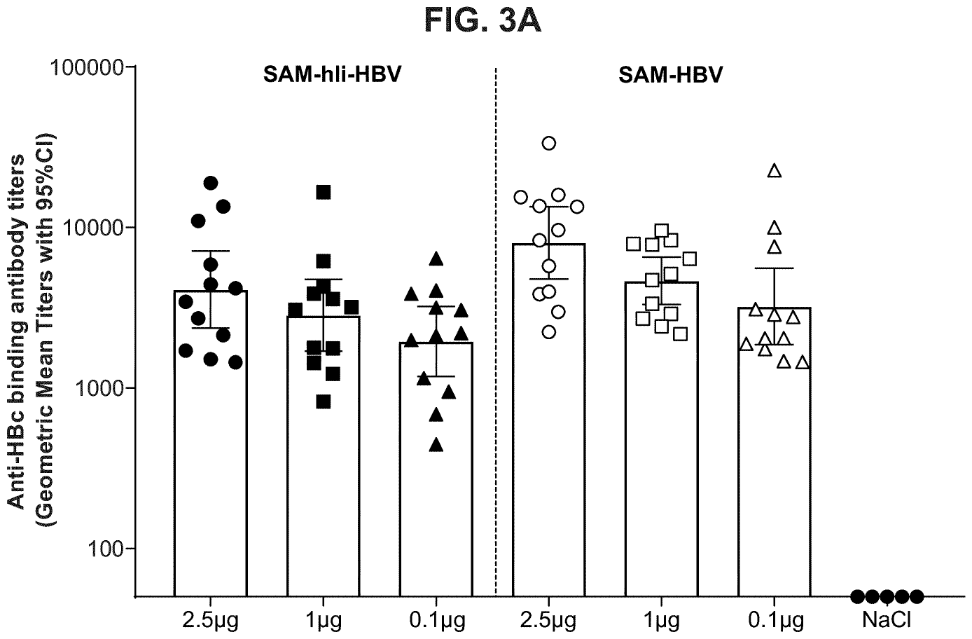

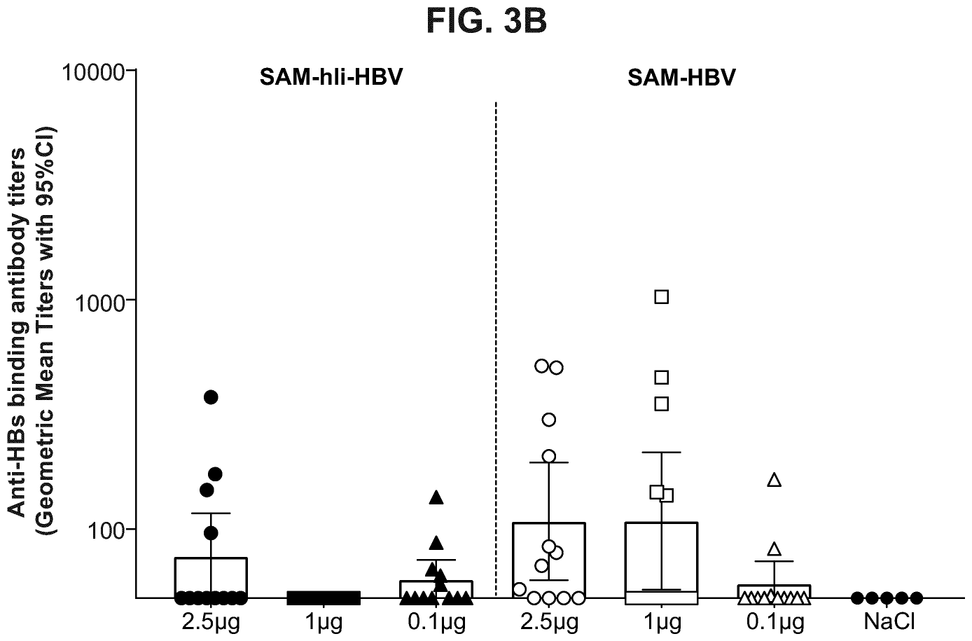

The HBc- and HBs-specific CD4+ and CD8+ T-cell responses and HBc- and HBs-specific antibody responses generated by Example 1 are shown in FIGS. 1, 2 and 3. FIG. 1 shows the CD4+ responses, FIG. 2 shows the CD8+ responses and FIG. 3 shows the antibody responses. In all of these figures, the “A” figure shows the Hepatitis B core antigen response, and the “B” figure shows the Hepatitis B surface antigen response.

As shown in FIGS. 2A and 2B, the SAM constructs comprising invariant chain (SAM-hli-HBV), were shown to induce significantly higher frequency of HBc-specific CD8+ T cell responses towards HBc (Geometric Mean Ratio, GMR=4.1 with 95% CIs [1.96-8.40]) compared to the constructs without invariant chain (SAM-HBV). First the geometric mean of HBc-specific CD8+ T cell responses was calculated for the group of mice immunized with SAM-hli-HBV and then for the group of mice immunized with SAM-HBV. Then the ratio of these 2 geometric means was calculated. In this case, we observed that SAM-hli-HBV induced 4-fold higher HBc-specific CD8+ T cell responses.

Efficient control of HBV infection is associated with the induction and persistence of CD4+ and CD8+ T cells targeting specifically HBV core and surface antigens which play a major role in control and resolution of HBV infection.

Several published studies comparing the HBV antigen-specific T-cells in different segments of patients affected by HBV (post-acute infection, patients recovering from a chronic infection, active chronic infection and inactive carriers) have highlighted the necessity to induce a strong multi-specific T-cell response to HBV antigens, particularly the HBc antigen, to promote the clearance of HBV infection. In line with this, comparing T cells from patients with a chronic HBV resolving infection versus patients with unresolved chronic HBV infection, has shown higher CD4+ T-cells and CD8+ T-cells specific to HBc antigen in patients with resolving infection [Boni, 2012; Li, 2011; Liang, 2011].

Furthermore, the role of functional CD8+ T-cells appears to be critical. Depletion of CD8+ T-cells in chimpanzees during acute HBV infection results in the persistence of the viremia [Thimme, 2003]. In humans, clearance of HBV during acute hepatitis B is associated with a strong, polyclonal, multi-specific CD8+ T-cell response to the viral nucleocapsid, envelop and polymerase proteins that persists for decades after clinical recovery. In contrast, CHB patients usually fail to mount a strong CD8+ T-cells response to the virus. CHB patients who experience a spontaneous or interferon-induced remission develop a CD8+ T-cell response to HBV that is similar in strength and specificity compared to the response from patients who have recovered from acute hepatitis [Rehermann, 1996].

As the SAM constructs comprising invariant chain (SAM-hli-HBV), were shown to induce a greater CD8+ T cell responses towards HBc and HBs antigens, these constructs were selected for use in Example 2.

### Example 2: Evaluation of the Replacement of MVA-HBV or Both ChAd155-Hli-HBV and MVA-HBV by SAM-Hli-HBV in in HLA.A2/DR1 Transduced Mice

Due to the maximum capacity of animals per experiment, two independent experiments were planned. Both experiments contained animals in every group detailed in Table 2.

In this experiment, male and female HLA.A2/DR1 transduced mice were used. The AAV2/8-HBV-transduced HLA.A2/DR1 murine model recapitulates virological and immunological characteristics of chronic HBV infection. This was selected to evaluate the immunogenicity of different vaccines regimens, the impact of liver infiltrating HBc-specific CD8+ T-cells, potentially targeting hepatocytes expressing the HBcAg and assessing the potential vaccine-associated liver inflammation by measuring serum activities of Aspartate aminotransferase (AST) and Alanine aminotransferase (ALT).

Thus, in these experiments, male and female HLA.A2/DR1 mice (groups 1-6 and group 8) were injected intravenously at day 0 with 10¹⁰viral genome (vg) of adeno-associated virus serotype 2/8 (AAV2/8 HBV) vector carrying a replication-competent HBV DNA genome.

The HLA.A2/DR1 mice were randomized before immunization in the 7 different groups (groups 1-6 and group 8) based on the level of HBs circulating antigen detected in the sera at day 21/22, the age and the gender proportion.

Mice from group 7 were not transduced with AAV2/8-HBV viral vector but intramuscularly (IM) immunized with the co-administration vaccine regimen. This group was used as positive control for the immunological read-outs.

The HLA.A2/DR1 transduced mice received an intramuscular (gastrocnemian muscle) injection on days 31 or 33 (first immunization), 59 or 61 (second immunization), 73 or 75 (third immunization) and 86 or 88 (fourth immunization) with various formulations containing HBc and HBs antigens (listed in Table 2). The results of the two separate experiments were combined for the presentation of figures and the statistical analysis of the results.

In all cases, the same doses of each composition were used:
ChAd155-hli-HBV was administered to the mice at a dose of 10⁸ vp/mouse,
The mice which were vaccinated with MVA-HBV received a dose of 10⁷ pfu/mouse,
SAM-hli-HBV was administered to the mice at a dose of 1 μg/mouse, and
The groups which received adjuvanted protein received a dose of 4 μg of HBc, 1 μg HBs and AS01 comprising 5 μg MPL and 5 μg QS21/mouse.

The objective of this experiment was to evaluate if SAM-hli-HBV can replace MVA-HBV or both ChAd155-hli-HBV and MVA-HBV in the sequential or in co-administration vaccine regimen by inducing at least the same level of HBc-specific CD8+ T cell responses as compared to the vaccine regimens with MVA-HBV.

TABLE 2

| | | Transduction | | | | | |
|---|---|---|---|---|---|---|---|
| Groups | N | Day 0 | Day 31/33 | Day 59/61 | Day 73/75 | Day 86/88 | Sacrifice |
| 1 | 30 | AAV2/8-HBV | ChAd155- | SAM-hli- | HBc-HBs/ | HBc-HBs/ | 13/14dpII |
| | | | hli-HBV | HBV | AS01 | AS01 | 22dpIV |
| 2 | 30 | AAV2/8-HBV | ChAd155- | SAM-hli- | SAM-hli- | SAM-hli- | 13/14dpII |
| | | | hli-HBV + | HBV + | HBV + | HBV + | 22dpIV |
| | | | HBc-HBs/ | HBc- | HBc- | HBc- | |
| | | | AS01 | HBs/AS01 | HBs/AS01 | HBs/AS01 | |
| 3 | 30 | AAV2/8-HBV | SAM-hli- | SAM-hli- | HBc-HBs/ | HBc-HBs/ | 13/14dpII |
| | | | HBV | HBV | AS01 | AS01 | 22dpIV |
| 4 | 30 | AAV2/8-HBV | SAM-hli- | SAM-hli- | SAM-hli- | SAM-hli- | 13/14dpII |
| | | | HBV + | HBV + | HBV + | HBV + | 22dpIV |
| | | | HBc-HBs/ | HBc- | HBc- | HBc- | |
| | | | AS01 | HBs/AS01 | HBs/AS01 | HBs/AS01 | |
| 5 CTRL | 30 | AAV2/8-HBV | ChAd155- | MVA-HBV | HBc-HBs/ | HBc-HBs/ | 13/14dpII |
| group | | | hli-HBV | | AS01 | AS01 | 22dpIV |
| 6 CTRL | 30 | AAV2/8-HBV | ChAd155- | MVA-HBV + | MVA-HBV + | MVA-HBV + | 13/14dpII |
| group | | | hli-HBV + | HBc- | HBc- | HBc- | 22dpIV |
| | | | HBc-HBs/ | HBs/AS01 | HBs/AS01 | HBs/AS01 | |
| | | | AS01 | | | | |
| 7 | 10 | — | ChAd155- | MVA-HBV + | MVA-HBV + | MVA-HBV + | 13/14dpII |
| | | | hli-HBV + | HBc- | HBc- | HBc- | 22dpIV |
| | | | HBc-HBs/ | HBs/AS01 | HBs/AS01 | HBs/AS01 | |
| | | | AS01 | | | | |
| 8 | 10 | AAV2/8-HBV | NaCl | NaCl | NaCl | NaCl | 13/14dpII |
| | | | | | | | 22dpIV |

Note:
(SAM-hli-HBV is the construct of SEQ ID NO: 17, and SAM-HBV is the construct of SEQ ID NO: 19)

31 or 33 days prior to the first injection, the mice of groups 1 to 6 and 8 were transduced with AAV2/8-HBV. As mentioned above, for practical reasons associated with running the experiments, each group was divided in two and two separate experiments were run. All of the results shown are the combined output from both experiments.

13 and 14 days post the second injection (13/14dpll), 15 mice from each of groups 1 to 6 (from each experiment), and 6 and 4 mice from groups 7 and 8 of experiment 20200719 and 4 and 5 mice from groups 7 and 8 of experiment 20200720 were sacrificed so that a spleen sample could be taken and sera samples were taken from all mice. 22 days post the fourth injection (22dPIV), the remaining animals were all sacrificed, and spleen, liver and sera samples taken.

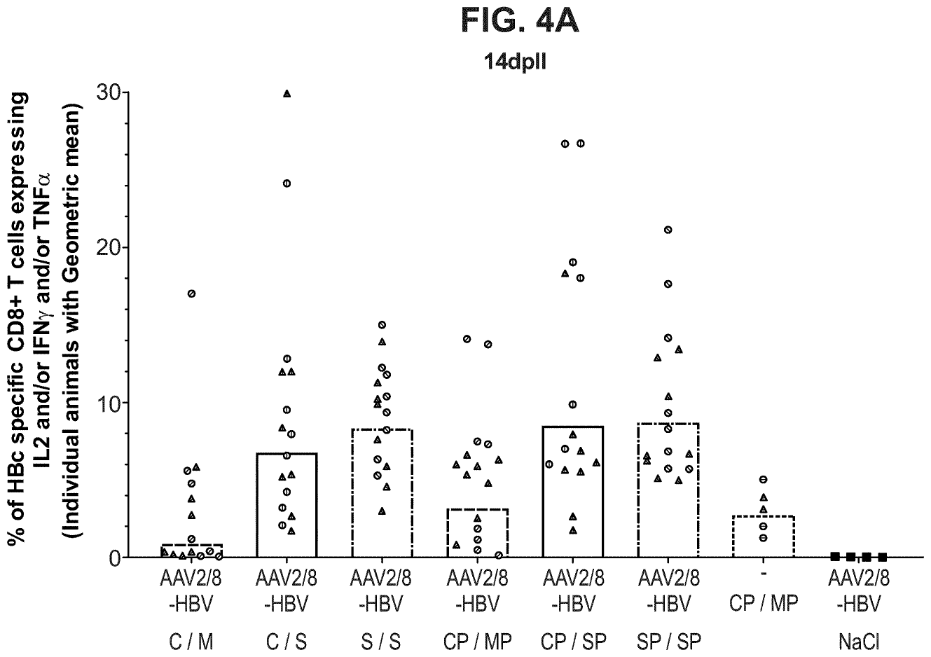

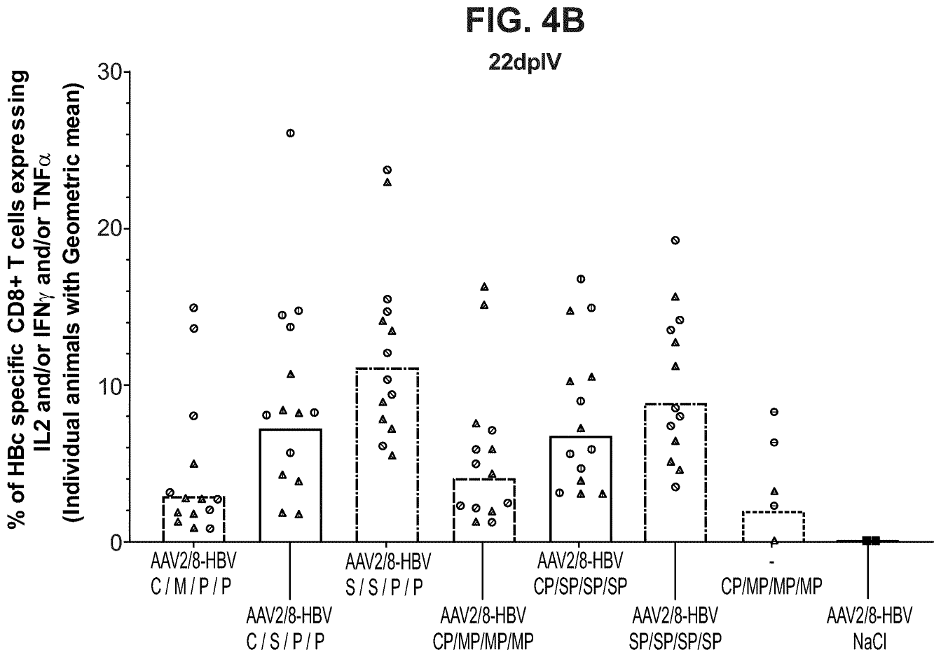

The CD8+ T-cell and antibody responses generated by the various groups of Example 2 are shown in FIGS. 4A and 4B. The results of FIG. 4 show that, 14 days post second immunization, priming with ChAd155-hli-HBV and boosting with SAM-hli-HBV produced a 7.36 fold higher HBc-specific CD8+ T-cell response (GMR=7.36 with 90% CIs [3.96-13.70]) than priming with ChAd155-hli-HBV and boosting with MVA-HBV. Similarly, the results also show that priming with SAM-hli-HBV and boosting with SAM-hli-HBV induced a 9 fold higher HBc-specific CD8+ T-cell response (GMR=9.07 with 90% CIs [4.87-16.87]) than priming with ChAd155-hli-HBV and boosting with MVA-HBV.

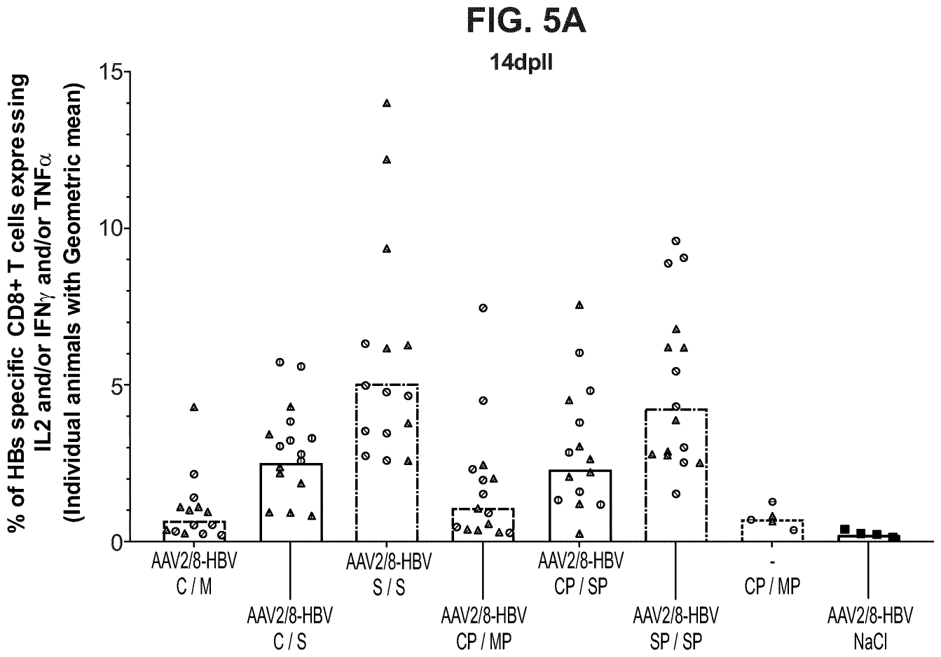

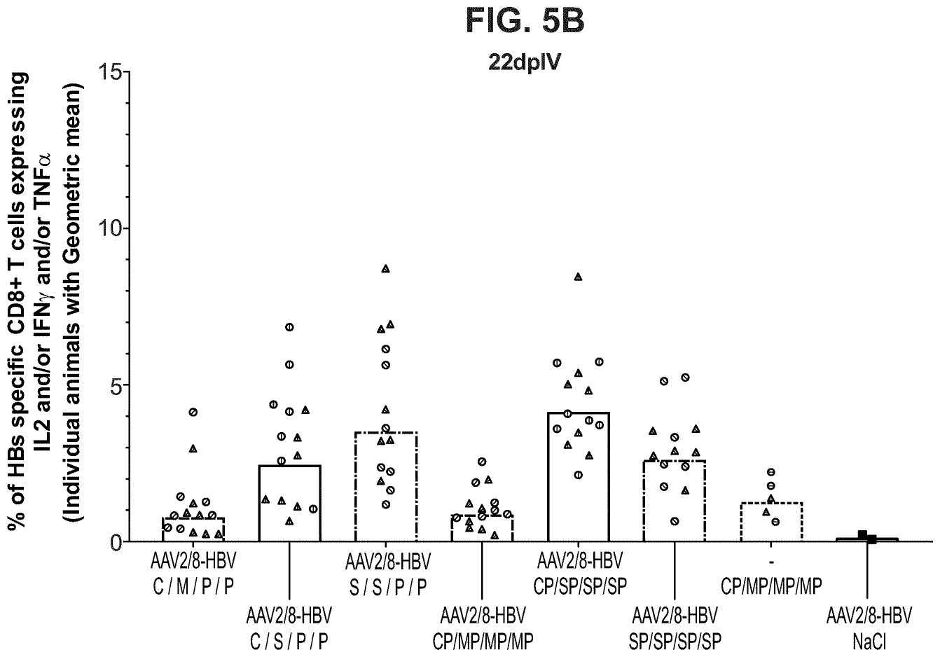

Similar CD8+ T-cell results are shown in FIGS. 5A and 5B. Here, priming with ChAd155-hli-HBV and boosting with SAM-hli-HBV induced a 3.6 fold greater HBs-specific CD8+ T-cell response (GMR=3.64 with 90% CIs [2.34-5.67]) than priming with ChAd155-hli-HBV and boosting with MVA-HBV after 14 days post second immunization. The results also show that priming with SAM-hli-HBV and boosting with SAM-hli-HBV induced a 7.78 fold higher HBs-specific CD8+ T-cell response (GMR=7.78 with 90% CIs [5-12.12]) than priming with ChAd155-hli-HBV and boosting with MVA-HBV.

Interestingly, replacing MVA-HBV or both ChAd155-hli-HBV and MVA-HBV by SAM-hli-HBV vaccine induced more polyfunctional HBV-specific CD8+ T-cells, as demonstrated by the cytokine co-expression profile (FIG. 13A, 13B). The majority of HBV-specific CD8+ T-cells expressed mainly IFN-g in combination with TNFα and this population has further increased when homologous prime boost with SAM-hli-HBV was used.

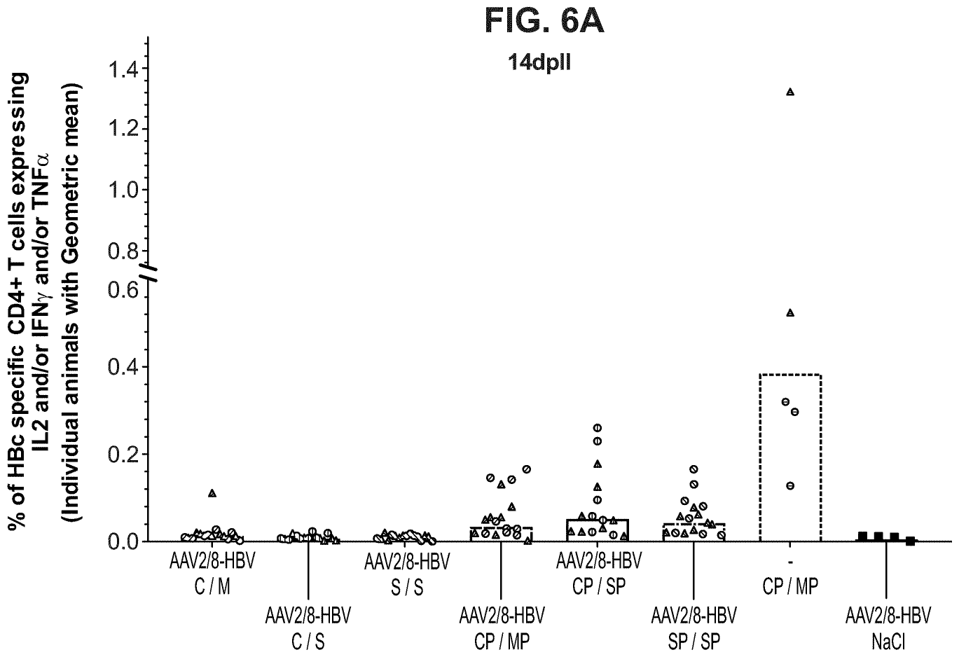

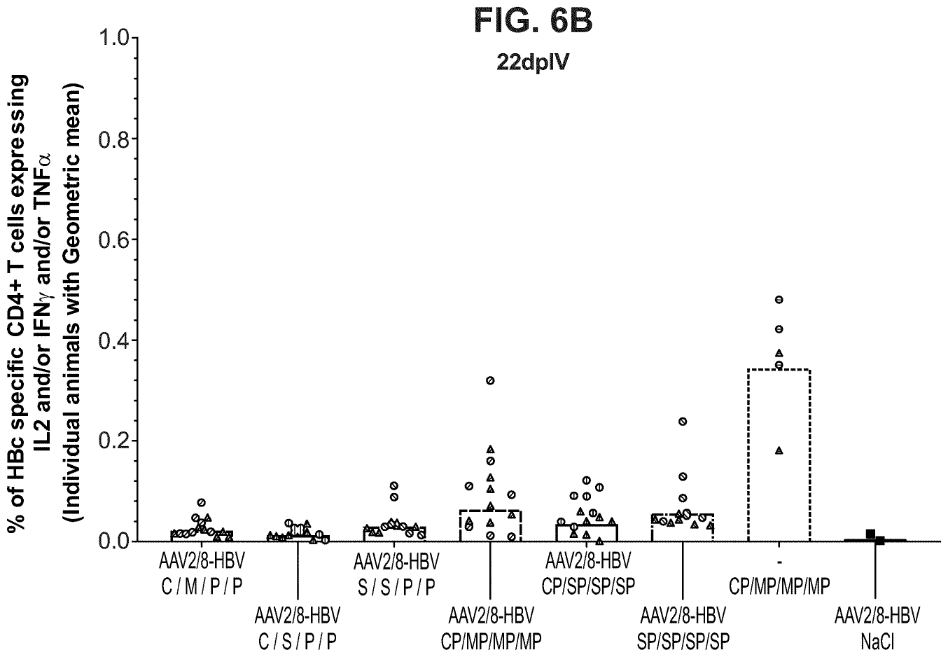

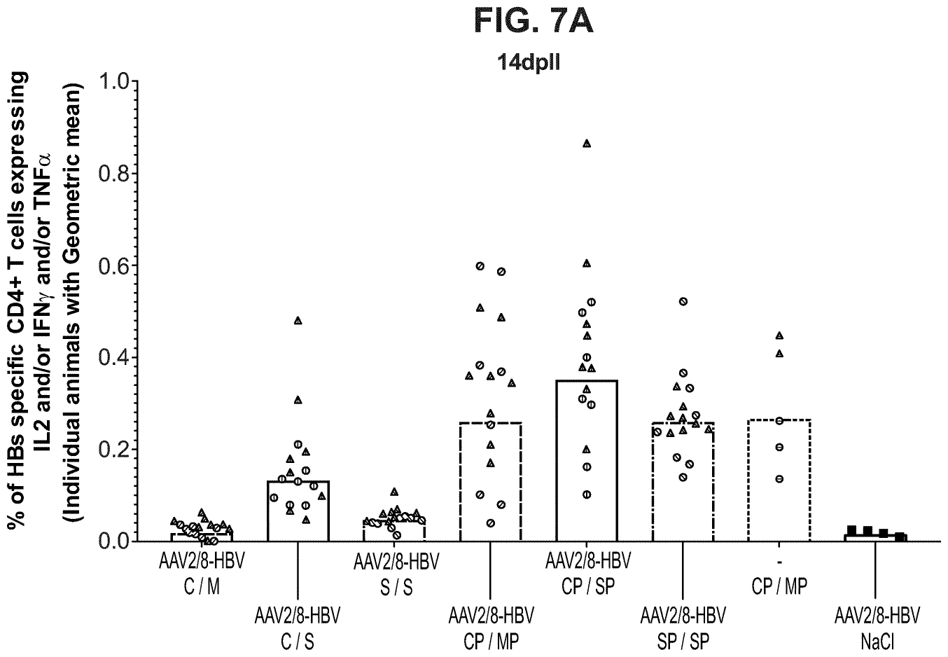

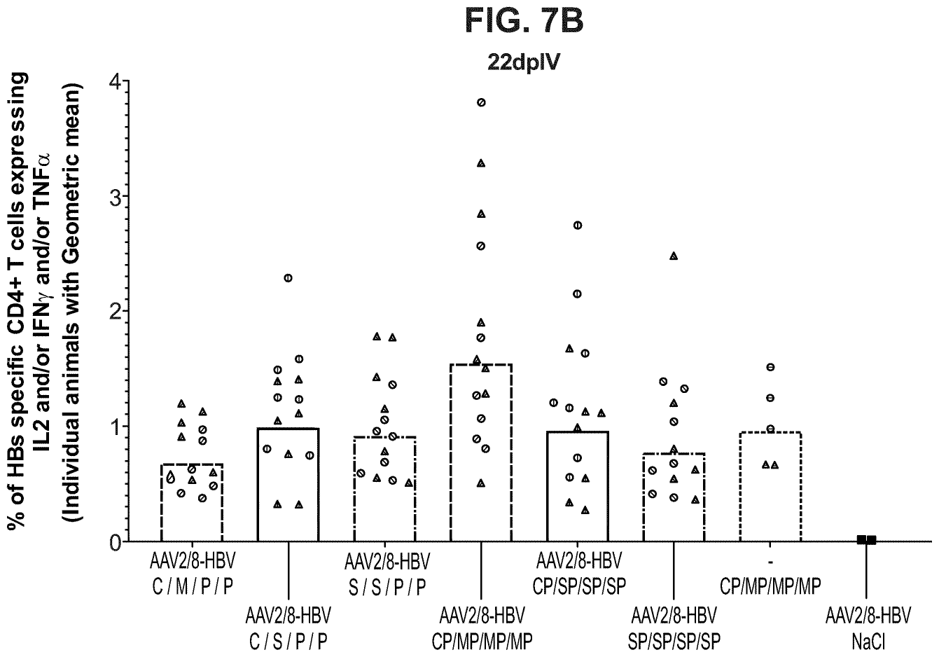

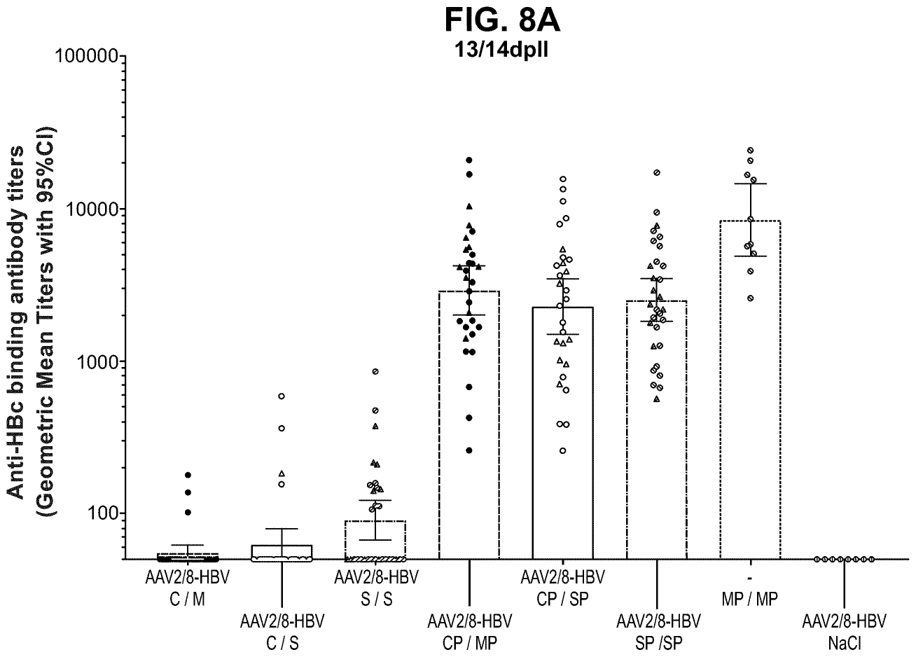

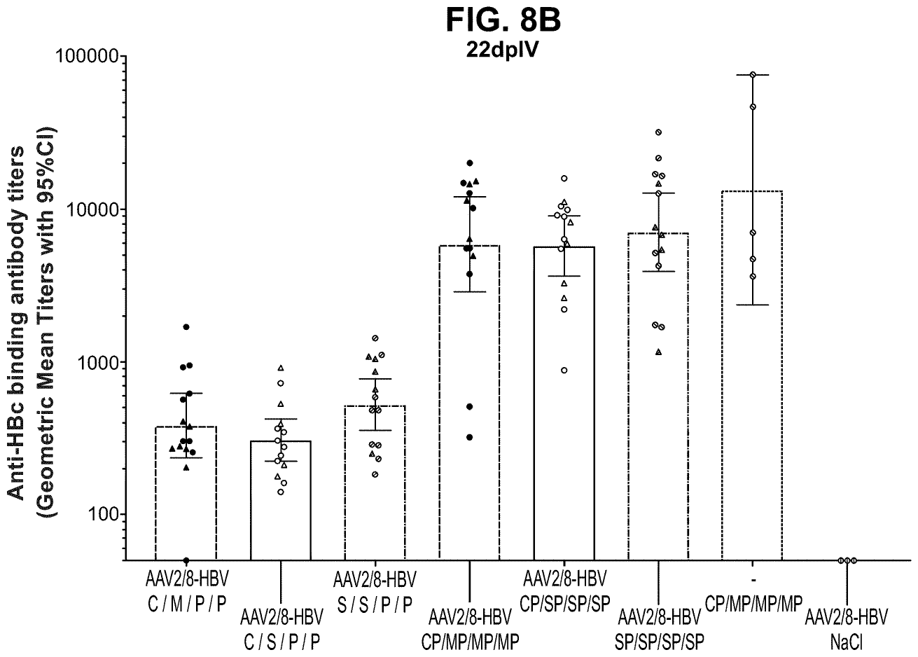

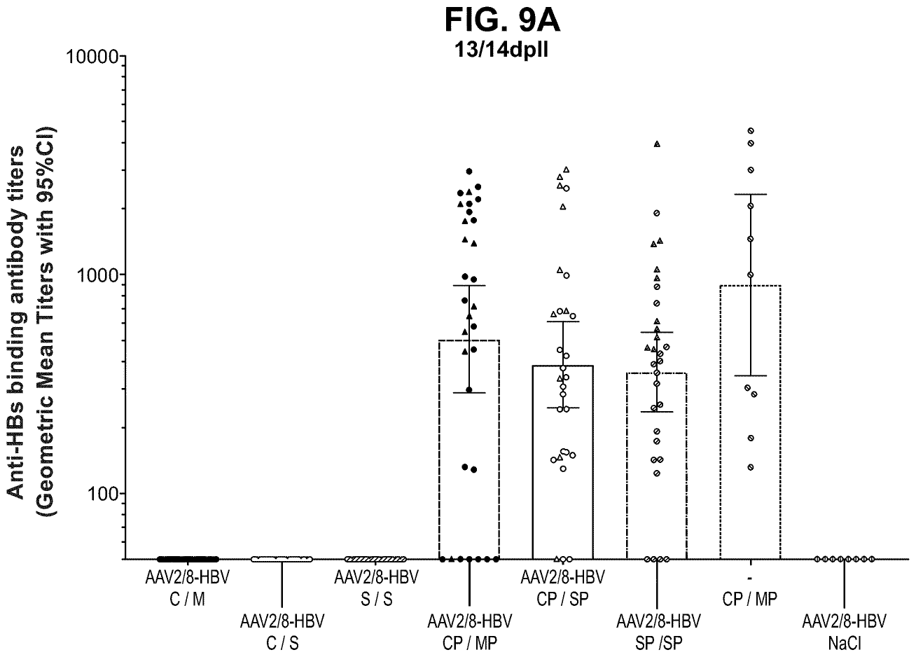

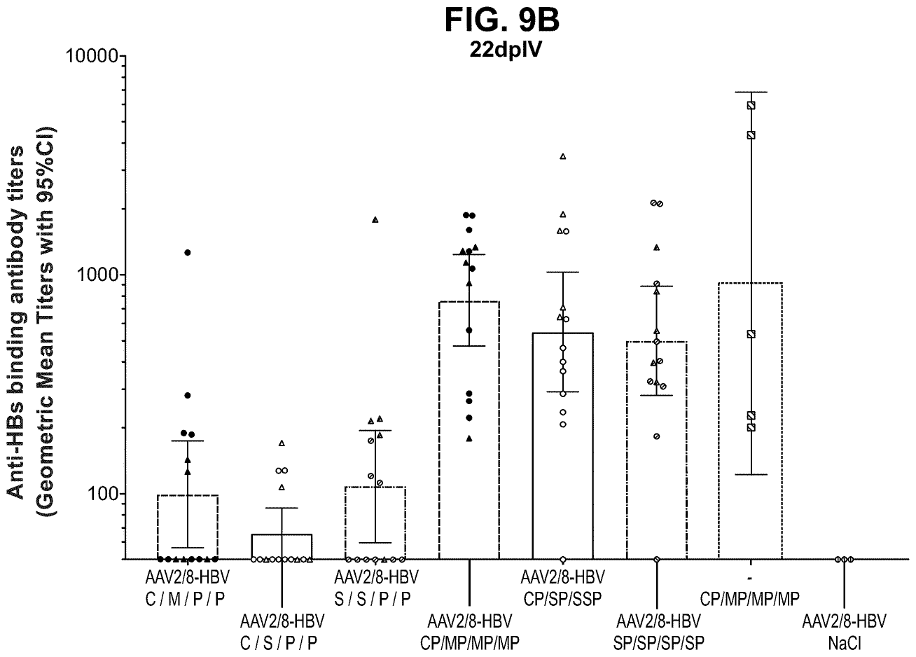

FIGS. 6 and 7 show the CD4+ responses measured in the spleen. At 14 days post second immunization, all vaccine regimens elicited very low to undetectable HBc-specific CD4+ T-cells (50,1%) in AAV2/8-HBV-transduced HLA-A2/DR1 mice and slightly higher HBc-specific CD4+ T cells were only induced after administration of HBc-HBs/AS01 either alone or in combination with both vectors.

Regarding anti-HBs-specific CD4+ T cell responses at 14 days post second immunization, priming with ChAd155-hli-HBV and boosting with SAM-hli-HBV induced a 7.12 fold higher HBs-specific CD4+ T-cell response (Geometric Mean Ratio (GMR)=7.12 with 90% CIs [4.61-11]) than priming with ChAd155-hli-HBV and boosting with MVA-HBV. To note that replacing both ChAd and MVA by SAM-hli-HBV did not induced higher responses, however, the results show that priming with SAM-hli-HBV and boosting with SAM-hli-HBV induced 2.56 fold higher HBs-specific CD4+ T-cell response (GMR=2.56 with 90% CIs [1.66-3.95]) than priming with ChAd155-hli-HBV and boosting with MVA-HBV. As previously observed for the HBc-specific CD4+ T cell responses, strong HBs-specific CD4+ T cells were only induced after administration of HBc-HBs/AS01 either alone or in combination with both vectors.

Regarding the humoral immune responses, replacing MVA-HBV or both ChAd155-hli-HBV and 20 MVA-HBV by SAM-hli-HBV did not impact the level of anti-HBc and anti-HBs antibody responses.

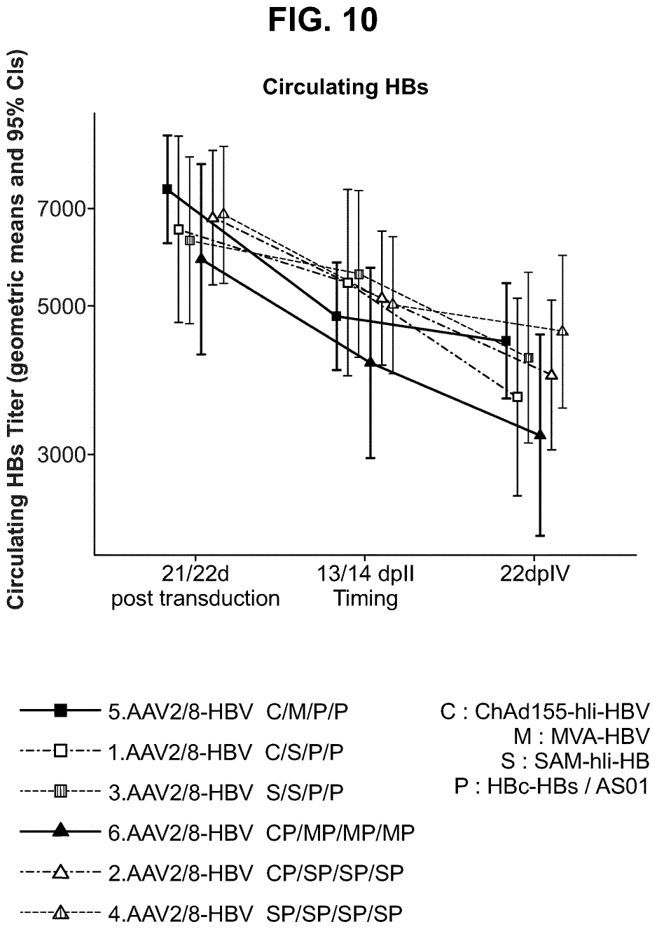

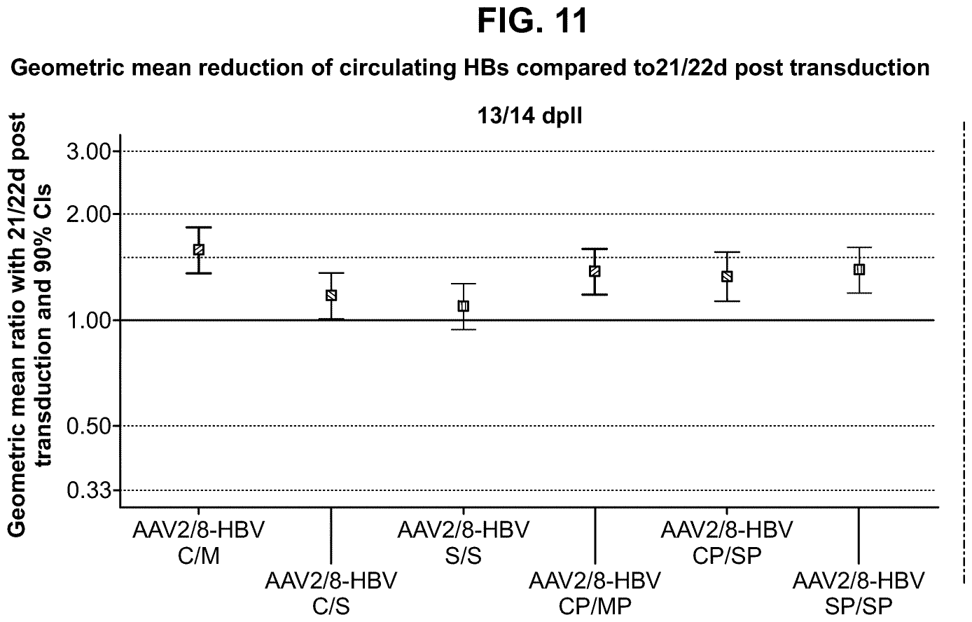

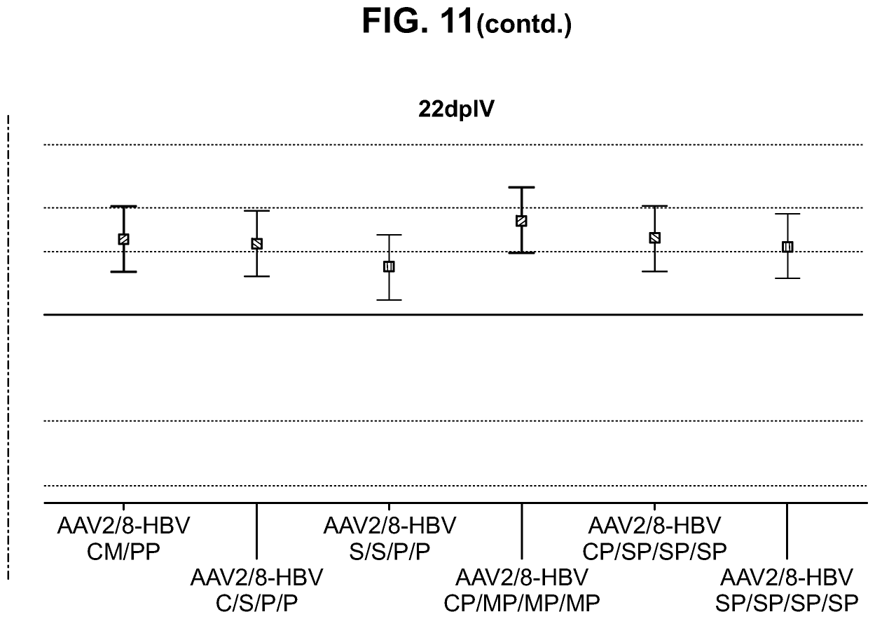

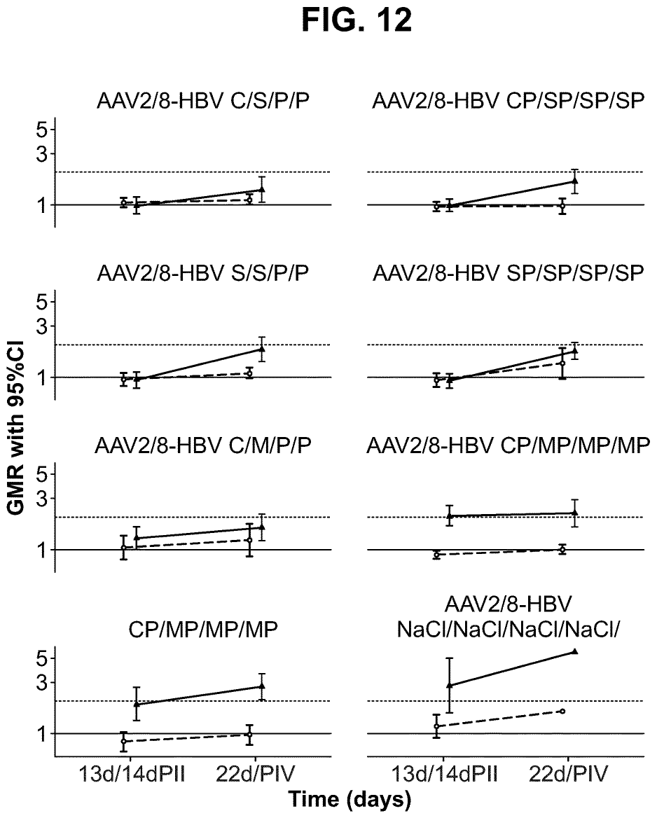

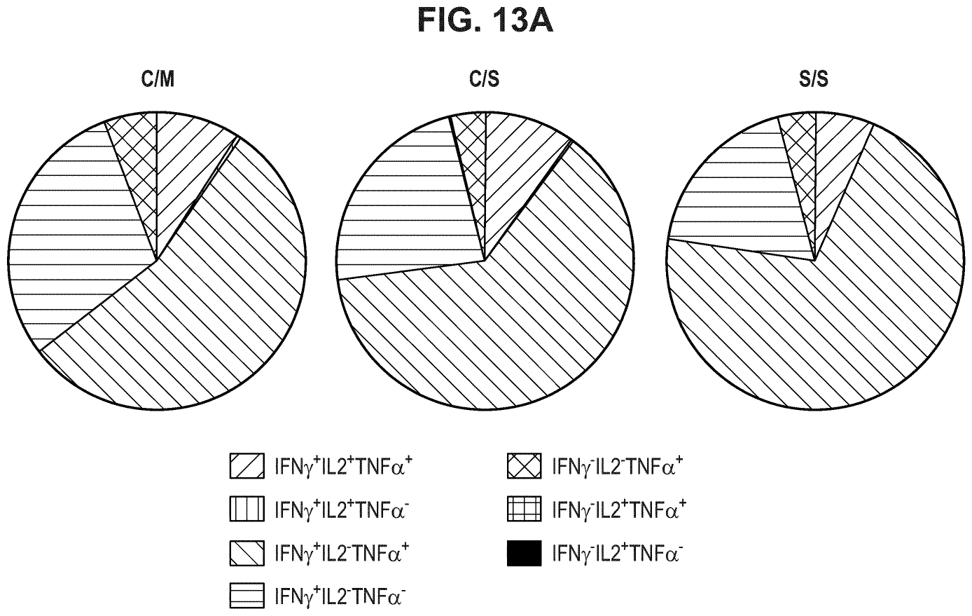

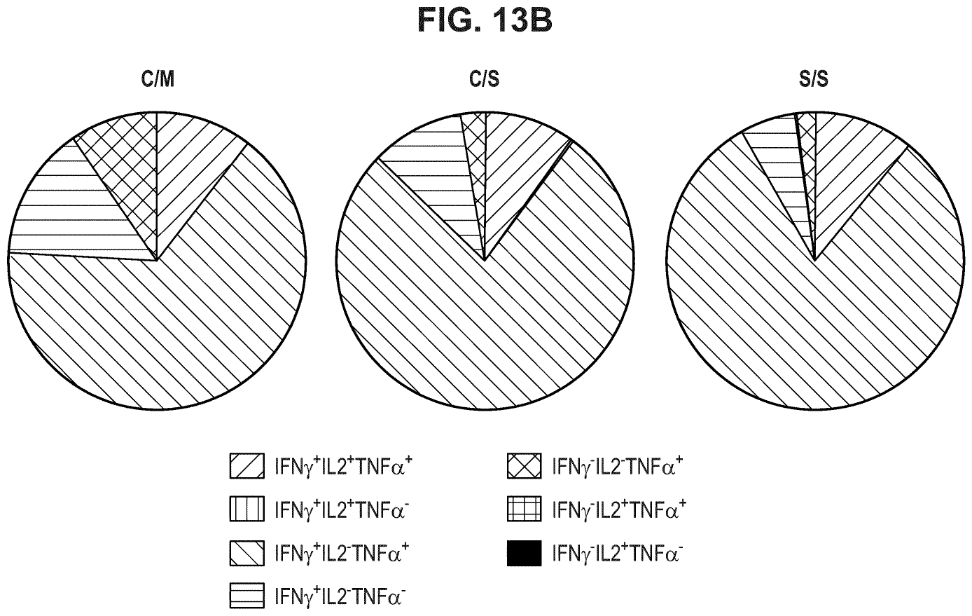

Interestingly, immunization with Th HBV vaccines regardless of the regimen used (sequential or co-administration) showed reductions of serum HBs antigen by ±1.5 fold in all groups with no between-group difference (FIGS. 10 and 11). Finally, as a liver-related inflammation parameter, the serum activities of AST and ALT were measured in mice sera after the second and fourth immunization. For each group, levels of ALT were stable during all the study and were not significantly impacted either by replacement of MVA-HBV or both ChAd155-hli-HBV and MVA-HBV by SAM-hli-HBV. A slightly higher AST levels were measured in all groups with no between-group difference (FIG. 12).

### Overall Conclusions

Replacing MVA-HBV or both ChAd155-hli-HBV and MVA-HBV by SAM-hli-HBV induced significantly higher HBc- and -HBs specific CD8+ T cell responses: SAM/SAM>ChAd/SAM>ChAd/MVA with a slight positive impact for HBs-specific CD4+ T cell responses. No significant impact was observed for the level of anti-HBc and -HBs IgG antibody responses.

Regarding the circulating HBs antigen, there is a tendency for reduction of circulating HBs antigen by ±1.5 fold in all groups with no between-group difference.

Furthermore, while assessing the potential vaccine-associated liver inflammation by measuring serum activities of Aspartate aminotransferase (AST) and Alanine aminotransferase (ALT), no increase in liver enzymes was detected in the vaccine groups when compared with the non-vaccinated one.

### Example 3: Immunogenicity Evaluation of Co-Administered LNP-mRNAs in HLA-A2/DRB1 Naive Mice

Details of this experiment are given in Table 3. The LNP-mRNA constructs contain the UTR4 backbone and RV39 LNPs. The formulation further comprises 1,2-distearoyl-sn-glycero-3-phosphocholine (DSPC), polyethylene glycol-conjugated (PEG-conjugated) lipid, and cholesterol.

Naïve HLA-A2/DRB1 mice (54 males, 47 females) of 8-12 weeks old were used in this experiment. The schedule involved injections on days 0, 21, 42 and 63 via an intramuscular route of immunization. The doses used were:
mRNA: as specified in the table,
ChAd155-hli-HBV: 10⁸ vp/mouse,
MVA-HBV: 10⁷ pfu/mouse,
HBc-HBs: 4-1 μg/AS01, i.e 4 μg HBc and 1 μg HBs (for the adjuvanted protein administered at the same time as the mRNA in group 7 of Table 3).

The ChAd155-hli-HBV vector encodes the hli-HBc-2A-HBs amino acid sequence of SEQ ID NO:15, and the MVA-HBV vector encodes the HBc-2A-HBs amino acid sequence of SEQ ID NO:5.

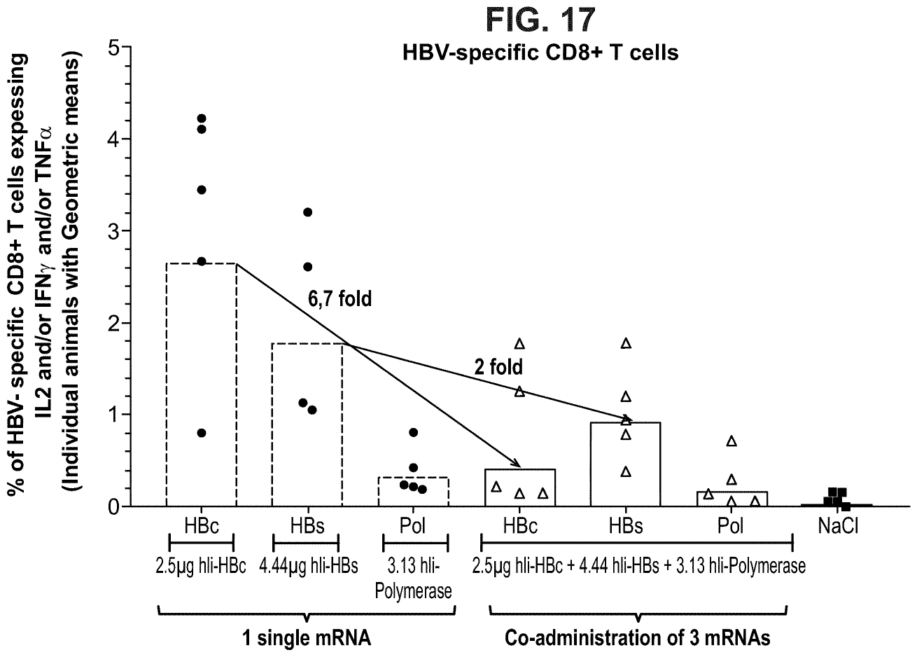

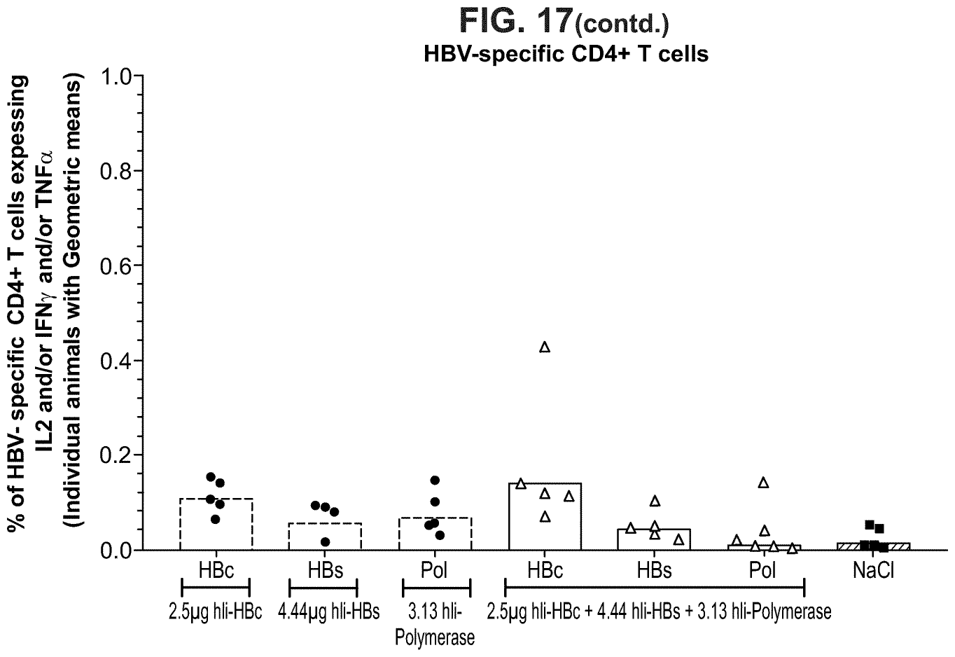

The main purpose of this experiment was to investigate the immune interference between co-administered hli-HBc and hli-HBs mRNAs (i.e. “hli-HBc+ hli-HBs”). An experiment was performed in which 3 co-administered mRNAs (including hli-HBc and hli-HBs) were compared to formulations containing only a single mRNA type. In this earlier experiment it was observed that co-administration negatively impacted the HBc- and HBs-specific CD8+ T cell responses (see FIG. 17). In particular, the HBc-specific response was 6.7-fold lower, and the HBs-specific response was 2-fold lower when these mRNAs were co-administered.

The first change made was to just co-administer the HBc and HBs mRNAs (i.e. the third mRNA was not used). In addition, as the impact was greatest for the HBc-specific CD8+ response, the amount of HBs mRNA was reduced relative to the HBc mRNA. This produced the different ratios of HBc mRNA to HBs mRNA observed in groups 2 to 5 of Table 3:
A composition containing 7 μg HBc mRNA and 7 μg HBs mRNA was administered to the mice of group 2,
For group 3, a 1.5-fold dilution of the HBs mRNA was performed resulting in 4.6 μg (as rounded down to 1 decimal point) of HBs mRNA being administered to the mice.
   The ratio of the mRNA's used in this composition was, therefore, 1.5 HBc mRNA: 1 HBs mRNA.
For group 4, a 1.5-fold dilution of the 4.6 μg HBs mRNA composition was performed, giving a composition with 3.1 μg (as rounded down to 1 decimal point) being administered to the mice.
   The ratio of the mRNA's used in this composition was, therefore, 2.3 HBc mRNA: 1 HBs mRNA.
For group 5, a further 1.5 dilution of the 3.1 μg HBs mRNA composition was performed, giving a composition with 2.0 μg (as rounded down to 1 decimal point) being administered to the mice.
   The ratio of the mRNA's used in this composition was, therefore, 3. 5 HBc mRNA: 1 HBs mRNA.

To select the co-administration (from groups 2, 3, 4 and 5) containing the ratio of LNP-mRNAs associated with the lowest level of immune interference as compared to LNP-mRNA alone (i.e. groups 8, 9, 10, 11 and 12). We, therefore:
Evaluated the potential negative impact on the HBc-specific CD8+ T cell responses.
Evaluated the potential negative impact on the HBs-specific CD8+ T cell responses.

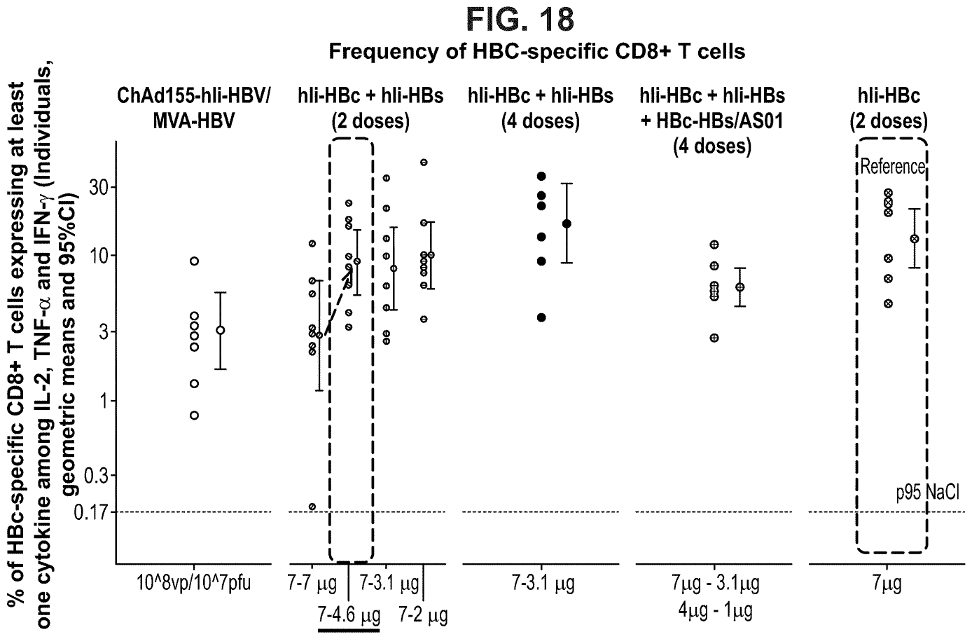

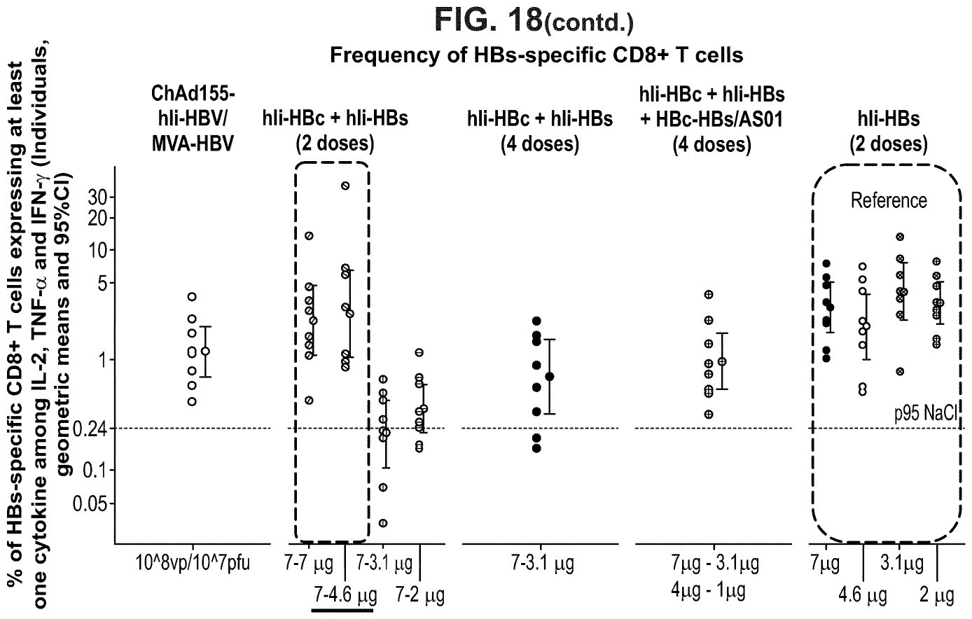

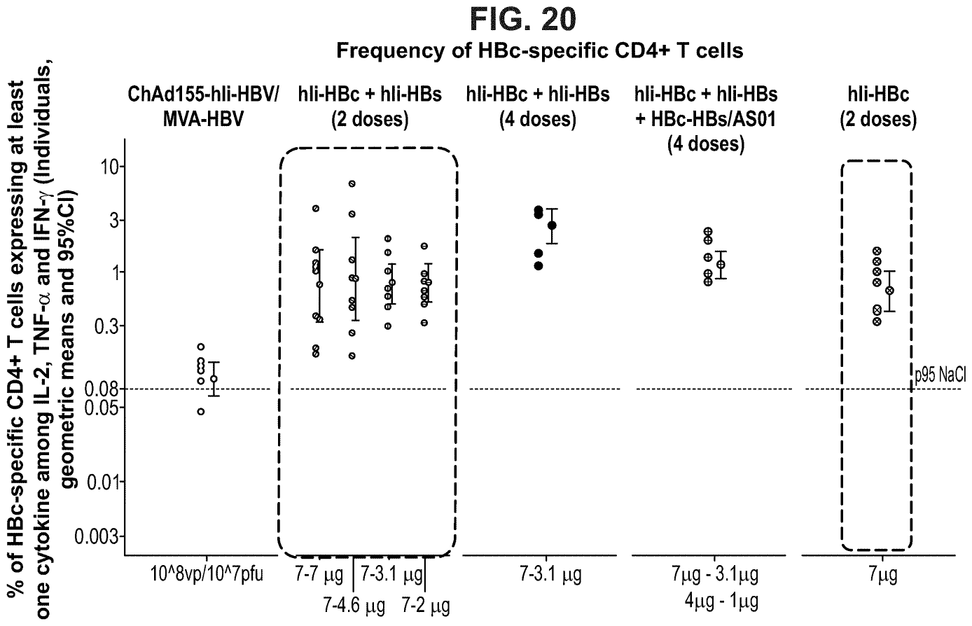

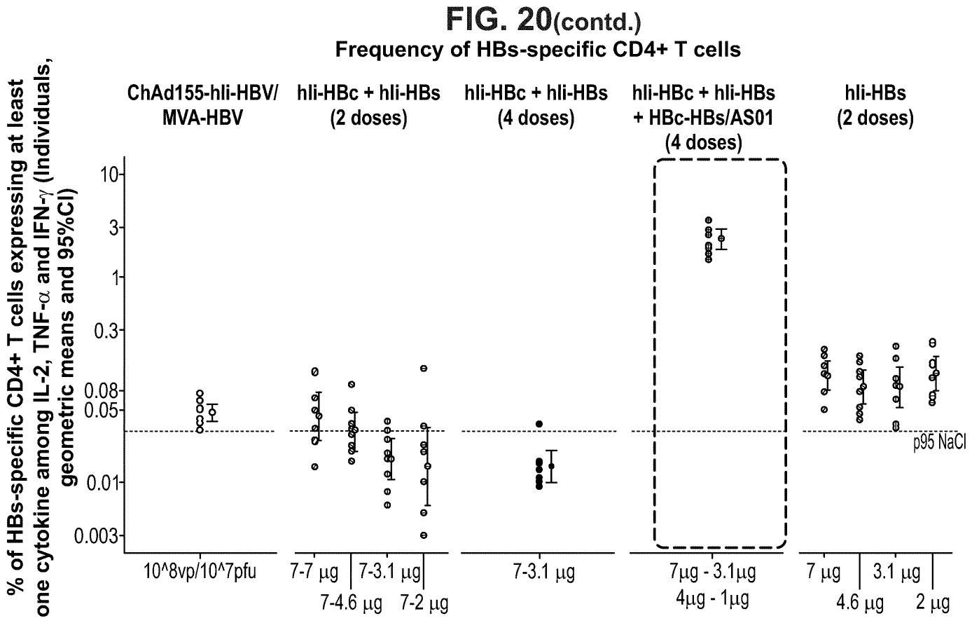

To assess the immune interference, the HBc- and HBs-specific CD8+ T cell responses in the spleen were measured at days 75 and 77 (see FIGS. 18, 19 and 20).

The success criteria was defined as: “To evaluate the non-inferiority of the co-administration of different LNP-mRNA ratios vs single LNP-mRNA formulation. The non-inferiority will be shown if the lower limit of the 90% confidence intervals of the geometric mean ratios are above 0.33. If non-inferiority will be statistically shown, the biological relevance will be assessed by the Scientist.”

With a SD lower than 0.36 with a sample size of 8 mice, a 3-fold non-inferiority can be shown with at least 80% of at 5% of level alpha. 8 mice are allocated per group and 5 mice to NaCl group for a total of 101 mice.

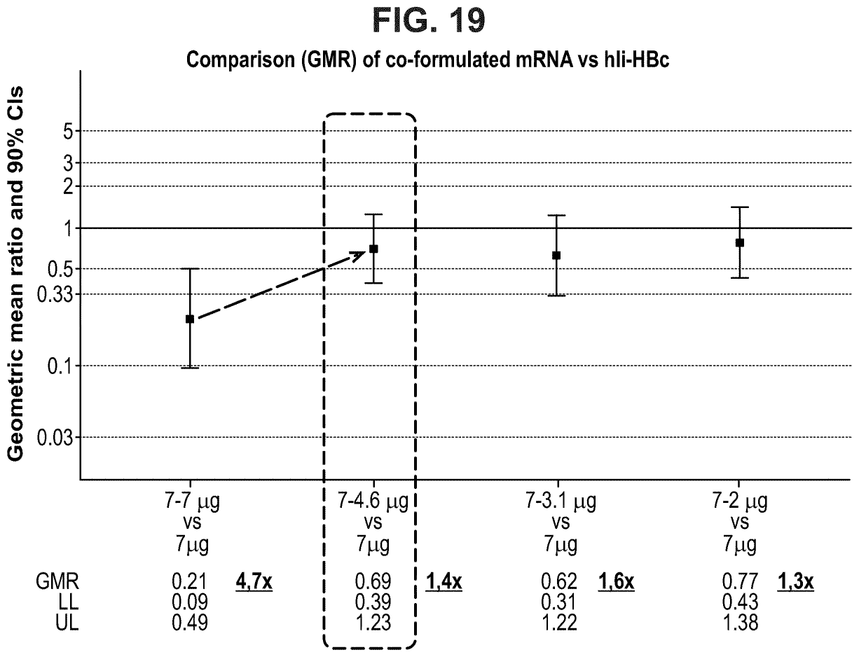

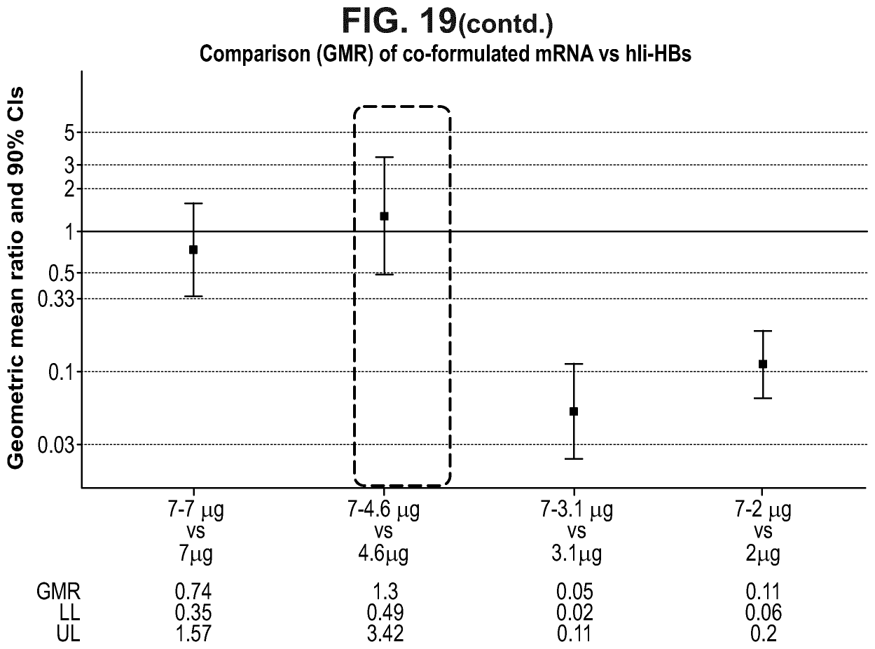

The results showed that reducing the amount of hli-HBs mRNA relative to the hli-HBc mRNA improved the HBc-specific CD8+ response as compared to control group (mice immunized with 7 μg of hli-HBc mRNA alone) (see FIG. 19). The highest HBc-specific CD8+ response was observed when 4.6 μg of the hli-HBs mRNA was used.

Interestingly, the composition with 7 μg hli-HBc and 4.6 μg hli-HBs was also found to produce similar level of HBs-specific CD8+ T-cell response than the composition with 7 μg hli-HBc and 7 μg hli-HBs and this response was similar to the one detected in group of mice immunized with 4,6 μg of HBs hli-HBs mRNA alone. Although the 7 μg-3.1 μg and 7 μg-2 μg compositions resulted in higher HBc-specific T cell responses than 7 μg-7 μg, a significant decrease in the HBs-specific CD8+ T-cell response was observed for these two compositions.

Therefore, the co-administration containing 7 μg hli-HBc-mRNA and 4.6 μg hli-HBs-mRNA (i.e. a ratio of 1.5 hli-HBc-mRNA: 1 hli-HBs-mRNA) was found to be the preferred composition as immune responses induced by co-administered mRNA were similar to the immune responses induced by each mRNA separately.

The results from this experiment also showed:
No Negative impact of co-administration on HBc-specific CD4+ T cell responses, and
No negative interference of co-administration (hli-HBc+ hli-HBs) on HBc-specific IgG responses.

The experiment was also designed to:
(i) To directly compare a prime-boost immunization using co-administration of mRNA (hli-HBc+ hli-HBs) vs ChAd/MVA (comparison between groups 1 vs 2, 3, 4 and 5 in Table 3).
(ii) To assess the immunogenicity of 4 doses vs 2 doses of co-administered mRNAs (hli-HBc+ hli-HBs) (comparison between groups 4 and 6 in Table 3).
(iii) To investigate any immune interference between co-administered mRNA (hli-HBc+ hli-HBs) and AS01-adjuvanted proteins (comparison of groups 6 and 7 of Table 3). In group 7, the “hli-HBc+ hli-HBs” and “HBc-HBs/AS01” compositions were administered at the same time but as two different injections into two different limbs of the mice.

The endpoints for these secondary objectives were the HBc- and HBs-specific CD4+ and CD8+ T cell responses in the spleen measured by ICS at days 75/77, and the HBc- and HBs-specific antibody responses at days 75 and 77 as measured by ELISA (see FIGS. 18, 20 and 21).

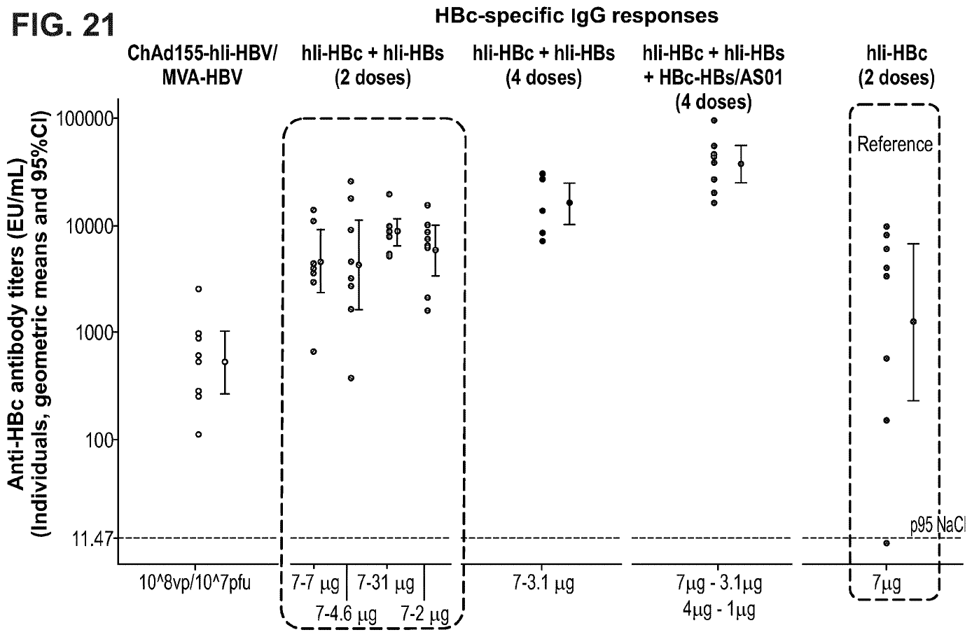

The respective results were as follows:
(i) For 2 doses of mRNA vs ChAd/MVA prime boost:
   Two doses of mRNA were found to induce higher CD8+ T cells responses than the ChAd/MVA prime boost. In particular, there was found to be a 3-fold increase in the CD8+ T cell response for HBc, and a 2-fold increase for HBs (see FIG. 18).
   Two doses of mRNA were also found to induce an 8-fold higher HBc-specific CD4+ T cell response. No HBs-CD4+ T-cell response was induced by either mRNA or ChAd/MVA (see FIG. 19).
   The two doses of mRNA were also found to induce an 8-fold higher HBc-specific IgG response (see FIG. 21). No HBs-specific IgG response was induced by either mRNA or ChAd/MVA.
(ii) For 4 doses vs 2 doses of mRNA:
   Two additional immunizations with co-administered mRNA were found to include the CD8+ T-cell responses. In particular, there was found to be a 2-fold increase in the CD8+ T cell response for HBc, and a 3-fold increase for HBs (see FIG. 18).
   Four doses of mRNA were also found to induce a 3.5-fold higher HBc-specific CD4+ T cell response. No HBs-specific CD4+ T-cell response was induced after either 4 or 2 doses (see FIG. 19).
   The four doses of mRNA were also found to induce a 2-fold higher HBc-specific IgG response (see FIG. 21). No HBs-specific IgG response was induced by either 4 or 2 doses.
(iii) For the co-administration of mRNA and adjuvanted proteins:
   The co-administration of adjuvanted proteins and co-administered mRNA (hli-HBc+ hli-HBs) was found to have a negative impact on the HBc-specific CD8+ T-cell response. However, this co-administration regimen was not found to negatively impact the HBs-specific CD8+ T-cell response (see FIG. 18).
   The co-administration regimen was found to negatively impact the HBc-specific CD4+ T-cell response. However, the inclusion of the adjuvanted proteins in the formulation also resulted in a HBs-specific CD4+ T-cell response being induced (see FIG. 19).
   The co-administration regimen was also found to have a positive impact on the HBc-specific IgG response (see FIG. 21). In addition, inclusion of the adjuvanted proteins in the formulation resulted in a HBs-specific IgG response being induced.

TABLE 3

| | | | | | Day | | Organ |
|---|---|---|---|---|---|---|---|
| | | | Immunisation | Immunisation | Immunisation | Immunisation | collection |
| | | | I | II | III | IV | day 75 & |
| Groups | N | Sex | Day 0 | Day 21 | Day 42 | Day 63 | 77 |
| 1 | 8 | 8F | — | — | ChAd155-hli- | MVA-HBV | Spleen |
| | | | | | HBV | | and |
| | | | | | | | serum |
| 2 | 8 | 4M- | — | — | 7 μg hli-HBc + | 7 μg hli-HBc + | Spleen |
| | | 4F | | | 7 μg hli-HBs | 7 μg hli-HBs | and |
| | | | | | | | serum |
| 3 | 8 | 5M- | — | — | 7 μg hli-HBc + | 7 μg hli-HBc + | Spleen |
| | | 3F | | | 4.6 μg hli-HBs | 4.6 μg hli-HBs | and |
| | | | | | | | serum |
| 4 | 8 | 5M- | — | — | 7 μg hli-HBc + | 7 μg hli-HBc + | Spleen |
| | | 3F | | | 3.1 μg hli-HBs | 3.1 μg hli-HBs | and |
| | | | | | | | serum |
| 5 | 8 | 5M- | — | — | 7 μg hli-HBc + | 7 μg hli-HBc + | Spleen |
| | | 3F | | | 2 μg hli-HBs | 2 μg hli-HBs | and |
| | | | | | | | serum |
| 6 | 8 | 5M- | 7 μg hli-HBc + | 7 μg hli-HBc + | 7 μg hli-HBc + | 7 μg hli-HBc + | Spleen |
| | | 3F | 3.1 μg hli-HBs | 3.1 μg hli-HBs | 3.1 μg hli-HBs | 3.1 μg hli-HBs | and |
| | | | | | | | serum |
| 7 | 8 | 5M- | 7 μg hli-HBc + | 7 μg hli-HBc + | 7 μg hli-HBc + | 7 μg hli-HBc + | Spleen |
| | | 3F | 3.1 μg hli-HBs | 3.1 μg hli-HBs | 3.1 μg hli-HBs | 3.1 μg hli-HBs | and |
| | | | & | & | & | & | serum |
| | | | HBc- | HBc- | HBc- | HBc- | |
| | | | HBs/AS01 | HBs/AS01 | HBs/AS01 | HBs/AS01 | |
| 8 | 8 | 5M- | — | — | 7 μg hli-HBc | 7 μg hli-HBc | Spleen |
| | | 3F | | | | | and |
| | | | | | | | serum |
| 9 | 8 | 5M- | — | — | 7 μg hli-HBs | 7 μg hli-HBs | Spleen |
| | | 3F | | | | | and |
| | | | | | | | serum |
| 10 | 8 | 5M- | — | — | 4.6 μg hli-HBs | 4.6 μg hli-HBs | Spleen |
| | | 3F | | | | | and |
| | | | | | | | serum |
| 11 | 8 | 5M- | — | — | 3.1 μg hli-HBs | 3.1 μg hli-HBs | Spleen |
| | | 3F | | | | | and |
| | | | | | | | serum |
| 12 | 8 | 5M- | — | — | 2 μg hli-HBs | 2 μg hli-HBs | Spleen |
| | | 3F | | | | | and |
| | | | | | | | serum |
| 13 | 5 | 2F- | NaCl | NaCl | NaCl | NaCl | Spleen |
| | | 3F | | | | | and |
| | | | | | | | serum |

### Embodiments of the Invention

Embodiments of the invention are described below, in the three following groups of embodiments. When appropriate, features from the three groups can be combined to form individual embodiments.

Group 1 of the embodiments describes:
Embodiment A. A composition for treating chronic hepatitis B infection comprising a mRNA encoding at least a hepatitis B virus core antigen (HBc), wherein the mRNA is encapsulated in a lipid nanoparticle (LNP).
Embodiment B. The composition of embodiment A, wherein the hepatitis B virus core antigen (HBc) comprises an amino acid sequence having at least 90%, 95%, 98% or 99% identity to the amino acid sequence set forth in SEQ ID NO:11.
Embodiment C. The composition of any preceding embodiment, wherein the hepatitis B virus core antigen (HBc) is fused to human invariant chain (hli).
Embodiment D. The composition of any preceding embodiment, wherein the composition further comprises an mRNA encoding a hepatitis B small surface protein (HBs).
Embodiment E. The composition of embodiment D, wherein the hepatitis B small surface protein (HBs) comprises an amino acid sequence having at least 90%, 95%, 98% or 99% identity to the amino acid sequence set forth in SEQ ID NO:1.
Embodiment F. The composition of any preceding embodiment, wherein the hepatitis B small surface antigen (HBs) is fused to human invariant chain (hli).
Embodiment G. A composition for treating chronic hepatitis B infection comprising a mRNA encoding at least a hepatitis B virus surface protein (HBsAg), wherein the mRNA is encapsulated in a lipid nanoparticle (LNP).
Embodiment H. The composition of embodiment G, wherein the HBsAg is hepatitis B small surface protein (HBs).
Embodiment I. The composition of embodiment H, wherein the HBs comprises an amino acid sequence having at least 90%, 95%, 98% or 99% identity to the amino acid sequence set forth in SEQ ID NO:1.
Embodiment J. The composition of any of embodiments G to I, wherein the HBsAg is fused to human invariant chain (hli).
Embodiment K. The composition of any preceding embodiment, wherein the human invariant chain (hli) comprises an amino acid sequence having at least 90%, 95%, 98% or 99% identity to the amino acid sequence set forth in SEQ ID NO: 7 or SEQ ID NO:12.
Embodiment L. The composition of embodiment K, wherein the human invariant chain (hli) comprises an amino acid sequence having at least 90%, 95%, 98% or 99% identity to the amino acid sequence set forth in SEQ ID NO:12.
Embodiment M. The composition of any preceding embodiment, wherein the composition is administered sequentially or concomitantly with one or more recombinant hepatitis B polypeptide(s).
Embodiment N. The composition of embodiment M, wherein the recombinant hepatitis B polypeptides comprise a recombinant hepatitis B core protein (HBc) and a recombinant hepatitis B small surface protein (HBs).
Embodiment O. The composition of embodiments M or N, wherein the HBc comprises an amino acid sequence an amino acid sequence having at least 90%, 95%, 98% or 99% identity to the amino acid sequence set forth in SEQ ID NO:2.
Embodiment P. The composition of embodiments M or N, wherein the HBs comprises an amino acid sequence an amino acid sequence having at least 90%, 95%, 98% or 99% identity 30 to the amino acid sequence set forth in SEQ ID NO:1.
Embodiment Q. The compositions of any one of embodiments M to P, wherein the recombinant hepatitis B polypeptide(s) is administered with an adjuvant.
Embodiment R. The composition of embodiment Q, wherein the adjuvant is AS01.
Embodiment S. A method of treating chronic hepatitis B infection comprising administering to a human a prime-boost regimen, wherein a mRNA encoding at least one hepatitis B virus antigen is administered as a priming dose, and one or more recombinant hepatitis B polypeptide(s) is administered as a booster dose.
Embodiment T. The method of embodiment S, wherein the mRNA encodes at least one hepatitis B virus antigen selected from the group consisting of hepatitis B core antigen (HBc) and hepatitis B surface antigen (HBsAg).
Embodiment U. The method of embodiment T, wherein the hepatitis B surface antigen (HBsAg) is hepatitis B small surface protein (HBs).
Embodiment V. The method of any one of embodiments S to U, wherein the hepatitis B virus antigen is fused to hli.
Embodiment W. The method of any one of embodiments S to U, wherein the recombinant hepatitis B polypeptides comprise a recombinant hepatitis B core protein (HBc) and a recombinant hepatitis B small surface protein (HBs).
Embodiment X. The method of any one of embodiments S to W, wherein the recombinant hepatitis B polypeptide(s) is administered with an adjuvant.
Embodiment Y. The method of embodiment X, wherein the adjuvant is AS01.

Group 2 of the embodiments describes:
Embodiment i. A composition for treating chronic hepatitis B infection comprising a first mRNA encoding at least a hepatitis B virus core antigen (HBc), wherein the first mRNA is encapsulated in a lipid nanoparticle (LNP).
Embodiment ii. The composition of embodiment ii, wherein the hepatitis B virus core antigen (HBc) comprises an amino acid sequence having at least 90%, 95%, 98% or 99% identity to the amino acid sequence set forth in SEQ ID NO:11.
Embodiment iii. The composition of any preceding embodiment, wherein the hepatitis B virus core antigen (HBc) is fused to human invariant chain (hli).
Embodiment iv. The composition of any preceding embodiment, wherein the composition further comprises a second mRNA encoding a hepatitis B small surface protein (HBs).
Embodiment v. The composition of embodiment iv, wherein the first mRNA encoding HBc (“HBc mRNA”) is encapsulated in different LNPs to the second mRNA encoding HBs (“HBs mRNA”).
Embodiment vi. The composition of embodiment iv, wherein the first mRNA encoding HBc (“HBc mRNA”) is encapsulated in the same LNPs to the second mRNA encoding HBs (“HBs mRNA”).
Embodiment vii. The composition of any one of embodiments iv to vi, wherein the hepatitis B small surface protein (HBs) comprises an amino acid sequence having at least 90%, 95%, 98% or 99% identity to the amino acid sequence set forth in SEQ ID NO:1.
Embodiment viii. The composition of any one of embodiments iv to vii, wherein the hepatitis B small surface antigen (HBs) is fused to human invariant chain (hli).
Embodiment ix. The composition of any of embodiments iv to viii, wherein there is more first mRNA than second mRNA in the composition by weight.
Embodiment x. The composition of any of embodiments iv to ix, wherein the first mRNA 20 and the second mRNA are respectively present at a ratio of 1.5:1 by weight.
Embodiment xi. A composition for treating chronic hepatitis B infection comprising a first mRNA encoding at least a hepatitis B small surface protein (HBs), wherein the first mRNA is encapsulated in a lipid nanoparticle (LNP).
Embodiment xii. The composition of embodiment xi, wherein the HBs comprises an amino acid sequence having at least 90%, 95%, 98% or 99% identity to the amino acid sequence set forth in SEQ ID NO:1.
Embodiment xiii. The composition of embodiments xi or xxii wherein the HBs is fused to human invariant chain (hli).
Embodiment xiv. The composition of any of embodiments xi to xiii, wherein the composition further comprises a second mRNA encoding a hepatitis B virus core antigen (HBc).
Embodiment xv. The composition of embodiment xii, wherein the hepatitis B virus core antigen (HBc) comprises an amino acid sequence having at least 90%, 95%, 98% or 99% identity to the amino acid sequence set forth in SEQ ID NO:11.
Embodiment xvi. The composition of embodiment xiv or xv, wherein the hepatitis B virus core antigen (HBc) is fused to human invariant chain (hli).
Embodiment xvii. The composition of any preceding embodiment, wherein the human invariant chain (hli) comprises an amino acid sequence having at least 90%, 95%, 98% or 99% identity to the amino acid sequence set forth in SEQ ID NO: 7 or SEQ ID NO:12.
Embodiment xviii. The composition of embodiment xvii, wherein the human invariant chain (hli) comprises an amino acid sequence having at least 90%, 95%, 98% or 99% identity to the amino acid sequence set forth in SEQ ID NO:12.
Embodiment xix. The composition of any preceding embodiment, wherein the composition is administered sequentially or concomitantly with one or more recombinant hepatitis B polypeptide(s).
Embodiment xx. The composition of embodiment xix, wherein the recombinant hepatitis B polypeptides comprise a recombinant hepatitis B core protein (HBc) and a recombinant hepatitis B small surface protein (HBs).
Embodiment xxi. The composition of embodiments xix or xx, wherein the HBc comprises an amino acid sequence an amino acid sequence having at least 90%, 95%, 98% or 99% identity to the amino acid sequence set forth in SEQ ID NO:2.
Embodiment xxii. The composition of embodiments xix or xx, wherein the HBs comprises an amino acid sequence an amino acid sequence having at least 90%, 95%, 98% or 99% identity to the amino acid sequence set forth in SEQ ID NO:1.
Embodiment xxiii. The compositions of any one of embodiments xix to xxii, wherein the recombinant hepatitis B polypeptide(s) is administered with an adjuvant.
Embodiment xxiv. The composition of embodiment xxiii, wherein the adjuvant is AS01.

Group 3 of the embodiments describes:
Embodiment 1. A composition for treating chronic hepatitis B infection comprising a mRNA encoding at least a hepatitis B virus core antigen (HBc), wherein the mRNA is encapsulated in a lipid nanoparticle (LNP).
Embodiment 2. The mRNA of any preceding embodiment, wherein the hepatitis B virus core antigen (HBc) comprises an amino acid sequence having at least 90%, 95%, 98% or 99% identity to the amino acid sequence set forth in SEQ ID NO:11.
Embodiment 3. The mRNA of any preceding embodiment, wherein the hepatitis B virus core antigen (HBc) is fused to human invariant chain (hli).
Embodiment 4. The mRNA of any preceding embodiment, wherein the mRNA further encodes a hepatitis B small surface protein (HBs).
Embodiment 5. The mRNA of any preceding embodiment, wherein the hepatitis B small surface protein (HBs) comprises an amino acid sequence having at least 90%, 95%, 98% or 99% identity to the amino acid sequence set forth in SEQ ID NO:1.
Embodiment 6. The mRNA of any of embodiments 4 or 5, wherein there is more mRNA encoding HBc (HBc mRNA) than mRNA encoding HBs (HBs mRNA) by weight.
Embodiment 7. The mRNA of any of embodiments 4 to 6, wherein the HBc mRNA and the HBs mRNA are respectively present at a ratio of 1.5:1 by weight.
Embodiment 8. A composition for treating chronic hepatitis B infection comprising a mRNA encoding at least a hepatitis B virus surface protein (HBsAg), wherein the mRNA is encapsulated in a lipid nanoparticle (LNP).
Embodiment 9. The mRNA of embodiment 8, wherein the HBsAg is hepatitis B small surface protein (HBs).
Embodiment 10. The mRNA of embodiment 9, wherein the HBs comprises an amino acid sequence having at least 90%, 95%, 98% or 99% identity to the amino acid sequence set forth in SEQ ID NO:1.
Embodiment 11. The mRNA of embodiment 8 to 10, wherein the HBsAg is fused to human invariant chain (hli).
Embodiment 12. The mRNA of any preceding embodiment, wherein the human invariant chain (hli) comprises an amino acid sequence having at least 90%, 95%, 98% or 99% identity to the amino acid sequence set forth in SEQ ID NO: 7 or SEQ ID NO:12.
Embodiment 13. The mRNA of any preceding embodiment, wherein the human invariant chain (hli) comprises an amino acid sequence having at least 90%, 95%, 98% or 99% identity to the amino acid sequence set forth in SEQ ID NO:12.
Embodiment 14. The mRNA of any preceding embodiment, wherein the composition is administered sequentially or concomitantly with one or more recombinant hepatitis B polypeptide(s).
Embodiment 15. The mRNA of any preceding embodiment, wherein the recombinant hepatitis B polypeptides comprise a recombinant hepatitis B core protein (HBc) and a recombinant hepatitis B small surface protein (HBs).
Embodiment 16. The mRNA of any preceding embodiment, wherein the HBc comprises an amino acid sequence an amino acid sequence having at least 90%, 95%, 98% or 99% identity to the amino acid sequence set forth in SEQ ID NO:2.
Embodiment 17. The mRNA of any preceding embodiment, wherein the HBs comprises an amino acid sequence an amino acid sequence having at least 90%, 95%, 98% or 99% identity to the amino acid sequence set forth in SEQ ID NO:1.
Embodiment 18. The mRNA of any preceding embodiment, wherein the recombinant hepatitis B polypeptide(s) is administered with an adjuvant.
Embodiment 19. The mRNA of embodiment 18, wherein the adjuvant is AS01.
Embodiment 20. A method of treating chronic hepatitis B infection comprising administering to a human a prime-boost regimen, wherein a mRNA encoding at least one hepatitis B virus antigen is administered as a priming dose, and the mRNA encoding at least one hepatitis B virus antigen administered as a booster dose.
Embodiment 21. The method of embodiment 20, comprising administering four sequential doses of mRNA to the human.
Embodiment 22. The method of embodiment 20 or 21, wherein a separate composition comprising adjuvanted recombinant hepatitis B polypeptide(s) is administered at the same time as the mRNA, wherein the recombinant hepatitis B polypeptides comprise a recombinant hepatitis B core protein (HBc) and a recombinant hepatitis B small surface protein (HBs).
Embodiment 23. A method of treating chronic hepatitis B infection comprising administering to a human a prime-boost regimen, wherein a mRNA encoding at least one hepatitis B virus antigen is administered as a priming dose, and one or more recombinant hepatitis B polypeptide(s) is administered as a booster dose.
Embodiment 24. The method of embodiment 23, wherein the hepatitis B virus antigen is selected from the group consisting of hepatitis B core antigen (HBc), or hepatitis B surface antigen (HBsAg).
Embodiment 25. The method of embodiment 23, wherein the hepatitis B surface antigen (HBsAg) is hepatitis B small surface protein (HBs).
Embodiment 26. The method of embodiment 23 to 25, wherein the hepatitis B virus antigen is fused to hli.
Embodiment 27. The method of embodiment 23 to 26, wherein the recombinant hepatitis B polypeptides comprise a recombinant hepatitis B core protein (HBc) and a recombinant hepatitis B small surface protein (HBs).
Embodiment 28. The method of embodiment 23 to 27, wherein the recombinant hepatitis B polypeptide(s) is administered with an adjuvant.
Embodiment 29. The mRNA of any preceding embodiment, wherein the LNP comprise a PEG-modified lipid, a non-cationic lipid, a sterol, and a non-ionisable cationic lipid.
Embodiment 30. The mRNA of any preceding embodiment, wherein the LNP comprise a PEG-modified lipid, a non-cationic lipid, a sterol, and an ionisable cationic lipid.
Embodiment 31. The mRNA of any preceding embodiment, wherein the non-cationic lipid is a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) or sphingomyelin (SM).
Embodiment 32. The mRNA of any preceding embodiment, wherein the sterol is cholesterol.
Embodiment 33. The mRNA of any preceding embodiment, wherein the LNP comprise a PEG-modified lipid at around 0.5 to 15 molar %, a non-cationic lipid at around 5 to 25 molar % a sterol at around 25 to 55 molar % and an ionisable cationic lipid at around 20 to 60 molar %.
Embodiment 34. The mRNA of any preceding embodiment, wherein the LNP are 50 to 200 μm in diameter.
Embodiment 35. The mRNA of any preceding embodiment, wherein the LNP have a polydispersity of 0.4 or less, such as 0.3 or less.
Embodiment 36. The mRNA of any preceding embodiment, wherein the ratio of nucleotide (N) to phospholipid (P) is in the range of 1N:1P to 20N:1P, 1N:1P to 10N:1P, 2N:1P to 8N:1P, 2N:1P to 6N:1P or 3N:1P to 5N:1P.
Embodiment 37. The mRNA of any preceding embodiment, wherein at least half of the mRNA is encapsulated in the LNP, suitably at least 85%, especially at least 95%, such as 100%.
Embodiment 38. The mRNA of any preceding embodiment, wherein the mRNA is non-replicating or self-replicating mRNA (SAM).
Embodiment 39. The mRNA of any preceding embodiment, wherein the self-replicating RNA molecule encodes (i) an RNA-dependent RNA polymerase which can transcribe RNA from the self-replicating RNA molecule and (ii) the hepatitis B polypeptide.
Embodiment 40. The mRNA of any preceding embodiment, wherein the mRNA has the configuration 5′cap-5′UTR-non-structural proteins (NSP) 1-4-subgenomic promoter-hepatitis B polypeptide-3′UTR-polyA.
Embodiment 41. The mRNA of any preceding embodiment, for administration to a human subject with chronic hepatitis B infection.
Embodiment 42. The mRNA of any of preceding embodiment, wherein the mRNA is non-replicating mRNA.
Embodiment 43. The mRNA of any preceding embodiment, wherein the one or more recombinant hepatitis B polypeptide is administered with AS01 adjuvant.
Embodiment 44. The mRNA of any preceding embodiment, wherein the method comprises first administering the mRNA, then administering the one or more recombinant hepatitis B polypeptide.
Embodiment 45. The mRNA of any preceding embodiment, wherein the method comprises a prime-boost regimen, wherein the mRNA is administered as a priming dose, and 10 the one or more recombinant hepatitis B polypeptide is administered as a booster dose.
Embodiment 46. The mRNA of embodiment 45, wherein the method comprises a single prime of the mRNA, and multiple subsequent booster doses of the at recombinant hepatitis B polypeptide.
Embodiment 47. The mRNA of embodiment 46, wherein the method comprises two or three subsequent booster doses of the one or more recombinant hepatitis B polypeptide.
Embodiment 48. The mRNA of embodiment 45, wherein the method comprises multiple priming doses of the mRNA, and multiple subsequent booster doses of the at recombinant hepatitis B polypeptide.
Embodiment 49. The mRNA of embodiment 48, wherein the method comprises two priming doses of the mRNA, and two subsequent booster doses of the at recombinant hepatitis 25 B polypeptide.
Embodiment 50. An immunogenic composition comprising the mRNA of any preceding embodiment.
Embodiment 51. An immunogenic composition of embodiment 50 further comprising the one or more recombinant hepatitis B polypeptide.
Embodiment 52. An immunogenic combination comprising:
   (a) the mRNA of any of embodiments 1 to 49; and
   (b) the one or more recombinant hepatitis B polypeptide with which the mRNA is administered
Embodiment 53. The immunogenic combination of embodiment 52, wherein the one or more recombinant hepatitis B polypeptide comprises a hepatitis B surface antigen (HBs), a hepatitis B virus core antigen (HBc) and an adjuvant.
Embodiment 54. The immunogenic combination of embodiment 53, wherein the one or more recombinant hepatitis B polypeptide is combined with AS01 adjuvant.
Embodiment 55. The immunogenic combination of any one of embodiments 52 to 54 further comprising an adenoviral vector (which may be a replication-defective chimpanzee adenoviral (ChAd) vector encoding a hepatitis B polypeptide.
Embodiment 56. The immunogenic combination of embodiment 55, wherein the adenoviral vector encodes a hepatitis B virus core antigen (HBc) fused to human invariant chain (hli).
Embodiment 57. The immunogenic combination of embodiment 56, wherein the adenoviral vector additionally encodes a hepatitis B virus surface antigen (HBs).
Embodiment 58. The immunogenic combination of any of embodiments 55 to 57, wherein adenoviral vector encodes a polypeptide comprising an amino acid sequence having at least 90%, 95%, 98% or 99% identity to the amino acid sequence set forth in SEQ ID NO:15.
Embodiment 59. The immunogenic combination of any of embodiments 55 to 58, wherein adenoviral vector encodes a polypeptide consisting of an amino acid sequence having at least 90%, 95%, 98% or 99% identity to the amino acid sequence set forth in SEQ ID NO:15.
Embodiment 60. The immunogenic combination of any of embodiments 55 to 59, wherein adenoviral vector encodes a polypeptide comprising the amino acid sequence set forth in SEQ ID NO:15.
Embodiment 61. The immunogenic combination of any of embodiments 55 to 60, wherein adenoviral vector encodes a polypeptide consisting of the amino acid sequence set forth in SEQ ID NO:15.
Embodiment 62. An immunogenic combination comprising:
   A first composition comprising an mRNA encoding a hepatitis B virus core antigen (HBc) encapsulated in a lipid nanoparticle (LNP), and an mRNA encoding a hepatitis B small surface protein (HBs) encapsulated in a lipid nanoparticle (LNP); and
   A second composition comprising recombinant hepatitis B core protein (HBc) and recombinant hepatitis B small surface protein (HBs) and an adjuvant, Embodiment 63. The immunogenic combination of embodiment 62, wherein second composition comprises AS01 adjuvant.
Embodiment 64. The combination of embodiment 62 or 63 for use in a method of treating chronic hepatitis B (CHB) by sequential or concomitant administration of the compositions.
Embodiment 65. A method of treating chronic hepatitis B (CHB) infection in a human, wherein the method comprises administering to the human the mRNA of any of embodiments 1 to 49 either sequentially or concomitantly with the one or more recombinant hepatitis B polypeptide.
Embodiment 66. The method of treating chronic hepatitis B infection (CHB) in a human of embodiment 62, wherein the one or more recombinant hepatitis B polypeptide is a recombinant hepatitis B virus core antigen (HBc).
Embodiment 67. The method of treating chronic hepatitis B infection (CHB) in a human of embodiment 63, wherein the composition further comprises a recombinant hepatitis B surface antigen (HBs), and an adjuvant.
Embodiment 68. The method of treating chronic hepatitis B infection (CHB) in a human of embodiment 62 or 63, wherein the composition further comprises an adjuvant.
Embodiment 69. The method of treating chronic hepatitis B infection (CHB) in a human of embodiment 65, wherein the adjuvant contains MPL and QS-21.
Embodiment 70. The method of treating chronic hepatitis B infection (CHB) in a human of embodiment 63, wherein the recombinant hepatitis B surface antigen (HBs) is a C-terminal truncated recombinant hepatitis B virus core antigen (HBc).
Embodiment 71. The method of treating chronic hepatitis B (CHB) infection in a human of an one of embodiments 62 to 67, wherein the method further comprises administering to the human an adenoviral vector comprising a polynucleotide encoding a hepatitis B polypeptide.
Embodiment 72. The method of treating chronic hepatitis B infection (CHB) in a human of embodiment 68, wherein the adenoviral vector is a replication-defective chimpanzee adenoviral (ChAd) vector.
Embodiment 73. The method of treating chronic hepatitis B infection (CHB) in a human of embodiment 68 or 69, wherein the adenoviral vector encodes a hepatitis B polypeptide fused to human invariant chain (hli).
Embodiment 74. The method of treating chronic hepatitis B infection (CHB) in a human of any of embodiments 68 to 70, wherein the adenoviral vector encodes a hepatitis B virus core antigen (HBc).
Embodiment 75. The method of treating chronic hepatitis B infection (CHB) in a human of embodiment 71, wherein the adenoviral vector additionally encodes a hepatitis B virus surface antigen (HBs).
Embodiment 76. Use of mRNA of any of embodiments 1 to 49, or the immunogenic combination of any of embodiments 50 to 61, in the treatment of HBV.
Embodiment 77. Use of mRNA of any of embodiments 1 to 49, or the immunogenic combination of any of embodiments 50 to 61, to reduce the levels of circulating hepatitis B surface antigen (HBs) in patients infected with HBV.
Embodiment 78. Use of mRNA of any of embodiments 1 to 49, or the immunogenic combination of any of embodiments 50 to 61, in the manufacture of a medicament.
Embodiment 79. Use of mRNA of any of embodiments 1 to 5449 or the immunogenic combination of any of embodiments 50 to 61, in the manufacture of a medicament for the treatment of HBV.
Embodiment 80. A kit comprising the following components:
   (a) the mRNA of any of embodiments 1 to 49; and
   (b) the one or more recombinant hepatitis B polypeptide with which the mRNA is administered

### REFERENCES

World Health Organization (WHO). Global Hepatitis report, 2017. http://apps.who.int/iris/bitstream/10665/255016/1/9789241565455-eng.pdf?ua=1. Accessed November 2017.
Block T M, Locarnini S, McMahon B J, Rehermann B, Peters M G. Use of Current and New Endpoints in the Evaluation of Experimental Hepatitis B Therapeutics. Clin Infect Dis. 2017; 64(9):1283-1288.
Cornberg M, Wong V W, Locarnini S, Brunetto M, Janssen H L, Chan H L. The role of quantitative hepatitis B surface antigen revisited. J Hepatol. 2017; 66(2):398-411.
EASL 2017. Clinical Practice Guidelines on the management of hepatitis B virus infection. J Hepatol 2017; 67(2), 370-398.
Borghese F, Clanchy F I. CD74: an emerging opportunity as a therapeutic target in cancer and autoimmune disease. Expert Opin Ther Targets. 2011; 15(3):237-51.
Jiang S, et al., Lipidoid-Coated Iron Oxide Nanoparticles for Efficient DNA and siRNA delivery Nano Lett., 13:1059-1064 (2013).
Zhang, C et al. Advances in mRNA vaccines for infectious diseases, Front. Immunol. 2019, 10:594.
Boni C, Laccabue D, Lampertico P, et al. Restored function of HBV-specific T cells afterlong-term effective therapy with nucleos(t)ide analogues. Gastroenterology. 2012; 143(4): 963-73.e9.
Rehermann B, Ferrari C, Pasquinelli C, Chisari F V. The hepatitis B virus persists for decades after patients' recovery from acute viral hepatitis despite active maintenance of a cytotoxic T-lymphocyte response. Nat Med. 1996; 2(10): 1104-8.
Thimme R, Wieland S, Steiger C, et al. CD8+ T Cells Mediate Viral Clearance and Disease Pathogenesis during Acute Hepatitis B Virus Infection. Journal of Virology 2003; 77 (1):68-76.
WO2005/113782
WO2011/076807
WO2012/006376
WO2012/006378
WO2012/006380
WO2012/030901
WO2012/031043
WO2012/031046
WO2013/006825
WO2013/006834
WO2013/006837
WO2013/033563
WO2014/136086
WO2015/095340
WO2015/095346
WO2016/037053
WO2017/070620
WO2017/162265

SEQUENCE LISTING
SEQ ID NO: 1:
Amino acid sequence of HBs
MENITSGFLGPLLVLQAGFFLLTRILTIPQSLDSWWTSLNFLGGSPVCLGQNSQSPTSNH
SPTSCPPICPGYRWMCLRRFIIFLFILLLCLIFLLVLLDYQGMLPVCPLIPGSTTTNTGP
CKTCTTPAQGNSMFPSCCCTKPTDGNCTCIPIPSSWAFAKYLWEWASVRFSWLSLLVPFV
QWFVGLSPTVWLSAIWMMWYWGPSLYSIVSPFIPLLPIFFCLWVYI
SEQ ID NO: 2:
Amino acid sequence of HBc truncate
MDIDPYKEFGATVELLSFLPSDFFPSVRDLLDTASALYREALESPEHCSPHHTALRQAIL
CWGELMTLATWVGNNLEDPASRDLVVNYVNTNMGLKIRQLLWFHISCLTFGRETVLEYLV
SFGVWIRTPPAYRPPNAPILSTLPETTVV
SEQ ID NO: 3:
Amino acid sequence of spacer incorporating 2A cleaving region of the
foot and mouth disease virus
APVKQTLNFDLLKLAGDVESNPGP
SEQ ID NO: 4:
Nucleotide sequence encoding spacer incorporating 2A cleavage region
of the foot and mouth disease virus
GCCCCTGTGAAGCAGACCCTGAACTTCGACCTGCTGAAGCTGGCCGGCGACGTGGAGAGC
AATCCCGGCCCT
SEQ ID NO: 5:
Amino acid sequence of HBc-2A-HBs
MDIDPYKEFGATVELLSFLPSDFFPSVRDLLDTASALYREALESPEHCSPHHTALRQAIL
CWGELMTLATWVGNNLEDPASRDLVVNYVNTNMGLKIRQLLWFHISCLTFGRETVLEYLV
SFGVWIRTPPAYRPPNAPILSTLPETTVVRRRDRGRSPRRRTPSPRRRRSQSPRRRRSQS
RESQCAPVKQTLNFDLLKLAGDVESNPGPMENITSGFLGPLLVLQAGFFLLTRILTIPQS
LDSWWTSLNFLGGSPVCLGQNSQSPTSNHSPTSCPPICPGYRWMCLRRFIIFLFILLLCL
IFLLVLLDYQGMLPVCPLIPGSTTTNTGPCKTCTTPAQGNSMFPSCCCTKPTDGNCTCIP
IPSSWAFAKYLWEWASVRFSWLSLLVPFVQWFVGLSPTVWLSAIWMMWYWGPSLYSIVSP
FIPLLPIFFCLWVYI
SEQ ID NO: 6:
Nucleotide sequence encoding HBc-2A-HBs
ATGGACATCGATCCCTACAAGGAATTTGGCGCCACCGTGGAGCTGCTGAGCTTCCTGCCC
AGCGACTTCTTCCCCAGCGTGAGGGACCTCCTGGACACCGCCAGCGCCCTGTACAGGGAG
GCCCTGGAATCTCCCGAGCACTGCAGCCCACACCACACCGCACTGAGGCAGGCCATCCTG
TGCTGGGGAGAGCTGATGACCCTCGCCACCTGGGTGGGCAACAACCTGGAGGACCCCGCC
AGCAGGGACCTGGTGGTGAACTACGTCAACACCAACATGGGCCTGAAGATCAGGCAGCTG
CTGTGGTTCCACATCAGCTGCCTGACCTTCGGCAGGGAGACCGTGCTGGAGTACCTGGTG
AGCTTCGGCGTGTGGATCAGGACACCTCCCGCCTACAGACCCCCCAACGCCCCCATCCTG
AGCACCCTGCCCGAGACCACAGTGGTGAGGAGGAGGGACAGGGGCAGGTCACCCAGGAGG
AGGACTCCAAGCCCCAGGAGGAGGAGGAGCCAGAGCCCCAGGAGAAGGAGGAGCCAGAGC
AGGGAGAGCCAGTGCGCCCCTGTGAAGCAGACCCTGAACTTCGACCTGCTGAAGCTGGCC
GGCGACGTGGAGAGCAATCCCGGCCCTATGGAGAACATCACCAGCGGCTTCCTGGGCCCC
CTGCTGGTGCTGCAGGCAGGCTTCTTCCTGCTGACCAGGATCCTGACCATCCCCCAGAGC
CTGGACAGCTGGTGGACCAGCCTGAACTTCCTCGGCGGGAGCCCCGTGTGCCTGGGCCAG
AACAGCCAGTCTCCCACCAGCAATCACAGCCCCACCAGCTGCCCCCCAATCTGTCCTGGC
TACCGGTGGATGTGCCTGAGGAGGTTCATCATCTTCCTGTTCATCCTGCTCCTGTGCCTG
ATCTTCCTGCTGGTGCTGCTGGACTACCAGGGAATGCTGCCAGTGTGTCCCCTGATCCCC
GGCTCAACCACCACTAACACCGGCCCCTGCAAAACCTGCACCACCCCCGCTCAGGGCAAC
AGCATGTTCCCAAGCTGCTGCTGCACCAAGCCCACCGACGGCAACTGCACCTGCATTCCC
ATCCCCAGCAGCTGGGCCTTCGCCAAGTATCTGTGGGAGTGGGCCAGCGTGAGGTTCAGC
TGGCTCAGCCTGCTGGTGCCCTTCGTCCAGTGGTTTGTGGGCCTGAGCCCCACCGTGTGG
CTGAGCGCCATCTGGATGATGTGGTACTGGGGCCCCAGCCTGTACTCCATCGTGAGCCCC
TTCATCCCCCTGCTGCCCATTTTCTTCTGCCTGTGGGTGTACATC
SEQ ID NO: 7:
Amino acid sequence of hli
MHRRRSRSCREDQKPVMDDQRDLISNNEQLPMLGRRPGAPESKCSRGALYTGFSILVTLL
LAGQATTAYFLYQQQGRLDKLTVTSQNLQLENLRMKLPKPKPVSKMRMATPLLMQALPMG
ALPQGPMQNATKYGNMTEDHVMHLLQNADPLKVYPPLKSFPENLRHLKNTMETIDWKVFE
SWMHHWLLFEMSRHSLEQKPTDAPPKESLELEDPSSGGVTKQDLGPVPM
SEQ ID NO: 8:
Nucleotide sequence encoding hli
ATGCACAGGAGGAGGAGCAGGAGCTGCAGGGAGGACCAGAAGCCCGTGATGGACGACCAG
CGCGACCTGATCAGCAACAACGAGCAGCTGCCAATGCTGGGCAGGAGGCCCGGAGCACCC
GAAAGCAAGTGCAGCAGGGGCGCCCTGTACACCGGCTTCAGCATCCTGGTGACCCTCCTG
CTGGCCGGCCAGGCCACCACCGCCTATTTCCTGTACCAGCAGCAGGGCAGGCTCGATAAG
CTGACCGTGACCTCCCAGAACCTGCAGCTGGAGAACCTGAGGATGAAGCTGCCCAAGCCC
CCCAAGCCCGTGAGCAAGATGAGGATGGCCACCCCCCTGCTGATGCAGGCTCTGCCCATG
GGGGCCCTGCCCCAGGGCCCCATGCAGAACGCCACCAAATACGGCAACATGACCGAGGAC
CACGTGATGCACCTGCTGCAGAACGCCGATCCTCTGAAGGTGTACCCACCCCTGAAAGGC
AGCTTCCCCGAGAACCTCAGGCACCTGAAGAACACCATGGAGACCATCGACTGGAAGGTG
TTCGAGAGCTGGATGCACCACTGGCTGCTGTTCGAGATGAGCCGGCACAGCCTGGAGCAG
AAGCCCACCGACGCCCCTCCCAAGGAGAGCCTCGAGCTCGAGGACCCAAGCAGCGGCCTG
GGCGTGACCAAGCAGGACCTGGGCCCCGTGCCCATG
SEQ ID NO: 9:
Amino acid sequence of hli-HBc-2A-HBs
MHRRRSRSCREDQKPVMDDQRDLISNNEQLPMLGRRPGAPESKCSRGALYTGFSILVTLL
LAGQATTAYFLYQQQGRLDKLTVTSQNLQLENLRMKLPKPKPVSKMRMATPLLMQALPMG
ALPQGPMQNATKYGNMTEDHVMHLLQNADPLKVYPPLKSFPENLRHLKNTMETIDWKVFE
SWMHHWLLFEMSRHSLEQKPTDAPPKESLELEDPSSGGVTKQDLGPVPMMDIDPYKEFGA
TVELLSFLPSDFFPSVRDLLDTASALYREALESPEHCSPHHTALRQAILCWGELMTLATW
VGNNLEDPASRDLVVNYVNTNMGLKIRQLLWFHISCLTFGRETVLEYLVSFGVWIRTPPA
YRPPNAPILSTLPETTVVAPVKQTLNFDLLKLAGDVESNPGPMENITSGFLGPLLVLQAG
FFLLTRILTIPQSLDSWWTSLNFLGGSPVCLGQNSQSPTSNHSPTSCPPICPGYRWMCLR
RFIIFLFILLLCLIFLLVLLDYQGMLPVCPLIPGSTTTNTGPCKTCTTPAQGNSMFPSCC
CTKPTDGNCTCIPIPSSWAFAKYLWEWASVRFSWLSLLVPFVQWFVGLSPTVWLSAIWMM
WYWGPSLYSIVSPFIPLLPIFFCLWVYI
SEQ ID NO: 10:
Nucleotide sequence encoding hli-HBc-2A-HBs
ATGCACAGGAGGAGGAGCAGGAGCTGCAGGGAGGACCAGAAGCCCGTGATGGACGACCAG
CGCGACCTGATCAGCAACAACGAGCAGCTGCCAATGCTGGGCAGGAGGCCCGGAGCACCC
GAAAGCAAGTGCAGCAGGGGCGCCCTGTACACCGGCTTCAGCATCCTGGTGACCCTCCTG
CTGGCCGGCCAGGCCACCACCGCCTATTTCCTGTACCAGCAGCAGGGCAGGCTCGATAAG
CTGACCGTGACCTCCCAGAACCTGCAGCTGGAGAACCTGAGGATGAAGCTGCCCAAGCCC
CCCAAGCCCGTGAGCAAGATGAGGATGGCCACCCCCCTGCTGATGCAGGCTCTGCCCATG
GGGGCCCTGCCCCAGGGCCCCATGCAGAACGCCACCAAATACGGCAACATGACCGAGGAC
CACGTGATGCACCTGCTGCAGAACGCCGATCCTCTGAAGGTGTACCCACCCCTGAAAGGC
AGCTTCCCCGAGAACCTCAGGCACCTGAAGAACACCATGGAGACCATCGACTGGAAGGTG
TTCGAGAGCTGGATGCACCACTGGCTGCTGTTCGAGATGAGCCGGCACAGCCTGGAGCAG
AAGCCCACCGACGCCCCTCCCAAGGAGAGCCTCGAGCTCGAGGACCCAAGCAGCGGCCTG
GGCGTGACCAAGCAGGACCTGGGCCCCGTGCCCATGGACATTGACCCCTACAAGGAGTTC
GGCGCCACCGTCGAACTGCTGAGCTTCCTCCCCAGCGACTTCTTCCCCTCCGTGAGGGAT
CTGCTGGACACAGCTAGCGCCCTGTACAGGGAGGCCCTGGAGAGCCCCGAGCACTGCAGC
CCCCACCACACAGCCCTGAGGCAGGCCATCCTCTGTTGGGGCGAGCTGATGACCCTGGCC
ACCTGGGTGGGCAATAACCTGGAGGACCCCGCCAGCAGGGACCTGGTGGTCAACTACGTG
AACACCAACATGGGCCTGAAGATCAGGCAGCTGCTGTGGTTCCACATCAGCTGCCTGACC
TTTGGCAGGGAGACCGTCCTGGAGTACCTGGTGAGCTTCGGCGTGTGGATCAGGACTCCC
CCAGCCTACAGGCCCCCTAACGCCCCCATCCTGTCTACCCTGCCCGAGACCACCGTGGTG
AGGAGGAGGGACAGGGGCAGAAGCCCCAGGAGAAGGACCCCTAGCCCCAGGAGGAGGAGG
AGCCAGAGCCCCAGGAGGAGGAGGAGCCAGAGCCGGGAGAGCCAGTGCGCCCCTGTGAAG
CAGACCCTGAACTTCGACCTGCTGAAGCTGGCCGGCGACGTGGAGAGCAATCCCGGCCCT
ATGGAAAACATCACCAGCGGCTTCCTGGGCCCCCTGCTGGTGCTGCAGGCCGGCTTCTTC
CTGCTGACCAGGATCCTGACCATTCCCCAGTCACTGGACAGCTGGTGGACCAGCCTGAAC
TTCCTCGGCGGGAGCCCCGTGTGCCTGGGCCAGAATAGCCAGAGCCCCACCAGCAACCAC
TCTCCCACTTCCTGCCCCCCTATCTGCCCCGGCTACAGGTGGATGTGCCTGAGGAGGTTC
ATCATCTTCCTGTTCATCCTGCTGCTGTGCCTGATCTTCCTGCTGGTGCTGCTGGACTAC
CAGGGAATGCTGCCCGTGTGTCCCCTGATCCCCGGAAGCACCACCACCAACACCGGCCCC
TGCAAGACCTGCACCACCCCCGCCCAGGGCAACTCTATGTTCCCCAGCTGCTGCTGCACC
AAGCCCACCGACGGCAACTGCACTTGCATTCCCATCCCCAGCAGCTGGGCCTTCGCCAAA
TATCTGTGGGAGTGGGCCAGCGTGAGGTTTAGCTGGCTGAGCCTGCTGGTGCCCTTCGTG
CAGTGGTTTGTGGGCCTGAGCCCCACCGTGTGGCTGAGCGCCATCTGGATGATGTGGTAC
TGGGGCCCCTCCCTGTACAGCATCGTGAGCCCCTTCATCCCCCTCCTGCCCATCTTCTTC
TGCCTGTGGGTGTACATC
SEQ ID NO: 11:
Amino acid sequence of HBc
MDIDPYKEFGATVELLSFLPSDFFPSVRDLLDTASALYREALESPEHCSPHHTALRQAIL
CWGELMTLATWVGNNLEDPASRDLVVNYVNTNMGLKIRQLLWFHISCLTFGRETVLEYLV
SFGVWIRTPPAYRPPNAPILSTLPETTVVRRRDRGRSPRRRTPSPRRRRSQSPRRRRSQS
RESQC
SEQ ID NO: 12:
Amino acid sequence of hli alternate variant
MHRRRSRSCREDQKPVMDDQRDLISNNEQLPMLGRRPGAPESKCSRGALYTGFSILVTLL
LAGQATTAYFLYQQQGRLDKLTVTSQNLQLENLRMKLPKPPKPVSKMRMATPLLMQALPM
GALPQGPMQNATKYGNMTEDHVMHLLQNADPLKVYPPLKGSFPENLRHLKNTMETIDWKV
FESWMHHWLLFEMSRHSLEQKPTDAPPKESLELEDPSSGLGVTKQDLGPVP
SEQ ID NO: 13:
Nucleotide sequence encoding hli alternate variant
ATGCACAGGAGGAGAAGCAGGAGCTGTCGGGAAGATCAGAAGCCAGTCATGGATGACCAG
CGCGACCTTATCTCCAACAATGAGCAACTGCCCATGCTGGGCCGGCGCCCTGGGGCCCCG
GAGAGCAAGTGCAGCCGCGGAGCCCTGTACACAGGCTTTTCCATCCTGGTGACTCTGCTC
CTCGCTGGCCAGGCCACCACCGCCTACTTCCTGTACCAGCAGCAGGGCCGGCTGGACAAA
CTGACAGTCACCTCCCAGAACCTGCAGCTGGAGAACCTGCGCATGAAGCTTCCCAAGCCT
CCCAAGCCTGTGAGCAAGATGCGCATGGCCACCCCGCTGCTGATGCAGGCGCTGCCCATG
GGAGCCCTGCCCCAGGGGCCCATGCAGAATGCCACCAAGTATGGCAACATGACAGAGGAC
CATGTGATGCACCTGCTCCAGAATGCTGACCCCCTGAAGGTGTACCCGCCACTGAAGGGG
AGCTTCCCGGAGAACCTGAGACACCTTAAGAACACCATGGAGACCATAGACTGGAAGGTC
TTTGAGAGCTGGATGCACCATTGGCTCCTGTTTGAAATGAGCAGGCACTCCTTGGAGCAA
AAGCCCACTGACGCTCCACCGAAAGAGTCACTGGAACTGGAGGACCCGTCTTCTGGGCTG
GGTGTGACCAAGCAGGATCTGGGCCCAGTCCCC
SEQ ID NO: 14:
Alternative nucleic acid sequence of hli-HBc-2A-HBs
ATGCACAGGAGGAGAAGCAGGAGCTGTCGGGAAGATCAGAAGCCAGTCATGGATGACCAG
CGCGACCTTATCTCCAACAATGAGCAACTGCCCATGCTGGGCCGGCGCCCTGGGGCCCCG
GAGAGCAAGTGCAGCCGCGGAGCCCTGTACACAGGCTTTTCCATCCTGGTGACTCTGCTC
CTCGCTGGCCAGGCCACCACCGCCTACTTCCTGTACCAGCAGCAGGGCCGGCTGGACAAA
CTGACAGTCACCTCCCAGAACCTGCAGCTGGAGAACCTGCGCATGAAGCTTCCCAAGCCT
CCCAAGCCTGTGAGCAAGATGCGCATGGCCACCCCGCTGCTGATGCAGGCGCTGCCCATG
GGAGCCCTGCCCCAGGGGCCCATGCAGAATGCCACCAAGTATGGCAACATGACAGAGGAC
CATGTGATGCACCTGCTCCAGAATGCTGACCCCCTGAAGGTGTACCCGCCACTGAAGGGG
AGCTTCCCGGAGAACCTGAGACACCTTAAGAACACCATGGAGACCATAGACTGGAAGGTC
TTTGAGAGCTGGATGCACCATTGGCTCCTGTTTGAAATGAGCAGGCACTCCTTGGAGCAA
AAGCCCACTGACGCTCCACCGAAAGAGTCACTGGAACTGGAGGACCCGTCTTCTGGGCTG
GGTGTGACCAAGCAGGATCTGGGCCCAGTCCCCATGGACATTGACCCTTATAAAGAATTT
GGAGCTACTGTGGAGTTACTCTCGTTTTTGCCTTCTGACTTCTTTCCTTCCGTCAGAGAT
CTCCTAGACACCGCCTCAGCTCTGTATCGAGAAGCCTTAGAGTCTCCTGAGCATTGCTCA
CCTCACCATACTGCACTCAGGCAAGCCATTCTCTGCTGGGGGGAATTGATGACTCTAGCT
ACCTGGGTGGGTAATAATTTGGAAGATCCAGCATCCAGGGATCTAGTAGTCAATTATGTT
AATACTAACATGGGTTTAAAGATCAGGCAACTATTGTGGTTTCATATATCTTGCCTTACT
TTTGGAAGAGAGACTGTACTTGAATATTTGGTCTCTTTCGGAGTGTGGATTCGCACTCCT
CCAGCCTATAGACCACCAAATGCCCCTATCTTATCAACACTTCCGGAAACTACTGTTGTT
AGACGACGGGACCGAGGCAGGTCCCCTAGAAGAAGAACTCCCTCGCCTCGCAGACGCAGA
TCTCAATCGCCGCGTCGCAGAAGATCTCAATCTCGGGAATCTCAATGTGCCCCTGTGAAG
CAGACCCTGAACTTCGACCTGCTGAAGCTGGCCGGCGACGTGGAGAGCAATCCCGGCCCT
ATGGAGAACATCACATCAGGATTCCTAGGACCCCTGCTCGTGTTACAGGCGGGGTTTTTC
TTGTTGACAAGAATCCTCACAATACCGCAGAGTCTAGACTCGTGGTGGACTTCTCTCAAT
TTTCTAGGGGGATCACCCGTGTGTCTTGGCCAAAATTCGCAGTCCCCAACCTCCAATCAC
TCACCAACCTCCTGTCCTCCAATTTGTCCTGGTTATCGCTGGATGTGTCTGCGGCGTTTT
ATCATATTCCTCTTCATCCTGCTGCTATGCCTCATCTTCTTATTGGTTCTTCTGGATTAT
CAAGGTATGTTGCCCGTTTGTCCTCTAATTCCAGGATCAACAACAACCAATACGGGACCA
TGCAAAACCTGCACGACTCCTGCTCAAGGCAACTCTATGTTTCCCTCATGTTGCTGTACA
AAACCTACGGATGGAAATTGCACCTGTATTCCCATCCCATCGTCCTGGGCTTTCGCAAAA
TACCTATGGGAGTGGGCCTCAGTCCGTTTCTCTTGGCTCAGTTTACTAGTGCCATTTGTT
CAGTGGTTCGTAGGGCTTTCCCCCACTGTTTGGCTTTCAGCTATATGGATGATGTGGTAT
TGGGGGCCAAGTCTGTACAGCATCGTGAGTCCCTTTATACCGCTGTTACCAATTTTCTTT
TGTCTCTGGGTATACATT
SEQ ID NO: 15:
Alternative amino acid sequence of hli-HBc-2A-HBs
MHRRRSRSCREDQKPVMDDQRDLISNNEQLPMLGRRPGAPESKCSRGALYTGFSILVTLL
LAGQATTAYFLYQQQGRLDKLTVTSQNLQLENLRMKLPKPPKPVSKMRMATPLLMQALPM
GALPQGPMQNATKYGNMTEDHVMHLLQNADPLKVYPPLKGSFPENLRHLKNTMETIDWKV
FESWMHHWLLFEMSRHSLEQKPTDAPPKESLELEDPSSGLGVTKQDLGPVPMDIDPYKEF
GATVELLSFLPSDFFPSVRDLLDTASALYREALESPEHCSPHHTALRQAILCWGELMTLA
TWVGNNLEDPASRDLVVNYVNTNMGLKIRQLLWFHISCLTFGRETVLEYLVSFGVWIRTP
PAYRPPNAPILSTLPETTVVRRRDRGRSPRRRTPSPRRRRSQSPRRRRSQSRESQCAPVK
QTLNFDLLKLAGDVESNPGPMENITSGFLGPLLVLQAGFFLLTRILTIPQSLDSWWTSLN
FLGGSPVCLGQNSQSPTSNHSPTSCPPICPGYRWMCLRRFIIFLFILLLCLIFLLVLLDY
QGMLPVCPLIPGSTTTNTGPCKTCTTPAQGNSMFPSCCCTKPTDGNCTCIPIPSSWAFAK
YLWEWASVRFSWLSLLVPFVQWFVGLSPTVWLSAIWMMWYWGPSLYSIVSPFIPLLPIFF
CLWVYI
SEQ ID NO: 16:
Nucleic acid sequence of an empty SAM vector
ATAGGCGGCGCATGAGAGAAGCCCAGACCAATTACCTACCCAAAATGGAGAAAGTTCACG
TTGACATCGAGGAAGACAGCCCATTCCTCAGAGCTTTGCAGCGGAGCTTCCCGCAGTTTG
AGGTAGAAGCCAAGCAGGTCACTGATAATGACCATGCTAATGCCAGAGCGTTTTCGCATC
TGGCTTCAAAACTGATCGAAACGGAGGTGGACCCATCCGACACGATCCTTGACATTGGAA
GTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGG
AAATAACTGATAAGGAATTGGACAAGAAAATGAAGGAGCTCGCCGCCGTCATGAGCGACC
CTGACCTGGAAACTGAGACTATGTGCCTCCACGACGACGAGTCGTGTCGCTACGAAGGGC
AAGTCGCTGTTTACCAGGATGTATACGCGGTTGACGGACCGACAAGTCTCTATCACCAAG
CCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAA
CGGCTCGTAACATAGGCCTATGCAGCTCTGACGTTATGGAGCGGTCACGTAGAGGGATGT
CCATTCTTAGAAAGAAGTATTTGAAACCATCCAACAATGTTCTATTCTCTGTTGGCTCGA
CCATCTACCACGAGAAGAGGGACTTACTGAGGAGCTGGCACCTGCCGTCTGTATTTCACT
TACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTA
CGATGCACCGCGAGGGATTCTTGTGCTGCAAAGTGACAGACACATTGAACGGGGAGAGGG
TCTCTTTTCCCGTGTGCACGTATGTGCCAGCTACATTGTGTGACCAAATGACTGGCATAC
TGGCAACAGATGTCAGTGCGGACGACGCGCAAAAACTGCTGGTTGGGCTCAACCAGCGTA
TAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAA
GGCCACTAGGACTACGAGATAGACAGTTAGTCATGGGGTGTTGTTGGGCTTTTAGAAGGC
ACAAGATAACATCTATTTATAAGCGCCCGGATACCCAAACCATCATCAAAGTGAACAGCG
ATTTCCACTCATTCGTGCTGCCCAGGATAGGCAGTAACACATTGGAGATCGGGCTGAGAA
CAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGT
TGCGCGCAGCTCTACCACCTTTGGCAGCTGATGTTGAGGAGCCCACTCTGGAAGCCGATG
TCGACTTGATGTTACAAGAGGCTGGGGCCGGCTCAGTGGAGACACCTCGTGGCTTGATAA
AGGTTACCAGCTACGATGGCGAGGACAAGATCGGCTCTTACGCTGTGCTTTCTCCGCAGG
CTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGG
TGCCAGAGGGACATGCAATACCCGTCCAGGACTTTCAAGCTCTGAGTGAAAGTGCCACCA
TTGTGTACAACGAACGTGAGTTCGTAAACAGGTACCTGCACCATATTGCCACACATGGAG
GAGCGCTGAACACTGATGAAGAATATTACAAAACTGTCAAGCCCAGCGAGCACGACGGCG
AATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAA
CACGACCAGCCGCTCCTTACCAAGTACCAACCATAGGGGTGTATGGCGTGCCAGGATCAG
GCAAGTCTGGCATCATTAAAAGCGCAGTCACCAAAAAAGATCTAGTGGTGAGCGCCAAGA
AAGAAAACTGTGCAGAAATTATAAGGGACGTCAAGAAAATGAAAGGGCTGGACGTCAATG
CCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGAC
CTAAAAAGGCAGTGCTCTGCGGGGATCCCAAACAGTGCGGTTTTTTTAACATGATGTGCC
TGAAAGTGCATTTTAACCACGAGATTTGCACACAAGTCTTCCACAAAAGCATCTCTCGCC
GTTGCACTAAATCTGTGACTTCGGTCGTCTCAACCTTGTTTTACGACAAAAAAATGAGAA
CGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACA
AAGGCAACGAAATAATGACGGCAGCTGCCTCTCAAGGGCTGACCCGTAAAGGTGTGTATG
CCGTTCGGTACAAGGTGAATGAAAATCCTCTGTACGCACCCACCTCAGAACATGTGAACG
TCCTACTGACCCGCACGGAGGACCGCATCGTGTGGAAAACACTAGCCGGCGACCCATGGA
TAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCC
AGAATAAGGCAAACGTGTGTTGGGCCAAGGCTTTAGTGCCGGTGCTGAAGACCGCTGGCA
TAGACATGACCACTGAACAATGGAACACTGTGGATTATTTTGAAACGGACAAAGCTCACT
CAGCAGAGATAGTATTGAACCAACTATGCGTGAGGTTCTTTGGACTCGATCTGGACTCCG
GTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACC
CACAACTGCCTCGGGCAGTTGCCACTGGAAGAGTCTATGACATGAACACTGGTACACTGC
GCAATTATGATCCGCGCATAAACCTAGTACCTGTAAACAGAAGACTGCCTCATGCTTTAG
TCCTCCACCATAATGAACACCCACAGAGTGACTTTTCTTCATTCGTCAGCAAATTGAAGG
GCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATG
TGCCCAAATATGACATAATATTTGTTAATGTGAGGACCCCATATAAATACCATCACTATC
AGCAGTGTGAAGACCATGCCATTAAGCTTAGCATGTTGACCAAGAAAGCTTGTCTGCATC
TGAATCCCGGCGGAACCTGTGTCAGCATAGGTTATGGTTACGCTGACAGGGCCAGCGAAA
GCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGC
ACAATCCTTACAAGCTTTCATCAACCTTGACCAACATTTATACAGGTTCCAGACTCCACG
AAGCCGGATGTGCACCCTCATATCATGTGGTGCGAGGGGATATTGCCACGGCCACCGAAG
GAGTGATTATAAATGCTGCTAACAGCAAAGGACAACCTGGCGGAGGGGTGTGCGGAGCGC
TGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTT
CGGAGGTTGAAGGTGACAAACAGTTGGCAGAGGCTTATGAGTCCATCGCTAAGATTGTCA
ACGATAACAATTACAAGTCAGTAGCGATTCCACTGTTGTCCACCGGCATCTTTTCCGGGA
ACAAAGATCGACTAACCCAATCATTGAACCATTTGCTGACAGCTTTAGACACCACTGATG
CAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTG
ATGCAGAGCTGGTGAGGGTGCATCCGAAGAGTTCTTTGGCTGGAAGGAAGGGCTACAGCA
CAAGCGATGGCAAAACTTTCTCATATTTGGAAGGGACCAAGTTTCACCAGGCGGCCAAGG
ATATAGCAGAAATTAATGCCATGTGGCCCGTTGCAACGGAGGCCAATGAGCAGGTATGCA
TGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAA
GAGTACAGCGCCTAAAAGCCTCACGTCCAGAACAAATTACTGTGTGCTCATCCTTTCCAT
TGCCGAAGTATAGAATCACTGGTGTGCAGAAGATCCAATGCTCCCAGCCTATATTGTTCT
CACCGAAAGTGCCTGCGTATATTCATCCAAGGAAGTATCTCGTGGAAACACCACCGGTAG
ACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCG
AGGCAGACATTCACGGGCCGCCCTCTGTATCTAGCTCATCCTGGTCCATTCCTCATGCAT
CCGACTTTGATGTGGACAGTTTATCCATACTTGACACCCTGGAGGGAGCTAGCGTGACCA
GCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAA
GAACACCGTCACTTGCACCCAGCAGGGCCTGCTCGAGAACCAGCCTAGTTTCCACCCCGC
CAGGCGTGAATAGGGTGATCACTAGAGAGGAGCTCGAGGCGCTTACCCCGTCACGCACTC
CTAGCAGGTCGGTCTCGAGAACCAGCCTGGTCTCCAACCCGCCAGGCGTAAATAGGGTGA
TTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAA
CGGTGCTATCCGAAGTGGTGTTGGAGAGGACCGAATTGGAGATTTCGTATGCCCCGCGCC
TCGACCAAGAAAAAGAAGAATTACTACGCAAGAAATTACAGTTAAATCCCACACCTGCTA
ACAGAAGCAGATACCAGTCCAGGAAGGTGGAGAACATGAAAGCCATAACAGCTAGACGTA
TTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCG
CAGTGGAAGCCTGTAACGCCATGTTGAAAGAGAACTTTCCGACTGTGGCTTCTTACTGTA
TTATTCCAGAGTACGATGCCTATTTGGACATGGTTGACGGAGCTTCATGCTGCTTAGACA
CTGCCAGTTTTTGCCCTGCAAAGCTGCGCAGCTTTCCAAAGAAACACTCCTATTTGGAAC
CCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGG
CGGCCTTTAATGTGGAATGCTTCAAGAAATATGCGTGTAATAATGAATATTGGGAAACGT
TTAAAGAAAACCCCATCAGGCTTACTGAAGAAAACGTGGTAAATTACATTACCAAATTAA
AAGGACCAAAAGCTGCTGCTCTTTTTGCGAAGACACATAATTTGAATATGTTGCAGGACA
TACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAG
CGTATCTGTGCGGAATCCACCGAGAGCTGGTTAGGAGATTAAATGCGGTCCTGCTTCCGA
ACATTCATACACTGTTTGATATGTCGGCTGAAGACTTTGACGCTATTATAGCCGAGCACT
TCCAGCCTGGGGATTGTGTTCTGGAAACTGACATCGCGTCGTTTGATAAAAGTGAGGACG
ACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTA
AATTTAAATTCGGAGCCATGATGAAATCTGGAATGTTCCTCACACTGTTTGTGAACACAG
TCATTAACATTGTAATCGCAAGCAGAGTGTTGAGAGAACGGCTAACCGGATCACCATGTG
CAGCATTCATTGGAGATGACAATATCGTGAAAGGAGTCAAATCGGACAAATTAATGGCAG
ACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCC
GTGTGGCAGACCCCCTAAAAAGGCTGTTTAAGCTTGGCAAACCTCTGGCAGCAGACGATG
AACATGATGATGACAGGAGAAGGGCATTGCATGAAGAGTCAACACGCTGGAACCGAGTGG
GTATTCTTTCAGAGCTGTGCAAGGCAGTAGAATCAAGGTATGAAACCGTAGGAACTTCCA
TCATAGTTATGGCCATGACTACTCTAGCTAGCAGTGTTAAATCATTCAGCTACCTGAGAG
GGGCCCCTATAACTCTCTACGGCTAACCTGAATGGACTACGACATAGTCTAGTCCGCCAA
G1TGATGAGGCGCGCCCACCCAGCGGCCGCATACAGCAGCAATTGGCAAGCTGCTTACAT
AGAACTCGCGGCGATTGGCATGCCGCCTTAAAATTTTTATTTTATTTTTCTTTTCTTTTC
CGAATCGGATTTTGTTTTTAATATTTCAAAAAAAAAAAAAAAAAAAAAAAAAAAAAAAAA
AAAAAAAGAAGAGCGTTTAAACACGTGATATCTGGCCTCATGGGCCTTCCTTTCACTGCC
CGCTTTCCAGTCGGGAAACCTGTCGTGCCAGCTGCATTAACATGGTCATAGCTGTTTCCT
TGCGTATTGGGCGCTCTCCGCTTCCTCGCTCACTGACTCGCTGCGCTCGGTCGTTCGGGT
AAAGCCTGGGGTGCCTAATGAGCAAAAGGCCAGCAAAAGGCCAGGAACCGTAAAAAGGCC
GCGTTGCTGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCCCCCTGGA
AGCTCCCTCGTGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCGCTGC
GCCTTATCCGGTAACTATCGTCTTGAGTCCAACCCGGTAAGACACGACTTATCGCCACTG
GCAGCAGCCACTGGTAACAGGATTAGCAGAGCGAGGTATGTAGGCGGTGCTACAGAGTTC
TTGAAGTGGTGGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAAAAAAGGATCT
CAAGAAGATCCTTTGATCTTTTCTACGGGGTCTGACGCTCAGTGGAACGAAAACTCACGT
TAAGGGATTTTGGTCATGAATACACGGTGCCTGACTGCGTTAGCAATTTAACTGTGATAA
ACTACCGCATTAAAGCTTATCGATGATAAGCTGTCAAACATGAGAATTCTTAGAAAAACT
CATCGAGCATCAAATGAAACTGCAATTTATTCATATCAGGATTATCAATACCATATTTTT
GAAAAAGCCGTTTCTGTAATGAAGGAGAAAACTCACCGAGGCAGTTCCATAGGATGGCAA
GATCCTGGTATCGGTCTGCGATTCCGACTCGTCCAACATCAATACAACCTATTAATTTCC
CCTCGTCAAAAATAAGGTTATCAAGTGAGAAATCACCATGAGTGACGACTGAATCCGGTG
AGAATGGCAAAAGCTTATGCATTTCTTTCCAGACTTGTTCAACAGGCCAGCCATTACGCT
CGTCATCAAAATCACTCGCATCAACCAAACCGTTATTCATTCGTGATTGCGCCTGAGCGA
GACGAAATACGCGATCGCTGTTAAAAGGACAATTACAAACAGGAATCGAATGCAACCGGC
GCAGGAACACTGCCAGCGCATCAACAATATTTTCACCTGAATCAGGATATTCTTCTAATA
CCTGGAATGCTGTTTTCCCGGGGATCGCAGTGGTGAGTAACCATGCATCATCAGGAGTAC
GGATAAAATGCTTGATGGTCGGAAGAGGCATAAATTCCGTCAGCCAGTTTAGTCTGACCA
TCTCATCTGTAACATCATTGGCAACGCTACCTTTGCCATGTTTCAGAAACAACTCTGGCG
CATCGGGCTTCCCATACAATCGATAGATTGTCGCACCTGATTGCCCGACATTATCGCGAG
CCCATTTATACCCATATAAATCAGCATCCATGTTGGAATTTAATCGCGGCCTCGAGCAAG
ACGTTTCCCGTTGAATATGGCTCATAACACCCCTTGTATTACTGTTTATGTAAGCAGACA
GTTTTATTGTTCATGAGCGGATACATATTTGAATGTATTTAGAAAAATAAACAAATAGGG
GTTCCGCGCACATTTCCCCGAAAAGTGCCACCTAAATTGTAAGCGTTAATATTTTGTTAA
AATTCGCGTTAAATTTTTGTTAAATCAGCTCATTTTTTAACCAATAGGCCGAAATCGGCA
AAATCCCTTATAAATCAAAAGAATAGACCGAGATAGGGTTGAGTGGCCGCTACAGGGCGC
TCCCATTCGCCATTCAGGCTGCGCAACTGTTGGGAAGGGCGTTTCGGTGCGGGCCTCTTC
GCTATTACGCCAGCTGGCGAAAGGGGGATGTGCTGCAAGGCGATTAAGTTGGGTAACGCC
AGGGTTTTCCCAGTCACACGCGTAATACGACTCACTATAG
Insert starts here after nucleotide 7561
SEQ ID NO: 17:
Human codon optimized (Genewiz) nucleic acid sequence encoding the
hli_HBc_2A_HBs SAM transgene
ATGCATAGAAGAAGGTCCAGAAGCTGCAGAGAAGACCAGAAACCCGTGATGGACGACCAG
AGAGACCTCATCTCCAACAACGAGCAGCTGCCTATGCTGGGCAGAAGGCCCGGCGCTCCC
GAATCCAAGTGTTCCAGAGGAGCCCTCTACACCGGCTTCAGCATCCTCGTCACACTGCTG
CTCGCTGGACAAGCCACCACCGCTTACTTTCTGTACCAGCAGCAAGGAAGACTGGATAAG
CTGACCGTCACCTCCCAGAATCTCCAACTGGAGAATCTGAGGATGAAACTCCCCAAACCT
CCTAAGCCCGTGTCCAAGATGAGGATGGCTACACCTCTGCTCATGCAAGCCCTCCCCATG
GGAGCTCTGCCCCAAGGACCCATGCAGAATGCCACCAAGTACGGCAACATGACAGAGGAC
CACGTGATGCATCTGCTCCAGAACGCCGATCCTCTCAAGGTCTATCCCCCTCTGAAGGGC
TCCTTCCCCGAGAATCTGAGGCATCTCAAAAACACCATGGAGACCATCGATTGGAAGGTG
TTCGAGAGCTGGATGCATCACTGGCTGCTGTTCGAGATGTCTAGGCACTCCCTCGAGCAG
AAGCCCACAGATGCCCCTCCCAAGGAGAGCCTCGAGCTGGAAGATCCCTCCAGCGGACTG
GGAGTCACAAAGCAAGACCTCGGCCCCGTCCCCATGGACATCGATCCCTACAAAGAGTTC
GGCGCTACCGTGGAGCTGCTGTCCTTTCTGCCTTCCGACTTTTTCCCCTCCGTCAGAGAT
CTGCTGGACACCGCCTCCGCTCTGTATAGGGAGGCCCTCGAGTCCCCCGAGCACTGTTCC
CCTCATCACACAGCTCTGAGACAAGCCATTCTGTGCTGGGGCGAGCTGATGACACTGGCC
ACATGGGTCGGCAACAACCTCGAAGATCCCGCCTCTAGGGATCTGGTGGTCAACTACGTG
AACACCAACATGGGACTGAAGATTAGACAGCTGCTGTGGTTCCACATTAGCTGTCTCACC
TTTGGCAGAGAAACCGTGCTGGAGTATCTGGTGAGCTTCGGAGTGTGGATCAGAACCCCC
CCCGCCTATAGACCTCCCAATGCCCCCATTCTGTCCACACTGCCCGAGACAACCGTCGTC
AGAAGGAGGGACAGAGGAAGATCCCCTAGAAGGAGAACCCCCAGCCCTAGAAGAAGGAGG
TCCCAGTCCCCCAGAAGAAGGAGAAGCCAGAGCAGAGAATCCCAGTGCGCTCCCGTCAAA
CAGACCCTCAACTTCGATCTGCTCAAGCTGGCCGGCGATGTGGAATCCAACCCCGGCCCT
ATGGAGAATATCACCAGCGGCTTTCTCGGCCCTCTGCTGGTCCTCCAAGCTGGCTTCTTT
CTGCTGACAAGGATTCTGACAATCCCCCAATCTCTGGACAGCTGGTGGACATCCCTCAAC
TTTCTGGGCGGAAGCCCCGTGTGCCTCGGCCAAAACTCCCAGAGCCCCACATCCAATCAC
TCCCCCACCAGCTGCCCCCCTATTTGCCCCGGCTACAGATGGATGTGTCTGAGAAGGTTC
ATCATCTTCCTCTTCATTCTCCTCCTCTGCCTCATCTTTCTGCTGGTGCTCCTCGACTAC
CAAGGCATGCTGCCCGTGTGCCCTCTGATCCCCGGCAGCACCACCACAAATACCGGCCCT
TGTAAGACATGCACCACACCCGCCCAAGGCAACAGCATGTTTCCTAGCTGCTGCTGCACA
AAGCCTACAGACGGCAACTGCACATGCATTCCTATCCCCAGCAGCTGGGCCTTCGCTAAG
TATCTGTGGGAATGGGCTTCCGTGAGGTTCAGCTGGCTCTCTCTGCTGGTGCCCTTCGTG
CAATGGTTTGTGGGCCTCAGCCCTACCGTGTGGCTGTCCGCTATTTGGATGATGTGGTAT
TGGGGACCCTCTCTGTACAGCATCGTGTCCCCCTTCATCCCTCTGCTGCCCATTTTTTTC
TGTCTGTGGGTGTATATTTGATGA
SEQ ID NO: 18:
hli_HBc_2A_HBs in AA098, SAM plasmid sequence
ATAGGCGGCGCATGAGAGAAGCCCAGACCAATTACCTACCCAAAATGGAGAAAGTTCACG
TTGACATCGAGGAAGACAGCCCATTCCTCAGAGCTTTGCAGCGGAGCTTCCCGCAGTTTG
AGGTAGAAGCCAAGCAGGTCACTGATAATGACCATGCTAATGCCAGAGCGTTTTCGCATC
TGGCTTCAAAACTGATCGAAACGGAGGTGGACCCATCCGACACGATCCTTGACATTGGAA
GTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGG
AAATAACTGATAAGGAATTGGACAAGAAAATGAAGGAGCTCGCCGCCGTCATGAGCGACC
CTGACCTGGAAACTGAGACTATGTGCCTCCACGACGACGAGTCGTGTCGCTACGAAGGGC
AAGTCGCTGTTTACCAGGATGTATACGCGGTTGACGGACCGACAAGTCTCTATCACCAAG
CCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAA
CGGCTCGTAACATAGGCCTATGCAGCTCTGACGTTATGGAGCGGTCACGTAGAGGGATGT
CCATTCTTAGAAAGAAGTATTTGAAACCATCCAACAATGTTCTATTCTCTGTTGGCTCGA
CCATCTACCACGAGAAGAGGGACTTACTGAGGAGCTGGCACCTGCCGTCTGTATTTCACT
TACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTA
CGATGCACCGCGAGGGATTCTTGTGCTGCAAAGTGACAGACACATTGAACGGGGAGAGGG
TCTCTTTTCCCGTGTGCACGTATGTGCCAGCTACATTGTGTGACCAAATGACTGGCATAC
TGGCAACAGATGTCAGTGCGGACGACGCGCAAAAACTGCTGGTTGGGCTCAACCAGCGTA
TAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAA
GGCCACTAGGACTACGAGATAGACAGTTAGTCATGGGGTGTTGTTGGGCTTTTAGAAGGC
ACAAGATAACATCTATTTATAAGCGCCCGGATACCCAAACCATCATCAAAGTGAACAGCG
ATTTCCACTCATTCGTGCTGCCCAGGATAGGCAGTAACACATTGGAGATCGGGCTGAGAA
CAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGT
TGCGCGCAGCTCTACCACCTTTGGCAGCTGATGTTGAGGAGCCCACTCTGGAAGCCGATG
TCGACTTGATGTTACAAGAGGCTGGGGCCGGCTCAGTGGAGACACCTCGTGGCTTGATAA
AGGTTACCAGCTACGATGGCGAGGACAAGATCGGCTCTTACGCTGTGCTTTCTCCGCAGG
CTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGG
TGCCAGAGGGACATGCAATACCCGTCCAGGACTTTCAAGCTCTGAGTGAAAGTGCCACCA
TTGTGTACAACGAACGTGAGTTCGTAAACAGGTACCTGCACCATATTGCCACACATGGAG
GAGCGCTGAACACTGATGAAGAATATTACAAAACTGTCAAGCCCAGCGAGCACGACGGCG
AATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAA
CACGACCAGCCGCTCCTTACCAAGTACCAACCATAGGGGTGTATGGCGTGCCAGGATCAG
GCAAGTCTGGCATCATTAAAAGCGCAGTCACCAAAAAAGATCTAGTGGTGAGCGCCAAGA
AAGAAAACTGTGCAGAAATTATAAGGGACGTCAAGAAAATGAAAGGGCTGGACGTCAATG
CCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGAC
CTAAAAAGGCAGTGCTCTGCGGGGATCCCAAACAGTGCGGTTTTTTTAACATGATGTGCC
TGAAAGTGCATTTTAACCACGAGATTTGCACACAAGTCTTCCACAAAAGCATCTCTCGCC
GTTGCACTAAATCTGTGACTTCGGTCGTCTCAACCTTGTTTTACGACAAAAAAATGAGAA
CGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACA
AAGGCAACGAAATAATGACGGCAGCTGCCTCTCAAGGGCTGACCCGTAAAGGTGTGTATG
CCGTTCGGTACAAGGTGAATGAAAATCCTCTGTACGCACCCACCTCAGAACATGTGAACG
TCCTACTGACCCGCACGGAGGACCGCATCGTGTGGAAAACACTAGCCGGCGACCCATGGA
TAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCC
AGAATAAGGCAAACGTGTGTTGGGCCAAGGCTTTAGTGCCGGTGCTGAAGACCGCTGGCA
TAGACATGACCACTGAACAATGGAACACTGTGGATTATTTTGAAACGGACAAAGCTCACT
CAGCAGAGATAGTATTGAACCAACTATGCGTGAGGTTCTTTGGACTCGATCTGGACTCCG
GTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACC
CACAACTGCCTCGGGCAGTTGCCACTGGAAGAGTCTATGACATGAACACTGGTACACTGC
GCAATTATGATCCGCGCATAAACCTAGTACCTGTAAACAGAAGACTGCCTCATGCTTTAG
TCCTCCACCATAATGAACACCCACAGAGTGACTTTTCTTCATTCGTCAGCAAATTGAAGG
GCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATG
TGCCCAAATATGACATAATATTTGTTAATGTGAGGACCCCATATAAATACCATCACTATC
AGCAGTGTGAAGACCATGCCATTAAGCTTAGCATGTTGACCAAGAAAGCTTGTCTGCATC
TGAATCCCGGCGGAACCTGTGTCAGCATAGGTTATGGTTACGCTGACAGGGCCAGCGAAA
GCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGC
ACAATCCTTACAAGCTTTCATCAACCTTGACCAACATTTATACAGGTTCCAGACTCCACG
AAGCCGGATGTGCACCCTCATATCATGTGGTGCGAGGGGATATTGCCACGGCCACCGAAG
GAGTGATTATAAATGCTGCTAACAGCAAAGGACAACCTGGCGGAGGGGTGTGCGGAGCGC
TGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTT
CGGAGGTTGAAGGTGACAAACAGTTGGCAGAGGCTTATGAGTCCATCGCTAAGATTGTCA
ACGATAACAATTACAAGTCAGTAGCGATTCCACTGTTGTCCACCGGCATCTTTTCCGGGA
ACAAAGATCGACTAACCCAATCATTGAACCATTTGCTGACAGCTTTAGACACCACTGATG
CAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTG
ATGCAGAGCTGGTGAGGGTGCATCCGAAGAGTTCTTTGGCTGGAAGGAAGGGCTACAGCA
CAAGCGATGGCAAAACTTTCTCATATTTGGAAGGGACCAAGTTTCACCAGGCGGCCAAGG
ATATAGCAGAAATTAATGCCATGTGGCCCGTTGCAACGGAGGCCAATGAGCAGGTATGCA
TGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAA
GAGTACAGCGCCTAAAAGCCTCACGTCCAGAACAAATTACTGTGTGCTCATCCTTTCCAT
TGCCGAAGTATAGAATCACTGGTGTGCAGAAGATCCAATGCTCCCAGCCTATATTGTTCT
CACCGAAAGTGCCTGCGTATATTCATCCAAGGAAGTATCTCGTGGAAACACCACCGGTAG
ACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCG
AGGCAGACATTCACGGGCCGCCCTCTGTATCTAGCTCATCCTGGTCCATTCCTCATGCAT
CCGACTTTGATGTGGACAGTTTATCCATACTTGACACCCTGGAGGGAGCTAGCGTGACCA
GCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAA
GAACACCGTCACTTGCACCCAGCAGGGCCTGCTCGAGAACCAGCCTAGTTTCCACCCCGC
CAGGCGTGAATAGGGTGATCACTAGAGAGGAGCTCGAGGCGCTTACCCCGTCACGCACTC
CTAGCAGGTCGGTCTCGAGAACCAGCCTGGTCTCCAACCCGCCAGGCGTAAATAGGGTGA
TTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAA
CGGTGCTATCCGAAGTGGTGTTGGAGAGGACCGAATTGGAGATTTCGTATGCCCCGCGCC
TCGACCAAGAAAAAGAAGAATTACTACGCAAGAAATTACAGTTAAATCCCACACCTGCTA
ACAGAAGCAGATACCAGTCCAGGAAGGTGGAGAACATGAAAGCCATAACAGCTAGACGTA
TTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCG
CAGTGGAAGCCTGTAACGCCATGTTGAAAGAGAACTTTCCGACTGTGGCTTCTTACTGTA
TTATTCCAGAGTACGATGCCTATTTGGACATGGTTGACGGAGCTTCATGCTGCTTAGACA
CTGCCAGTTTTTGCCCTGCAAAGCTGCGCAGCTTTCCAAAGAAACACTCCTATTTGGAAC
CCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGG
CGGCCTTTAATGTGGAATGCTTCAAGAAATATGCGTGTAATAATGAATATTGGGAAACGT
TTAAAGAAAACCCCATCAGGCTTACTGAAGAAAACGTGGTAAATTACATTACCAAATTAA
AAGGACCAAAAGCTGCTGCTCTTTTTGCGAAGACACATAATTTGAATATGTTGCAGGACA
TACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAG
CGTATCTGTGCGGAATCCACCGAGAGCTGGTTAGGAGATTAAATGCGGTCCTGCTTCCGA
ACATTCATACACTGTTTGATATGTCGGCTGAAGACTTTGACGCTATTATAGCCGAGCACT
TCCAGCCTGGGGATTGTGTTCTGGAAACTGACATCGCGTCGTTTGATAAAAGTGAGGACG
ACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTA
AATTTAAATTCGGAGCCATGATGAAATCTGGAATGTTCCTCACACTGTTTGTGAACACAG
TCATTAACATTGTAATCGCAAGCAGAGTGTTGAGAGAACGGCTAACCGGATCACCATGTG
CAGCATTCATTGGAGATGACAATATCGTGAAAGGAGTCAAATCGGACAAATTAATGGCAG
ACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCC
GTGTGGCAGACCCCCTAAAAAGGCTGTTTAAGCTTGGCAAACCTCTGGCAGCAGACGATG
AACATGATGATGACAGGAGAAGGGCATTGCATGAAGAGTCAACACGCTGGAACCGAGTGG
GTATTCTTTCAGAGCTGTGCAAGGCAGTAGAATCAAGGTATGAAACCGTAGGAACTTCCA
TCATAGTTATGGCCATGACTACTCTAGCTAGCAGTGTTAAATCATTCAGCTACCTGAGAG
GGGCCCctataactctctacggctaacctgaatggactacgacatagtctagtccgccaa
gATGCATAGAAGAAGGTCCAGAAGCTGCAGAGAAGACCAGAAACCCGTGATGGACGACCA
GAGAGACCTCATCTCCAACAACGAGCAGCTGCCTATGCTGGGCAGAAGGCCCGGCGCTCC
CGAATCCAAGTGTTCCAGAGGAGCCCTCTACACCGGCTTCAGCATCCTCGTCACACTGCT
GCTCGCTGGACAAGCCACCACCGCTTACTTTCTGTACCAGCAGCAAGGAAGACTGGATAA
GCTGACCGTCACCTCCCAGAATCTCCAACTGGAGAATCTGAGGATGAAACTCCCCAAACC
TCCTAAGCCCGTGTCCAAGATGAGGATGGCTACACCTCTGCTCATGCAAGCCCTCCCCAT
GGGAGCTCTGCCCCAAGGACCCATGCAGAATGCCACCAAGTACGGCAACATGACAGAGGA
CCACGTGATGCATCTGCTCCAGAACGCCGATCCTCTCAAGGTCTATCCCCCTCTGAAGGG
CTCCTTCCCCGAGAATCTGAGGCATCTCAAAAACACCATGGAGACCATCGATTGGAAGGT
GTTCGAGAGCTGGATGCATCACTGGCTGCTGTTCGAGATGTCTAGGCACTCCCTCGAGCA
GAAGCCCACAGATGCCCCTCCCAAGGAGAGCCTCGAGCTGGAAGATCCCTCCAGCGGACT
GGGAGTCACAAAGCAAGACCTCGGCCCCGTCCCCATGGACATCGATCCCTACAAAGAGTT
CGGCGCTACCGTGGAGCTGCTGTCCTTTCTGCCTTCCGACTTTTTCCCCTCCGTCAGAGA
TCTGCTGGACACCGCCTCCGCTCTGTATAGGGAGGCCCTCGAGTCCCCCGAGCACTGTTC
CCCTCATCACACAGCTCTGAGACAAGCCATTCTGTGCTGGGGCGAGCTGATGACACTGGC
CACATGGGTCGGCAACAACCTCGAAGATCCCGCCTCTAGGGATCTGGTGGTCAACTACGT
GAACACCAACATGGGACTGAAGATTAGACAGCTGCTGTGGTTCCACATTAGCTGTCTCAC
CTTTGGCAGAGAAACCGTGCTGGAGTATCTGGTGAGCTTCGGAGTGTGGATCAGAACCCC
CCCCGCCTATAGACCTCCCAATGCCCCCATTCTGTCCACACTGCCCGAGACAACCGTCGT
CAGAAGGAGGGACAGAGGAAGATCCCCTAGAAGGAGAACCCCCAGCCCTAGAAGAAGGAG
GTCCCAGTCCCCCAGAAGAAGGAGAAGCCAGAGCAGAGAATCCCAGTGCGCTCCCGTCAA
ACAGACCCTCAACTTCGATCTGCTCAAGCTGGCCGGCGATGTGGAATCCAACCCCGGCCC
TATGGAGAATATCACCAGCGGCTTTCTCGGCCCTCTGCTGGTCCTCCAAGCTGGCTTCTT
TCTGCTGACAAGGATTCTGACAATCCCCCAATCTCTGGACAGCTGGTGGACATCCCTCAA
CTTTCTGGGCGGAAGCCCCGTGTGCCTCGGCCAAAACTCCCAGAGCCCCACATCCAATCA
CTCCCCCACCAGCTGCCCCCCTATTTGCCCCGGCTACAGATGGATGTGTCTGAGAAGGTT
CATCATCTTCCTCTTCATTCTCCTCCTCTGCCTCATCTTTCTGCTGGTGCTCCTCGACTA
CCAAGGCATGCTGCCCGTGTGCCCTCTGATCCCCGGCAGCACCACCACAAATACCGGCCC
TTGTAAGACATGCACCACACCCGCCCAAGGCAACAGCATGTTTCCTAGCTGCTGCTGCAC
AAAGCCTACAGACGGCAACTGCACATGCATTCCTATCCCCAGCAGCTGGGCCTTCGCTAA
GTATCTGTGGGAATGGGCTTCCGTGAGGTTCAGCTGGCTCTCTCTGCTGGTGCCCTTCGT
GCAATGGTTTGTGGGCCTCAGCCCTACCGTGTGGCTGTCCGCTATTTGGATGATGTGGTA
TTGGGGACCCTCTCTGTACAGCATCGTGTCCCCCTTCATCCCTCTGCTGCCCATTTTTTT
CTGTCTGTGGGTGTATATTTGATGAggcgcgcccacccaGCGGCCGCATACAGCAGCAAT
TGGCAAGCTGCTTACATAGAACTCGCGGCGATTGGCATGCCGCCTTAAAATTTTTATTTT
ATTTTTCTTTTCTTTTCCGAATCGGATTTTGTTTTTAATATTTCAAAAAAAAAAAAAAAA
AAAAAAAAAAAAAAAAAAAAAAAAGAAGAGCGTTTAAACACGTGATATCTGGCCTCATGG
GCCTTCCTTTCACTGCCCGCTTTCCAGTCGGGAAACCTGTCGTGCCAGCTGCATTAACAT
GGTCATAGCTGTTTCCTTGCGTATTGGGCGCTCTCCGCTTCCTCGCTCACTGACTCGCTG
CGCTCGGTCGTTCGGGTAAAGCCTGGGGTGCCTAATGAGCAAAAGGCCAGCAAAAGGCCA
GGAACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTA
GGTATCTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGAT
CCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGT
GGAACGAAAACTCACGTTAAGGGATTTTGGTCATGAATACACGGTGCCTGACTGCGTTAG
CAATTTAACTGTGATAAACTACCGCATTAAAGCTTATCGATGATAAGCTGTCAAACATGA
GAATTCTTAGAAAAACTCATCGAGCATCAAATGAAACTGCAATTTATTCATATCAGGATT
ATCAATACCATATTTTTGAAAAAGCCGTTTCTGTAATGAAGGAGAAAACTCACCGAGGCA
GTTCCATAGGATGGCAAGATCCTGGTATCGGTCTGCGATTCCGACTCGTCCAACATCAAT
ACAACCTATTAATTTCCCCTCGTCAAAAATAAGGTTATCAAGTGAGAAATCACCATGAGT
GACGACTGAATCCGGTGAGAATGGCAAAAGCTTATGCATTTCTTTCCAGACTTGTTCAAC
AGGCCAGCCATTACGCTCGTCATCAAAATCACTCGCATCAACCAAACCGTTATTCATTCG
TGATTGCGCCTGAGCGAGACGAAATACGCGATCGCTGTTAAAAGGACAATTACAAACAGG
AATCGAATGCAACCGGCGCAGGAACACTGCCAGCGCATCAACAATATTTTCACCTGAATC
AGGATATTCTTCTAATACCTGGAATGCTGTTTTCCCGGGGATCGCAGTGGTGAGTAACCA
TGCATCATCAGGAGTACGGATAAAATGCTTGATGGTCGGAAGAGGCATAAATTCCGTCAG
CCAGTTTAGTCTGACCATCTCATCTGTAACATCATTGGCAACGCTACCTTTGCCATGTTT
CAGAAACAACTCTGGCGCATCGGGCTTCCCATACAATCGATAGATTGTCGCACCTGATTG
CCCGACATTATCGCGAGCCCATTTATACCCATATAAATCAGCATCCATGTTGGAATTTAA
TCGCGGCCTCGAGCAAGACGTTTCCCGTTGAATATGGCTCATAACACCCCTTGTATTACT
GTTTATGTAAGCAGACAGTTTTATTGTTCATGAGCGGATACATATTTGAATGTATTTAGA
AAAATAAACAAATAGGGGTTCCGCGCACATTTCCCCGAAAAGTGCCACCTAAATTGTAAG
CGTTAATATTTTGTTAAAATTCGCGTTAAATTTTTGTTAAATCAGCTCATTTTTTAACCA
ATAGGCCGAAATCGGCAAAATCCCTTATAAATCAAAAGAATAGACCGAGATAGGGTTGAG
TGGCCGCTACAGGGCGCTCCCATTCGCCATTCAGGCTGCGCAACTGTTGGGAAGGGCGTT
TCGGTGCGGGCCTCTTCGCTATTACGCCAGCTGGCGAAAGGGGGATGTGCTGCAAGGCGA
TTAAGTTGGGTAACGCCAGGGTTTTCCCAGTCACACGCGTAATACGACTCACTATAG
SEQ ID NO: 19:
Human codon optimized (Genewiz) nucleic acid sequence encoding the
HBc_2A_HBs SAM transgene
ATGGACATCGATCCCTACAAAGAGTTCGGCGCTACCGTGGAGCTGCTGTCCTTTCTGCCT
TCCGACTTTTTCCCCTCCGTCAGAGATCTGCTGGACACCGCCTCCGCTCTGTATAGGGAG
GCCCTCGAGTCCCCCGAGCACTGTTCCCCTCATCACACAGCTCTGAGACAAGCCATTCTG
TGCTGGGGCGAGCTGATGACACTGGCCACATGGGTCGGCAACAACCTCGAAGATCCCGCC
TCTAGGGATCTGGTGGTCAACTACGTGAACACCAACATGGGACTGAAGATTAGACAGCTG
CTGTGGTTCCACATTAGCTGTCTCACCTTTGGCAGAGAAACCGTGCTGGAGTATCTGGTG
AGCTTCGGAGTGTGGATCAGAACCCCCCCCGCCTATAGACCTCCCAATGCCCCCATTCTG
TCCACACTGCCCGAGACAACCGTCGTCAGAAGGAGGGACAGAGGAAGATCCCCTAGAAGG
AGAACCCCCAGCCCTAGAAGAAGGAGGTCCCAGTCCCCCAGAAGAAGGAGAAGCCAGAGC
AGAGAATCCCAGTGCGCTCCCGTCAAACAGACCCTCAACTTCGATCTGCTCAAGCTGGCC
GGCGATGTGGAATCCAACCCCGGCCCTATGGAGAATATCACCAGCGGCTTTCTCGGCCCT
CTGCTGGTCCTCCAAGCTGGCTTCTTTCTGCTGACAAGGATTCTGACAATCCCCCAATCT
CTGGACAGCTGGTGGACATCCCTCAACTTTCTGGGCGGAAGCCCCGTGTGCCTCGGCCAA
AACTCCCAGAGCCCCACATCCAATCACTCCCCCACCAGCTGCCCCCCTATTTGCCCCGGC
TACAGATGGATGTGTCTGAGAAGGTTCATCATCTTCCTCTTCATTCTCCTCCTCTGCCTC
ATCTTTCTGCTGGTGCTCCTCGACTACCAAGGCATGCTGCCCGTGTGCCCTCTGATCCCC
GGCAGCACCACCACAAATACCGGCCCTTGTAAGACATGCACCACACCCGCCCAAGGCAAC
AGCATGTTTCCTAGCTGCTGCTGCACAAAGCCTACAGACGGCAACTGCACATGCATTCCT
ATCCCCAGCAGCTGGGCCTTCGCTAAGTATCTGTGGGAATGGGCTTCCGTGAGGTTCAGC
TGGCTCTCTCTGCTGGTGCCCTTCGTGCAATGGTTTGTGGGCCTCAGCCCTACCGTGTGG
CTGTCCGCTATTTGGATGATGTGGTATTGGGGACCCTCTCTGTACAGCATCGTGTCCCCC
TTCATCCCTCTGCTGCCCATTTTTTTCTGTCTGTGGGTGTATATTTGATGA
SEQ ID NO: 20:
HBc_2A_HBs in AA098, SAM plasmid sequence
ATAGGCGGCGCATGAGAGAAGCCCAGACCAATTACCTACCCAAAATGGAGAAAGTTCACG
TTGACATCGAGGAAGACAGCCCATTCCTCAGAGCTTTGCAGCGGAGCTTCCCGCAGTTTG
AGGTAGAAGCCAAGCAGGTCACTGATAATGACCATGCTAATGCCAGAGCGTTTTCGCATC
TGGCTTCAAAACTGATCGAAACGGAGGTGGACCCATCCGACACGATCCTTGACATTGGAA
GTGCGCCCGCCCGCAGAATGTATTCTAAGCACAAGTATCATTGTATCTGTCCGATGAGAT
GTGCGGAAGATCCGGACAGATTGTATAAGTATGCAACTAAGCTGAAGAAAAACTGTAAGG
AAATAACTGATAAGGAATTGGACAAGAAAATGAAGGAGCTCGCCGCCGTCATGAGCGACC
CTGACCTGGAAACTGAGACTATGTGCCTCCACGACGACGAGTCGTGTCGCTACGAAGGGC
AAGTCGCTGTTTACCAGGATGTATACGCGGTTGACGGACCGACAAGTCTCTATCACCAAG
CCAATAAGGGAGTTAGAGTCGCCTACTGGATAGGCTTTGACACCACCCCTTTTATGTTTA
AGAACTTGGCTGGAGCATATCCATCATACTCTACCAACTGGGCCGACGAAACCGTGTTAA
CGGCTCGTAACATAGGCCTATGCAGCTCTGACGTTATGGAGCGGTCACGTAGAGGGATGT
CCATTCTTAGAAAGAAGTATTTGAAACCATCCAACAATGTTCTATTCTCTGTTGGCTCGA
CCATCTACCACGAGAAGAGGGACTTACTGAGGAGCTGGCACCTGCCGTCTGTATTTCACT
TACGTGGCAAGCAAAATTACACATGTCGGTGTGAGACTATAGTTAGTTGCGACGGGTACG
TCGTTAAAAGAATAGCTATCAGTCCAGGCCTGTATGGGAAGCCTTCAGGCTATGCTGCTA
CGATGCACCGCGAGGGATTCTTGTGCTGCAAAGTGACAGACACATTGAACGGGGAGAGGG
TCTCTTTTCCCGTGTGCACGTATGTGCCAGCTACATTGTGTGACCAAATGACTGGCATAC
TGGCAACAGATGTCAGTGCGGACGACGCGCAAAAACTGCTGGTTGGGCTCAACCAGCGTA
TAGTCGTCAACGGTCGCACCCAGAGAAACACCAATACCATGAAAAATTACCTTTTGCCCG
TAGTGGCCCAGGCATTTGCTAGGTGGGCAAAGGAATATAAGGAAGATCAAGAAGATGAAA
GGCCACTAGGACTACGAGATAGACAGTTAGTCATGGGGTGTTGTTGGGCTTTTAGAAGGC
ACAAGATAACATCTATTTATAAGCGCCCGGATACCCAAACCATCATCAAAGTGAACAGCG
ATTTCCACTCATTCGTGCTGCCCAGGATAGGCAGTAACACATTGGAGATCGGGCTGAGAA
CAAGAATCAGGAAAATGTTAGAGGAGCACAAGGAGCCGTCACCTCTCATTACCGCCGAGG
ACGTACAAGAAGCTAAGTGCGCAGCCGATGAGGCTAAGGAGGTGCGTGAAGCCGAGGAGT
TGCGCGCAGCTCTACCACCTTTGGCAGCTGATGTTGAGGAGCCCACTCTGGAAGCCGATG
TCGACTTGATGTTACAAGAGGCTGGGGCCGGCTCAGTGGAGACACCTCGTGGCTTGATAA
AGGTTACCAGCTACGATGGCGAGGACAAGATCGGCTCTTACGCTGTGCTTTCTCCGCAGG
CTGTACTCAAGAGTGAAAAATTATCTTGCATCCACCCTCTCGCTGAACAAGTCATAGTGA
TAACACACTCTGGCCGAAAAGGGCGTTATGCCGTGGAACCATACCATGGTAAAGTAGTGG
TGCCAGAGGGACATGCAATACCCGTCCAGGACTTTCAAGCTCTGAGTGAAAGTGCCACCA
TTGTGTACAACGAACGTGAGTTCGTAAACAGGTACCTGCACCATATTGCCACACATGGAG
GAGCGCTGAACACTGATGAAGAATATTACAAAACTGTCAAGCCCAGCGAGCACGACGGCG
AATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAA
CACGACCAGCCGCTCCTTACCAAGTACCAACCATAGGGGTGTATGGCGTGCCAGGATCAG
GCAAGTCTGGCATCATTAAAAGCGCAGTCACCAAAAAAGATCTAGTGGTGAGCGCCAAGA
AAGAAAACTGTGCAGAAATTATAAGGGACGTCAAGAAAATGAAAGGGCTGGACGTCAATG
CCAGAACTGTGGACTCAGTGCTCTTGAATGGATGCAAACACCCCGTAGAGACCCTGTATA
TTGACGAAGCTTTTGCTTGTCATGCAGGTACTCTCAGAGCGCTCATAGCCATTATAAGAC
CTAAAAAGGCAGTGCTCTGCGGGGATCCCAAACAGTGCGGTTTTTTTAACATGATGTGCC
TGAAAGTGCATTTTAACCACGAGATTTGCACACAAGTCTTCCACAAAAGCATCTCTCGCC
GTTGCACTAAATCTGTGACTTCGGTCGTCTCAACCTTGTTTTACGACAAAAAAATGAGAA
CGACGAATCCGAAAGAGACTAAGATTGTGATTGACACTACCGGCAGTACCAAACCTAAGC
AGGACGATCTCATTCTCACTTGTTTCAGAGGGTGGGTGAAGCAGTTGCAAATAGATTACA
AAGGCAACGAAATAATGACGGCAGCTGCCTCTCAAGGGCTGACCCGTAAAGGTGTGTATG
CCGTTCGGTACAAGGTGAATGAAAATCCTCTGTACGCACCCACCTCAGAACATGTGAACG
TCCTACTGACCCGCACGGAGGACCGCATCGTGTGGAAAACACTAGCCGGCGACCCATGGA
TAAAAACACTGACTGCCAAGTACCCTGGGAATTTCACTGCCACGATAGAGGAGTGGCAAG
CAGAGCATGATGCCATCATGAGGCACATCTTGGAGAGACCGGACCCTACCGACGTCTTCC
AGAATAAGGCAAACGTGTGTTGGGCCAAGGCTTTAGTGCCGGTGCTGAAGACCGCTGGCA
TAGACATGACCACTGAACAATGGAACACTGTGGATTATTTTGAAACGGACAAAGCTCACT
CAGCAGAGATAGTATTGAACCAACTATGCGTGAGGTTCTTTGGACTCGATCTGGACTCCG
GTCTATTTTCTGCACCCACTGTTCCGTTATCCATTAGGAATAATCACTGGGATAACTCCC
CGTCGCCTAACATGTACGGGCTGAATAAAGAAGTGGTCCGTCAGCTCTCTCGCAGGTACC
CACAACTGCCTCGGGCAGTTGCCACTGGAAGAGTCTATGACATGAACACTGGTACACTGC
GCAATTATGATCCGCGCATAAACCTAGTACCTGTAAACAGAAGACTGCCTCATGCTTTAG
TCCTCCACCATAATGAACACCCACAGAGTGACTTTTCTTCATTCGTCAGCAAATTGAAGG
GCAGAACTGTCCTGGTGGTCGGGGAAAAGTTGTCCGTCCCAGGCAAAATGGTTGACTGGT
TGTCAGACCGGCCTGAGGCTACCTTCAGAGCTCGGCTGGATTTAGGCATCCCAGGTGATG
TGCCCAAATATGACATAATATTTGTTAATGTGAGGACCCCATATAAATACCATCACTATC
AGCAGTGTGAAGACCATGCCATTAAGCTTAGCATGTTGACCAAGAAAGCTTGTCTGCATC
TGAATCCCGGCGGAACCTGTGTCAGCATAGGTTATGGTTACGCTGACAGGGCCAGCGAAA
GCATCATTGGTGCTATAGCGCGGCAGTTCAAGTTTTCCCGGGTATGCAAACCGAAATCCT
CACTTGAAGAGACGGAAGTTCTGTTTGTATTCATTGGGTACGATCGCAAGGCCCGTACGC
ACAATCCTTACAAGCTTTCATCAACCTTGACCAACATTTATACAGGTTCCAGACTCCACG
AAGCCGGATGTGCACCCTCATATCATGTGGTGCGAGGGGATATTGCCACGGCCACCGAAG
GAGTGATTATAAATGCTGCTAACAGCAAAGGACAACCTGGCGGAGGGGTGTGCGGAGCGC
TGTATAAGAAATTCCCGGAAAGCTTCGATTTACAGCCGATCGAAGTAGGAAAAGCGCGAC
TGGTCAAAGGTGCAGCTAAACATATCATTCATGCCGTAGGACCAAACTTCAACAAAGTTT
CGGAGGTTGAAGGTGACAAACAGTTGGCAGAGGCTTATGAGTCCATCGCTAAGATTGTCA
ACGATAACAATTACAAGTCAGTAGCGATTCCACTGTTGTCCACCGGCATCTTTTCCGGGA
ACAAAGATCGACTAACCCAATCATTGAACCATTTGCTGACAGCTTTAGACACCACTGATG
CAGATGTAGCCATATACTGCAGGGACAAGAAATGGGAAATGACTCTCAAGGAAGCAGTGG
CTAGGAGAGAAGCAGTGGAGGAGATATGCATATCCGACGACTCTTCAGTGACAGAACCTG
ATGCAGAGCTGGTGAGGGTGCATCCGAAGAGTTCTTTGGCTGGAAGGAAGGGCTACAGCA
CAAGCGATGGCAAAACTTTCTCATATTTGGAAGGGACCAAGTTTCACCAGGCGGCCAAGG
ATATAGCAGAAATTAATGCCATGTGGCCCGTTGCAACGGAGGCCAATGAGCAGGTATGCA
TGTATATCCTCGGAGAAAGCATGAGCAGTATTAGGTCGAAATGCCCCGTCGAAGAGTCGG
AAGCCTCCACACCACCTAGCACGCTGCCTTGCTTGTGCATCCATGCCATGACTCCAGAAA
GAGTACAGCGCCTAAAAGCCTCACGTCCAGAACAAATTACTGTGTGCTCATCCTTTCCAT
TGCCGAAGTATAGAATCACTGGTGTGCAGAAGATCCAATGCTCCCAGCCTATATTGTTCT
CACCGAAAGTGCCTGCGTATATTCATCCAAGGAAGTATCTCGTGGAAACACCACCGGTAG
ACGAGACTCCGGAGCCATCGGCAGAGAACCAATCCACAGAGGGGACACCTGAACAACCAC
CACTTATAACCGAGGATGAGACCAGGACTAGAACGCCTGAGCCGATCATCATCGAAGAGG
AAGAAGAGGATAGCATAAGTTTGCTGTCAGATGGCCCGACCCACCAGGTGCTGCAAGTCG
AGGCAGACATTCACGGGCCGCCCTCTGTATCTAGCTCATCCTGGTCCATTCCTCATGCAT
CCGACTTTGATGTGGACAGTTTATCCATACTTGACACCCTGGAGGGAGCTAGCGTGACCA
GCGGGGCAACGTCAGCCGAGACTAACTCTTACTTCGCAAAGAGTATGGAGTTTCTGGCGC
GACCGGTGCCTGCGCCTCGAACAGTATTCAGGAACCCTCCACATCCCGCTCCGCGCACAA
GAACACCGTCACTTGCACCCAGCAGGGCCTGCTCGAGAACCAGCCTAGTTTCCACCCCGC
CAGGCGTGAATAGGGTGATCACTAGAGAGGAGCTCGAGGCGCTTACCCCGTCACGCACTC
CTAGCAGGTCGGTCTCGAGAACCAGCCTGGTCTCCAACCCGCCAGGCGTAAATAGGGTGA
TTACAAGAGAGGAGTTTGAGGCGTTCGTAGCACAACAACAATGACGGTTTGATGCGGGTG
CATACATCTTTTCCTCCGACACCGGTCAAGGGCATTTACAACAAAAATCAGTAAGGCAAA
CGGTGCTATCCGAAGTGGTGTTGGAGAGGACCGAATTGGAGATTTCGTATGCCCCGCGCC
TCGACCAAGAAAAAGAAGAATTACTACGCAAGAAATTACAGTTAAATCCCACACCTGCTA
ACAGAAGCAGATACCAGTCCAGGAAGGTGGAGAACATGAAAGCCATAACAGCTAGACGTA
TTCTGCAAGGCCTAGGGCATTATTTGAAGGCAGAAGGAAAAGTGGAGTGCTACCGAACCC
TGCATCCTGTTCCTTTGTATTCATCTAGTGTGAACCGTGCCTTTTCAAGCCCCAAGGTCG
CAGTGGAAGCCTGTAACGCCATGTTGAAAGAGAACTTTCCGACTGTGGCTTCTTACTGTA
TTATTCCAGAGTACGATGCCTATTTGGACATGGTTGACGGAGCTTCATGCTGCTTAGACA
CTGCCAGTTTTTGCCCTGCAAAGCTGCGCAGCTTTCCAAAGAAACACTCCTATTTGGAAC
CCACAATACGATCGGCAGTGCCTTCAGCGATCCAGAACACGCTCCAGAACGTCCTGGCAG
CTGCCACAAAAAGAAATTGCAATGTCACGCAAATGAGAGAATTGCCCGTATTGGATTCGG
CGGCCTTTAATGTGGAATGCTTCAAGAAATATGCGTGTAATAATGAATATTGGGAAACGT
TTAAAGAAAACCCCATCAGGCTTACTGAAGAAAACGTGGTAAATTACATTACCAAATTAA
AAGGACCAAAAGCTGCTGCTCTTTTTGCGAAGACACATAATTTGAATATGTTGCAGGACA
TACCAATGGACAGGTTTGTAATGGACTTAAAGAGAGACGTGAAAGTGACTCCAGGAACAA
AACATACTGAAGAACGGCCCAAGGTACAGGTGATCCAGGCTGCCGATCCGCTAGCAACAG
CGTATCTGTGCGGAATCCACCGAGAGCTGGTTAGGAGATTAAATGCGGTCCTGCTTCCGA
ACATTCATACACTGTTTGATATGTCGGCTGAAGACTTTGACGCTATTATAGCCGAGCACT
TCCAGCCTGGGGATTGTGTTCTGGAAACTGACATCGCGTCGTTTGATAAAAGTGAGGACG
ACGCCATGGCTCTGACCGCGTTAATGATTCTGGAAGACTTAGGTGTGGACGCAGAGCTGT
TGACGCTGATTGAGGCGGCTTTCGGCGAAATTTCATCAATACATTTGCCCACTAAAACTA
AATTTAAATTCGGAGCCATGATGAAATCTGGAATGTTCCTCACACTGTTTGTGAACACAG
TCATTAACATTGTAATCGCAAGCAGAGTGTTGAGAGAACGGCTAACCGGATCACCATGTG
CAGCATTCATTGGAGATGACAATATCGTGAAAGGAGTCAAATCGGACAAATTAATGGCAG
ACAGGTGCGCCACCTGGTTGAATATGGAAGTCAAGATTATAGATGCTGTGGTGGGCGAGA
AAGCGCCTTATTTCTGTGGAGGGTTTATTTTGTGTGACTCCGTGACCGGCACAGCGTGCC
GTGTGGCAGACCCCCTAAAAAGGCTGTTTAAGCTTGGCAAACCTCTGGCAGCAGACGATG
AACATGATGATGACAGGAGAAGGGCATTGCATGAAGAGTCAACACGCTGGAACCGAGTGG
GTATTCTTTCAGAGCTGTGCAAGGCAGTAGAATCAAGGTATGAAACCGTAGGAACTTCCA
TCATAGTTATGGCCATGACTACTCTAGCTAGCAGTGTTAAATCATTCAGCTACCTGAGAG
GGGCCCctataactctctacggctaacctgaatggactacgacatagtctagtccgccaa
gATGGACATCGATCCCTACAAAGAGTTCGGCGCTACCGTGGAGCTGCTGTCCTTTCTGCC
TTCCGACTTTTTCCCCTCCGTCAGAGATCTGCTGGACACCGCCTCCGCTCTGTATAGGGA
GGCCCTCGAGTCCCCCGAGCACTGTTCCCCTCATCACACAGCTCTGAGACAAGCCATTCT
GTGCTGGGGCGAGCTGATGACACTGGCCACATGGGTCGGCAACAACCTCGAAGATCCCGC
CTCTAGGGATCTGGTGGTCAACTACGTGAACACCAACATGGGACTGAAGATTAGACAGCT
GCTGTGGTTCCACATTAGCTGTCTCACCTTTGGCAGAGAAACCGTGCTGGAGTATCTGGT
GAGCTTCGGAGTGTGGATCAGAACCCCCCCCGCCTATAGACCTCCCAATGCCCCCATTCT
GTCCACACTGCCCGAGACAACCGTCGTCAGAAGGAGGGACAGAGGAAGATCCCCTAGAAG
GAGAACCCCCAGCCCTAGAAGAAGGAGGTCCCAGTCCCCCAGAAGAAGGAGAAGCCAGAG
CAGAGAATCCCAGTGCGCTCCCGTCAAACAGACCCTCAACTTCGATCTGCTCAAGCTGGC
CGGCGATGTGGAATCCAACCCCGGCCCTATGGAGAATATCACCAGCGGCTTTCTCGGCCC
TCTGCTGGTCCTCCAAGCTGGCTTCTTTCTGCTGACAAGGATTCTGACAATCCCCCAATC
TCTGGACAGCTGGTGGACATCCCTCAACTTTCTGGGCGGAAGCCCCGTGTGCCTCGGCCA
AAACTCCCAGAGCCCCACATCCAATCACTCCCCCACCAGCTGCCCCCCTATTTGCCCCGG
CTACAGATGGATGTGTCTGAGAAGGTTCATCATCTTCCTCTTCATTCTCCTCCTCTGCCT
CATCTTTCTGCTGGTGCTCCTCGACTACCAAGGCATGCTGCCCGTGTGCCCTCTGATCCC
CGGCAGCACCACCACAAATACCGGCCCTTGTAAGACATGCACCACACCCGCCCAAGGCAA
CAGCATGTTTCCTAGCTGCTGCTGCACAAAGCCTACAGACGGCAACTGCACATGCATTCC
TATCCCCAGCAGCTGGGCCTTCGCTAAGTATCTGTGGGAATGGGCTTCCGTGAGGTTCAG
CTGGCTCTCTCTGCTGGTGCCCTTCGTGCAATGGTTTGTGGGCCTCAGCCCTACCGTGTG
GCTGTCCGCTATTTGGATGATGTGGTATTGGGGACCCTCTCTGTACAGCATCGTGTCCCC
CTTCATCCCTCTGCTGCCCATTTTTTTCTGTCTGTGGGTGTATATTTGATGAggcgcgcc
cacccaGCGGCCGCATACAGCAGCAATTGGCAAGCTGCTTACATAGAACTCGCGGCGATT
GGCATGCCGCCTTAAAATTTTTATTTTATTTTTCTTTTCTTTTCCGAATCGGATTTTGTT
TTTAATATTTCAAAAAAAAAAAAAAAAAAAAAAAAAAAAAAAAAAAAAAAAGAAGAGCGT
TTAAACACGTGATATCTGGCCTCATGGGCCTTCCTTTCACTGCCCGCTTTCCAGTCGGGA
AACCTGTCGTGCCAGCTGCATTAACATGGTCATAGCTGTTTCCTTGCGTATTGGGCGCTC
TCCGCTTCCTCGCTCACTGACTCGCTGCGCTCGGTCGTTCGGGTAAAGCCTGGGGTGCCT
AATGAGCAAAAGGCCAGCAAAAGGCCAGGAACCGTAAAAAGGCCGCGTTGCTGGCGTTTT
TCCATAGGCTCCGCCCCCCTGACGAGCATCACAAAAATCGACGCTCAAGTCAGAGGTGGC
GAAACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAGCG
TGGCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTA
ACTACGGCTACACTAGAAGAACAGTATTTGGTATCTGCGCTCTGCTGAAGCCAGTTACCT
TCGGAAAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTCA
TGAATACACGGTGCCTGACTGCGTTAGCAATTTAACTGTGATAAACTACCGCATTAAAGC
TTATCGATGATAAGCTGTCAAACATGAGAATTCTTAGAAAAACTCATCGAGCATCAAATG
AAACTGCAATTTATTCATATCAGGATTATCAATACCATATTTTTGAAAAAGCCGTTTCTG
TAATGAAGGAGAAAACTCACCGAGGCAGTTCCATAGGATGGCAAGATCCTGGTATCGGTC
TGCGATTCCGACTCGTCCAACATCAATACAACCTATTAATTTCCCCTCGTCAAAAATAAG
GTTATCAAGTGAGAAATCACCATGAGTGACGACTGAATCCGGTGAGAATGGCAAAAGCTT
ATGCATTTCTTTCCAGACTTGTTCAACAGGCCAGCCATTACGCTCGTCATCAAAATCACT
CGCATCAACCAAACCGTTATTCATTCGTGATTGCGCCTGAGCGAGACGAAATACGCGATC
GCTGTTAAAAGGACAATTACAAACAGGAATCGAATGCAACCGGCGCAGGAACACTGCCAG
CGCATCAACAATATTTTCACCTGAATCAGGATATTCTTCTAATACCTGGAATGCTGTTTT
CCCGGGGATCGCAGTGGTGAGTAACCATGCATCATCAGGAGTACGGATAAAATGCTTGAT
GGTCGGAAGAGGCATAAATTCCGTCAGCCAGTTTAGTCTGACCATCTCATCTGTAACATC
ATTGGCAACGCTACCTTTGCCATGTTTCAGAAACAACTCTGGCGCATCGGGCTTCCCATA
CAATCGATAGATTGTCGCACCTGATTGCCCGACATTATCGCGAGCCCATTTATACCCATA
TAAATCAGCATCCATGTTGGAATTTAATCGCGGCCTCGAGCAAGACGTTTCCCGTTGAAT
ATGGCTCATAACACCCCTTGTATTACTGTTTATGTAAGCAGACAGTTTTATTGTTCATGA
GCGGATACATATTTGAATGTATTTAGAAAAATAAACAAATAGGGGTTCCGCGCACATTTC
CCCGAAAAGTGCCACCTAAATTGTAAGCGTTAATATTTTGTTAAAATTCGCGTTAAATTT
TTGTTAAATCAGCTCATTTTTTAACCAATAGGCCGAAATCGGCAAAATCCCTTATAAATC
AAAAGAATAGACCGAGATAGGGTTGAGTGGCCGCTACAGGGCGCTCCCATTCGCCATTCA
GGCTGCGCAACTGTTGGGAAGGGCGTTTCGGTGCGGGCCTCTTCGCTATTACGCCAGCTG
GCGAAAGGGGGATGTGCTGCAAGGCGATTAAGTTGGGTAACGCCAGGGTTTTCCCAGTCA
CACGCGTAATACGACTCACTATAG
SEQ ID NO: 21:
Amino acid sequence of hli-HBc
MHRRRSRSCREDQKPVMDDQRDLISNNEQLPMLGRRPGAPESKCSRGALYTGFSILVTLL
LAGQATTAYFLYQQQGRLDKLTVTSQNLQLENLRMKLPKPPKPVSKMRMATPLLMQALPM
GALPQGPMQNATKYGNMTEDHVMHLLQNADPLKVYPPLKGSFPENLRHLKNTMETIDWKV
FESWMHHWLLFEMSRHSLEQKPTDAPPKESLELEDPSSGLGVTKQDLGPVPMDIDPYKEF
GATVELLSFLPSDFFPSVRDLLDTASALYREALESPEHCSPHHTALRQAILCWGELMTLA
TWVGNNLEDPASRDLVVNYVNTNMGLKIRQLLWFHISCLTFGRETVLEYLVSFGVWIRTP
PAYRPPNAPILSTLPETTVVRRRDRGRSPRRRTPSPRRRRSQSPRRRRSQSRESQC
SEQ ID NO: 22:
Nucleotide sequence encoding hli-HBc
ATGCACAGAAGAAGAAGCAGATCATGCAGGGAGGATCAGAAACCAGTGATGGACGACCAG
AGGGACCTGATCAGCAACAATGAGCAACTGCCAATGCTGGGCCGAAGACCCGGCGCCCCC
GAGTCAAAGTGCAGCCGAGGCGCCTTATACACCGGCTTCAGCATCCTGGTTACGCTGCTC
CTGGCAGGCCAGGCCACCACCGCATATTTCCTGTACCAACAGCAAGGCAGACTGGACAAG
CTAACCGTGACCTCACAGAACCTACAGCTGGAAAATCTGAGGATGAAGCTCCCAAAGCCC
CCTAAGCCCGTGAGCAAGATGAGAATGGCCACACCCCTGCTGATGCAGGCCCTCCCCATG
GGAGCCCTGCCACAGGGACCCATGCAGAATGCCACCAAGTACGGCAACATGACCGAGGAC
CACGTGATGCACCTGCTGCAGAACGCCGACCCCCTGAAGGTGTACCCCCCCCTGAAGGGC
AGCTTCCCCGAGAACCTGAGACACCTGAAGAACACCATGGAGACCATCGACTGGAAGGTG
TTCGAGAGCTGGATGCACCACTGGCTGCTGTTCGAGATGAGCAGACACAGCCTGGAGCAG
AAGCCCACCGACGCCCCCCCCAAGGAGTCCCTGGAGCTGGAGGACCCCAGCAGCGGCCTG
GGCGTGACCAAGCAGGACCTGGGCCCCGTGCCCATGGACATCGACCCCTACAAGGAGTTC
GGCGCCACAGTGGAGCTGCTGAGCTTCCTGCCCAGCGACTTCTTCCCCAGCGTGAGAGAC
CTGCTGGACACCGCCTCCGCCCTGTACAGAGAGGCCCTGGAGAGCCCCGAGCACTGCAGC
CCCCACCACACCGCCCTGAGACAGGCCATCCTGTGCTGGGGGGAGCTGATGACCCTGGCC
ACCTGGGGGGCAACAACCTGGAGGACCCCGCCAGCAGAGACCTGGTGGTGAACTACGTGA
ACACCAACATGGGCCTGAAGATCAGGCAGCTGCTGTGGTTCCACATCAGCTGCTTGACCT
TCGGCAGAGAGACCGTGCTGGAGTACCTGGTGAGCTTCGGCGTGTGGATCCGCACCCCTC
CCGCATACAGACCCCCCAACGCCCCCATCCTGAGCACCCTGCCCGAGACCACCGTGGTGA
GACGCAGAGACAGAGGCCGGAGCCCCCGCAGAAGAACCCCCAGCCCCAGGAGAAGACGGA
GCCAGAGCCCCAGAAGACGGCGCAGCCAGAGCAGAGAGAGCCAGTGC
SEQ ID NO: 23:
hli-HBc plasmid sequence (UTR4)
ACGCGTGCCTAAATTAATACGACTCACTATAAGGAGAAGCTGTCTATCGGGCTCCAGCGG
TCATGCACAGAAGAAGAAGCAGATCATGCAGGGAGGATCAGAAACCAGTGATGGACGACC
AGAGGGACCTGATCAGCAACAATGAGCAACTGCCAATGCTGGGCCGAAGACCCGGCGCCC
CCGAGTCAAAGTGCAGCCGAGGCGCCTTATACACCGGCTTCAGCATCCTGGTTACGCTGC
TCCTGGCAGGCCAGGCCACCACCGCATATTTCCTGTACCAACAGCAAGGCAGACTGGACA
AGCTAACCGTGACCTCACAGAACCTACAGCTGGAAAATCTGAGGATGAAGCTCCCAAAGC
CCCCTAAGCCCGTGAGCAAGATGAGAATGGCCACACCCCTGCTGATGCAGGCCCTCCCCA
TGGGAGCCCTGCCACAGGGACCCATGCAGAATGCCACCAAGTACGGCAACATGACCGAGG
ACCACGTGATGCACCTGCTGCAGAACGCCGACCCCCTGAAGGTGTACCCCCCCCTGAAGG
GCAGCTTCCCCGAGAACCTGAGACACCTGAAGAACACCATGGAGACCATCGACTGGAAGG
TGTTCGAGAGCTGGATGCACCACTGGCTGCTGTTCGAGATGAGCAGACACAGCCTGGAGC
AGAAGCCCACCGACGCCCCCCCCAAGGAGTCCCTGGAGCTGGAGGACCCCAGCAGCGGCC
TGGGCGTGACCAAGCAGGACCTGGGCCCCGTGCCCATGGACATCGACCCCTACAAGGAGT
TCGGCGCCACAGTGGAGCTGCTGAGCTTCCTGCCCAGCGACTTCTTCCCCAGCGTGAGAG
ACCTGCTGGACACCGCCTCCGCCCTGTACAGAGAGGCCCTGGAGAGCCCCGAGCACTGCA
GCCCCCACCACACCGCCCTGAGACAGGCCATCCTGTGCTGGGGGGAGCTGATGACCCTGG
CCACCTGGGTGGGCAACAACCTGGAGGACCCCGCCAGCAGAGACCTGGTGGTGAACTACG
TGAACACCAACATGGGCCTGAAGATCAGGCAGCTGCTGTGGTTCCACATCAGCTGCTTGA
CCTTCGGCAGAGAGACCGTGCTGGAGTACCTGGTGAGCTTCGGCGTGTGGATCCGCACCC
CTCCCGCATACAGACCCCCCAACGCCCCCATCCTGAGCACCCTGCCCGAGACCACCGTGG
TGAGACGCAGAGACAGAGGCCGGAGCCCCCGCAGAAGAACCCCCAGCCCCAGGAGAAGAC
GGAGCCAGAGCCCCAGAAGACGGCGCAGCCAGAGCAGAGAGAGCCAGTGCTGATAAGCCG
CCGCTCCAGCTTTGCACGTTTCGATCCCAAAGGCCCTTTTTAGGGCCGACCATTCATTGC
AAAAAAAAAAAAAAAAAAAAAAAAAAAAAAGCATATGACTAAAAAAAAAAAAAAAAAAAA
AAAAAAAAAAAAAAAAAAGAAGAGCGTTTAAACACGTGATATCTGGCCTCATGGGCCTTC
CTTTCACTGCCCGCTTTCCAGTCGGGAAACCTGTCGTGCCAGCTGCATTAACATGGTCAT
AGCTGTTTCCTTGCGTATTGGGCGCTCTCCGCTTCCTCGCTCACTGACTCGCTGCGCTCG
GTCGTTCGGGTAAAGCCTGGGGTGCCTAATGAGCAAAAGGCCAGCAAAAGGCCAGGAACC
GTAAAAAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACGACT
TATCGCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCCGGCA
AACAAACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAGTGGAACG
AAAACTCACGTTAAGGGATTTTGGTCATGAATACACGGTGCCTGACTGCGTTAGCAATTT
AACTGTGATAAACTACCGCATTAAAGCTTATCGATGATAAGCTGTCAAACATGAGAATTC
TTAGAAAAACTCATCGAGCATCAAATGAAACTGCAATTTATTCATATCAGGATTATCAAT
ACCATATTTTTGAAAAAGCCGTTTCTGTAATGAAGGAGAAAACTCACCGAGGCAGTTCCA
TAGGATGGCAAGATCCTGGTATCGGTCTGCGATTCCGACTCGTCCAACATCAATACAACC
TATTAATTTCCCCTCGTCAAAAATAAGGTTATCAAGTGAGAAATCACCATGAGTGACGAC
TGAATCCGGTGAGAATGGCAAAAGCTTATGCATTTCTTTCCAGACTTGTTCAACAGGCCA
GCCATTACGCTCGTCATCAAAATCACTCGCATCAACCAAACCGTTATTCATTCGTGATTG
CGCCTGAGCGAGACGAAATACGCGATCGCTGTTAAAAGGACAATTACAAACAGGAATCGA
ATGCAACCGGCGCAGGAACACTGCCAGCGCATCAACAATATTTTCACCTGAATCAGGATA
TTCTTCTAATACCTGGAATGCTGTTTTCCCGGGGATCGCAGTGGTGAGTAACCATGCATC
ATCAGGAGTACGGATAAAATGCTTGATGGTCGGAAGAGGCATAAATTCCGTCAGCCAGTT
TAGTCTGACCATCTCATCTGTAACATCATTGGCAACGCTACCTTTGCCATGTTTCAGAAA
CAACTCTGGCGCATCGGGCTTCCCATACAATCGATAGATTGTCGCACCTGATTGCCCGAC
ATTATCGCGAGCCCATTTATACCCATATAAATCAGCATCCATGTTGGAATTTAATCGCGG
CCTCGAGCAAGACGTTTCCCGTTGAATATGGCTCATAACACCCCTTGTATTACTGTTTAT
GTAAGCAGACAGTTTTATTGTTCATGAGCGGATACATATTTGAATGTATTTAGAAAAATA
AACAAATAGGGGTTCCGCGCACATTTCCCCGAAAAGTGCCACCTAAATTGTAAGCGTTAA
TATTTTGTTAAAATTCGCGTTAAATTTTTGTTAAATCAGCTCATTTTTTAACCAATAGGC
CGAAATCGGCAAAATCCCTTATAAATCAAAAGAATAGACCGAGATAGGGTTGAGTGGCCG
CTACAGGGCGCTCCCATTCGCCATTCAGGCTGCGCAACTGTTGGGAAGGGCGTTTCGGTG
CGGGCCTCTTCGCTATTACGCCAGCTGGCGAAAGGGGGATGTGCTGCAAGGCGATTAAGT
TGGGTAACGCCAGGGTTTTCCCAGTCAC
SEQ ID NO: 24:
Nucleotide sequence encoding HBs
ATGGAGAATATCACCAGCGGCTTCCTCGGCCCCCTCTTAGTGCTGCAGGCCGGCTTCTTC
CTCCTGACACGGATCCTGACCATCCCGCAGTCCCTGGACTCATGGTGGACCTCCCTGAAC
TTCCTGGGCGGCTCCCCCGTGTGCCTGGGCCAGAACTCCCAGAGCCCCACCAGCAACCAC
TCCCCCACCAGCTGCCCCCCCATCTGCCCCGGCTACAGGTGGATGTGCCTGCGGCGGTTC
ATCATCTTCCTGTTCATCCTGCTGCTGTGCCTGATCTTCCTGCTGGTGCTGCTGGACTAC
CAGGGCATGCTGCCCGTGTGCCCCCTGATCCCCGGCTCCACCACCACCAACACCGGCCCC
TGCAAGACCTGCACCACCCCCGCCCAGGGGAACAGCATGTTCCCCTCCTGCTGCTGCACC
AAGCCCACCGACGGCAACTGCACCTGCATCCCCATCCCCTCCAGCTGGGCCTTCGCCAAG
TACCTGTGGGAGTGGGCCTCCGTGCGGTTCAGCTGGCTGAGCCTGCTGGTGCCCTTCGTG
CAGTGGTTCGTGGGCCTGTCCCCCACCGTGTGGCTGTCCGCCATCTGGATGATGTGGTAC
TGGGGCCCCAGCCTGTACAGCATCGTGAGCCCCTTCATCCCCCTGCTGCCCATCTTCTTC
TGCCTGTGGGTGTACATC
SEQ ID NO: 25:
HBs plasmid sequence (UTR4)
ACGCGTGCCTAAATTAATACGACTCACTATAAGGAGAAGCTGTCTATCGGGCTCCAGCGG
TCATGGAGAATATCACCAGCGGCTTCCTCGGCCCCCTCTTAGTGCTGCAGGCCGGCTTCT
TCCTCCTGACACGGATCCTGACCATCCCGCAGTCCCTGGACTCATGGTGGACCTCCCTGA
ACTTCCTGGGCGGCTCCCCCGTGTGCCTGGGCCAGAACTCCCAGAGCCCCACCAGCAACC
ACTCCCCCACCAGCTGCCCCCCCATCTGCCCCGGCTACAGGTGGATGTGCCTGCGGCGGT
TCATCATCTTCCTGTTCATCCTGCTGCTGTGCCTGATCTTCCTGCTGGTGCTGCTGGACT
ACCAGGGCATGCTGCCCGTGTGCCCCCTGATCCCCGGCTCCACCACCACCAACACCGGCC
CCTGCAAGACCTGCACCACCCCCGCCCAGGGGAACAGCATGTTCCCCTCCTGCTGCTGCA
CCAAGCCCACCGACGGCAACTGCACCTGCATCCCCATCCCCTCCAGCTGGGCCTTCGCCA
AGTACCTGTGGGAGTGGGCCTCCGTGCGGTTCAGCTGGCTGAGCCTGCTGGTGCCCTTCG
TGCAGTGGTTCGTGGGCCTGTCCCCCACCGTGTGGCTGTCCGCCATCTGGATGATGTGGT
ACTGGGGCCCCAGCCTGTACAGCATCGTGAGCCCCTTCATCCCCCTGCTGCCCATCTTCT
TCTGCCTGTGGGTGTACATCTGATAAGCCGCCGCTCCAGCTTTGCACGTTTCGATCCCAA
AGGCCCTTTTTAGGGCCGACCATTCATTGCAAAAAAAAAAAAAAAAAAAAAAAAAAAAAA
GCATATGACTAAAAAAAAAAAAAAAAAAAAAAAAAAAAAAAAAAAAAAGAAGAGCGTTTA
AACACGTGATATCTGGCCTCATGGGCCTTCCTTTCACTGCCCGCTTTCCAGTCGGGAAAC
CTGTCGTGCCAGCTGCATTAACATGGTCATAGCTGTTTCCTTGCGTATTGGGCGCTCTCC
GCTTCCTCGCTCACTGACTCGCTGCGCTCGGTCGTTCGGGTAAAGCCTGGGGTGCCTAAT
GAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAACT
ACGGCTACACTAGAAGAACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTTTTT
TTGTTTGCAAGCAGCAGATTACGCGCAGAAAAAAAGGATCTCAAGAAGATCCTTTGATCT
TTTCTACGGGGTCTGACGCTCAGTGGAACGAAAACTCACGTTAAGGGATTTTGGTCATGA
ATACACGGTGCCTGACTGCGTTAGCAATTTAACTGTGATAAACTACCGCATTAAAGCTTA
TCGATGATAAGCTGTCAAACATGAGAATTCTTAGAAAAACTCATCGAGCATCAAATGAAA
CTGCAATTTATTCATATCAGGATTATCAATACCATATTTTTGAAAAAGCCGTTTCTGTAA
TGAAGGAGAAAACTCACCGAGGCAGTTCCATAGGATGGCAAGATCCTGGTATCGGTCTGC
GATTCCGACTCGTCCAACATCAATACAACCTATTAATTTCCCCTCGTCAAAAATAAGGTT
ATCAAGTGAGAAATCACCATGAGTGACGACTGAATCCGGTGAGAATGGCAAAAGCTTATG
CATTTCTTTCCAGACTTGTTCAACAGGCCAGCCATTACGCTCGTCATCAAAATCACTCGC
ATCAACCAAACCGTTATTCATTCGTGATTGCGCCTGAGCGAGACGAAATACGCGATCGCT
GTTAAAAGGACAATTACAAACAGGAATCGAATGCAACCGGCGCAGGAACACTGCCAGCGC
ATCAACAATATTTTCACCTGAATCAGGATATTCTTCTAATACCTGGAATGCTGTTTTCCC
GGGGATCGCAGTGGTGAGTAACCATGCATCATCAGGAGTACGGATAAAATGCTTGATGGT
CGGAAGAGGCATAAATTCCGTCAGCCAGTTTAGTCTGACCATCTCATCTGTAACATCATT
GGCAACGCTACCTTTGCCATGTTTCAGAAACAACTCTGGCGCATCGGGCTTCCCATACAA
TCGATAGATTGTCGCACCTGATTGCCCGACATTATCGCGAGCCCATTTATACCCATATAA
ATCAGCATCCATGTTGGAATTTAATCGCGGCCTCGAGCAAGACGTTTCCCGTTGAATATG
GCTCATAACACCCCTTGTATTACTGTTTATGTAAGCAGACAGTTTTATTGTTCATGAGCG
GATACATATTTGAATGTATTTAGAAAAATAAACAAATAGGGGTTCCGCGCACATTTCCCC
GAAAAGTGCCACCTAAATTGTAAGCGTTAATATTTTGTTAAAATTCGCGTTAAATTTTTG
TTAAATCAGCTCATTTTTTAACCAATAGGCCGAAATCGGCAAAATCCCTTATAAATCAAA
AGAATAGACCGAGATAGGGTTGAGTGGCCGCTACAGGGCGCTCCCATTCGCCATTCAGGC
TGCGCAACTGTTGGGAAGGGCGTTTCGGTGCGGGCCTCTTCGCTATTACGCCAGCTGGCG
AAAGGGGGATGTGCTGCAAGGCGATTAAGTTGGGTAACGCCAGGGTTTTCCCAGTCAC
SEQ ID NO: 26:
Amino acid sequence of hli-HBs
MHRRRSRSCREDQKPVMDDQRDLISNNEQLPMLGRRPGAPESKCSRGALYTGFSILVTLL
LAGQATTAYFLYQQQGRLDKLTVTSQNLQLENLRMKLPKPPKPVSKMRMATPLLMQALPM
GALPQGPMQNATKYGNMTEDHVMHLLQNADPLKVYPPLKGSFPENLRHLKNTMETIDWKV
FESWMHHWLLFEMSRHSLEQKPTDAPPKESLELEDPSSGLGVTKQDLGPVPMENITSGFL
GPLLVLQAGFFLLTRILTIPQSLDSWWTSLNFLGGSPVCLGQNSQSPTSNHSPTSCPPIC
PGYRWMCLRRFIIFLFILLLCLIFLLVLLDYQGMLPVCPLIPGSTTTNTGPCKTCTTPAQ
GNSMFPSCCCTKPTDGNCTCIPIPSSWAFAKYLWEWASVRFSWLSLLVPFVQWFVGLSPT
VWLSAIWMMWYWGPSLYSIVSPFIPLLPIFFCLWVYI
SEQ ID NO: 27:
Nucleotide sequence encoding hli-HBs
ATGCACAGAAGACGCTCCAGGAGCTGCCGAGAGGATCAGAAACCTGTGATGGACGACCAG
CGCGACCTGATCAGCAACAATGAGCAGTTGCCCATGCTGGGACGCCGCCCCGGCGCACCG
GAGAGCAAGTGCTCCCGCGGCGCCTTATACACCGGATTCAGCATCCTCGTTACCCTGTTA
CTGGCCGGGCAGGCCACCACGGCCTACTTCCTCTACCAACAACAGGGCAGGTTAGACAAG
CTGACCGTGACCTCACAGAATCTCCAGTTAGAGAACCTGAGAATGAAGCTGCCAAAGCCA
CCTAAGCCTGTGAGCAAGATGCGGATGGCCACCCCCCTGCTGATGCAGGCCCTGCCTATG
GGCGCCCTGCCACAGGGCCCCATGCAGAATGCCACCAAGTACGGCAATATGACCGAGGAC
CACGTGATGCACCTGCTGCAGAATGCCGACCCTCTGAAGGTGTACCCTCCTCTGAAGGGC
TCCTTCCCAGAGAATCTGCGGCACCTGAAGAACACCATGGAGACCATCGACTGGAAGGTG
TTCGAGAGCTGGATGCACCACTGGCTGCTGTTCGAGATGTCCAGGCACTCTCTGGAGCAG
AAGCCCACCGACGCCCCTCCAAAGGAGAGCCTGGAGCTGGAGGACCCCAGCAGCGGCCTG
GGCGTGACCAAGCAGGACCTGGGCCCAGTGCCAATGGAGAACATCACCAGCGGCTTCCTG
GGCCCCCTGCTGGTGCTGCAGGCCGGCTTCTTCCTGCTGACCAGGATCCTGACCATCCCT
CAGAGCCTGGACAGCTGGTGGACCAGCCTGAATTTCCTGGGCGGCAGCCCAGTGTGCCTG
GGCCAGAACAGCCAGTCCCCAACCAGCAACCACAGCCCTACCAGCTGCCCTCCCATCTGC
CCAGGCTACAGGTGGATGTGCCTGAGGCGGTTCATCATCTTCCTGTTCATCCTGCTGCTG
TGCCTGATCTTCCTGCTGGTGCTGCTGGACTACCAGGGCATGCTGCCAGTGTGCCCCCTG
ATCCCCGGCAGCACCACCACCAACACCGGCCCCTGCAAGACCTGCACCACCCCCGCCCAG
GGCAACAGCATGTTCCCCTCGTGCTGCTGCACCAAGCCCACCGACGGCAATTGCACCTGC
ATCCCCATCCCCAGCAGCTGGGCCTTCGCCAAGTACCTGTGGGAGTGGGCCAGCGTGCGG
TTCAGCTGGCTCAGCCTGCTGGTGCCCTTCGTGCAGTGGTTCGTGGGCCTGTCCCCTACC
GTGTGGCTGTCCGCAATCTGGATGATGTGGTACTGGGGCCCATCCCTCTACAGCATCGTG
AGCCCATTCATCCCACTGCTGCCTATCTTCTTCTGCTTGTGGGTGTACATC
SEQ ID NO: 28:
hli-HBs plasmid sequence (UTR4)
ACGCGTGCCTAAATTAATACGACTCACTATAAGGAGAAGCTGTCTATCGGGCTCCAGCGG
TCATGCACAGAAGACGCTCCAGGAGCTGCCGAGAGGATCAGAAACCTGTGATGGACGACC
AGCGCGACCTGATCAGCAACAATGAGCAGTTGCCCATGCTGGGACGCCGCCCCGGCGCAC
CGGAGAGCAAGTGCTCCCGCGGCGCCTTATACACCGGATTCAGCATCCTCGTTACCCTGT
TACTGGCCGGGCAGGCCACCACGGCCTACTTCCTCTACCAACAACAGGGCAGGTTAGACA
AGCTGACCGTGACCTCACAGAATCTCCAGTTAGAGAACCTGAGAATGAAGCTGCCAAAGC
CACCTAAGCCTGTGAGCAAGATGCGGATGGCCACCCCCCTGCTGATGCAGGCCCTGCCTA
TGGGCGCCCTGCCACAGGGCCCCATGCAGAATGCCACCAAGTACGGCAATATGACCGAGG
ACCACGTGATGCACCTGCTGCAGAATGCCGACCCTCTGAAGGTGTACCCTCCTCTGAAGG
GCTCCTTCCCAGAGAATCTGCGGCACCTGAAGAACACCATGGAGACCATCGACTGGAAGG
TGTTCGAGAGCTGGATGCACCACTGGCTGCTGTTCGAGATGTCCAGGCACTCTCTGGAGC
AGAAGCCCACCGACGCCCCTCCAAAGGAGAGCCTGGAGCTGGAGGACCCCAGCAGCGGCC
TGGGCGTGACCAAGCAGGACCTGGGCCCAGTGCCAATGGAGAACATCACCAGCGGCTTCC
TGGGCCCCCTGCTGGTGCTGCAGGCCGGCTTCTTCCTGCTGACCAGGATCCTGACCATCC
CTCAGAGCCTGGACAGCTGGTGGACCAGCCTGAATTTCCTGGGCGGCAGCCCAGTGTGCC
TGGGCCAGAACAGCCAGTCCCCAACCAGCAACCACAGCCCTACCAGCTGCCCTCCCATCT
GCCCAGGCTACAGGTGGATGTGCCTGAGGCGGTTCATCATCTTCCTGTTCATCCTGCTGC
TGTGCCTGATCTTCCTGCTGGTGCTGCTGGACTACCAGGGCATGCTGCCAGTGTGCCCCC
TGATCCCCGGCAGCACCACCACCAACACCGGCCCCTGCAAGACCTGCACCACCCCCGCCC
AGGGCAACAGCATGTTCCCCTCGTGCTGCTGCACCAAGCCCACCGACGGCAATTGCACCT
GCATCCCCATCCCCAGCAGCTGGGCCTTCGCCAAGTACCTGTGGGAGTGGGCCAGCGTGC
GGTTCAGCTGGCTCAGCCTGCTGGTGCCCTTCGTGCAGTGGTTCGTGGGCCTGTCCCCTA
CCGTGTGGCTGTCCGCAATCTGGATGATGTGGTACTGGGGCCCATCCCTCTACAGCATCG
TGAGCCCATTCATCCCACTGCTGCCTATCTTCTTCTGCTTGTGGGTGTACATCTGATAAG
CCGCCGCTCCAGCTTTGCACGTTTCGATCCCAAAGGCCCTTTTTAGGGCCGACCATTCAT
TGCAAAAAAAAAAAAAAAAAAAAAAAAAAAAAAGCATATGACTAAAAAAAAAAAAAAAAA
AAAAAAAAAAAAAAAAAAAAAGAAGAGCGTTTAAACACGTGATATCTGGCCTCATGGGCC
TTCCTTTCACTGCCCGCTTTCCAGTCGGGAAACCTGTCGTGCCAGCTGCATTAACATGGT
CATAGCTGTTTCCTTGCGTATTGGGCGCTCTCCGCTTCCTCGCTCACTGACTCGCTGCGC
TCGGTCGTTCGGGTAAAGCCTGGGGTGCCTAATGAGCAAAAGGCCAGCAAAAGGCCAGGA
ACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTC
AGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGAACAGTATTTG
GTATCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGCGCA
GAAAAAAAGGATCTCAAGAAGATCCTTTGATCTTTTCTACGGGGTCTGACGCTCAGTGGA
ACGAAAACTCACGTTAAGGGATTTTGGTCATGAATACACGGTGCCTGACTGCGTTAGCAA
TTTAACTGTGATAAACTACCGCATTAAAGCTTATCGATGATAAGCTGTCAAACATGAGAA
TTCTTAGAAAAACTCATCGAGCATCAAATGAAACTGCAATTTATTCATATCAGGATTATC
AATACCATATTTTTGAAAAAGCCGTTTCTGTAATGAAGGAGAAAACTCACCGAGGCAGTT
CCATAGGATGGCAAGATCCTGGTATCGGTCTGCGATTCCGACTCGTCCAACATCAATACA
ACCTATTAATTTCCCCTCGTCAAAAATAAGGTTATCAAGTGAGAAATCACCATGAGTGAC
GACTGAATCCGGTGAGAATGGCAAAAGCTTATGCATTTCTTTCCAGACTTGTTCAACAGG
CCAGCCATTACGCTCGTCATCAAAATCACTCGCATCAACCAAACCGTTATTCATTCGTGA
TTGCGCCTGAGCGAGACGAAATACGCGATCGCTGTTAAAAGGACAATTACAAACAGGAAT
CGAATGCAACCGGCGCAGGAACACTGCCAGCGCATCAACAATATTTTCACCTGAATCAGG
ATATTCTTCTAATACCTGGAATGCTGTTTTCCCGGGGATCGCAGTGGTGAGTAACCATGC
ATCATCAGGAGTACGGATAAAATGCTTGATGGTCGGAAGAGGCATAAATTCCGTCAGCCA
GTTTAGTCTGACCATCTCATCTGTAACATCATTGGCAACGCTACCTTTGCCATGTTTCAG
AAACAACTCTGGCGCATCGGGCTTCCCATACAATCGATAGATTGTCGCACCTGATTGCCC
GACATTATCGCGAGCCCATTTATACCCATATAAATCAGCATCCATGTTGGAATTTAATCG
CGGCCTCGAGCAAGACGTTTCCCGTTGAATATGGCTCATAACACCCCTTGTATTACTGTT
TATGTAAGCAGACAGTTTTATTGTTCATGAGCGGATACATATTTGAATGTATTTAGAAAA
ATAAACAAATAGGGGTTCCGCGCACATTTCCCCGAAAAGTGCCACCTAAATTGTAAGCGT
TAATATTTTGTTAAAATTCGCGTTAAATTTTTGTTAAATCAGCTCATTTTTTAACCAATA
GGCCGAAATCGGCAAAATCCCTTATAAATCAAAAGAATAGACCGAGATAGGGTTGAGTGG
CCGCTACAGGGCGCTCCCATTCGCCATTCAGGCTGCGCAACTGTTGGGAAGGGCGTTTCG
GTGCGGGCCTCTTCGCTATTACGCCAGCTGGCGAAAGGGGGATGTGCTGCAAGGCGATTA
AGTTGGGTAACGCCAGGGTTTTCCCAGTCAC
SEQ ID NO: 29:
IRES nucleotide sequence
TGATTAAAACAGCTGTGGGTTGTTCCCACCCACAGGGCCCACTGGGCGCTAGCACTCTGA
TTTTACGAAATCCTTGTGCGCCTGTTTTATATCCCTTCCCTAATTCGAAACGTAGAAGCA
ATGCGCACCACTGATCAATAGTAGGCGTAACGCGCCAGTTACGTCATGATCAAGCATATC
TGTTCCCCCGGACTGAGTATCAATAGACTGCTTACGCGGTTGAAGGAGAAAACGTTCGTT
ATCCGGCTAACTACTTCGAGAAGCCCAGTAACACCATGGAAGCTGCAGGGTGTTTCGCTC
AGCACTTCCCCCGTGTAGATCAGGTCGATGAGCCACTGCAATCCCCACAGGTGACTGTGG
CAGTGGCTGCGTTGGCGGCCTGCCTATGGGGAGACCCATAGGACGCTCTAATGTGGACAT
GGTGCGAAGAGTCTATTGAGCTAGTTAGTAGTCCTCCGGCCCCTGAATGCGGCTAATCCT
AACTGCGGAGCACATGCCTTCAACCCAGAGGGTAGTGTGTCGTAACGGGCAACTCTGCAG
CGGAACCGACTACTTTGGGTGTCCGTGTTTCTTTTTTATTCTTATATTGGCTGCTTATGG
TGACAATTACAGAATTGTTACCATATAGCTATTGGATTGGCCATCCGGTGTGTAATAGAG
CTGTTATATACCTATTTGTTGGCTTTGTACCACTAACTTTAAAATCTATAACTACCCTCA
ACTTTATATTAACCCTCAATACAGTTGACC
SEQ ID NO: 30:
Human codon optimized (CodeRNA2) nucleic acid sequence encoding
the hli_HBc mRNA transgene
ATGCACAGAAGAAGAAGCAGATCATGCAGGGAGGATCAGAAACCAGTGATGGACGACCAG
AGGGACCTGATCAGCAACAATGAGCAACTGCCAATGCTGGGCCGAAGACCCGGCGCCCCC
GAGTCAAAGTGCAGCCGAGGCGCCTTATACACCGGCTTCAGCATCCTGGTTACGCTGCTC
CTGGCAGGCCAGGCCACCACCGCATATTTCCTGTACCAACAGCAAGGCAGACTGGACAAG
CTAACCGTGACCTCACAGAACCTACAGCTGGAAAATCTGAGGATGAAGCTCCCAAAGCCC
CCTAAGCCCGTGAGCAAGATGAGAATGGCCACACCCCTGCTGATGCAGGCCCTCCCCATG
GGAGCCCTGCCACAGGGACCCATGCAGAATGCCACCAAGTACGGCAACATGACCGAGGAC
CACGTGATGCACCTGCTGCAGAACGCCGACCCCCTGAAGGTGTACCCCCCCCTGAAGGGC
AGCTTCCCCGAGAACCTGAGACACCTGAAGAACACCATGGAGACCATCGACTGGAAGGTG
TTCGAGAGCTGGATGCACCACTGGCTGCTGTTCGAGATGAGCAGACACAGCCTGGAGCAG
AAGCCCACCGACGCCCCCCCCAAGGAGTCCCTGGAGCTGGAGGACCCCAGCAGCGGCCTG
GGCGTGACCAAGCAGGACCTGGGCCCCGTGCCCATGGACATCGACCCCTACAAGGAGTTC
GGCGCCACAGTGGAGCTGCTGAGCTTCCTGCCCAGCGACTTCTTCCCCAGCGTGAGAGAC
CTGCTGGACACCGCCTCCGCCCTGTACAGAGAGGCCCTGGAGAGCCCCGAGCACTGCAGC
CCCCACCACACCGCCCTGAGACAGGCCATCCTGTGCTGGGGGGAGCTGATGACCCTGGCC
ACCTGGGGGGCAACAACCTGGAGGACCCCGCCAGCAGAGACCTGGTGGTGAACTACGTGA
ACACCAACATGGGCCTGAAGATCAGGCAGCTGCTGTGGTTCCACATCAGCTGCTTGACCT
TCGGCAGAGAGACCGTGCTGGAGTACCTGGTGAGCTTCGGCGTGTGGATCCGCACCCCTC
CCGCATACAGACCCCCCAACGCCCCCATCCTGAGCACCCTGCCCGAGACCACCGTGGTGA
GACGCAGAGACAGAGGCCGGAGCCCCCGCAGAAGAACCCCCAGCCCCAGGAGAAGACGGA
GCCAGAGCCCCAGAAGACGGCGCAGCCAGAGCAGAGAGAGCCAGTGC
SEQ ID NO: 31:
Human codon optimized (CodeRNA2) nucleic acid sequence encoding
the HBs mRNA transgene
ATGGAGAATATCACCAGCGGCTTCCTCGGCCCCCTCTTAGTGCTGCAGGCCGGCTTCTTC
CTCCTGACACGGATCCTGACCATCCCGCAGTCCCTGGACTCATGGTGGACCTCCCTGAAC
TTCCTGGGCGGCTCCCCCGTGTGCCTGGGCCAGAACTCCCAGAGCCCCACCAGCAACCAC
TCCCCCACCAGCTGCCCCCCCATCTGCCCCGGCTACAGGTGGATGTGCCTGCGGCGGTTC
ATCATCTTCCTGTTCATCCTGCTGCTGTGCCTGATCTTCCTGCTGGTGCTGCTGGACTAC
CAGGGCATGCTGCCCGTGTGCCCCCTGATCCCCGGCTCCACCACCACCAACACCGGCCCC
TGCAAGACCTGCACCACCCCCGCCCAGGGGAACAGCATGTTCCCCTCCTGCTGCTGCACC
AAGCCCACCGACGGCAACTGCACCTGCATCCCCATCCCCTCCAGCTGGGCCTTCGCCAAG
TACCTGTGGGAGTGGGCCTCCGTGCGGTTCAGCTGGCTGAGCCTGCTGGTGCCCTTCGTG
CAGTGGTTCGTGGGCCTGTCCCCCACCGTGTGGCTGTCCGCCATCTGGATGATGTGGTAC
TGGGGCCCCAGCCTGTACAGCATCGTGAGCCCCTTCATCCCCCTGCTGCCCATCTTCTTC
TGCCTGTGGGTGTACATC
SEQ ID NO: 32:
Human codon optimized (CodeRNA2) nucleic acid sequence encoding
the hli_HBs mRNA transgene
ATGCACAGAAGACGCTCCAGGAGCTGCCGAGAGGATCAGAAACCTGTGATGGACGACCAG
CGCGACCTGATCAGCAACAATGAGCAGTTGCCCATGCTGGGACGCCGCCCCGGCGCACCG
GAGAGCAAGTGCTCCCGCGGCGCCTTATACACCGGATTCAGCATCCTCGTTACCCTGTTA
CTGGCCGGGCAGGCCACCACGGCCTACTTCCTCTACCAACAACAGGGCAGGTTAGACAAG
CTGACCGTGACCTCACAGAATCTCCAGTTAGAGAACCTGAGAATGAAGCTGCCAAAGCCA
CCTAAGCCTGTGAGCAAGATGCGGATGGCCACCCCCCTGCTGATGCAGGCCCTGCCTATG
GGCGCCCTGCCACAGGGCCCCATGCAGAATGCCACCAAGTACGGCAATATGACCGAGGAC
CACGTGATGCACCTGCTGCAGAATGCCGACCCTCTGAAGGTGTACCCTCCTCTGAAGGGC
TCCTTCCCAGAGAATCTGCGGCACCTGAAGAACACCATGGAGACCATCGACTGGAAGGTG
TTCGAGAGCTGGATGCACCACTGGCTGCTGTTCGAGATGTCCAGGCACTCTCTGGAGCAG
AAGCCCACCGACGCCCCTCCAAAGGAGAGCCTGGAGCTGGAGGACCCCAGCAGCGGCCTG
GGCGTGACCAAGCAGGACCTGGGCCCAGTGCCAATGGAGAACATCACCAGCGGCTTCCTG
GGCCCCCTGCTGGTGCTGCAGGCCGGCTTCTTCCTGCTGACCAGGATCCTGACCATCCCT
CAGAGCCTGGACAGCTGGTGGACCAGCCTGAATTTCCTGGGCGGCAGCCCAGTGTGCCTG
GGCCAGAACAGCCAGTCCCCAACCAGCAACCACAGCCCTACCAGCTGCCCTCCCATCTGC
CCAGGCTACAGGTGGATGTGCCTGAGGCGGTTCATCATCTTCCTGTTCATCCTGCTGCTG
TGCCTGATCTTCCTGCTGGTGCTGCTGGACTACCAGGGCATGCTGCCAGTGTGCCCCCTG
ATCCCCGGCAGCACCACCACCAACACCGGCCCCTGCAAGACCTGCACCACCCCCGCCCAG
GGCAACAGCATGTTCCCCTCGTGCTGCTGCACCAAGCCCACCGACGGCAATTGCACCTGC
ATCCCCATCCCCAGCAGCTGGGCCTTCGCCAAGTACCTGTGGGAGTGGGCCAGCGTGCGG
TTCAGCTGGCTCAGCCTGCTGGTGCCCTTCGTGCAGTGGTTCGTGGGCCTGTCCCCTACC
GTGTGGCTGTCCGCAATCTGGATGATGTGGTACTGGGGCCCATCCCTCTACAGCATCGTG
AGCCCATTCATCCCACTGCTGCCTATCTTCTTCTGCTTGTGGGTGTACATC

1. A composition for treating chronic hepatitis B infection comprising a mRNA encoding at least a hepatitis B virus core antigen (HBc), wherein the mRNA is encapsulated in a lipid nanoparticle (LNP).

2. The composition of claim 1, wherein the hepatitis B virus core antigen (HBc) comprises an amino acid sequence having at least 90%, 95%, 98% or 99% identity to the amino acid sequence set forth in SEQ ID NO:11.

3. The composition of claim 1, wherein the hepatitis B virus core antigen (HBc) is fused to human invariant chain (hli).

4. The composition of claim 1, wherein the composition further comprises an mRNA encoding a hepatitis B small surface protein (HBs).

5. The composition of claim 4, wherein the hepatitis B small surface protein (HBs) comprises an amino acid sequence having at least 90%, 95%, 98% or 99% identity to the amino acid sequence set forth in SEQ ID NO:1.

6. The composition of claim 4, wherein the hepatitis B small surface antigen (HBs) is fused to human invariant chain (hli).

7. The composition of claim 4, wherein the composition comprises more mRNA encoding HBc (HBc-mRNA) than mRNA encoding HBs (HBs-mRNA) by weight.

8. The composition of claim 4, wherein the mRNA encoding HBc and the mRNA encoding HBs mRNA are respectively present at a ratio of 1.5:1 by weight.

9. A composition for treating chronic hepatitis B infection comprising an mRNA encoding a hepatitis B small surface protein (HBs), wherein the mRNA is encapsulated in a lipid nanoparticle (LNP).

10. The composition of claim 9, wherein the HBs comprises an amino acid sequence having at least 90%, 95%, 98% or 99% identity to the amino acid sequence set forth in SEQ ID NO:1.

11. The composition of claim 9, wherein the HBs is fused to human invariant chain (hli).

12. The composition of claim 3, wherein the human invariant chain (hli) comprises an amino acid sequence having at least 90%, 95%, 98% or 99% identity to the amino acid sequence set forth in SEQ ID NO: 7 or SEQ ID NO:12.

13. The composition of claim 12, wherein the human invariant chain (hli) comprises an amino acid sequence having at least 90%, 95%, 98% or 99% identity to the amino acid sequence set forth in SEQ ID NO:12.

14-19. (canceled)

20. A method of treating chronic hepatitis B infection comprising administering to a human a prime-boost regimen, wherein an mRNA encoding at least one hepatitis B virus antigen is administered as a priming dose, and one or more recombinant hepatitis B polypeptide(s) is administered as a booster dose.

21. The method of claim 20, wherein the mRNA encodes at least one hepatitis B virus antigen selected from the group consisting of hepatitis B core antigen (HBc), or hepatitis B small surface protein (HBs).

22. The method of claim 20, wherein the hepatitis B virus antigen is fused to hli.

23. The method of claim 20, wherein the recombinant hepatitis B polypeptides comprise a recombinant hepatitis B core protein (HBc) and a recombinant hepatitis B small surface protein (HBs).

24. The method of claim 20, wherein the recombinant hepatitis B polypeptide(s) is administered with an adjuvant.

25. The method of claim 24, wherein the adjuvant is AS01.
